(19) United States
(12) Patent Application Publication
(10) Pub. No.: US 2020/0038392 A1
(43) Pub. Date: Feb. 6, 2020

(54) TOPICAL COMPOSITIONS AND METHODS OF PREPARATION AND USE

(71) Applicant: Aldeyra Therapeutics, Inc., Lexington, MA (US)

(72) Inventors: Todd Brady, Lexington, MA (US); David Clark, Lexington, MA (US); Susan Macdonald, Danvers, MA (US); Stephen Gitu Machatha, Wilmington, MA (US); Cameron Robert Stevenson, Guildford (GB); Charles Rodney Greenaway Evans, Worthing (GB)

(21) Appl. No.: 16/530,705

(22) Filed: Aug. 2, 2019

Related U.S. Application Data
(60) Provisional application No. 62/714,277, filed on Aug. 3, 2018.

(51) Int. Cl. A61K 31/47; A61K 31/4184; A61P 17/00; A61K 9/00; A61K 9/06

(52) U.S. Cl. CPC A61K 31/47; A61K 31/4184; A61K 9/06; A61K 9/0014; A61P 17/00

(57) ABSTRACT

The present invention provides topical, e.g., dermal, pharmaceutical compositions comprising an active agent. The present invention further provides methods of preparing such compositions and methods of treating a disease, disorder, or condition, such as a skin disease, using the same.

### TECHNICAL FIELD

The present subject matter relates to improved topical, e.g. dermal, pharmaceutical compositions comprising an active agent, and methods of making and using same to treat, ameliorate, or prevent a disease or condition.

### CROSS-REFERENCE TO RELATED APPLICATIONS

This application claims the benefit of U.S. Provisional Patent Application No. 62/714,277, filed on Aug. 3, 2018, the entire contents of which are hereby incorporated by reference.

### BACKGROUND OF THE INVENTION

Topical compositions may be used to deliver an active agent for the treatment of various conditions and diseases. Formulating topical compositions presents several challenges. For example, it may be difficult to formulate topical compositions that will cause less irritation upon application of the same as compared to other topical compositions comprising the same active agent or active agents. In addition, it is often challenging to prepare storage stable topical compositions that do not phase separate upon storage, do not discolor, and which fully solubilize the active agent(s), all while delivering the active agent(s) to the skin and/or through the skin (i.e., transdermally) or to other topical areas. Patient compliance concerns also arise when a topical formulation irritates the skin or has an unpleasant texture, consistency, or sensation when administered to the skin or other exposed body surface. For example, a topical composition should be aesthetically pleasing in terms of texture, color, and consistency (viscosity). Balancing these concerns is a significant and unmet need.

Many skin and other conditions are known for which improved topical formulations would offer much-needed treatment options for patients. As one example, no satisfactory treatment options exist, topical or otherwise, for ichthyosis associated with Sjögren-Larsson Syndrome (SLS). SLS (ICD Q87.1) is a rare, chronically-debilitating, autosomal-recessive disorder characterized by generalized ichthyosis, cognitive deficit in a majority of patients, and spastic diplegia or tetraplegia. SLS is caused by mutations in the ALDH3A2 gene that encodes fatty aldehyde dehydrogenase (FALDH), an enzyme that catalyzes the oxidation of aliphatic aldehydes to fatty acids.

In most patients, ichthyosis (dry, thickened, scaly, erythematous skin that is pruritic and, due to frequent excoriation, friable) is moderate or severe, particularly prominent in flexure areas, and involves much of the body surface except the face and glabrous skin. The dermal symptoms are generally recalcitrant to therapy, and are associated with significant daily physical and emotional burden and social stigma for patients and caregivers.

In SLS, fatty aldehydes disrupt dermal function, particularly the epidermal fatty moisture barrier, resulting in pathologic water loss, cutaneous desiccation, and compensatory keratinocytic hypertrophy. In epidermal cells, FALDH deficiency results in impaired oxidation of long-chain fatty aldehydes to fatty acids. The consequent accumulation of aldehydes disrupts the normal function and secretion of lamellar bodies and leads to intercellular lipid deposits in the stratum corneum and a defective water barrier in the skin layer, resulting in the cutaneous symptoms of SLS.

Accordingly, there remains a need to develop more effective topical gels, creams, and pastes for SLS and other conditions, such as those described below.

### BRIEF DESCRIPTION OF THE FIGURES

FIG. 1 depicts the comparison formulation under Leica DME light microscope with polarized and non-polarized light with a magnification of 400×. Although the formulation nominally contains 1.0% ADX-102, the compound is not fully soluble in the formulation. As shown in the images, crystals of the compound have formed, indicating that considerably less than 1.0% w/w of the compound remains dissolved in the formulation.

FIG. 2 depicts a design of experiment (DOE) matrix modeled using the ADX-102 saturated solubility data in solvent systems containing water, Transcutol HP, super refined PEG 400, propylene glycol and ethanol. The matrix was designed using JMP software. Red area indicates the composition of solvent system where solubility between 7.8-9% ADX-102 is not achieved. White area indicates the composition of solvent system where 7.8-9% ADX-102 is achieved.

FIG. 3 depicts the mean cumulative amount of ADX-102 in the receiver fluid following the application of the ADX-102 1% w/w formulation to cellulose acetate over 48 h. The receiver fluids employed for investigation were receiver fluid system 1 (ethanol and water (50:50 v/v)—blue line) and receiver fluid system 4 (20% PEG 400 in ethanol and water (50:50 v/v)—red line). Data are shown as mean (2 replicates) and error bars denote the range of values.

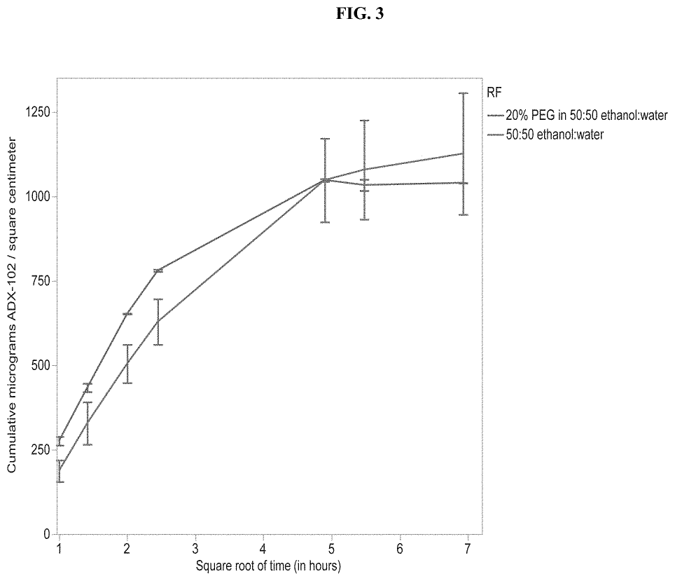

FIG. 4 depicts the mean cumulative amount of ADX-102 in the receiver fluid with respect to the square root of time following the application of the ADX-102 1% w/w formulation to cellulose acetate over (a) the 48 h sample period and (b) the initial 6 h sampling period. The receiver fluids employed for investigation were receiver fluid system 1 (ethanol and water (50:50 v/v)) and receiver fluid system 4 (20% PEG 400 in ethanol and water (50:50 v/v)). Data are shown as mean (2 replicates) and error bars denote the range of values.

FIG. 5 depicts the mean cumulative amount of ADX-102 in the receiver fluid with respect to the square root of time following the application of the 10 formulations to cellulose acetate over the 6 h sample period. Data are shown as mean (6 replicates) and error bars denote the standard deviation.

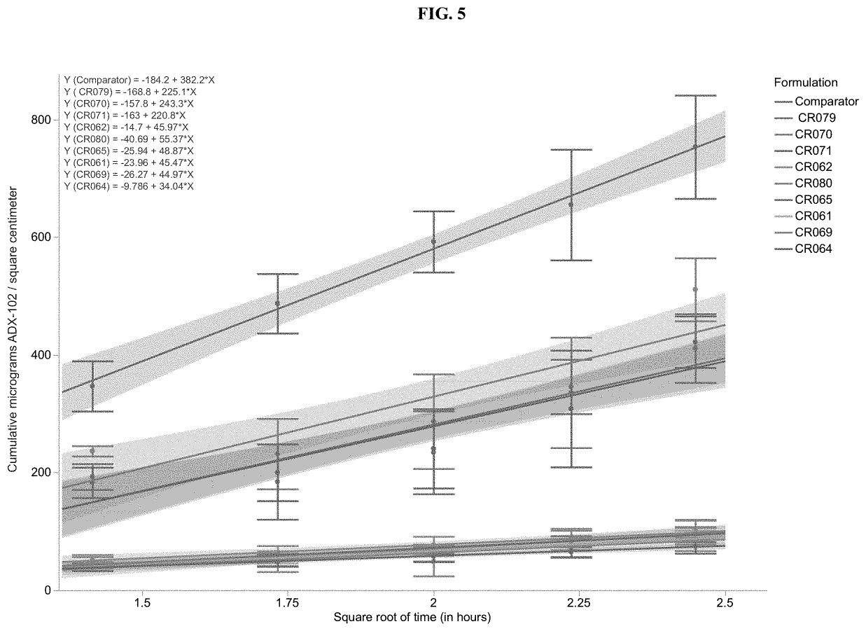

FIG. 6 depicts the cumulative amount of ADX-102 released per unit area (μg/cm²) through a cellulose acetate membrane from CR070 (1.2%, 1% and 0.8% w/w ADX-102) presented as √time. Data is presented as mean±SD n=6 replicates.

FIG. 7 depicts the mean cumulative amount of ADX-102 in the receiver fluid with respect to the square root of time following the application of 10 different dermal formulations to a human skin sample over a 24 h sample period.

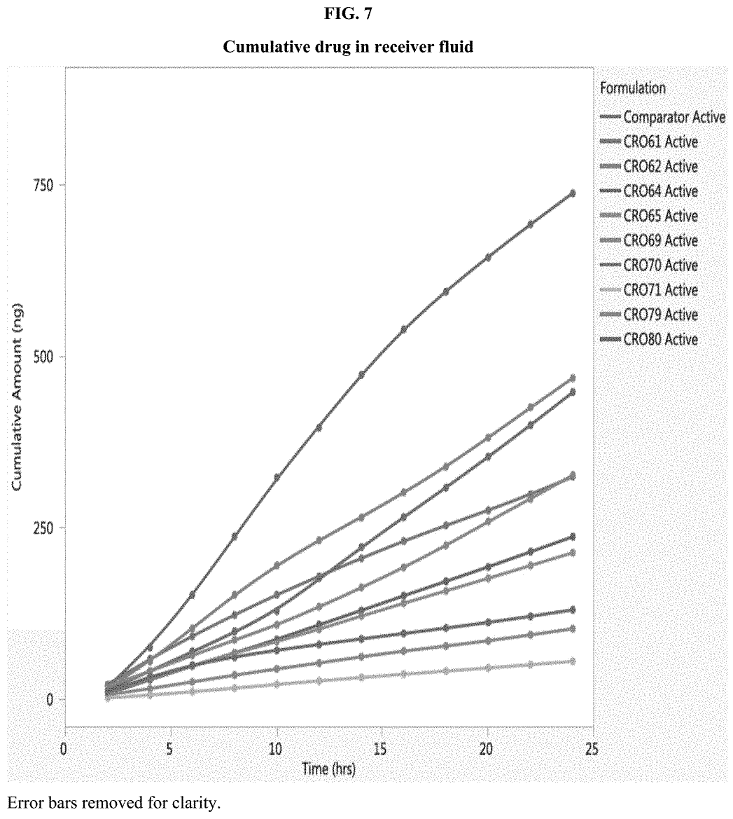

FIG. 8 depicts a one-way analysis of the flux of ADX-102 delivered by 10 different dermal formulations across a human skin membrane.

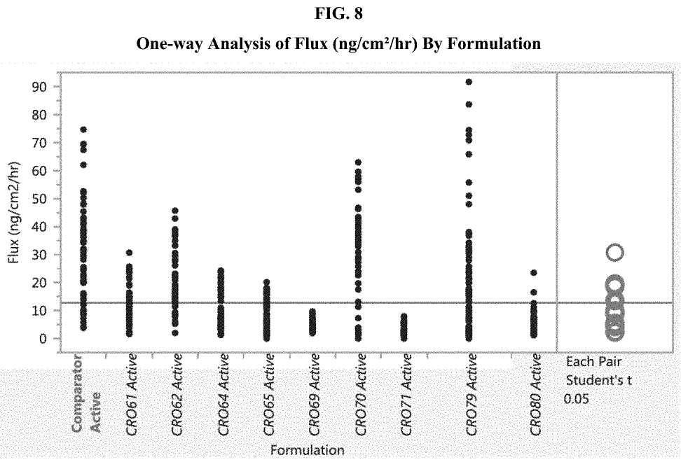

FIG. 9 depicts bar graphs of the total quantity of ADX-102 in dermis and epidermis delivered by 10 different dermal formulations across a human skin membrane. Each error bar is constructed using 1 standard error from the mean.

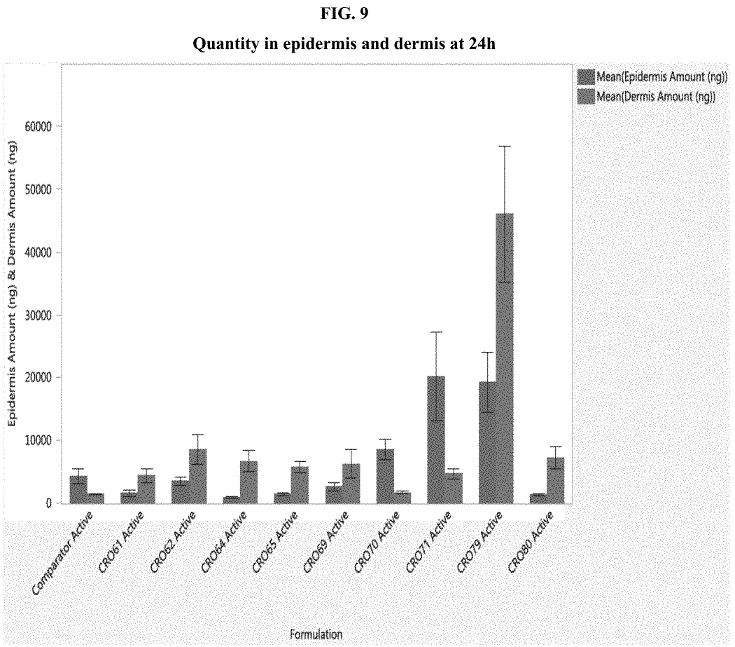

FIG. 10 depicts a one-way analysis of the total quantity of ADX-102 delivered to the epidermis of a human skin membrane by 10 different dermal formulations.

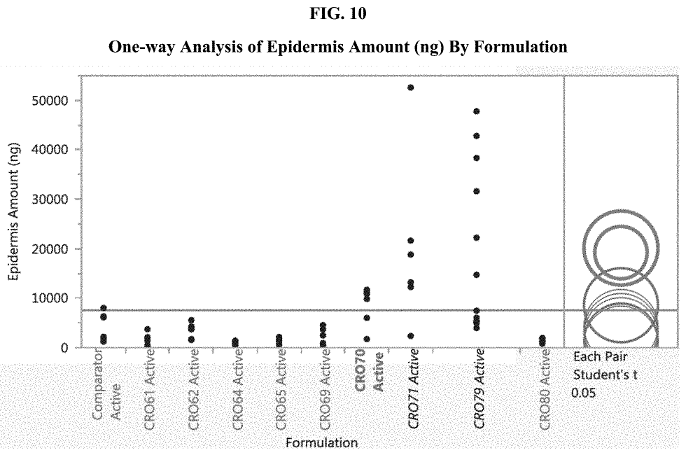

FIG. 11 depicts a one-way analysis of the total quantity of ADX-102 delivered to the dermis of a human skin membrane by 10 different dermal formulations.

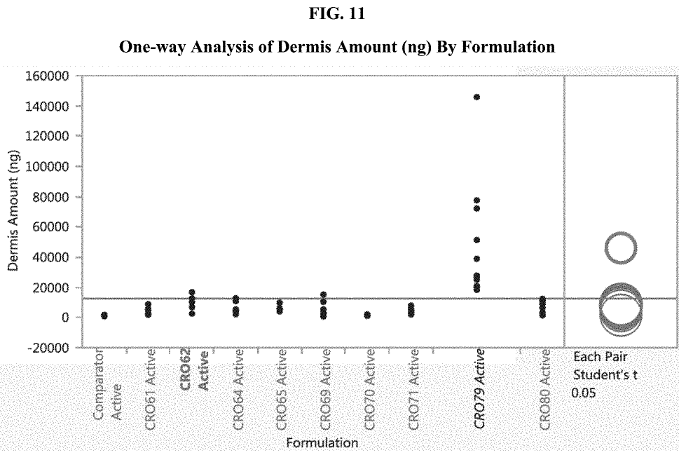

FIG. 12 depicts a graph showing the percentage of the total applied dose of ADX-102 delivered to the receiving fluid across a human skin sample.

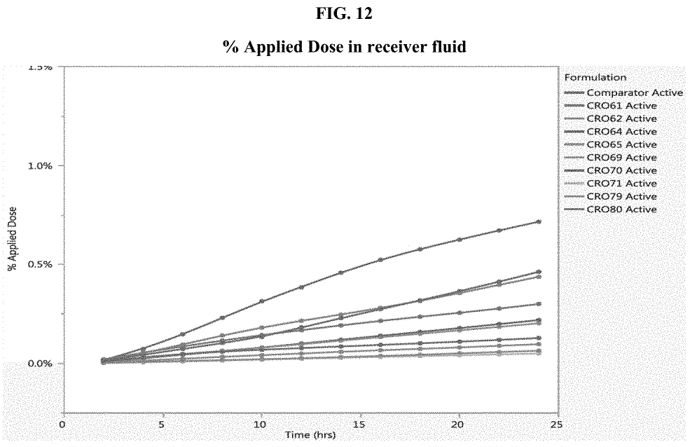

FIG. 13 depicts bar graphs of the percentage of the total applied dose of ADX-102 in the dermis and epidermis of a human skin sample.

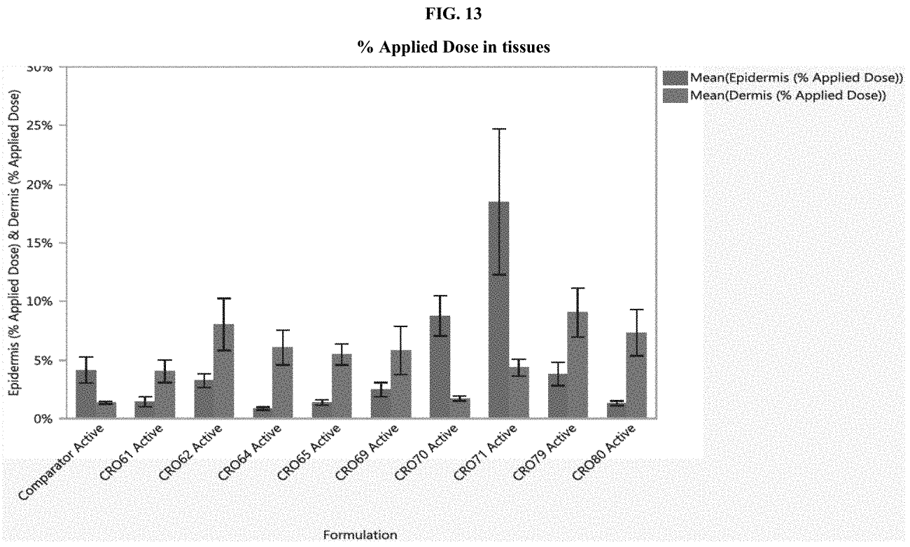

FIG. 14 depicts the total amount in ng of ADX-102 delivered by various formulations to the dermis, epidermis, and receiving fluid for a human skin sample.

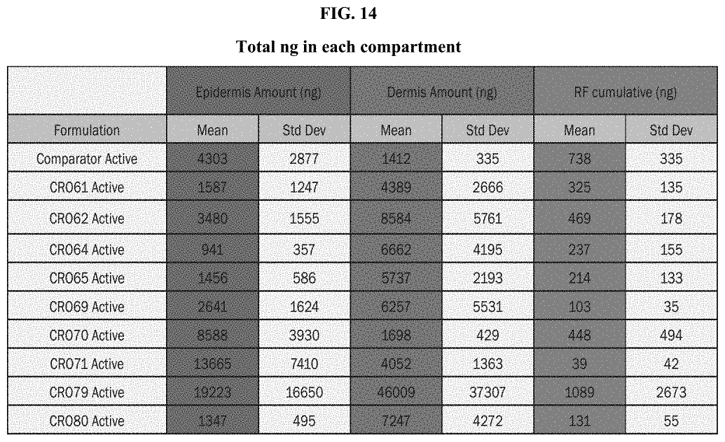

FIG. 15 depicts the cumulative amount in ng of ADX-102 delivered to receiving fluid for 10, 20, and 40 mg doses of ADX-102 in the CR070 formulation. Each error bar depicts one standard deviation from the mean.

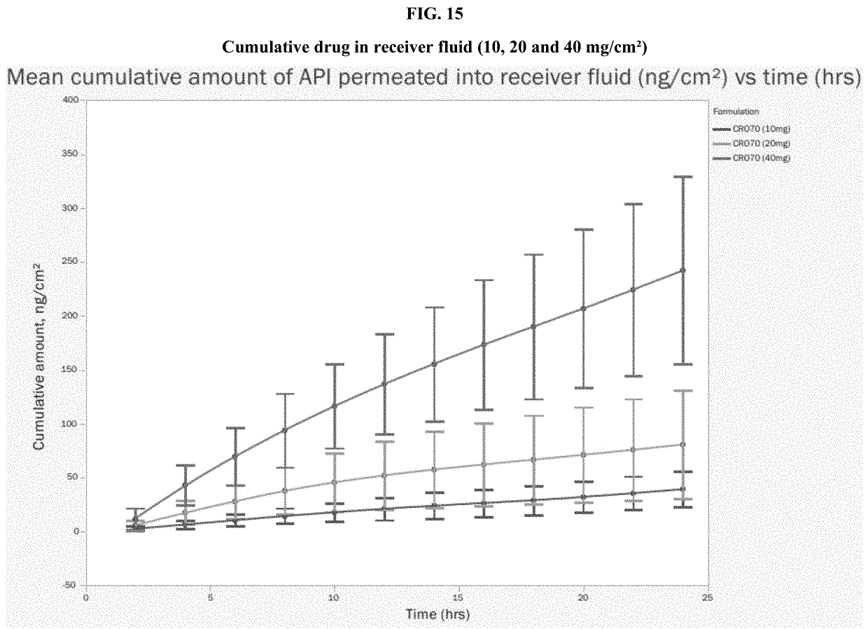

FIG. 16 depicts the total amount in ng of ADX-102 delivered by various formulations to the dermis and epidermis after 24 h for a human skin sample. Each error bar is constructed using 1 standard deviation from the mean.

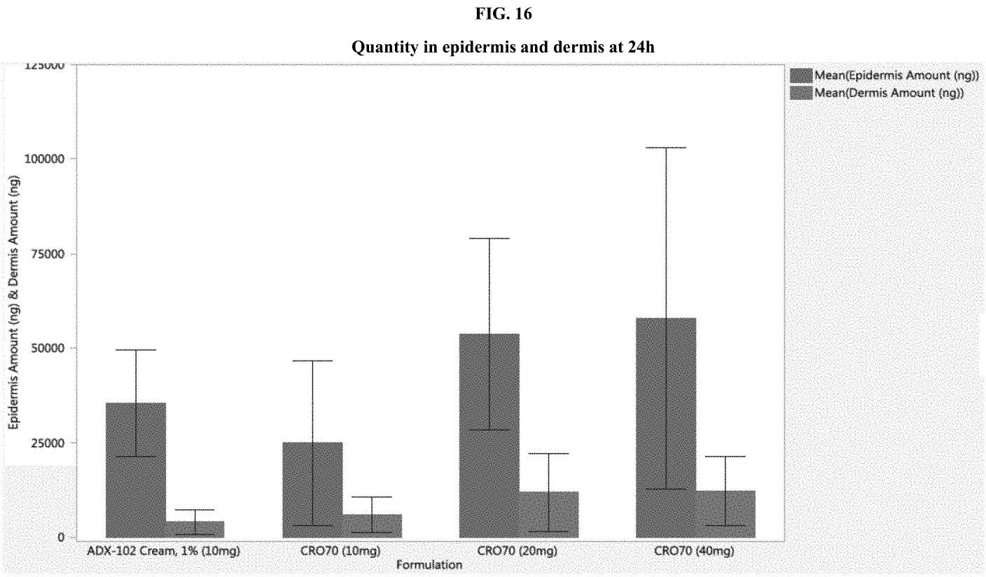

FIG. 17 depicts the total amount in ng of ADX-102 delivered by various formulations to the dermis, epidermis, and receiving fluid for a human skin sample.

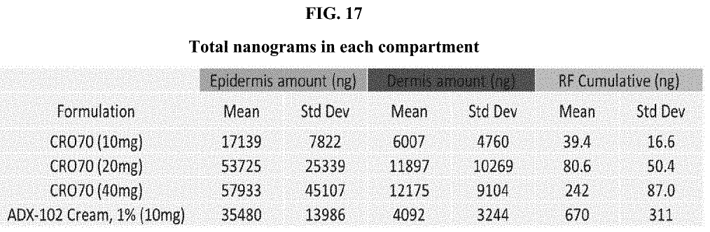

### DETAILED DESCRIPTION OF THE INVENTION

As described above, there exists and urgent need for improved topical, e.g. dermal, compositions that are significantly more effective than known pharmaceutical compositions. The present invention provides a topical, e.g. dermal, pharmaceutical composition such as a cream, paste, gel, or liquid with surprisingly improved solubility, stability, and/or delivery of an active agent. In some embodiments, a disclosed composition is effective for treating, inter alia, dermal conditions such as SLS.

Topical (e.g., Dermal) Compositions:

In some embodiments, the present invention provides a topical composition comprising or consisting essentially of:

(i) an active agent or pharmaceutically acceptable salt thereof;

(ii) a first solvent;

(iii) optionally a second solvent;

(iv) a penetration enhancer;

(v) optionally one or more of a preservative, solubilizing agent, thickening agent, stabilizing agent, surfactant, skin conditioner, humectant, or second skin penetration enhancer;

(vi) optionally an antioxidant; and

optionally one or more of: an emulsifying agent, a diluent, a pH adjuster, a chelating agent, a coloring agent, or a fragrance. Exemplary such topical compositions are described in further detail below and herein.

In some embodiments, the solvent is selected from benzyl alcohol, super refined PEG 400, propylene carbonate, castor oil, glycerol, IPM, IPP, Miglyol 810, Transcutol HP, propylene glycol, ethanol, or a water-alcohol mixture (e.g., 75:25 v/v WFI:ethanol and 75:25 v/v deionised water:ethanol), or a mixture thereof.

In some embodiments, the present invention provides a topical (e.g., dermal) composition, comprising about 0.1 to 7.5% of an active agent; about 12.0 to 48.0% of a first solvent; about 2.0 to 15.0% of a second solvent; about 1.0 to 29.0% of a penetration enhancer; optionally up to about 3.0% of a preservative; optionally up to about 20.0% of a thickening agent; about 1.0 to 15.0% of a surfactant; optionally up to about 10.0% of a stabilizing agent; and optionally up to about 1.0% of an antioxidant.

In some embodiments, the composition comprises up to about 40.0% water. In some embodiments, the composition comprises about 28.0 to 40.0% water.

In some embodiments, the first solvent comprises a polyethylene glycol, polyoxyethylene alkyl ether, polyoxyethylene sorbitan fatty acid ester, or polyoxyethylene stearate.

In some embodiments, the second solvent is present and comprises a glycol solvent (e.g., propylene glycol); and an alkyl alcohol solvent (e.g., ethanol).

In some embodiments, the penetration enhancer is selected from one or more of an dimethyl sulfoxide, a glycol, glyceryl monooleate, glycofurol, isopropyl myristate, isopropyl palmitate, lanolin, light mineral oil, linoleic acid, menthol, myristic acid, myristyl alcohol, oleic acid, oleyl alcohol, palmitic acid, a polyoxyethylene alkyl ether, a polyoxylglyceride, a pyrrolidone, sodium lauryl sulfate, thymol, tricaprylin, triolein, acetyltributyl citrate, ethylene vinyl acetate, a polymethacrylate, polyvinyl alcohol, sesame oil, sodium carboxymethyl guar, or stearyl alcohol.

In some embodiments, the surfactant is selected from emulsifying wax USP, glyceryl monooleate, a phospholipid, a polyoxyethylene alkyl ether, polyoxyethylene castor oil derivative, a polyoxyethylene sorbitan fatty acid ester, polyoxyethylene stearates, polyoxylglycerides (such as Caprylocaproyl Polyoxylglycerides, Lauroyl Polyoxylglycerides, Linoleoyl Polyoxylglycerides, Oleoyl Polyoxylglycerides, Stearoyl Polyoxylglycerides), polysorbate, a sorbitan ester, triethyl citrate, vitamin E polyethylene glycol succinate, miglyol, a Steareth (Brij) surfactant, a Cetomacrogol, a Myrj, a Span, a Tween, or a Laureth surfactant.

In some embodiments, the surfactant is selected from Steareth 21 (Brij s721), Steareth 2 (Brij S2), Cetomacrogol 1000, Myrj S40, Brij CS25 (Ceteareth 25), Steareth 20 (Brij S20), Span 20, Geleol mono and diglycerides, Span 60, or Tween 60, or a combination thereof.

In some embodiments, the surfactant is a combination of Steareth 2 and Mrij S40; Steareth 2 and Brij CS25; Steareth 2 and Cetomacrogol 1000; Steareth 2 and Steareth 20/Brij S20; Steareth 2 and Steareth 21; Span 20 and Geleol; Span 60 and Tween 60; Cetomacrogol 1000 and Geleol; Brij S20 and Brij S2; or Geleol, Span 20, and Steareth 20/Brij S20.

In some embodiments, the thickening agent is a high molecular weight polyethylene glycol such as PEG 1500 or PEG 4000.

In some embodiments, the preservative is selected from vitamin C, butylated hydroxytoluene (BHT), a sulfite, vitamin E, ascorbyl palmitate, or propyl gallate. In certain embodiments, the preservative is benzyl alcohol or phenoxyethanol.

In some embodiments, the antioxidant is a combination of BHT and butylated hydroxyanisole (BHA).

### Definitions

Herein we describe exemplary embodiments of the presently disclosed topical formulations. One of ordinary skill will appreciate that numerous variations of pharmaceutical excipients are possible, and that a particular excipient may be exchanged for another equivalent excipient while providing similar properties to the composition as a whole. Accordingly, such equivalent excipients are intended to be encompassed by the present invention. Equivalent excipients are described, for example, in R. C. Rowe et al., Handbook of Pharmaceutical Excipients, 6^{th} Ed., Pharmaceutical Press, 2009, which is hereby incorporated by reference.

As used herein, the term “preservative” refers to any known pharmaceutically acceptable preservative that functions by inhibiting bacteria, fungi, yeast, mold, other microbe, and/or by inhibiting oxidation. Suitable preservatives include but are not limited to antimicrobial agents and/or antioxidants. Suitable antimicrobial agents can include but are not limited to benzoates, benzyl alcohol, sodium benzoate, sorbates, propionates, and nitrites. Suitable antioxidants can include but are not limited to vitamin C, butylated hydroxytoluene (BHT), a sulphite, and vitamin E.

As used herein, the term “solvent” refers to any pharmaceutically acceptable medium which is a liquid (or is a liquid when mixed with another solvent or organic compound) at ambient temperature, in which one or more solutes can be dissolved, or one or more substances can be partially dissolved or suspended. In some embodiments, the medium is present in a provided composition in an amount of about 10 wt % or more. Numerous solvents are well known in the chemical and pharmaceutical arts and include those described herein.

The term “chelating agent” as used herein refers to any known pharmaceutically acceptable chelating agents. Suitable chelating agents can include but are not limited to any one or more of ethylenediaminetetraacetic acid (EDTA) and derivatives thereof, ethylene glycol-bis-(2-aminoethyl)-N,N,N′,N′-tetraacetic acid (EGTA) and derivatives thereof, cyclohexanediamine tetraacetic acid (CDTA) and derivatives thereof, hydroxyethylethylenediamine triacetic acid (HEDTA) and derivatives thereof, diethylenetriamine pentaacetic acid (DTPA) and derivatives thereof, dimercaptopropane sulfonic acid (DMPS) and derivatives thereof, dimercaptosuccinic acid (DMSA) and derivatives thereof, aminotrimethylene phosphonic acid (ATPA) and derivatives thereof, N,N-bis(carboxymethyl)glycine (NTA) and derivatives thereof, nitrilotriacetic acid and derivatives thereof, citric acid and derivatives thereof, niacinamide and derivatives thereof, sodium desoxycholate and derivatives thereof, polyphosphates; porphine; and any pharmaceutically acceptable salts thereof.

As used herein, the term “diluent” refers to water, saline, or a non-toxic buffered aqueous solution.

As used herein, the phrases an “effective amount” or a “therapeutically effective amount” of an active agent or ingredient, or pharmaceutically active agent or ingredient, refer to an amount of the pharmaceutically active agent sufficient enough to have a therapeutic effect upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors. For example, the presently described compositions can be topically applied in an amount sufficient to cover an affected area plus a margin of healthy skin or tissue surrounding the affected area, for example, a margin of about 0.5 inches, at a frequency, for example, of once, twice, or three times a day. An exemplary effective amount is a dose of a topical cream containing 1% w/w ADX-102 of approximately 1-80 mg/cm², at a frequency of about once per day.

As used herein, the term “pH adjuster” refers to any pharmaceutically acceptable composition, compound, or agent, suitable for adjusting the pH of the presently described topical pharmaceutical compositions without negatively affecting any property thereof. Suitable pH adjusters can include any pharmaceutically acceptable acid or base. Suitable pH adjusters can include but are not limited to hydrochloric acid, sulfuric acid, citric acid, acetic acid, formic acid, phosphoric acid, tartric acid, trolamine, sodium hydroxide and potassium hydroxide.

As used herein, the term “solubilizing agent” refers to any pharmaceutically acceptable liquid medium, surfactant, and/or emulsifier that is present in a provided composition, in some embodiments, in an amount of less than about 10 wt %. One of skill in the chemical and pharmaceutical arts will readily appreciate that certain of the above-described solvents may also be used in substantially lower amounts such that they are characterized herein as solubilizing agents rather than solvents. Accordingly, in some embodiments, a solubilizing agent is any one of the above-listed solvents present in a provided formulation in an amount less than 10 wt % of the formulation. For example, in some embodiments, a solubilizing agent is a dialkylene glycol monoalkyl ether, such as, e.g., diethylene glycol monoethyl ether, present in an amount of less than 10 wt %. In some embodiments, a solubilizing agent is an alcohol, for instance, ethanol, present in an amount less than 10 wt %. Exemplary other such solubilizing agents are described below and herein.

The phrase “substantially pure” as used herein refers to an individual compound form, which is substantially devoid of all other forms, as well as degradation products of a form, and any residual solvent, and is at least 85% pure on a % weight basis, unless otherwise specified. The compound form can have at least 90% purity on a % weight basis, at least 93% purity on a % weight basis, at least 95% purity on a % weight basis, or at least 97%, 98%, 99%, 99.5%, or 99.9% or greater purity on a % weight basis.

Unless otherwise specified, all percentages, for example a % value of a disclosed composition, refers to a percentage by weight (wt. %) of the composition, mixture, solution, or formulation.

### Solvents

As defined above, a topical composition of the present invention comprises one or more solvents as described above and defined herein. For instance, in some embodiments, a provided topical composition comprises only one solvent, such as a water-soluble or water-miscible organic solvent, e.g., a polyethylene glycol, polyoxyethylene alkyl ether, polyoxyethylene sorbitan fatty acid ester, or polyoxyethylene stearate. In other embodiments, a topical composition of the present invention comprises a mixture of more than one solvent. In some embodiments, the solvent mixture is a mixture of: a polyethylene glycol, polyoxyethylene alkyl ether, polyoxyethylene sorbitan fatty acid ester, or polyoxyethylene stearate; a glycol solvent (e.g., propylene glycol); and an alkyl alcohol solvent (e.g., ethanol). In some embodiments wherein a topical composition of the present invention comprises more than one solvent, the present invention refers to one of the more than one solvents as a “first solvent” and another of the more than one solvents as a “second solvent.” In such instances, by “first solvent” is meant any of the solvents described above and herein; by “second solvent” is meant any of the solvents described above and herein other than the “first solvent.” Likewise, also contemplated herein are additional solvents, e.g., a “third solvent,” a “fourth solvent,” etc., which solvents are also characterized in that each is a different solvent from the others. In some embodiments, a first or second solvent is, for example, a glycol solvent. In some embodiments, a first or second solvent is, for example, an alcohol solvent. Exemplary such one or more solvents, and combinations thereof, are contemplated by the present invention and described herein.

In some embodiments, a solvent is a non-volatile solvent such as a non-volatile organic solvent. In some embodiments, the solvent is a non-volatile, water-miscible organic solvent. In some embodiments, a solvent is a volatile solvent, such as a volatile organic solvent. In some embodiments, the first solvent is a non-volatile, water-miscible organic solvent and the second solvent is a volatile, water-miscible organic solvent. The solvents are generally selected so as to be non-toxic and non-irritating, or present in small enough amounts to be non-toxic and non-irritating.

In some embodiments, the first solvent is selected from a polyethylene glycol, polyoxyethylene alkyl ether, polyoxyethylene sorbitan fatty acid ester, or polyoxyethylene stearate. In some embodiments, the first solvent is selected from Transcutol, PEG 400, S.R. PEG 400, PEG 200, PEG 300, PEG 540, PEG 600, PEG 900, propylene carbonate, butyl stearate, castor oil, IPM, IPP, Miglyol 810, or a mixture thereof. In some embodiments, the first solvent is Transcutol, Transcutol HP, PEG 400 or SR PEG 400.

In some embodiments, the second solvent is a volatile alcohol. In some embodiments, the second solvent is an alkyl alcohol. In some embodiments, the second solvent is selected from methanol, ethanol, n-propanol, isopropanol, n-butanol, s-butanol, isobutanol, t-butanol, or n-pentanol. In some embodiments, the alcohol solvent is selected from one or more of methanol, ethanol, n-propyl alcohol, isopropyl alcohol, n-butyl alcohol, t-butyl alcohol, 2-butanol, iso-butanol, pentanol, hexanol, cyclohexanol, and hexadecan-1-ol. In some embodiments, the alcohol solvent is methanol, ethanol, n-propyl alcohol, or isopropyl alcohol. In certain embodiments, the alcohol solvent is ethanol. In some embodiments, a solvent is a mixture of one or more such alcohols.

As used herein and unless otherwise indicated, the term “ethanol” refers to 190 proof USP grade ethanol. In some embodiments, USP grade ethanol is ethanol containing NLT 92.3% and NMT 93.8%, by weight, corresponding to NLT 94.9% and NMT 96.0%, by volume, at 15.56%, of C₂H₅OH.

As used herein, a solvent is considered “volatile” if it has a high vapor pressure at room temperature. In some embodiments, a volatile solvent has a boiling point that is about 250° C. or less, about 120° C. or less, about 100° C. or less, or about 80° C. or less at standard atmospheric pressure. As used herein, a solvent is considered “non-volatile” if its boiling point is more than about 80° C., about 100° C. or more, about 120° C. or more, about 250° C. or less, at standard atmospheric pressure.

In some embodiments, a solvent is, e.g., a triacetin and/or diol and/or polyol solvent. Diol solvents can include, but are not limited to, glycol solvents. In certain embodiments, a solvent is an alkylene glycol solvent. For instance, in some embodiments, the alkylene glycol solvent is ethylene glycol, propylene glycol, butylene glycol, or the like. In certain embodiments, the glycol solvent is propylene glycol.

In some embodiments, a solvent is a glycol ether. For instance, in some embodiments, a solvent is a dialkylene glycol monoalkyl ether, such as, e.g., diethylene glycol monoethyl ether. Other such glycol ethers are known in the chemical and pharmaceutical arts and are contemplated by the present invention.

In some embodiments, a solvent is selected from phenoxyethanol, ethanol, isopropanol, SR PEG 400, Arlasolve DMI, diisopropyl adipate, IPM, Transcutol HP, benzyl alcohol, oleyl alcohol, castor oil, butyl stearate, isopropyl palmitate (IPP), miglyol 810, or a combination thereof.

In some embodiments, a solvent is selected from phenoxyethanol, ethanol, isopropanol, SR PEG 400, Arlasolve DMI, diisopropyl adipate, IPM, Transcutol HP, or benzyl alcohol.

In some embodiments, a solvent is selected from ethanol, PEG 400, SR PEG 400, Transcutol HP, or propylene glycol.

In some embodiments, a disclosed composition comprises the following solvents: Transcutol® HP: 0-20%; SR PEG 400: 0-60%; Ethanol: 0-20%; and Propylene glycol: 0-15%.

In some embodiments, a solvent is present in a topical composition in an amount greater than 10 wt % to about 50 wt %; from greater than 10 wt % to about 45 wt %; from greater than 10 wt % to about 35 wt %; from greater than 10 wt % to about 30 wt %; from greater than 10 wt % to about 25 wt %; from greater than 10 wt % to about 20 wt %; from greater than 10 wt % to about 15 wt %; from about 15 wt % to about 30 wt %; from about 15 wt % to about 25 wt %; from about 15 wt % to about 20 wt %; from about 20 wt % to about 45 wt %; from about 25 wt % to about 45 wt %; from about 30 wt % to about 40 wt %; from about 16 wt % to about 24 wt %; from about 17 wt % to about 23 wt %; from about 18 wt % to about 24 wt %; from about 18 wt % to about 23 wt %; from about 18 wt % to about 22 wt %; from about 18 wt % to about 21 wt %; from about 18 wt % to about 20 wt %; from about 18.5 wt % to about 19.5 wt %; from about 19 wt % to about 20 wt %; from about 19 wt % to about 21 wt %; from about 19 wt % to about 22 wt %; from about 19 wt % to about 23 wt %; from about 19 wt % to about 24 wt %; from about 19 wt % to about 25 wt %; about 11 wt %; about 12 wt %; about 13 wt %; about 14 wt %; about 15 wt %; about 16 wt %; about 17 wt %; about 18 wt %; about 19 wt %; about 20 wt %; about 21 wt %; about 22 wt %; about 23 wt %; about 24 wt %; about 25 wt %; about 26 wt %; about 27 wt %; about 28 wt %; about 29 wt %; about 30 wt %; about 31 wt %; about 32 wt %; about 33 wt %; about 34 wt %; about 35 wt %; about 36 wt %; about 37 wt %; about 38 wt %; about 39 wt %; about 40 wt %; about 41 wt %; about 42 wt %; about 43 wt %; about 44 wt %; or about 45 wt %.

In some embodiments, a solvent is present in a topical composition in an amount of from about 16 wt % to about 24 wt %. In some embodiments, a solvent is present in a topical composition in an amount of from about 17 wt % to about 23 wt %. In some embodiments, a solvent is present in a topical composition in an amount of from about 18 wt % to about 22 wt %. In some embodiments, a solvent is present in a topical composition in an amount of from about 18 wt % to about 21 wt %. In certain embodiments, a solvent is present in a topical composition in an amount of about 18 wt %. In certain embodiments, a solvent is present in a topical composition in an amount of about 19 wt %. In certain embodiments, a solvent is present in a topical composition in an amount of about 20 wt %.

In some embodiments, a solvent is present in a topical composition in an amount of from about 20 wt % to about 45 wt %. In some embodiments, a solvent is present in a topical composition in an amount of from about 25 wt % to about 45 wt %. In some embodiments, a solvent is present in a topical composition in an amount of from about 30 wt % to about 45 wt %. In some embodiments, a solvent is present in a topical composition in an amount of from about 35 wt % to about 45 wt %. In some embodiments, a solvent is present in a topical composition in an amount of about 35 wt %; about 36 wt %; about 37 wt %; about 38 wt %; about 39 wt %; about 40 wt %; about 41 wt %; about 42 wt %; about 43 wt %; about 44 wt %; or about 45 wt %. In certain embodiments, a solvent is present in a topical composition in an amount of about 41%.

In some embodiments, the solvent is selected from PEG 400, super refined (SR) PEG 400, propylene carbonate, castor oil, glycerol, IPM, IPP, Miglyol 810, Transcutol HP, propylene glycol, ethanol, or a water-alcohol mixture (e.g., 75:25 v/v WFI:ethanol and 75:25 v/v deionised water:ethanol), or a mixture thereof. In some embodiments, the solvent is selected from phenoxyethanol, ethanol, isopropanol, PEG 400, SR PEG 400, Arlasolve DMI, diisopropyl adipate, IPM, Transcutol HP, or benzyl alcohol.

In certain embodiments, a topical composition of the present invention comprises more than one solvent, wherein at least one of the more than one solvents is a glycol solvent.

In certain embodiments, a topical composition of the present invention comprises more than one solvent, wherein at least one of the more than one solvents is an alcohol solvent.

In certain embodiments, a topical composition of the present invention comprises more than one solvent, wherein at least one of the more than one solvents is an alcohol solvent and one of the more than one solvents is a glycol solvent. In certain embodiments, an alcohol solvent and a glycol solvent are each present in an amount of about 15 wt % to about 35 wt %. In certain embodiments, an alcohol solvent and a glycol solvent are each present in an amount of about 15 wt % to about 30 wt %. In certain embodiments, an alcohol solvent and a glycol solvent are each present in an amount of about 15 wt % to about 25 wt %. In certain embodiments, an alcohol solvent and a glycol solvent are each present in an amount of about 18 wt % to about 21 wt %. In some embodiments, the alcohol solvent is an alkyl alcohol (e.g., ethanol) present in an amount of about 15-25 wt % and the glycol solvent is an alkylene glycol (e.g., propylene glycol) present in an amount of about 15-25 wt %. In some embodiments, the alcohol solvent is ethanol and is present in an amount of about 18-20 wt % and the glycol solvent is propylene glycol and is present in an amount of about 18-20 wt %.

In some embodiments, no more than 60% water is present in the composition. In some embodiments, about 1% to about 60% water is present in the composition. In some embodiments, about 5% to about 55% water is present in the composition. In some embodiments, about 10% to about 50% water is present in the composition. In some embodiments, about 15% to about 45% water is present in the composition. In some embodiments, about 20% to about 40%, about 20% to about 30%, or about 30% to about 40% water is present in the composition. In some embodiments, about 20% to about 50%, about 25% to about 50%, about 30% to about 50%, about 33% to about 47%, about 35% to about 44%, about 37% to about 44%, about 40% to about 50%, about 42% to about 45%, about 45% to about 50%; or about 25%, about 28%, about 30%, about 35%, about 40%, or about 45% water is present in the composition.

In some embodiments, a presently disclosed topical composition comprises a mixture of solvents according to one of those mixtures provided in the Tables below:

TABLE A
Exemplary composition of solvent systems for disclosed active agents

| Excipient | Composition of solvent system (% w/w) | | | | | | |
|---|---|---|---|---|---|---|---|
| Water | 39 | 41 | 44 | 38 | 38 | 38 | 42 |
| Transcutol ® HP | 2 | 18 | 2 | 12 | 1 | 18 | 1 |
| SR PEG 400 | 59 | 31 | 44 | 40 | 51 | 24 | 37 |
| Ethanol | 0 | 0 | 0 | 10 | 10 | 10 | 10 |
| Propylene glycol | 0 | 10 | 10 | 0 | 0 | 10 | 10 |
| Total | 100 | 100 | 100 | 100 | 100 | 100 | 100 |
| Exemplary active | 4.4 | 4.5 | 2.5 | 8.1 | 6.8 | 7.8 | 3.8 |
| agent | | | | | | | |
| concentration | | | | | | | |

TABLE B
Exemplary composition of solvent systems for disclosed
active agents, with preservative, antioxidant and humectants

| | |
|---|---|
| | Composition of |
| Excipient | solvent systems (% w/w) |

| | | | | |
|---|---|---|---|---|
| H₂O | 38 | 38 | 38 | 38 |
| Transcutol ® HP | — | 1 | 1 | 1 |
| SR PEG 400 | 50.8 | 39.8 | 39.8 | 49.8 |
| Ethanol | 10 | 10 | 10 | 10 |
| Propylene glycol | — | — | — | — |
| Glycerol | — | 10 | — | — |
| DIPA | — | — | 10 | — |
| BHT | 0.1 | 0.1 | 0.1 | 0.1 |
| BHA | 0.1 | 0.1 | 0.1 | 0.1 |
| Phenoxyethanol | 1 | 1 | 1 | 1 |
| Total | 100 | 100 | 100 | 100 |
| Exemplary active | 6.6 | 7.8 | 1.0-2.0 | 6.8 |
| agent concentration | | | | |

TABLE C
Exemplary composition of solvent systems for disclosed
active agents

| | |
|---|---|
| | Composition of |
| Excipient | solvent system % w/w |

| | | | | |
|---|---|---|---|---|
| ADX-102 or other | 1.00 | 1.00 | 1.00 | 1.00 |
| active agent | | | | |
| Transcutol ® HP | 8.65 | 9.60 | 8.65 | 1.68 |
| Propylene Glycol | 5.50 | 0.00 | 5.50 | 0.00 |
| Purified Water | 34.93 | 30.40 | 30.40 | 30.40 |
| SR. PEG 400 | 19.65 | 28.60 | 24.18 | 36.12 |
| Ethanol | 7.86 | 8.00 | 7.86 | 8.40 |
| Phenoxyethanol | 1.00 | 1.00 | 1.00 | 1.00 |
| BHT | 0.05 | 0.10 | 0.10 | 0.10 |
| BHA | 0.10 | 0.10 | 0.10 | 0.10 |
| Total | 78.74* | 78.80* | 78.79* | 78.80* |

*note that compositions do not add up to 100% as these amounts do not include the oil phase present in the final topical formulations. In some embodiments, the oil phase comprises a wax or oleaginous mixture, such as beeswax, paraffin, white petrolatum, or a mixture thereof.

Solubilizing Agents

In some embodiments, the composition comprises a solubilizing agent. In some embodiments, the solubilizing agent is a glycol ether. In some embodiments, the solubilizing agent is selected from benzalkonium chloride, benzethonium chloride, benzyl alcohol, benzyl benzoate, cetylpyridinium chloride, a cyclodextrin, glycerin monostearate, hydroxpropyl betadex, hypromellose, inulin, lecithin, meglumine, nonionic emulsifying wax, a phospholipid, poloxamer, polyoxyethylene alkyl ethers, a polyoxyethylene castor oil derivative, a polyoxyethylene sorbitan fatty acid ester, a polyoxyethylene stearates, a polyoxylglyceride, povidone, pyrrolidone, sodium bicarbonate, a sorbitan ester, starch, stearic acid, a sulfobutylether beta-cyclodextrin, tricaprylin, triolein, vitamin E polyethylene glycol succinate, or a mixture thereof.

In some embodiments, a solubilizing agent is a dialkylene glycol monoalkyl ether. In certain embodiments, a solubilizing agent is diethylene glycol monoethyl ether. In some embodiments, a solubilizing agent is a glycol ether present in an amount of less than about 8 wt %. In some embodiments, a solubilizing agent is a glycol ether present in an amount of less than about 7 wt %. In some embodiments, a solubilizing agent is a glycol ether present in an amount of less than about 6 wt %. In some embodiments, a solubilizing agent is a glycol ether present in an amount of less than about 5 wt %. In some embodiments, a solubilizing agent is a glycol ether present in an amount ranging from about 0.5 wt % to about 3.0 wt %.

In some embodiments, the solubilizing agent is present in an amount of about 0.5 wt %. In some embodiments, a solubilizing agent is present in an amount of about 0.75 wt %. In some embodiments, a solubilizing agent is present in an amount of about 1.0 wt %. In some embodiments, a solubilizing agent is present in an amount of about 1.25 wt %. In some embodiments, a solubilizing agent is present in an amount of about 1.5 wt %. In some embodiments, a solubilizing agent is present in an amount of about 1.75 wt %. In some embodiments, a solubilizing agent is present in an amount of about 2.0 wt %. In some embodiments, a solubilizing agent is present in an amount of about 2.25 wt %. In some embodiments, a solubilizing agent is present in an amount of about 2.5 wt %. In some embodiments, a solubilizing agent is present in an amount of about 2.75 wt %. In some embodiments, a solubilizing agent is present in an amount of about 3.0 wt %. In some embodiments, a solubilizing agent is present in an amount of about 3.25 wt %. In some embodiments, a solubilizing agent is present in an amount of about 3.5 wt %. In some embodiments, a solubilizing agent is present in an amount of about 3.75 wt %. In some embodiments, a solubilizing agent is present in an amount of about 4.0 wt %. In some embodiments, a solubilizing agent is present in an amount of about 4.25 wt %. In some embodiments, a solubilizing agent is present in an amount of about 4.5 wt %. In some embodiments, a solubilizing agent is present in an amount of about 4.75 wt %. In some embodiments, a solubilizing agent is present in an amount of about 5.0 wt %. In some embodiments, a solubilizing agent is present in an amount of about 6.0 wt %. In some embodiments, a solubilizing agent is present in an amount of about 7.0 wt %. In some embodiments, a solubilizing agent is present in an amount of about 8.0 wt %. In some embodiments, a solubilizing agent is present in an amount of about 9.0 wt %. In some embodiments, a solubilizing agent is present in an amount of less than about 10.0 wt %.

In some embodiments, the solubilizing agent is present. In some embodiments, the solubilizing agent is an alcohol solubilizing agent. In certain embodiments, the alcohol solubilizing reagent is present in an amount ranging from about 1 wt % to about 9 wt %. Exemplary such alcohol solubilizing agents are described above and herein and include, e.g., methanol, ethanol, propanol, and the like. In certain embodiments, the topical composition is as described above, and either a second or third solubilizing agent is a glycol ether solubilizing agent. In certain embodiments, the glycol ether solubilizing reagent is present in an amount ranging from about 1 wt % to about 9 wt %. In certain embodiments, the glycol ether solubilizing agent is present in an amount ranging from about 1 wt % to about 5 wt %. In certain embodiments, the glycol ether solubilizing agent is present in an amount of about 1 wt % to about 3 wt %. In certain embodiments, the glycol ether solubilizing agent is present in an amount of less than about 1 wt %. Exemplary such glycol ether solubilizing agents are described above and herein and include dialkylene glycol monoalkylethers such as, e.g., diethylene glycol monoethylether.

Penetration Enhancers

One long-standing approach for improving transdermal drug delivery uses penetration enhancers (also called sorption promoters or accelerants), which penetrate into skin to reversibly decrease the barrier resistance. Numerous compounds are known in the art as penetration enhancers. In some embodiments, the topical composition includes a penetration enhancer such as a sulfoxide (such as dimethylsulfoxide, DMSO), an azone (e.g. laurocapram), a pyrrolidone (for example 2-pyrrolidone, 2P), an alcohol or alkanol (ethanol, or decanol), a fatty acid (such as lauric acid, myristic acid and capric acid), surfactant (such as polyoxyethylene-2-oleyl ether, polyoxy ethylene-2-stearly ether), a glycol (for example propylene glycol, ethylene glycol, or tetraethylene glycol), a terpene or terpenoid, an oxazolidinone such as 4-decyloxazolidin-2-one, or a urea (such as a cyclic urea).

In some embodiments, the penetration enhancer is selected from one or more of an alkanol, dimethyl sulfoxide, a glycol, glyceryl monooleate, glycofurol, isopropyl myristate, isopropyl palmitate, lanolin, light mineral oil, linoleic acid, menthol, myristic acid, myristyl alcohol, oleic acid, oleyl alcohol, palmitic acid, a polyoxyethylene alkyl ether, a polyoxylglyceride, a pyrrolidone, sodium lauryl sulfate, thymol, tricaprylin, triolein, acetyltributyl citrate, ethylene vinyl acetate, a polymethacrylate, polyvinyl alcohol, sesame oil, sodium carboxymethyl guar, or stearyl alcohol.

In some embodiments, the penetration enhancer is selected from Transcutol HP, propylene glycol, or a mixture thereof.

Surfactants

A variety of surfactants may be used in the presently described compositions. For example, nonionic, ionic, zwitterionic, anionic, and cationic surfactants can be used to serve the general purpose of solubilizing or homogenizing hydrophobic and hydrophilic components of the composition and/or ensuring a uniform texture and/or preventing phase separation.

In some embodiments, the surfactant is selected from a polyoxyethylene alkyl ether or the like. The polyoxyethylene alkyl ethers are a series of polyoxyethylene glycol ethers of n-alcohols (lauryl, oleyl, myristyl, cetyl, and stearyl alcohol). Examples from the USP-NF include Polyoxyl 20 Cetostearyl Ether, Polyoxyl 10 Oleyl Ether, Polyoxyl Lauryl Ether, and Polyoxyl Stearyl Ether. Polyoxyethylene alkyl ethers are employed extensively in cosmetics, where the CTFA names laureth-N, myreth-N, ceteth-N, and steareth-N are commonly used. In this nomenclature, N is the number of ethylene oxide groups, e.g. steareth-20.

Polyoxyethylene alkyl ethers are nonionic surfactants produced by the polyethoxylation of linear fatty alcohols. Products tend to be mixtures of polymers of slightly varying molecular weights, and the numbers used to describe polymer lengths are average values. Two systems of nomenclature are used to describe these materials. The number “10” in the name Volpo N10 refers to the approximate polymer length in oxyethylene units. The number “1000” in the name “cetomacrogol 1000” refers to the average molecular weight of the polymer chain.

Nonionic surfactants are used as emulsifying agents for water-in-oil and oil-in-water emulsions, and the stabilization of microemulsions and multiple emulsions. Polyoxyethylene alkyl ethers are used as solubilizing agents for essential oils, perfumery chemicals, vitamin oils, and drugs of low water solubility such as cortisone acetate, griseofulvin, menadione, chlordiazepoxide and cholesterol. They have applications as antidusting agents for powders; wetting and dispersing agents for coarse-particle liquid dispersions; and detergents, especially in shampoos, face washes and similar cosmetic cleaning preparations. They are used as gelling and foaming agents (e.g. Brij 72 gives a quick-breaking foam, while Brij 97 (15-20%), Volpo N series and Cremophor A25 (21-30%) give clear gels). Polyoxyethylene alkyl ethers have also been used in suppository formulations to increase the drug release from the suppository bases.

In some embodiments, the surfactant is selected from emulsifying wax USP, glyceryl monooleate, a phospholipid, a polyoxyethylene alkyl ether, polyoxyethylene castor oil derivative, a polyoxyethylene sorbitan fatty acid ester, polyoxyethylene stearates, polyoxylglycerides (such as Caprylocaproyl Polyoxylglycerides, Lauroyl Polyoxylglycerides, Linoleoyl Polyoxylglycerides, Oleoyl Polyoxylglycerides, Stearoyl Polyoxylglycerides), polysorbate, a sorbitan ester, triethyl citrate, vitamin E polyethylene glycol succinate, miglyol, a Steareth (Brij) surfactant, a Cetomacrogol, a Myrj, a Span, a Tween, or a Laureth surfactant.

In some embodiments, the surfactant is selected from Steareth 21 (Brij s721), Steareth 2 (Brij S2), Cetomacrogol 1000, Myrj S40, Brij CS25 (Ceteareth 25), Steareth 20 (Brij S20), Span 20, Geleol mono and diglycerides, Span 60, or Tween 60, or a combination thereof.

Thickening Agents and Stabilizing Agents

In some embodiments, the composition comprises a thickening agent, such as acacia, guar gum, alginate, poloxamer 188, ceresin wax, fructose, pectin, polyethylene oxide, PEG 1500, or PEG 4000. In certain embodiments, a thickening agent such as PEG 1500 or PEG 4000 is present in an amount of about 1.0 wt % to about 45 wt %. In certain embodiments, a thickening agent is present in an amount of about 5.0 wt % to about 30 wt %. In certain embodiments, a thickening agent is present in an amount of about 15 wt % to about 25 wt %. In certain embodiments, a thickening agent is present in an amount of about 8.0 wt % to about 21 wt %. In some embodiments, the thickening agent is PEG 1500 or PEG 4000 present in an amount of about 0.5-25 wt %.

In some embodiments, the composition comprises a stabilizing agent, whether or not a thickening agent is present. Exemplary stabilizing agents include white wax, yellow wax (beeswax), ceresin wax, liquid paraffin, white petrolatum, and the like. Stabilizing agents like yellow wax are used in food, cosmetics, and confectionery products. The main use of yellow wax, for example, is in topical pharmaceutical formulations, where it is used at a concentration of 5-20% as a stiffening agent in ointments and creams. Yellow wax is also employed in emulsions because it enables water to be incorporated into water-in-oil emulsions.

In some embodiments, the stabilizing agent comprises beeswax, liquid paraffin, white petrolatum, or a combination of two or all three thereof. In some embodiments, the stabilizing agent is present in the composition in an amount of about 20.0-20.0%, 30.0-18.0%, 40.0-17.0%, 4.0-16.0%, 4.5-15.0%, 4.5-14.0%, 5.0-13.0%, 5.0-12.0%, or 6.0-10.0% w/w. In some embodiments, about 2.0% 3.0%, 4.0%, 5.0%, 6.0%, 7.0%, 8.0%, 9.0%, 10.0%, 120.0%, 130.0%, 140.0%, 15.0%, 16.0%, 17.0%, 18.0%, 19.0%, or 20.0% w/w of a stabilizing agent is present. In some embodiments, about 3.0% white petrolatum and about 5.0% or about 7.1% liquid paraffin or about 3.0% or about 6.1% beeswax is present in the composition.

Preservatives

The preservatives for use in the presently described topical compositions can include those described herein, including any known pharmaceutically acceptable preservative that functions by inhibiting bacteria and/or fungi, and/or by inhibiting oxidation. Suitable preservatives can include but are not limited to antimicrobial agents and/or antioxidants. Suitable antimicrobial agents can include but are not limited to benzoates, benzyl alcohol, sodium benzoate, sorbates, propionates, and nitrites. Suitable antioxidants can include but are not limited to vitamin C, butylated hydroxytoluene (BHT), sulphites, and vitamin E, ascorbyl palmitate, α-tocopherol, and propyl gallate as well as any known pharmaceutically acceptable preservative. In certain embodiments, the preservative is benzyl alcohol or phenoxyethanol.

In some embodiments, a preservative and antioxidant are present in the composition. In some embodiments, the preservative and antioxidant comprise phenoxyethanol, BHT, butylated hydroxyanisole (BHA), or a mixture of two or all three thereof.

In some embodiments, the preservative and antioxidant comprise at least one, two, three, or four selected from phenoxyethanol, BHT, butylated hydroxyanisole (BHA), α-tocopherol, propyl gallate, or ascorbyl palmitate.

In some embodiments, the preservative and antioxidant comprise one of the following combinations:

(i) phenoxyethanol, BHT, and α-tocopherol;

(ii) phenoxyethanol, BHT, α-tocopherol, and ascorbyl palmitate;

(iii) phenoxyethanol, α-tocopherol, and ascorbyl palmitate;

(iv) phenoxyethanol, propyl gallate, and ascorbyl palmitate; or

(v) phenoxyethanol, BHT, and propyl gallate.

The described preservatives and/or antioxidants can be present in the described topical compositions in an amount of, for example, from about 0.001 wt % to about 15 wt %; from about 0.01 wt % to about 5 wt %; from about 0.2 wt % to about 4 wt %; from about 0.3 wt % to about 4 wt %; from about 0.4 wt % to about 4 wt %; from about 0.5 wt % to about 4 wt %; from about 0.6 wt % to about 4 wt %; from about 0.7 wt % to about 3 wt %; from about 0.8 wt % to about 2 wt %; from about 0.9 wt % to about 1.5 wt %; from about 0.9 wt % to about 1.1 wt %; about 0.9 wt %; about 1 wt %; or about 1.1 wt % preservative.

In certain embodiments, a topical composition of the present invention comprises benzyl alcohol or phenoxyethanol in an amount ranging from about 0.1 wt % to about 2.1 wt %. In certain embodiments, a topical composition of the present invention comprises benzyl alcohol or phenoxyethanol in an amount of about 1.0 wt %. In some embodiments, benzyl alcohol is absent from a topical composition of the present invention.

pH Adjusters

pH adjusters for use in the presently described topical compositions can include any pharmaceutically acceptable composition, compound, or agent, suitable for adjusting the pH of the presently described topical pharmaceutical compositions without negatively affecting any property thereof. Suitable pH adjusters can include any pharmaceutically acceptable acid or base. Suitable pH adjusters include those described herein.

In some embodiments, topical compositions of the present invention comprise a basic pH adjuster. For example, in certain embodiments, the pH adjuster is an amine base. Exemplary such amine bases are known in the chemical and pharmaceutical arts and include, e.g., triethanolamine (i.e., Trolamine). In certain embodiments, a topical composition of the present invention comprises from about 0.05 wt % to about 0.25 wt % of a pH adjuster such as triethanolamine.

In some embodiments, a basic pH adjuster is hydroxide. In certain embodiments, the hydroxide is in the form of a salt of an alkali or alkaline earth metal. For instance, in some embodiments, a hydroxide salt is sodium hydroxide, potassium hydroxide, or calcium hydroxide. In some embodiments, a pH adjuster is carbonate. In certain embodiments, the carbonate is in the form of a salt of an alkali or alkaline earth metal. For instance, in some embodiments, a carbonate salt is sodium carbonate, potassium carbonate, or calcium carbonate. Exemplary other such hydroxide and carbonate bases and the like are well-known in the chemical and pharmaceutical arts and contemplated herein.

The described pH adjusters can be present in a described topical compositions in an amount of from >0.01 wt % to about 1 wt %; from >0.05 wt % to about 1 wt %; from about 0.05 wt % to about 0.5 wt %; from about 0.08 wt % to about 0.4 wt %; from about 0.08 wt % to about 0.35 wt %; from about 0.08 wt % to about 0.3 wt %; from about 0.08 wt % to about 0.25 wt %; from about 0.09 wt % to about 0.4 wt %; from about 0.09 wt % to about 0.3 wt %; from about 0.09 wt % to about 0.25 wt %; from about 0.1 wt % to about 0.25 wt %; from about 0.11 wt % to about 0.24 wt %; from about 0.12 wt % to about 0.23 wt %; from about 0.13 wt % to about 0.22 wt %; from about 0.14 wt % to about 0.21 wt %; from about 0.15 wt % to about 0.2 wt %; from about 0.16 wt % to about 0.19 wt %; from about 0.16 wt % to about 0.18 wt %; from about 0.165 wt % to about 0.175 wt %; about 0.16 wt %; about 0.17 wt %; or about 0.18 wt % pH adjuster.

In some embodiments, a presently described topical composition has a pH of from about 5.0 to about 8.5; from about 6.0 to about 7.0; from about 6.1 to about 6.9; from about 6.2 to about 6.8; from about 6.3 to about 6.7; from about 6.4 to about 6.6; about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, or about 8.2.

In some embodiments, a presently described topical composition has a pH of from about 6.0 to about 8.5. In some embodiments, a presently described topical composition has a pH of about 6.5, about 6.6, about 6, 7, about 6.7, about 6.8, about 6.9, about 7.0, about 7.1, about 7.2, about 7.3, about 7.5, about 7.5, about 7.6, about 7.7, about 7.8, about 7.9, about 8.0, about 8.1, about 8.2, about 8.3, about 8.4, or about 8.5. For example, the pH of the presently described topical composition comprising about 1.0 wt % active agent, for example ADX-102, at 23° C.±2° C. can be from about 6.6 to about 8.4.

Chelating Agents

Chelating agents for use in the presently described topical compositions can include any known pharmaceutically acceptable chelating agents. Suitable chelating agents can include but are not limited to any one or more of ethylenediaminetetraacetic acid (EDTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), a polyphosphate, or porphine; or a pharmaceutically acceptable salt thereof. In certain embodiments, a suitable chelating agent is disodium EDTA.

The described chelating agents can be present in the described topical pharmaceutical compositions in an amount of, for example, from about 0.001 wt % to about 10 wt %; from about 0.005 wt % to about 5 wt %; from about 0.005 wt % to about 0.5 wt %; from about 0.001 wt % to about 1 wt %; from about 0.01 to about 5 wt %; from about 0.006 wt % to about 0.04 wt %; from about 0.007 wt % to about 0.035 wt %; from about 0.008 wt % to about 0.035 wt %; from about 0.009 wt % to about 0.035 wt %; from about 0.01 wt % to about 0.03 wt %; from about 0.015 wt % to about 0.025 wt %; from about 0.018 wt % to about 0.022 wt %; from about 0.019 wt % to about 0.021 wt %; about 0.019 wt %; about 0.02 wt %; or about 0.021 wt % chelating agent.

Active Agents

The presently disclosed topical (e.g., dermal) pharmaceutical compositions are advantageous for delivery of a variety of aminocarbinol-containing compounds, such as those described herein.

The disclosed compositions provide stable solubilization, storage, and delivery of the active agent at a variety of concentrations. The active agent may be present in the topical pharmaceutical compositions in an amount of from 0.001 wt % to about 15% wt %; 0.01 wt % to about 12 wt %; from 0.01 wt % to about 11 wt %; from 0.01 wt % to about 10 wt %; from 0.01 wt % to about 9 wt %; from 0.01 wt % to about 8 wt %; from 0.01 wt % to about 7 wt %; from 0.01 wt % to about 6 wt %; from 0.01 wt % to about 5 wt %; from 0.01 wt % to about 4.5 wt %; from 0.01 wt % to about 4 wt %; from 0.01 wt % to about 3.5 wt %; from 0.01 wt % to about 3 wt %; from 0.01 wt % to about 2.5 wt %; from 0.01 wt % to about 2 wt %; from 0.01 wt % to about 2.25 wt %; from 0.01 wt % to about 2 wt %; from about 0.01% to about 4 wt %; from about 0.01 wt % to about 3 wt %; from about 0.01 wt % to about 2.5 wt %; from about 0.01 wt % to about 2.25 wt %; from about 0.01 wt % to about 2 wt %; from about 0.01 wt % to about 1.5 wt %; from about 0.01 wt % to about 1 wt %; from about 0.01 wt % to about 0.5 wt %; from 0.1 wt % to about 12 wt %; from 0.1 wt % to about 11 wt %; from 0.1 wt % to about 10 wt %; from 0.1 wt % to about 9 wt %; from 0.1 wt % to about 8 wt %; from 0.1 wt % to about 7 wt %; from 0.1 wt % to about 6 wt %; from 0.1 wt % to about 5 wt %; from 0.1 wt % to about 4.5 wt %; from 0.1 wt % to about 4 wt %; from 0.1 wt % to about 3.5 wt %; from 0.1 wt % to about 3 wt %; from 0.1 wt % to about 2.5 wt %; from 0.1 wt % to about 2 wt %; from 0.1 wt % to about 2.25 wt %; from 0.1 wt % to about 2 wt %; from about 0.1% to about 4 wt %; from about 0.1 wt % to about 3 wt %; from about 0.1 wt % to about 2.5 wt %; from about 0.1 wt % to about 2.25 wt %; from about 0.1 wt % to about 2 wt %; from about 0.1 wt % to about 1.5 wt %; from about 0.1 wt % to about 1 wt %; from about 0.1 wt % to about 0.5 wt %; from 0.7 wt % to about 10 wt %; from 0.7 wt % to about 9 wt %; from 0.7 wt % to about 8 wt %; from 0.7 wt % to about 7 wt %; from 0.7 wt % to about 6 wt %; from 0.7 wt % to about 5 wt %; from 0.7 wt % to about 4.5 wt %; from 0.7 wt % to about 4 wt %; from 0.7 wt % to about 3.5 wt %; from 0.7 wt % to about 3 wt %; from 0.7 wt % to about 2.5 wt %; from 0.7 wt % to about 2 wt %; from 0.7 wt % to about 2.25 wt %; from 0.7 wt % to about 2 wt %; from about 0.7% to about 4 wt %; from about 0.7 wt % to about 3 wt %; from about 0.7 wt % to about 2.5 wt %; from about 0.7 wt % to about 2.25 wt %; from about 0.7 wt % to about 2 wt %; from about 0.7 wt % to about 1.5 wt %; from about 0.7 wt % to about 1 wt %; from 0.9 wt % to about 10 wt %; from 0.9 wt % to about 9 wt %; from 0.9 wt % to about 8 wt %; from 0.9 wt % to about 7 wt %; from 0.9 wt % to about 6 wt %; from 0.9 wt % to about 5 wt %; from 0.9 wt % to about 4.5 wt %; from 0.9 wt % to about 4 wt %; from 0.9 wt % to about 3.5 wt %; from 0.9 wt % to about 3 wt %; from 0.9 wt % to about 2.5 wt %; from 0.9 wt % to about 2 wt %; from 0.9 wt % to about 2.25 wt %; from 0.9 wt % to about 2 wt %; from about 0.9% to about 4 wt %; from about 0.9 wt % to about 3 wt %; from about 0.9 wt % to about 2.5 wt %; from about 0.9 wt % to about 2.25 wt %; from about 0.9 wt % to about 2 wt %; from about 0.9 wt % to about 1.5 wt %; from about 0.9 wt % to about 1 wt %; from 1 wt % to about 15 wt %; from 1 wt % to about 12 wt %; from 1 wt % to about 11 wt %; from 1 wt % to about 10 wt %; from 1 wt % to about 9 wt %; from 1 wt % to about 8 wt %; from 1 wt % to about 7 wt %; from 1 wt % to about 6 wt %; from 1 wt % to about 5 wt %; from 1 wt % to about 4.5 wt %; from 1 wt % to about 4 wt %; from 1 wt % to about 3.5 wt %; from 1 wt % to about 3 wt %; from 1 wt % to about 2.5 wt %; from 1 wt % to about 2 wt %; from 1 wt % to about 2.25 wt %; from 1 wt % to about 2 wt %; from about 1% to about 4 wt %; from about 1 wt % to about 3 wt %; from about 1 wt % to about 2.5 wt %; from about 1 wt % to about 2.25 wt %; from about 1 wt % to about 2 wt %; from about 1 wt % to about 1.5 wt %; from about 1.5 wt % to about 10 wt %; from about 1.5 wt % to about 9 wt %; from about 1.5 wt % to about 8 wt %; from about 1.5 wt % to about 7 wt %; from about 1.5 wt % to about 6 wt %; from about 1.5 wt % to about 5 wt %; from about 1.5 wt % to about 4.5 wt %; from about 1.5 wt % to about 4 wt %; from about 1.5 wt % to about 3.5 wt %; from about 1.5 wt % to about 3 wt %; from about 1.5 wt % to about 2.5 wt %; %; from about 1.5 wt % to about 2.25 wt %; from about 1.5 wt % to about 2 wt %; from about 3 wt % to about 10 wt %; from about 3 wt % to about 9 wt %; from about 3 wt % to about 8 wt %; from about 3 wt % to about 7 wt %; from about 3 wt % to about 6 wt %; from about 3 wt % to about 5 wt %; from about 3 wt % to about 4.5 wt %; from about 3 wt % to about 4 wt %; from about 3 wt % to about 3.5 wt %; or in any other amount within any of the above ranges.

In some embodiments, the active agent or pharmaceutically acceptable salt thereof is present in the topical pharmaceutical compositions in an amount of from about 0.1 wt % to about 2.5 wt %; from about 0.1 wt % to about 2.0 wt %; from about 0.1 wt % to about 1.8 wt %; from about 0.1 wt % to about 1.7 wt %; from about 0.1 wt % to about 1.6 wt %; from about 0.1 wt % to about 1.5 wt %; from about 0.1 wt % to about 1.4 wt %; from about 0.1 wt % to about 1.30 wt %; from about 0.1 wt % to about 1.25 wt %; from about 0.1 wt % to about 1.20 wt %; from about 0.1 wt % to about 1.15 wt %; from about 0.1 wt % to about 1.1 wt %; from about 0.1 wt % to about 1.05 wt %; from about 0.1 wt % to about 1.0 wt %; from about 0.1 wt % to about 0.95 wt %; from about 0.1 wt % to about 0.9% wt; 0.2 wt % to about 2.5 wt %; from about 0.2 wt % to about 2.0 wt %; from about 0.2 wt % to about 1.8 wt %; from about 0.2 wt % to about 1.7 wt %; from about 0.2 wt % to about 1.6 wt %; from about 0.2 wt % to about 1.5 wt %; from about 0.2 wt % to about 1.4 wt %; from about 0.2 wt % to about 1.30 wt %; from about 0.2 wt % to about 1.25 wt %; from about 0.2 wt % to about 1.20 wt %; from about 0.2 wt % to about 1.15 wt %; from about 0.2 wt % to about 1.1 wt %; from about 0.2 wt % to about 1.05 wt %; from about 0.2 wt % to about 1.0 wt %; 0.3 wt % to about 2.5 wt %; from about 0.3 wt % to about 2.0 wt %; from about 0.3 wt % to about 1.8 wt %; from about 0.3 wt % to about 1.7 wt %; from about 0.3 wt % to about 1.6 wt %; from about 0.3 wt % to about 1.5 wt %; from about 0.3 wt % to about 1.4 wt %; from about 0.3 wt % to about 1.30 wt %; from about 0.3 wt % to about 1.25 wt %; from about 0.3 wt % to about 1.20 wt %; from about 0.3 wt % to about 1.15 wt %; from about 0.3 wt % to about 1.1 wt %; from about 0.3 wt % to about 1.05 wt %; from about 0.3 wt % to about 1.0 wt %; 0.4 wt % to about 2.5 wt %; from about 0.4 wt % to about 2.0 wt %; from about 0.4 wt % to about 1.8 wt %; from about 0.4 wt % to about 1.7 wt %; from about 0.4 wt % to about 1.6 wt %; from about 0.4 wt % to about 1.5 wt %; from about 0.4 wt % to about 1.4 wt %; from about 0.4 wt % to about 1.30 wt %; from about 0.4 wt % to about 1.25 wt %; from about 0.4 wt % to about 1.20 wt %; from about 0.4 wt % to about 1.15 wt %; from about 0.4 wt % to about 1.1 wt %; from about 0.4 wt % to about 1.05 wt %; from about 0.4 wt % to about 1.0 wt %; 0.5 wt % to about 2.5 wt %; from about 0.5 wt % to about 2.0 wt %; from about 0.5 wt % to about 1.8 wt %; from about 0.5 wt % to about 1.7 wt %; from about 0.5 wt % to about 1.6 wt %; from about 0.5 wt % to about 1.5 wt %; from about 0.5 wt % to about 1.4 wt %; from about 0.5 wt % to about 1.30 wt %; from about 0.5 wt % to about 1.25 wt %; from about 0.5 wt % to about 1.20 wt %; from about 0.5 wt % to about 1.15 wt %; from about 0.5 wt % to about 1.1 wt %; from about 0.5 wt % to about 1.05 wt %; from about 0.5 wt % to about 1.0 wt %; 0.6 wt % to about 2.5 wt %; from about 0.6 wt % to about 2.0 wt %; from about 0.6 wt % to about 1.8 wt %; from about 0.6 wt % to about 1.7 wt %; from about 0.6 wt % to about 1.6 wt %; from about 0.6 wt % to about 1.5 wt %; from about 0.6 wt % to about 1.4 wt %; from about 0.6 wt % to about 1.30 wt %; from about 0.6 wt % to about 1.25 wt %; from about 0.6 wt % to about 1.20 wt %; from about 0.6 wt % to about 1.15 wt %; from about 0.6 wt % to about 1.1 wt %; from about 0.6 wt % to about 1.05 wt %; from about 0.6 wt % to about 1.0 wt %; 0.7 wt % to about 2.5 wt %; from about 0.7 wt % to about 2.0 wt %; from about 0.7 wt % to about 1.8 wt %; from about 0.7 wt % to about 1.7 wt %; from about 0.7 wt % to about 1.6 wt %; from about 0.7 wt % to about 1.5 wt %; from about 0.7 wt % to about 1.4 wt %; from about 0.7 wt % to about 1.30 wt %; from about 0.7 wt % to about 1.25 wt %; from about 0.7 wt % to about 1.20 wt %; from about 0.7 wt % to about 1.15 wt %; from about 0.7 wt % to about 1.1 wt %; from about 0.7 wt % to about 1.05 wt %; from about 0.7 wt % to about 1.0 wt %; 0.8 wt % to about 2.5 wt %; from about 0.8 wt % to about 2.0 wt %; from about 0.8 wt % to about 1.8 wt %; from about 0.8 wt % to about 1.7 wt %; from about 0.8 wt % to about 1.6 wt %; from about 0.8 wt % to about 1.5 wt %; from about 0.8 wt % to about 1.4 wt %; from about 0.8 wt % to about 1.30 wt %; from about 0.8 wt % to about 1.25 wt %; from about 0.8 wt % to about 1.20 wt %; from about 0.8 wt % to about 1.15 wt %; from about 0.8 wt % to about 1.1 wt %; from about 0.8 wt % to about 1.05 wt %; or from about 0.8 wt % to about 1.0 wt %.

The described active agent and/or a pharmaceutically acceptable salt thereof can be present in a topical composition of the present invention in an amount of from about 0.1 wt % to about 7.5 wt %, about 0.05 wt % to about 6.0 wt %, of from about 0.1 wt % to about 6.0 wt %, of from about 0.5 wt % to about 6.0 wt %, of from about 0.6 wt % to about 6.0 wt %, of from about 0.7 wt % to about 6.0 wt %, of from about 0.8 wt % to about 6.0 wt %, of from about 0.9 wt % to about 6.0 wt %, of from about 1.0 wt % to about 6.0 wt %, of from about 1.5 wt % to about 6.0 wt %, of from about 2.0 wt % to about 6.0 wt %, of from about 2.5 wt % to about 6.0 wt %, of from about 3.0 wt % to about 6.0 wt %, of from about 3.5 wt % to about 6.0 wt %, of from about 4.0 wt % to about 6.0 wt %, of from about 4.5 wt % to about 6.0 wt %, of from about 5.0 wt % to about 6.0 wt %, of from about 5.5 wt % to about 6.0 wt %, of from about 0.05 wt % to about 3.0 wt %, of from about 0.1 wt % to about 2.9 wt %, of from about 0.2 wt % to about 2.7 wt %, of from about 0.3 wt % to about 2.5 wt %, of from about 0.4 wt % to about 2.3 wt %, of from about 0.5 wt % to about 2.1 wt %, of from about 0.6 wt % to about 1.9 wt %, of from about 0.7 wt % to about 1.7 wt %, of from about 0.8 wt % to about 1.5 wt %, of from about 0.9 wt % to about 1.3 wt %, of from about 1 wt % to about 1.1 wt %, of from about 0.05 wt % to about 1.0 wt %, of from about 0.06 wt % to about 1.0 wt %, of from about 0.07 wt % to about 1.0 wt %, of from about 0.08 wt % to about 1.0 wt %, of from about 0.09 wt % to about 1.0 wt %, of from about 0.1 wt % to about 1.0 wt %, of from about 0.15 wt % to about 1.0 wt %, of from about 0.2 wt % to about 1.0 wt %, of from about 0.25 wt % to about 1.0 wt %, of from about 0.3 wt % to about 1.0 wt %, of from about 0.35 wt % to about 1.0 wt %, of from about 0.4 wt % to about 1.0 wt %, of from about 0.45 wt % to about 1.0 wt %, of from about 0.5 wt % to about 1.0 wt %, of from about 0.55 wt % to about 1.0 wt %, of from about 0.6 wt % to about 1.0 wt %, of from about 0.65 wt % to about 1.0 wt %, of from about 0.7 wt % to about 1.0 wt %, of from about 0.75 wt % to about 1.0 wt %, of from about 0.8 wt % to about 1.0 wt %, of from about 0.85 wt % to about 1.0 wt %, of from about 0.9 wt % to about 1.0 wt %, of from about 0.95 wt % to about 1.0 wt %, of from about 0.5 wt % to about 4.0 wt %, of from about 0.75 wt % to about 4.0 wt %, of from about 1 wt % to about 4.0 wt %, of from about 1.25 wt % to about 4.0 wt %, of from about 1.5 wt % to about 4.0 wt %, of from about 2 wt % to about 4.0 wt %, of from about 2.25 wt % to about 4.0 wt %, of from about 2.5 wt % to about 4.0 wt %, of from about 2.75 wt % to about 4.0 wt %, of from about 3 wt % to about 4.0 wt %, of from about 3.75 wt % to about 4.0 wt %, of from about 1.5 wt % to about 3.0 wt %, from about 1.5 wt % to about 2.5 wt %, from about 1.6 wt % to about 2.4 wt %, from about 1.7 wt % to about 2.3 wt %, from about 1.75 wt % to about 2.25 wt %, from about 1.8 wt % to about 2.2 wt %, from about 1.9 wt % to about 2.1 wt %, about 2.0 wt %, about 2.5 wt %, from about 1.9 wt % to about 3.0 wt %, from about 2.0 wt % to about 3.0 wt %, from about 2.1 wt % to about 3.0 wt %, from about 2.2 wt % to about 3.0 wt %, from about 2.3 wt % to about 3.0 wt %, from about 2.4 wt % to about 3.0 wt %, from about 2.5 wt % to about 3.0 wt %, from about 2.6 wt % to about 3.0 wt %, from about 2.7 wt % to about 3.0 wt %, from about 2.8 wt % to about 3.0 wt %, from about 2.9 wt % to about 3.0 wt %, or in any other amount within any of the above ranges.

The described active agent and/or a pharmaceutically acceptable salt thereof can be present in a topical composition of the present invention in an amount of from about 0.05 wt % to about 6.0 wt %, of from about 0.1 wt % to about 6.0 wt %, of from about 0.5 wt % to about 6.0 wt %, of from about 0.6 wt % to about 6.0 wt %, of from about 0.7 wt % to about 6.0 wt %, of from about 0.8 wt % to about 6.0 wt %, of from about 0.9 wt % to about 6.0 wt %, of from about 1.0 wt % to about 6.0 wt %, of from about 1.5 wt % to about 6.0 wt %, of from about 2.0 wt % to about 6.0 wt %, of from about 2.5 wt % to about 6.0 wt %, of from about 3.0 wt % to about 6.0 wt %, of from about 3.5 wt % to about 6.0 wt %, of from about 4.0 wt % to about 6.0 wt %, of from about 4.5 wt % to about 6.0 wt %, of from about 5.0 wt % to about 6.0 wt %, of from about 5.5 wt % to about 6.0 wt %, of from about 0.05 wt % to about 3.0 wt %, of from about 0.1 wt % to about 2.9 wt %, of from about 0.2 wt % to about 2.7 wt %, of from about 0.3 wt % to about 2.5 wt %, of from about 0.4 wt % to about 2.3 wt %, of from about 0.5 wt % to about 2.1 wt %, of from about 0.6 wt % to about 1.9 wt %, of from about 0.7 wt % to about 1.7 wt %, of from about 0.8 wt % to about 1.5 wt %, of from about 0.9 wt % to about 1.3 wt %, of from about 1 wt % to about 1.1 wt %, of from about 0.05 wt % to about 1.0 wt %, of from about 0.06 wt % to about 1.0 wt %, of from about 0.07 wt % to about 1.0 wt %, of from about 0.08 wt % to about 1.0 wt %, of from about 0.09 wt % to about 1.0 wt %, of from about 0.1 wt % to about 1.0 wt %, of from about 0.15 wt % to about 1.0 wt %, of from about 0.2 wt % to about 1.0 wt %, of from about 0.25 wt % to about 1.0 wt %, of from about 0.3 wt % to about 1.0 wt %, of from about 0.35 wt % to about 1.0 wt %, of from about 0.4 wt % to about 1.0 wt %, of from about 0.45 wt % to about 1.0 wt %, of from about 0.5 wt % to about 1.0 wt %, of from about 0.55 wt % to about 1.0 wt %, of from about 0.6 wt % to about 1.0 wt %, of from about 0.65 wt % to about 1.0 wt %, of from about 0.7 wt % to about 1.0 wt %, of from about 0.75 wt % to about 1.0 wt %, of from about 0.8 wt % to about 1.0 wt %, of from about 0.85 wt % to about 1.0 wt %, of from about 0.9 wt % to about 1.0 wt %, of from about 0.95 wt % to about 1.0 wt %, of from about 0.5 wt % to about 4.0 wt %, of from about 0.75 wt % to about 4.0 wt %, of from about 1 wt % to about 4.0 wt %, of from about 1.25 wt % to about 4.0 wt %, of from about 1.5 wt % to about 4.0 wt %, of from about 2 wt % to about 4.0 wt %, of from about 2.25 wt % to about 4.0 wt %, of from about 2.5 wt % to about 4.0 wt %, of from about 2.75 wt % to about 4.0 wt %, of from about 3 wt % to about 4.0 wt %, of from about 3.75 wt % to about 4.0 wt %, of from about 1.5 wt % to about 3.0 wt %, from about 1.5 wt % to about 2.5 wt %, from about 1.6 wt % to about 2.4 wt %, from about 1.7 wt % to about 2.3 wt %, from about 1.75 wt % to about 2.25 wt %, from about 1.8 wt % to about 2.2 wt %, from about 1.9 wt % to about 2.1 wt %, about 2.0 wt %, about 2.5 wt %, from about 1.9 wt % to about 3.0 wt %, from about 2.0 wt % to about 3.0 wt %, from about 2.1 wt % to about 3.0 wt %, from about 2.2 wt % to about 3.0 wt %, from about 2.3 wt % to about 3.0 wt %, from about 2.4 wt % to about 3.0 wt %, from about 2.5 wt % to about 3.0 wt %, from about 2.6 wt % to about 3.0 wt %, from about 2.7 wt % to about 3.0 wt %, from about 2.8 wt % to about 3.0 wt %, from about 2.9 wt % to about 3.0 wt %, about 3.0 wt %, 2.0 wt %, 2.5 wt %, or 3.0 wt %.

In some embodiments, the active agent or pharmaceutically acceptable salt thereof is present in a topical composition in an amount of about 1.0 wt %. In some embodiments, the active agent or pharmaceutically acceptable salt thereof is present in an amount of about 0.01 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.2 wt %, about 2.5 wt %, about 2.7 wt %, about 2.9 wt %, about 3.0 wt %, about 3.25 wt %, about 3.5 wt %, about 3.75 wt %, about 4.0 wt %, about 4.25 wt %, about 4.5 wt %, about 4.75 wt %, about 5.0 wt %, about 5.25 wt %, about 5.5 wt %, about 5.75 wt %, about 6.0 wt %, about 6.5 wt %, about 7.0 wt %, or about 7.5 wt %.

Compounds described herein include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments, the term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. In certain embodiments of the compounds, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned for the compounds herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O (CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR)R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂₀H, —(CH₂)₀₋₂₀R^{∘}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR′, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*2))₂₋₃S—, wherein each independent occurrence of R* is selected from hydrogen, C₁₋₆ aliphatic which may be substituted as defined below, and an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, besylate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds described herein are within the scope of the the present disclosure.

In some embodiments, the compound is of formula:

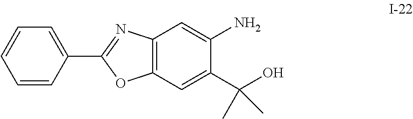 [SMILES: CC(C)(O)c1cc2oc(-c3ccccc3)nc2cc1N]

or a pharmaceutically acceptable salt thereof.

In some embodiments, the compound is of formula:

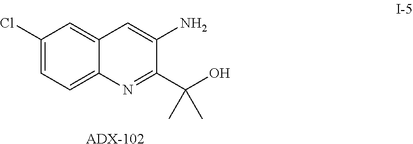

or a pharmaceutically acceptable salt thereof.

In some embodiments, the compound is of formula:

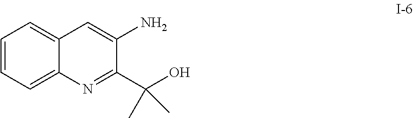

or a pharmaceutically acceptable salt thereof.

In some embodiments, the compound is of formula:

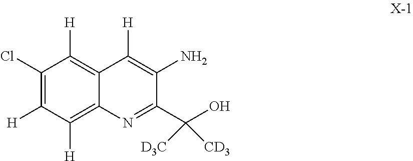 [SMILES: [2H]C([2H])([2H])C(O)(c1nc2ccc(Cl)cc2cc1N)C([2H])([2H])[2H]]

or a pharmaceutically acceptable salt thereof.

### Description of Embodiments

As described above, compounds having amino-carbinol moiety can be used to react with and trap aldehydes. Such aldehydes may be generated as part of an inflammatory response or genetic disorder such that sequestering the aldehydes can ameliorate or attenuate the inflammatory response. Accordingly, in some embodiments, a method of treating a skin disease or inflammatory disease or disorder in a subject comprises administering to a subject in need thereof a therapeutically effective amount of an aldehyde trapping compound. In some embodiments, the compound is selected from the compounds recited in U.S. Pat. No. 7,973,025 and published international patent application nos. WO2014/116836, WO 2018/039192, WO 2018/039197, or WO2017/035077, the entireties of which are incorporated herein by reference.

In some embodiments, the compound is of formula I:

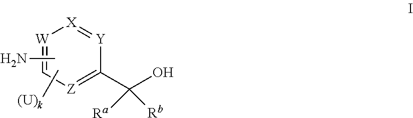

or a pharmaceutically acceptable salt thereof, wherein:
each W, X, Y, or Z is independently selected from N, O, S, CU, CH and C—NH₂, wherein one of W, X, Y, or Z is C—NH₂;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;
k is 0, 1, 2, 3, or 4;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

As defined above and described herein, W is independently selected from N, O, S, CU, CH and C—NH₂. In some embodiments, W is N. In some embodiments, W is O. In some embodiments, W is S. In some embodiments, W is CU. In some embodiments, W is CH. In some embodiments, W is C—NH₂.

As defined above and described herein, X is independently selected from N, O, S, CU, CH and C—NH₂. In some embodiments, X is N. In some embodiments, X is O. In some embodiments, X is S. In some embodiments, X is CU. In some embodiments, X is CH. In some embodiments, X is C—NH₂.

As defined above and described herein, Y is independently selected from N, O, S, CU, CH and C—NH₂. In some embodiments, Y is N. In some embodiments, Y is O. In some embodiments, Y is S. In some embodiments, Y is CU. In some embodiments, Y is CH. In some embodiments, Y is C—NH₂.

As defined above and described herein, Z is independently selected from N, O, S, CU, CH and C—NH₂. In some embodiments, Z is N. In some embodiments, Z is O. In some embodiments, Z is S. In some embodiments, Z is CU. In some embodiments, Z is CH. In some embodiments, Z is C—NH₂.

As defined above and described herein, k is 0, 1, 2, 3, or 4. In some embodiments k is 0. In some embodiments, k is 1. In some embodiments, k is 2. In some embodiments, k is 3. In some embodiments, k is 4.

As defined above and described herein, each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, U is halogen. In some embodiments, U is fluorine. In some embodiments, U is chlorine. In some embodiments, U is bromine.

In some embodiments, U is —R. In some embodiments, U is hydrogen. In some embodiments, U is deuterium. In some embodiments, U is optionally substituted C₁₋₆ aliphatic. In some embodiments, U is an optionally substituted 3- to 8-membered saturated or partially unsaturated monocyclic carbocyclic ring. In some embodiments, U is an optionally substituted 8- to 10-membered bicyclic aryl ring. In some embodiments, U is an optionally substituted 3- to 8-membered saturated or partially unsaturated monocyclic heterocyclic ring having 1-4 heteroatoms independently selected from nitrogen, oxygen, and sulfur. In some embodiments, U is an optionally substituted 5- to 6-membered monocyclic heteroaryl ring having 1-4 heteroatoms independently selected from nitrogen, oxygen, and sulfur. In some embodiments, U is an optionally substituted 6- to 10-membered bicyclic saturated or partially unsaturated heterocyclic ring having 1-5 heteroatoms independently selected from nitrogen, oxygen, and sulfur. In some embodiments, U is an optionally substituted 7- to 10-membered bicyclic heteroaryl ring having 1-5 heteroatoms independently selected from nitrogen, oxygen, and sulfur.

In some embodiments, U is —S(O)₂R. In some embodiments, U is —S(O)₂CH₃.

In some embodiments, U is an optionally substituted phenyl ring. In some embodiments, U is a phenyl ring, optionally substituted with halogen. In some embodiments, U is a phenyl ring, optionally substituted with fluorine. In some embodiments, U is a phenyl ring, optionally substituted with chlorine.

As defined above and described herein, two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur.

In some embodiments, two occurrences of U on adjacent carbon atoms form a fused phenyl ring. In some embodiments, two occurrences of U on adjacent carbon atoms form an optionally substituted fused phenyl ring. In some embodiments, two occurrences of U on adjacent carbon atoms form a fused phenyl ring, optionally substituted with 1 or more halogen atoms. In some embodiments, two occurrences of U on adjacent carbon atoms form a fused phenyl ring, optionally substituted with one halogen atom. In some embodiments, two occurrences of U on adjacent carbon atoms form a fused phenyl ring, optionally substituted with fluorine. In some embodiments, two occurrences of U on adjacent carbon atoms form a fused phenyl ring, optionally substituted with chlorine. In some embodiments, two occurrences of U on adjacent carbon atoms form a fused phenyl ring, optionally substituted with 2 halogen atoms. In some embodiments, two occurrences of U on adjacent carbon atoms form a fused phenyl ring, optionally substituted with 2 fluorines. In some embodiments, two occurrences of U on adjacent carbon atoms form a fused phenyl ring, optionally substituted with 2 chlorines. In some embodiments, two occurrences of U on adjacent carbon atoms form a fused phenyl ring, optionally substituted with fluorine and chlorine.

In some embodiments, two occurrences of U on adjacent carbon atoms form a fused 5- to 6-membered heteroaryl ring containing 1-3 heteroatoms independently selected from nitrogen, oxygen, and sulfur. In some embodiments, two occurrences of U on adjacent carbon atoms form an optionally substituted fused 5- to 6-membered heteroaryl ring containing 1-3 heteroatoms independently selected from nitrogen, oxygen, and sulfur.

In some embodiments, two occurrences of U on adjacent carbon atoms form a fused 5-membered heteroaryl ring containing 1-3 heteroatoms independently selected from nitrogen, oxygen, and sulfur. In some embodiments, two occurrences of U on adjacent carbon atoms form an optionally substituted fused 5-membered heteroaryl ring containing 1-3 heteroatoms independently selected from nitrogen, oxygen, and sulfur.

In some embodiments, two occurrences of U on adjacent carbon atoms form a fused 5-membered heteroaryl ring containing one nitrogen and one oxygen heteroatom. In some embodiments, two occurrences of U on adjacent carbon atoms form an optionally substituted fused 5-membered heteroaryl ring containing one nitrogen and one oxygen heteroatom. In some embodiments, two occurrences of U on adjacent carbon atoms form a fused 5-membered heteroaryl ring containing one nitrogen and one oxygen heteroatom, optionally substituted with phenyl. In some embodiments, two occurrences of U on adjacent carbon atoms form a fused 5-membered heteroaryl ring containing one nitrogen and one oxygen heteroatom, optionally substituted with tosyl. In some embodiments, two occurrences of U on adjacent carbon atoms form a fused 5-membered heteroaryl ring containing one nitrogen and one oxygen heteroatom, optionally substituted with C₁₋₆ aliphatic. In some embodiments, two occurrences of U on adjacent carbon atoms form a fused 5-membered heteroaryl ring containing one nitrogen and one oxygen heteroatom, optionally substituted with C₁₋₆ alkyl. In some embodiments, two occurrences of U on adjacent carbon atoms form a fused 5-membered heteroaryl ring containing one nitrogen and one oxygen heteroatom, optionally substituted with cyclopropyl.

In some embodiments, two occurrences of U on adjacent carbon atoms form a fused 5-membered heteroaryl ring containing one nitrogen and one sulfur heteroatom. In some embodiments, two occurrences of U on adjacent carbon atoms form an optionally substituted fused 5-membered heteroaryl ring containing one nitrogen and one sulfur heteroatom. In some embodiments, two occurrences of U on adjacent carbon atoms form a fused 5-membered heteroaryl ring containing one nitrogen and one sulfur heteroatom, optionally substituted with phenyl.

In some embodiments, two occurrences of U on adjacent carbon atoms form a fused 5-membered heteroaryl ring containing two nitrogen heteroatoms. In some embodiments, two occurrences of U on adjacent carbon atoms form an optionally substituted fused 5-membered heteroaryl ring containing two nitrogen heteroatoms. In some embodiments, two occurrences of U on adjacent carbon atoms form a fused 5-membered heteroaryl ring containing two nitrogen heteroatoms, optionally substituted with phenyl.

In some embodiments, two occurrences of U on adjacent carbon atoms form a fused 6-membered heteroaryl ring containing 1-3 heteroatoms independently selected from nitrogen, oxygen, and sulfur. In some embodiments, two occurrences of U on adjacent carbon atoms form an optionally substituted fused 6-membered heteroaryl ring containing 1-3 heteroatoms independently selected from nitrogen, oxygen, and sulfur.

In some embodiments, two occurrences of U on adjacent carbon atoms form a fused 6-membered heteroaryl ring containing one nitrogen heteroatom. In some embodiments, two occurrences of U on adjacent carbon atoms form an optionally substituted fused 6-membered heteroaryl ring containing one nitrogen heteroatom. In some embodiments, two occurrences of U on adjacent carbon atoms form a fused 6-membered heteroaryl ring containing two nitrogen heteroatoms. In some embodiments, two occurrences of U on adjacent carbon atoms form an optionally substituted fused 6-membered heteroaryl ring containing two nitrogen heteroatoms.

In some embodiments, the fused ring system formed by two occurrences of U on adjacent carbon atoms is quinazolinyl. In some embodiments, the fused ring system formed by two occurrences of U on adjacent carbon atoms is an optionally substituted quinazolinyl.

In some embodiments, the fused ring system formed by two occurrences of U on adjacent carbon atoms is quinolinyl. In some embodiments, the fused ring system formed by two occurrences of U on adjacent carbon atoms is optionally substituted quinolinyl. In some embodiments, the fused ring system formed by two occurrences of U on adjacent carbon atoms is quinolinyl, optionally substituted with 1-2 halogen atoms. In some embodiments, the fused ring system formed by two occurrences of U on adjacent carbon atoms is quinolinyl, optionally substituted with 1 halogen atom. In some embodiments, the fused ring system formed by two occurrences of U on adjacent carbon atoms is quinolinyl, optionally substituted with fluorine. In some embodiments, the fused ring system formed by two occurrences of U on adjacent carbon atoms quinolinyl, optionally substituted with chlorine.

In some embodiments, the fused ring system formed by two occurrences of U on adjacent carbon atoms is benzoxazolyl. In some embodiments, the fused ring system formed by two occurrences of U on adjacent carbon atoms is optionally substituted benzoxazolyl. In some embodiments, the fused ring system formed by two occurrences of U on adjacent carbon atoms is benzoxazolyl, optionally substituted with phenyl. In some embodiments, the fused ring system formed by two occurrences of U on adjacent carbon atoms is benzoxazolyl, optionally substituted with phenyl and a halogen atom. In some embodiments, the fused ring system formed by two occurrences of U on adjacent carbon atoms is benzoxazolyl, optionally substituted with phenyl and chlorine. In some embodiments, the fused ring system formed by two occurrences of U on adjacent carbon atoms is benzoxazolyl, optionally substituted with tosyl and chlorine.

In some embodiments, the fused ring system formed by two occurrences of U on adjacent carbon atoms is benzisoxazolyl. In some embodiments, the fused ring system formed by two occurrences of U on adjacent carbon atoms is optionally substituted benzisoxazolyl. In some embodiments, the fused ring system formed by two occurrences of U on adjacent carbon atoms is benzisoxazolyl, optionally substituted with phenyl. In some embodiments, the fused ring system formed by two occurrences of U on adjacent carbon atoms is benzisoxazolyl, optionally substituted with cyclopropyl and a halogen atom. In some embodiments, the fused ring system formed by two occurrences of U on adjacent carbon atoms is benzisoxazolyl, optionally substituted with cyclopropyl and chlorine.

In some embodiments, the fused ring system formed by two occurrences of U on adjacent carbon atoms is benzothiazolyl. In some embodiments, the fused ring system formed by two occurrences of U on adjacent carbon atoms is optionally substituted benzothiazolyl. In some embodiments, the fused ring system formed by two occurrences of U on adjacent carbon atoms is benzothiazolyl, optionally substituted with phenyl.

In some embodiments, the fused ring system formed by two occurrences of U on adjacent carbon atoms is benzisothiazolyl. In some embodiments, the fused ring system formed by two occurrences of U on adjacent carbon atoms is optionally substituted benzisothiazolyl. In some embodiments, the fused ring system formed by two occurrences of U on adjacent carbon atoms is benzisothiazolyl, optionally substituted with phenyl.

In some embodiments, the fused ring system formed by two occurrences of U on adjacent carbon atoms is benzimidazolyl. In some embodiments, the fused ring system formed by two occurrences of U on adjacent carbon atoms is optionally substituted benzimidazolyl. In some embodiments, the fused ring system formed by two occurrences of U on adjacent carbon atoms is benzimidazolyl, optionally substituted with phenyl.

In some embodiments, W, X, Y, and Z provide a phenyl ring. In some embodiments, W, X, Y, and Z provide a phenyl ring, substituted with k occurrences of U. In some embodiments where W, X, Y, and Z provide a phenyl ring, one of W, X, Y, and Z is —C—NH₂.

In some embodiments, W, X, Y, and Z provide a pyridinyl ring. In some embodiments, W, X, Y, and Z provide a pyridinyl ring, substituted with k occurrences of U. In some embodiments where W, X, Y, and Z provide a pyridinyl ring, one of W, X, Y, and Z is —C—NH₂.

In some embodiments, one of W, X, Y, and Z is —C—NH₂, one or more of the other of W, X, Y, and Z are CH; and k is 0. In some embodiments, one of W, X, and Y is —C—NH₂, one or more of the other of W, X, or Y are CH; Z is N; and k is 0.

In some embodiments, one of W, X, Y, and Z is —C—NH₂, one or more of the other of W, X, Y, and Z are CH; k is 1; and U is halogen. In some embodiments, one of W, X, Y, and Z is —C—NH₂, one or more of the other of W, X, Y, and Z are CH; k is 1; and U is fluorine. In some embodiments, one of W, X, Y, and Z is —C—NH₂, one or more of the other of W, X, Y, and Z are CH; k is 1; and U is chlorine. In some embodiments, one of W, X, Y, and Z is —C—NH₂, one or more of the other of W, X, Y, and Z are CH; k is 1; and U is bromine.

In some embodiments, one of W, X, and Y is —C—NH₂, one or more of the other of W, X, and Y are CH; Z is N; k is 1; and U optionally substituted phenyl. In some embodiments, one of W, X, and Y is —C—NH₂; one or more of the other of W, X, and Y are CH; Z is N; k is 1; and U is phenyl, optionally substituted with halogen. In some embodiments, one of W, X, and Y is —C—NH₂, one or more of the other of W, X, and Y are CH; Z is N; k is 1; and U is phenyl, optionally substituted with chlorine. In some embodiments, one of W, X, and Y is —C—NH₂, one or more of the other of W, X, and Y are CH; Z is N; k is 1; and U is phenyl, optionally substituted with fluorine.

In some embodiments, W is N; one of X, Y, and Z is —C—NH₂; the other of X, Y, and Z are CH; k is 1; and U is optionally substituted phenyl. In some embodiments, W is N; one of X, Y, and Z is —C—NH₂; the other of X, Y, and Z are CH; k is 1; and U is phenyl, optionally substituted with halogen. In some embodiments, W is N; one of X, Y, and Z is —C—NH₂; the other of X, Y, and Z are CH; k is 1; and U is phenyl, optionally substituted with chlorine. In some embodiments, W is N; one of X, Y, and Z is —C—NH₂; the other of X, Y, and Z are CH; k is 1; and U is phenyl, optionally substituted with fluorine.

In some embodiments, one of W, X, and Y is —C—NH₂; one or more of the other of W, X, and Y are CH; Z is N; k is 2; and the two occurrences of U on adjacent carbon atoms form a fused phenyl ring. In some embodiments, one of W, X, and Y is —C—NH₂; one or more of the other of W, X, and Y are CH; Z is N; k is 2; and the two occurrences of U on adjacent carbon atoms form an optionally substituted fused phenyl ring. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with halogen. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with chlorine. In some embodiments, one of W, X, and Y is —C—NH₂; one or more of the other of W, X, and Y are CH; Z is N; k is 2; and the two occurrences of U on adjacent carbon atoms form a fused phenyl ring, optionally substituted with fluorine.

In some embodiments, W is N; one of X, Y, and Z is —C—NH₂; the other of X, Y, and Z are CH; k is 2; and the two occurrences of U on adjacent carbon atoms form a fused phenyl ring. In some embodiments, W is N; one of X, Y, and Z is —C—NH₂; the other of X, Y, and Z are CH; k is 2; and the two occurrences of U on adjacent carbon atoms form an optionally substituted fused phenyl ring. In some embodiments, W is N; one of X, Y, and Z is —C—NH₂; the other of X, Y, and Z are CH; k is 2; and the two occurrences of U on adjacent carbon atoms form a fused phenyl ring, optionally substituted with halogen. In some embodiments, W is N; one of X, Y, and Z is —C—NH₂; the other of X, Y, and Z are CH; k is 2; and the two occurrences of U on adjacent carbon atoms form a fused phenyl ring, optionally substituted with fluorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine and fluorine. In some embodiments, W is N; one of X, Y, and Z is —C—NH₂; the other of X, Y, and Z are CH; k is 2; and the two occurrences of U on adjacent carbon atoms form a fused phenyl ring, optionally substituted with chlorine at 2 positions.

In some embodiments, one of W, X, Y, and Z is —C—NH₂; one or more of the other of W, X, Y, and Z are CH; k is 2; and the two occurrences of U on adjacent carbon atoms form a fused 5- to 6-membered heteroaryl ring containing 1-3 heteroatoms independently selected from nitrogen, oxygen, and sulfur. In some embodiments, one of W, X, Y, and Z is —C—NH₂; one or more of the other of W, X, Y, and Z are CH; k is 2; and the two occurrences of U on adjacent carbon atoms form an optionally substituted fused 5- to 6-membered heteroaryl ring containing 1-3 heteroatoms independently selected from nitrogen, oxygen, and sulfur.

In some embodiments, one of W, X, Y, and Z is —C—NH₂; one or more of the other of W, X, Y, and Z are CH; k is 2; and the two occurrences of U on adjacent carbon atoms form an optionally substituted fused 6-membered heteroaryl ring containing one nitrogen heteroatom. In some embodiments, one of W, X, Y, and Z is —C—NH₂; one or more of the other of W, X, Y, and Z are CH; k is 2; and the two occurrences of U on adjacent carbon atoms form a fused pyridine ring. In some embodiments, one of W, X, Y, and Z is —C—NH₂; one or more of the other of W, X, Y, and Z are CH; k is 2; and the two occurrences of U on adjacent carbon atoms form an optionally substituted fused pyridine ring. In some embodiments, one of W, X, Y, and Z is —C—NH₂; one or more of the other of W, X, Y, and Z are CH; k is 2; and the two occurrences of U on adjacent carbon atoms form an optionally substituted fused 6-membered heteroaryl ring containing two nitrogen heteroatoms. In some embodiments, one of W, X, Y, and Z is —C—NH₂; one or more of the other of W, X, Y, and Z are CH; k is 2; and the two occurrences of U on adjacent carbon atoms form a fused pyrimidine ring. In some embodiments, one of W, X, Y, and Z is —C—NH₂; one or more of the other of W, X, Y, and Z are CH; k is 2; and the two occurrences of U on adjacent carbon atoms form an optionally substituted fused pyrimidine ring.

In some embodiments, one of W, X, Y, and Z is —C—NH₂; one or more of the other of W, X, Y, and Z are CH; k is 2; and the two occurrences of U on adjacent carbon atoms form fused aryl ring with 2 heteroatoms. In some embodiments, one of W, X, Y, and Z is —C—NH₂; one or more of the other of W, X, Y, and Z are CH; k is 2; and the two occurrences of U on adjacent carbon atoms form a 5-membered fused oxazole ring. In some embodiments, one of W, X, Y, and Z is —C—NH₂; one or more of the other of W, X, Y, and Z are CH; k is 2; and the two occurrences of U on adjacent carbon atoms form a 5-membered fused oxazole ring, optionally substituted with phenyl.

In some embodiments, one of W, X, Y, and Z is —C—NH₂; one or more of the other of W, X, Y, and Z is CH; k is 2; and the two occurrences of U on adjacent carbon atoms form an optionally substituted fused 5-membered heteroaryl ring containing one nitrogen and one oxygen heteroatom. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one oxygen heteroatoms, optionally substituted with phenyl. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one oxygen heteroatoms, optionally substituted with tosyl. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one oxygen heteroatoms, optionally substituted with cyclopropyl.

In some embodiments, one of W, X, Y, and Z is —C—NH₂; one or more of the other of W, X, Y, and Z are CH; k is 2; and the two occurrences of U on adjacent carbon atoms form an optionally substituted fused oxazole ring. In some embodiments, one of W, X, Y, and Z is —C—NH₂; one or more of the other of W, X, Y, and Z are CH; k is 2; and the two occurrences of U on adjacent carbon atoms form a fused oxazole ring, optionally substituted with phenyl. In some embodiments, one of W, X, Y, and Z is —C—NH₂; one or more of the other of W, X, Y, and Z are CH; k is 2; and the two occurrences of U on adjacent carbon atoms form a fused oxazole ring, optionally substituted with tosyl.

In some embodiments, one of W, X, Y, and Z is —C—NH₂; one or more of the other of W, X, Y, and Z are CH; k is 2; and the two occurrences of U on adjacent carbon atoms form an optionally substituted fused isoxazole ring. In some embodiments, one of W, X, Y, and Z is —C—NH₂; one or more of the other of W, X, Y, and Z are CH; k is 2; and the two occurrences of U on adjacent carbon atoms form a fused isoxazole ring, optionally substituted with phenyl. In some embodiments, one of W, X, Y, and Z is —C—NH₂; one or more of the other of W, X, Y, and Z are CH; k is 2; and the two occurrences of U on adjacent carbon atoms form a fused isoxazole ring, optionally substituted with cyclopropyl.

In some embodiments, one of W, X, Y, and Z is —C—NH₂; one or more of the other of W, X, Y, and Z are CH; k is 2; and the two occurrences of U on adjacent carbon atoms form an optionally substituted fused 5-membered heteroaryl ring containing one nitrogen and one sulfur heteroatom. In some embodiments, one of W, X, Y, and Z is —C—NH₂; one or more of the other of W, X, Y, and Z is CH; k is 2; and the two occurrences of U on adjacent carbon atoms form a fused 5-membered heteroaryl ring containing one nitrogen and one sulfur heteroatom, optionally substituted by phenyl.

In some embodiments, one of W, X, Y, and Z is —C—NH₂; one or more of the other of W, X, Y, and Z are CH; k is 2; and the two occurrences of U on adjacent carbon atoms form an optionally substituted fused thiazole ring. In some embodiments, one of W, X, Y, and Z is —C—NH₂; one or more of the other of W, X, Y, and Z are CH; k is 2; and the two occurrences of U on adjacent carbon atoms form a fused thiazole ring, optionally substituted with phenyl.

In some embodiments, one of W, X, Y, and Z is —C—NH₂; one or more of the other of W, X, Y, and Z are CH; k is 2; and the two occurrences of U on adjacent carbon atoms form an optionally substituted fused 5 membered heteroaryl ring containing two nitrogen heteroatoms. In some embodiments, one of W, X, Y, and Z is —C—NH₂; one or more of the other of W, X, Y, and Z are CH; k is 2; and the two occurrences of U on adjacent carbon atoms form an optionally substituted fused imidazole ring. In some embodiments, one of W, X, Y, and Z is —C—NH₂; one or more of the other of W, X, Y, and Z are CH; k is 2; and the two occurrences of U on adjacent carbon atoms form a fused imidazole ring, optionally substituted with phenyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused 5-membered heteroaryl ring containing one nitrogen and one oxygen heteroatom. In some embodiments, one of W, X, Y, and Z is —C—NH₂; one or more of the other of W, X, Y, and Z are CH; k is 3; U₁ is chlorine and U₂ and U₃ on adjacent carbon atoms form a fused 5-membered heteroaryl ring containing one nitrogen and one oxygen heteroatom, optionally substituted with phenyl. In some embodiments, one of W, X, Y, and Z is —C—NH₂; one or more of the other of W, X, Y, and Z are CH; k is 3; U₁ is chlorine and U₂ and U₃ on adjacent carbon atoms form a fused 5-membered heteroaryl ring containing one nitrogen and one oxygen heteroatom, optionally substituted with tosyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused oxazole ring. In some embodiments, one of W, X, Y, and Z is —C—NH₂; one or more of the other of W, X, Y, and Z are CH; k is 3; U₁ is chlorine and U₂ and U₃ on adjacent carbon atoms form a fused oxazole ring, optionally substituted with phenyl. In some embodiments, one of W, X, Y, and Z is —C—NH₂; one or more of the other of W, X, Y, and Z are CH; k is 3; U₁ is chlorine and U₂ and U₃ on adjacent carbon atoms form a fused oxazole ring, optionally substituted with tosyl.

In some embodiments, one of W, X, Y, and Z is —C—NH₂; one or more of the other of W, X, Y, and Z are CH; k is 3; U₁ is chlorine and U₂ and U₃ on adjacent carbon atoms form an optionally substituted fused isoxazole ring. In some embodiments, one of W, X, Y, and Z is —C—NH₂; one or more of the other of W, X, Y, and Z are CH; k is 3; U₁ is chlorine and U₂ and U₃ adjacent carbon atoms form a fused isoxazole ring, optionally substituted with cyclopropyl.

As defined above and described herein, each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments, R is hydrogen. In some embodiments, R is deuterium. In some embodiments, R is C₁₋₆ aliphatic. In some embodiments R is methyl. In some embodiments, R is ethyl. In some embodiments, R is optionally substituted C₁₋₆ aliphatic. In some embodiments, R is optionally substituted methyl. In some embodiments, R is optionally substituted ethyl. In some embodiments, R is phenyl. In some embodiments, R is optionally substituted phenyl. In some embodiments, R is phenyl, optionally substituted with halogen. In some embodiments, R is phenyl, optionally substituted with fluorine.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁-4 alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments, R^{a} and R^{b}, taken together with the carbon atom to which they are attached, form a 3- to 8-membered cycloalkyl. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the compound is of formula II:

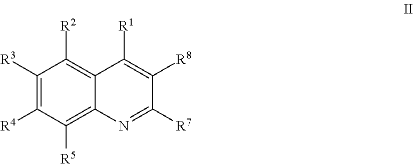

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

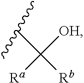

wherein one of R¹, R⁷ and R⁸ is —NH₂ and other one of R¹ R⁷ and R⁸ is

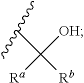

R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments of formula II, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments of formula II, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments of formula II, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments of formula II, R^{b} is C₁₋₄ alkyl. In some embodiments, R^{b} is C₁-4 alkyl optionally substituted with 1, 2, or 3 deuterium or halogen atoms. In some embodiments, R^{b} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments of formula II, R^{a} and R^{b}, taken together with the carbon atom to which they are attached, form a 3- to 8-membered cycloalkyl. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments of formula II, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments of formula II, the —NH₂ on one of R¹, R⁷, and R⁸ and the carbinol on the other of R¹, R⁷, and R⁸ are on adjacent carbon atoms of the pyridine moiety.

In some embodiments, the compound is a compound of formula II-a, II-b, or II-c:

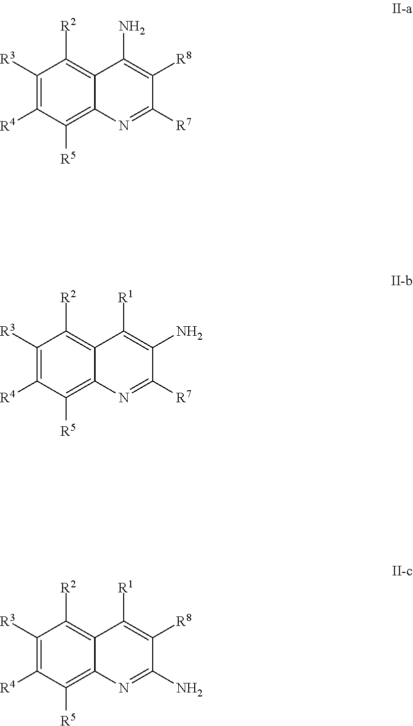

or a pharmaceutically acceptable salt thereof, wherein:
each of R¹, R⁷, and R⁸ when present is independently H, D, halogen, —CN, —OR, —SR, optionally substituted C₁₋₆ aliphatic, or

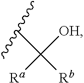

wherein one of R¹, R⁷, and R⁸ is

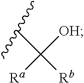

and
R², R³, R⁴, R⁵, R^{a}, R^{b} and R are as defined for formula II.

In some embodiments, the compound is a compound of formula II-d, II-e, II-f or II-g:

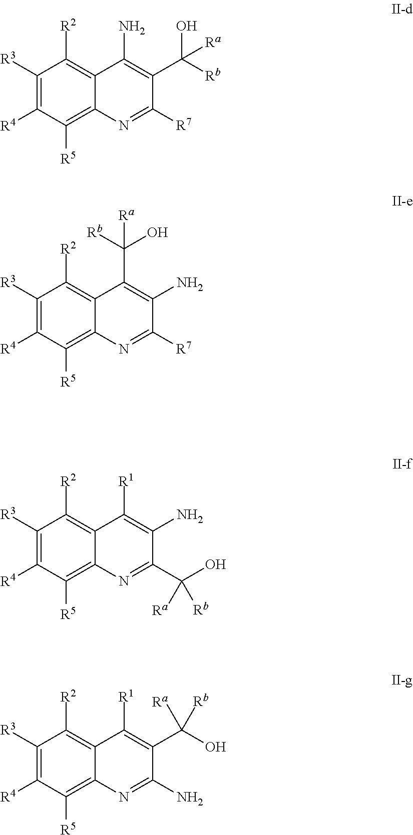

or a pharmaceutically acceptable salt thereof, wherein;
R¹ and R⁷ is independently H, D, halogen, —CN, —OR, —SR, optionally substituted C₁₋₆ aliphatic; and
R², R³, R⁴, R⁵, R^{a}, R^{b} and R are as defined for formula II.

In some embodiments, the compound is a compound of formula III:

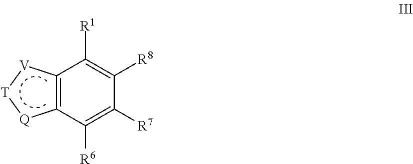

or a pharmaceutically acceptable salt thereof, wherein:
Q, T and V are independently S, N, O, or —C—R;
each of R¹, R⁶, R⁷, and R⁸ is independently H, D, halogen, —NH₂, —CN, —OR, —SR, optionally substituted C₁₋₆ aliphatic, or

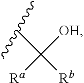

wherein one of R¹, R⁶, R⁷, and R⁸ is —NH₂ and other one of R¹, R⁶, R⁷, and R⁸ is

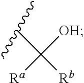

R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms selected from nitrogen, oxygen, and sulfur; and
each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In some embodiments of formula III, the —NH₂ on one of R¹, R⁶, R⁷, and R⁸ and the carbinol on the other of R¹, R⁶, R⁷, and R⁸ are on adjacent carbon atoms of the phenyl moiety.

In some embodiments of formula III, one of Q, T and V is N, and other of Q, T and V is O. In some embodiments, Q is O, V is N, and T is C—R. In some embodiments, Q is N, T is O and V is C—R.

In some embodiments of formula III, the compound is a compound of formula III-a or III-b:

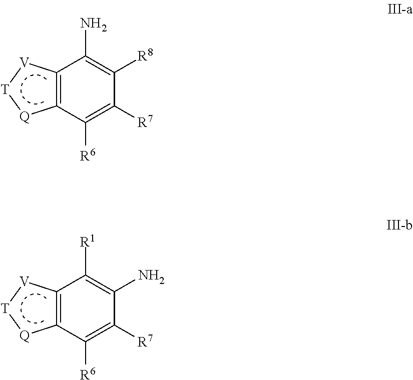

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic, or

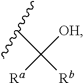

wherein one of R¹, R⁶, R⁷, and R⁸ is

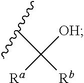

and
Q, T, V, R, R^{a} and R^{b} are as defined in formula III.

In some embodiments of formula III, the compound is a compound of formula III-c, III-d or III-e:

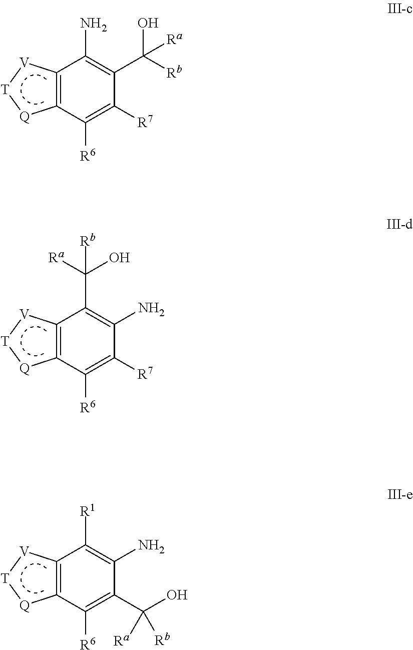

or a pharmaceutically acceptable salt thereof, wherein:
each of R¹, R⁶, and R⁷ when present is independently H, D, halogen, —CN, —OR, —SR, optionally substituted C₁₋₆ aliphatic; and
Q, T, V, R, R^{a} and R^{b} are as defined in formula III.

In some embodiments of formula III, the compound is a compound of formula III-f, III-g, III-h or III-i:

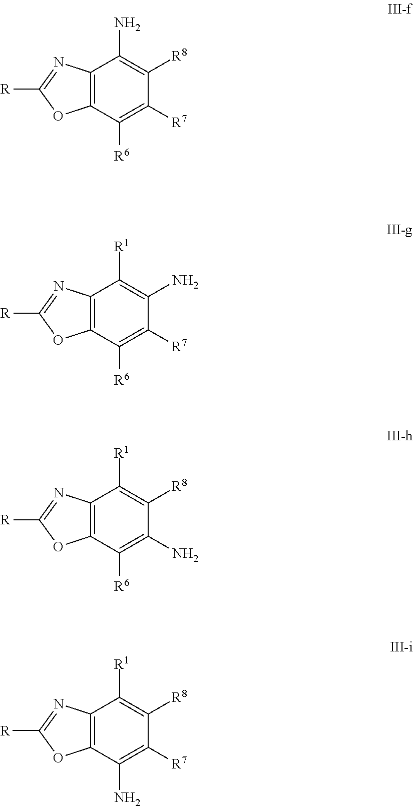

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic or

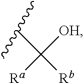

wherein one of R¹, R⁶, R⁷, and R⁸ is

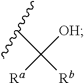

and
R, R^{a} and R^{b} are as defined in formula III.

In some embodiments of formula III, the compound is a compound of formula III-j, III-k, III-l or III-m:

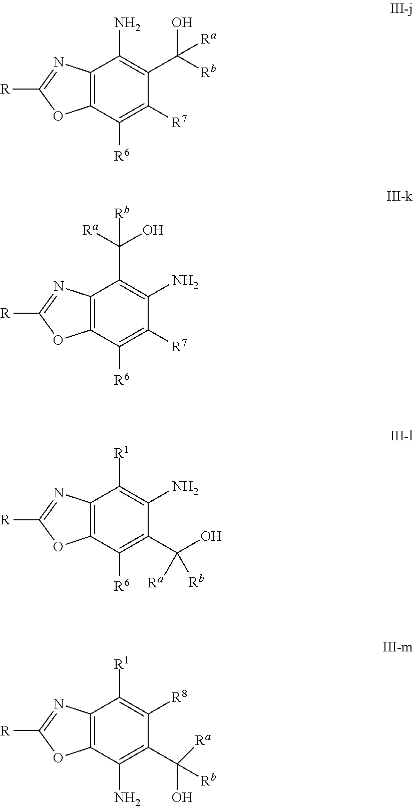

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic; and
R, R^{a} and R^{b} are as defined in formula III.

In some embodiments of formula III, the compound is a compound of formula III-n:

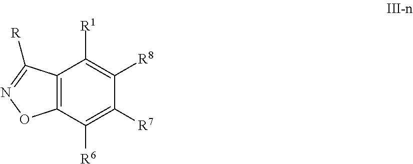

or a pharmaceutically acceptable salt thereof, wherein:
each of R¹, R⁶, R⁷, and R⁸ is independently H, D, halogen, —NH₂, —CN, —OR, —SR, optionally substituted C₁₋₆ aliphatic, or

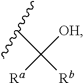

wherein one of R¹, R⁶, R⁷, and R⁸ is —NH₂ and other one of R¹, R⁶, R⁷, and R⁸ is

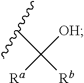

and
R, R^{a}, and R^{b} are as defined in formula III.

In some embodiments of formula III, the compound is a compound of formula III-o, III-p, III-q or III-r:

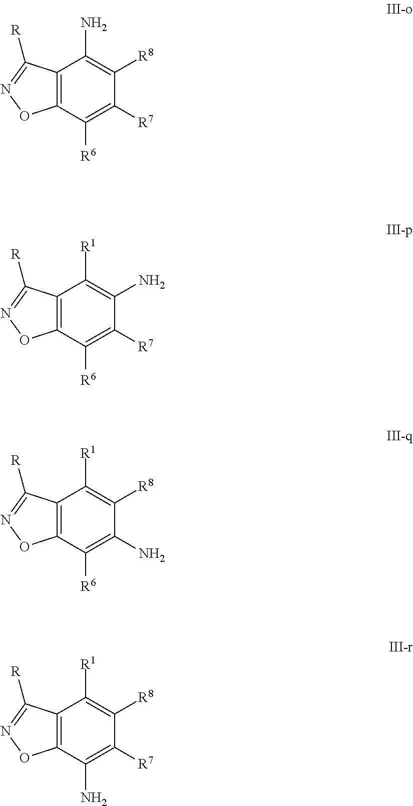

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic, or

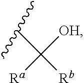

wherein one of R¹, R⁶, R⁷, and R⁸ is

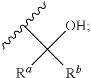

and
R, R^{a}, and R^{b} are as defined in formula III.

In some embodiments of formula III, the compound is a compound of formula III-s, III-t, III-u, III-v, III-w, or III-x:

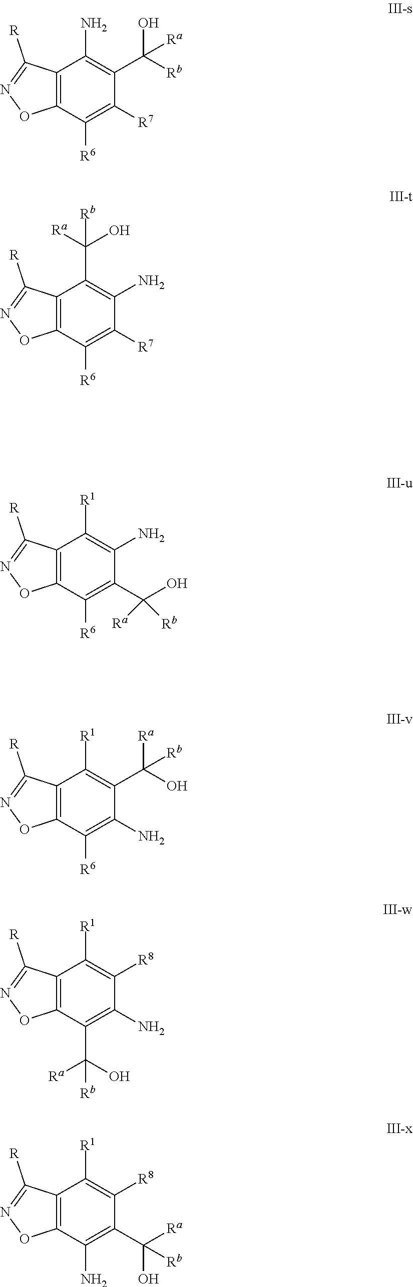

or a pharmaceutically acceptable salt thereof, wherein:
each of R¹, R⁶, R⁷, and R⁸ when present is independently H, D, halogen, —CN, —OR, —SR, optionally substituted C₁₋₆ aliphatic; and
R, R^{a} and R^{b} are as defined in formula III.

In some embodiments, the compound is a compound of formula IV:

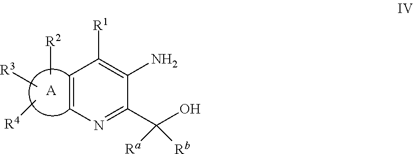

or a pharmaceutically acceptable salt thereof, wherein:
Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, and sulfur; or a 7-membered partially unsaturated heterocyclic or heteroaromatic ring containing 1-3 heteroatoms independently selected from nitrogen, oxygen, and sulfur;
R¹ is H, D, halogen, —CN, —OR, —SR, or optionally substituted C₁₋₆ aliphatic;
each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur;
R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms; or R⁶ and R⁷, taken together with the carbon atom to which they are attached, form a 3- to 8-membered cycloalkyl or heterocyclyl ring containing 1-2 heteroatoms selected from nitrogen, oxygen, and sulfur.

As defined generally above, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, and sulfur; or a 7-membered partially unsaturated heterocyclic or heteroaromatic ring containing 1-3 heteroatoms independently selected from nitrogen, oxygen, and sulfur.

In some embodiments, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom. In some embodiments, Ring A is a 6-membered partially unsaturated heterocyclic or heteroaromatic ring containing 1-3 heteroatoms independently selected from nitrogen, oxygen, and sulfur. In some embodiments, Ring A is a 7-membered partially unsaturated heterocyclic or heteroaromatic ring containing 1-3 heteroatoms independently selected from nitrogen, oxygen, and sulfur.

In some embodiments, Ring A is imidazole or triazole. In some embodiments, Ring A is thiazole. In some embodiments, Ring A is thiophene or furan. In some embodiments, Ring A is pyridine, pyrimidine, pyrazine, pyridazine, or 1,2,4-triazine. In some embodiments, Ring A is pyridine.

As defined generally above, R¹ is H, D, halogen, —CN, —OR, —SR, or optionally substituted C₁₋₆ aliphatic.

In some embodiments, R¹ is H. In some embodiments, R¹ is D. In some embodiments, R¹ is halogen. In some embodiments, R¹ is —CN. In some embodiments, R¹ is —OR. In some embodiments, R¹ is —SR. In some embodiments, R¹ is optionally substituted C₁₋₆ aliphatic.

As described generally above, R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R² is absent. In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3- to 8-membered saturated or partially unsaturated monocyclic carbocyclic ring. In some embodiments, R² is an optionally substituted phenyl. In some embodiments, R² is an optionally substituted 8- to 10-membered bicyclic aryl ring. In some embodiments, R² is an optionally substituted 3- to 8-membered saturated or partially unsaturated monocyclic heterocyclic ring having 1-4 heteroatoms independently selected from nitrogen, oxygen, and sulfur. In some embodiments, R² is an optionally substituted 5- to 6-membered monocyclic heteroaryl ring having 1-4 heteroatoms independently selected from nitrogen, oxygen, and sulfur. In some embodiments, R² is an optionally substituted 6- to 10-membered bicyclic saturated or partially unsaturated heterocyclic ring having 1-5 heteroatoms independently selected from nitrogen, oxygen, and sulfur. In some embodiments, R² is an optionally substituted 7- to 10-membered bicyclic heteroaryl ring having 1-5 heteroatoms independently selected from nitrogen, oxygen, and sulfur.

In some embodiments, R² is Cl or Br. In some embodiments, R² is Cl.

As defined generally above, R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R³ is absent. In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3- to 8-membered saturated or partially unsaturated monocyclic carbocyclic ring. In some embodiments, R³ is an optionally substituted phenyl. In some embodiments, R³ is an optionally substituted 8- to 10-membered bicyclic aryl ring. In some embodiments, R³ is an optionally substituted 3- to 8-membered saturated or partially unsaturated monocyclic heterocyclic ring having 1-4 heteroatoms independently selected from nitrogen, oxygen, and sulfur. In some embodiments, R³ is an optionally substituted 5- to 6-membered monocyclic heteroaryl ring having 1-4 heteroatoms independently selected from nitrogen, oxygen, and sulfur. In some embodiments, R³ is an optionally substituted 6- to 10-membered bicyclic saturated or partially unsaturated heterocyclic ring having 1-5 heteroatoms independently selected from nitrogen, oxygen, and sulfur. In some embodiments, R³ is an optionally substituted 7- to 10-membered bicyclic heteroaryl ring having 1-5 heteroatoms independently selected from nitrogen, oxygen, and sulfur.

In some embodiments, R³ is Cl or Br. In some embodiments, R³ is Cl.

As defined generally above, R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁴ is absent. In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3- to 8-membered saturated or partially unsaturated monocyclic carbocyclic ring. In some embodiments, R⁴ is an optionally substituted phenyl. In some embodiments, R⁴ is an optionally substituted 8- to 10-membered bicyclic aryl ring. In some embodiments, R⁴ is an optionally substituted 3- to 8-membered saturated or partially unsaturated monocyclic heterocyclic ring having 1-4 heteroatoms independently selected from nitrogen, oxygen, and sulfur. In some embodiments, R⁴ is an optionally substituted 5- to 6-membered monocyclic heteroaryl ring having 1-4 heteroatoms independently selected from nitrogen, oxygen, and sulfur. In some embodiments, R⁴ is an optionally substituted 6- to 10-membered bicyclic saturated or partially unsaturated heterocyclic ring having 1-5 heteroatoms independently selected from nitrogen, oxygen, and sulfur. In some embodiments, R⁴ is an optionally substituted 7- to 10-membered bicyclic heteroaryl ring having 1-5 heteroatoms independently selected from nitrogen, oxygen, and sulfur.

In some embodiments, R⁴ is Cl or Br. In some embodiments, R⁴ is Cl.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁-4 alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments, R^{a} and R^{b}, taken together with the carbon atom to which they are attached, form a 3- to 8-membered cycloalkyl. In some embodiments, R^{a} and R^{b}, taken together with the carbon atom to which they are attached, form a 3- to 8-membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the compound is of formula V:

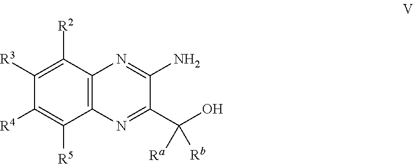

or a pharmaceutically acceptable salt therefor, wherein:
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
each R is independently selected from hydrogen, deuterium, and an optionally substituted group selected from: C₁₋₆ aliphatic, a 3- to 8-membered saturated or partially unsaturated monocyclic carbocyclic ring, phenyl, an 8- to 10-membered bicyclic aryl ring, a 3- to 8-membered saturated or partially unsaturated monocyclic heterocyclic ring having 1-4 heteroatoms independently selected from nitrogen, oxygen, and sulfur, a 5- to 6-membered monocyclic heteroaryl ring having 1-4 heteroatoms independently selected from nitrogen, oxygen, and sulfur, a 6- to 10-membered bicyclic saturated or partially unsaturated heterocyclic ring having 1-5 heteroatoms independently selected from nitrogen, oxygen, and sulfur; or a 7- to 10-membered bicyclic heteroaryl ring having 1-5 heteroatoms independently selected from nitrogen, oxygen, and sulfur;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

As described generally above, R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3- to 8-membered saturated or partially unsaturated monocyclic carbocyclic ring. In some embodiments, R² is an optionally substituted phenyl. In some embodiments, R² is an optionally substituted 8- to 10-membered bicyclic aryl ring. In some embodiments, R² is an optionally substituted 3- to 8-membered saturated or partially unsaturated monocyclic heterocyclic ring having 1-4 heteroatoms independently selected from nitrogen, oxygen, and sulfur. In some embodiments, R² is an optionally substituted 5- to 6-membered monocyclic heteroaryl ring having 1-4 heteroatoms independently selected from nitrogen, oxygen, and sulfur. In some embodiments, R² is an optionally substituted 6- to 10-membered bicyclic saturated or partially unsaturated heterocyclic ring having 1-5 heteroatoms independently selected from nitrogen, oxygen, and sulfur. In some embodiments, R² is an optionally substituted 7- to 10-membered bicyclic heteroaryl ring having 1-5 heteroatoms independently selected from nitrogen, oxygen, and sulfur.

In some embodiments, R² is Cl or Br. In some embodiments, R² is Cl.

As defined generally above, R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3- to 8-membered saturated or partially unsaturated monocyclic carbocyclic ring. In some embodiments, R³ is an optionally substituted phenyl. In some embodiments, R³ is an optionally substituted 8- to 10-membered bicyclic aryl ring. In some embodiments, R³ is an optionally substituted 3- to 8-membered saturated or partially unsaturated monocyclic heterocyclic ring having 1-4 heteroatoms independently selected from nitrogen, oxygen, and sulfur. In some embodiments, R³ is an optionally substituted 5- to 6-membered monocyclic heteroaryl ring having 1-4 heteroatoms independently selected from nitrogen, oxygen, and sulfur. In some embodiments, R³ is an optionally substituted 6- to 10-membered bicyclic saturated or partially unsaturated heterocyclic ring having 1-5 heteroatoms independently selected from nitrogen, oxygen, or sulfur. In some embodiments, R³ is an optionally substituted 7- to 10-membered bicyclic heteroaryl ring having 1-5 heteroatoms independently selected from nitrogen, oxygen, and sulfur.

In some embodiments, R³ is Cl or Br. In some embodiments, R³ is Cl.

As defined generally above, R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3- to 8-membered saturated or partially unsaturated monocyclic carbocyclic ring. In some embodiments, R⁴ is an optionally substituted phenyl. In some embodiments, R⁴ is an optionally substituted 8- to 10-membered bicyclic aryl ring. In some embodiments, R⁴ is an optionally substituted 3- to 8-membered saturated or partially unsaturated monocyclic heterocyclic ring having 1-4 heteroatoms independently selected from nitrogen, oxygen, and sulfur. In some embodiments, R⁴ is an optionally substituted 5- to 6-membered monocyclic heteroaryl ring having 1-4 heteroatoms independently selected from nitrogen, oxygen, and sulfur. In some embodiments, R⁴ is an optionally substituted 6- to 10-membered bicyclic saturated or partially unsaturated heterocyclic ring having 1-5 heteroatoms independently selected from nitrogen, oxygen, and sulfur. In some embodiments, R⁴ is an optionally substituted 7- to 10-membered bicyclic heteroaryl ring having 1-5 heteroatoms independently selected from nitrogen, oxygen, and sulfur.

In some embodiments, R⁴ is Cl or Br. In some embodiments, R⁴ is Cl

As defined generally above, R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R.

In some embodiments, R⁵ is —R. In some embodiments, R⁵ is halogen. In some embodiments, R⁵ is —CN. In some embodiments, R⁵ is —OR. In some embodiments, R⁵ is —SR. In some embodiments, R⁵ is —N(R)₂. In some embodiments, R⁵ is —N(R)C(O)R. In some embodiments, R⁵ is —C(O)N(R)₂. In some embodiments, R⁵ is —N(R)C(O)N(R)₂. In some embodiments, R⁵ is —N(R)C(O)OR. In some embodiments, R⁵ is —OC(O)N(R)₂. In some embodiments, R⁵ is —N(R)S(O)₂R. In some embodiments, R⁵ is —SO₂N(R)₂. In some embodiments, R⁵ is —C(O)R. In some embodiments, R⁵ is —C(O)OR. In some embodiments, R⁵ is —OC(O)R. In some embodiments, R⁵ is —S(O)R. In some embodiments, R⁵ is —S(O)₂R.

In some embodiments, R⁵ is hydrogen. In some embodiments, R⁵ is deuterium. In some embodiments, R⁵ is an optionally substituted C₁₋₆ aliphatic. In some embodiments, R⁵ is an optionally substituted 3- to 8-membered saturated or partially unsaturated monocyclic carbocyclic ring. In some embodiments, R⁵ is an optionally substituted phenyl. In some embodiments, R⁵ is an optionally substituted 8- to 10-membered bicyclic aryl ring. In some embodiments, R⁵ is an optionally substituted 3- to 8-membered saturated or partially unsaturated monocyclic heterocyclic ring having 1-4 heteroatoms independently selected from nitrogen, oxygen, and sulfur. In some embodiments, R⁵ is an optionally substituted 5- to 6-membered monocyclic heteroaryl ring having 1-4 heteroatoms independently selected from nitrogen, oxygen, and sulfur. In some embodiments, R⁵ is an optionally substituted 6- to 10-membered bicyclic saturated or partially unsaturated heterocyclic ring having 1-5 heteroatoms independently selected from nitrogen, oxygen, and sulfur. In some embodiments, R⁵ is an optionally substituted 7- to 10-membered bicyclic heteroaryl ring having 1-5 heteroatoms independently selected from nitrogen, oxygen, and sulfur.

In some embodiments, R⁵ is Cl or Br. In some embodiments, R⁵ is Cl.

As described generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{a} is methyl or ethyl optionally substituted with 1, 2, or 3 halogen atoms. In some embodiments, R^{a} is methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{a} is C₁₋₄ alkyl optionally substituted with 1, 2, or 3 halogen atoms. In some embodiments, R^{b} is methyl or ethyl optionally substituted with 1, 2, or 3 halogen atoms. In some embodiments, R^{b} is methyl.

As defined generally above, in some embodiments, R^{a} and R^{b}, taken together with the carbon atom to which they are attached, form a 3- to 8-membered cycloalkyl or heterocyclyl ring containing 1-2 heteroatoms selected from nitrogen, oxygen, and sulfur.

In some embodiments, R^{a} and R^{b}, taken together with the carbon atom to which they are attached, form a 3- to 8-membered cycloalkyl. In some embodiments, R^{a} and R^{b}, taken together with the carbon atom to which they are attached, form a 3- to - membered heterocyclyl ring containing 1-2 heteroatoms selected from nitrogen, oxygen, and sulfur.

In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are methyl.

In some embodiments, the compound is of formula VI-a, VI-b, VI-c, or VI-d:

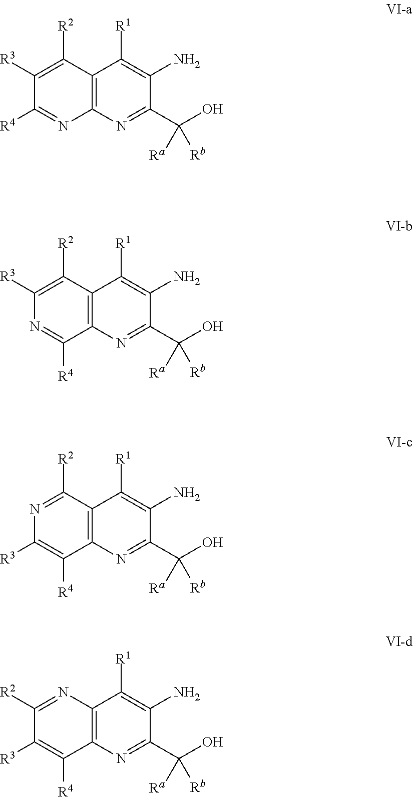

or a pharmaceutically acceptable salt thereof, wherein:
each of R, R¹, R², R³, R⁴, R^{a}, and R^{b} is as defined is as defined above and described in embodiments herein, both singly and in combination.

In some embodiments, the compound is of formula VI-a above.

In some embodiments, R¹ and R⁴ are H.

In some embodiments, R² is H.

In some embodiments, R^{a} and R^{b} are C₁₋₄ alkyl optionally substituted with 1, 2, or 3 deuterium or halogen atoms, or R^{a} and R^{b} are taken together with the carbon to which they are attached to form a 3-8 membered cycloalkyl ring.

In some embodiments, R³ is H, C₁₋₄ alkyl, halogen, —NR, —OR, —SR, —CO₂R, or —C(O)R, wherein R is H, optionally substituted C₁₋₄ alkyl, or optionally substituted phenyl.

In another aspect, the compound is of formula VI-e, VI-f, VI-g, or VI-h:

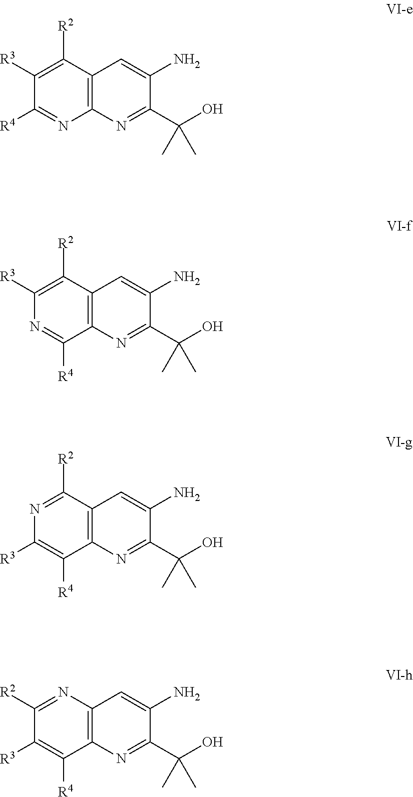

or a pharmaceutically acceptable salt thereof, wherein:
each of R, R¹, R², R³, and R⁴ is as defined is as defined above and described in embodiments herein, both singly and in combination.

In another aspect, the compound is a compound of formula VI-i, VI-j, VI-k, VI-l, VI-m, or VI-n:

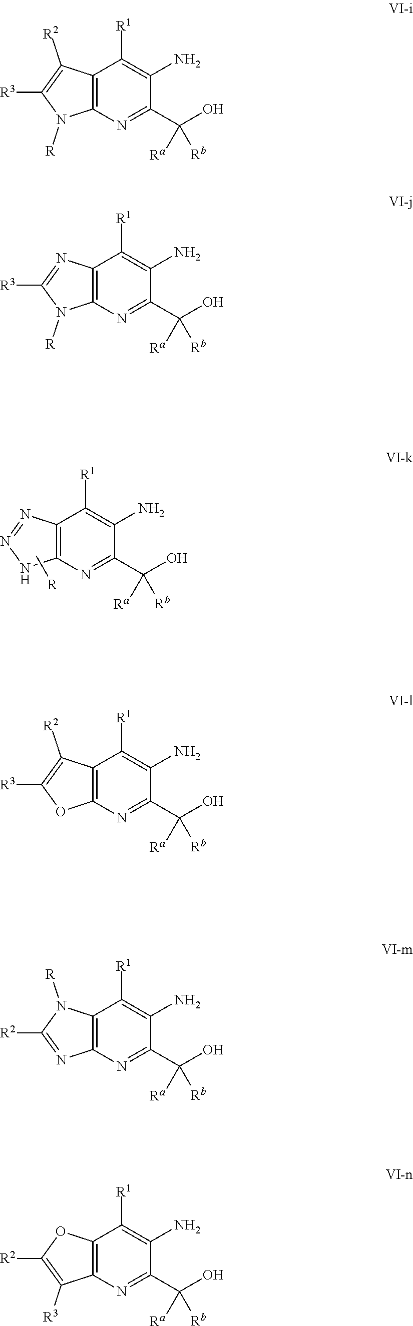

or a pharmaceutically acceptable salt thereof, wherein:
each of R, R¹, R², R³, R⁴, R⁶, and R⁷ is as defined is as defined above and described in embodiments herein, both singly and in combination.

In another aspect, the compound is a compound of formula VII-a:

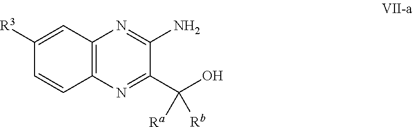

or a pharmaceutically acceptable salt thereof, wherein:
each of R, R³, R⁶, and R⁷ is as defined is as defined above and described in embodiments herein, both singly and in combination.

In some embodiments, the compound is a compound of formula I selected from those depicted in Table 1, below:

TABLE 1
Exemplary Compounds of Formula I

| | |
|---|---|
| 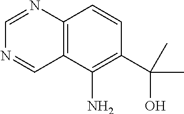 | I-1 |
| 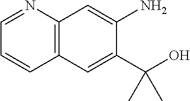 | I-2 |
| 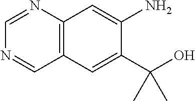 | I-3 |
| 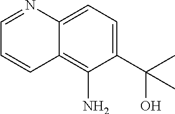 | I-4 |
| 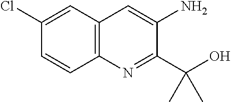 [SMILES: CC(C)(O)c1nc2ccc(Cl)cc2cc1N] | I-5 |
| 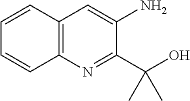 | I-6 |
| 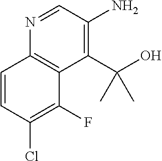 [SMILES: CC(C)(O)c1c(N)cnc2ccc(Cl)c(F)c12] | I-7 |
| 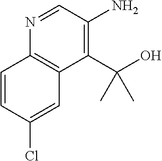 [SMILES: CC(C)(O)c1c(N)cnc2ccc(Cl)cc12] | I-8 |
| 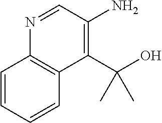 | I-9 |
| 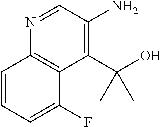 [SMILES: CC(C)(O)c1c(N)cnc2cccc(F)c12] | I-10 |
| 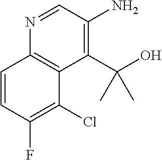 [SMILES: CC(C)(O)c1c(N)cnc2ccc(F)c(Cl)c12] | I-11 |
| 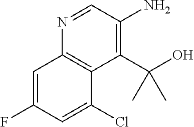 [SMILES: CC(C)(O)c1c(N)cnc2cc(F)cc(Cl)c12] | I-12 |
| 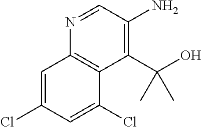 [SMILES: CC(C)(O)c1c(N)cnc2cc(Cl)cc(Cl)c12] | I-13 |
| 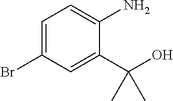 | I-14 |
| 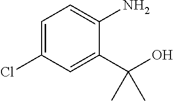 | I-15 |
| 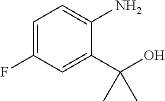 | I-16 |
| 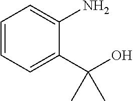 | I-17 |
| 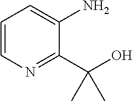 | I-18 |
| 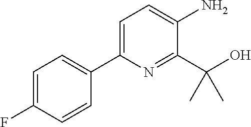 [SMILES: CC(C)(O)c1nc(-c2ccc(F)cc2)ccc1N] | I-19 |
| 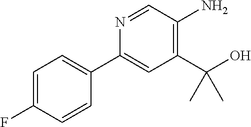 [SMILES: CC(C)(O)c1cc(-c2ccc(F)cc2)ncc1N] | I-20 |
| 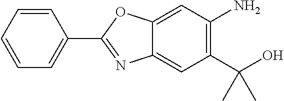 [SMILES: CC(C)(O)c1cc2nc(-c3ccccc3)oc2cc1N] | I-21 |
| 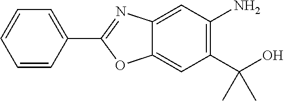 [SMILES: CC(C)(O)c1cc2oc(-c3ccccc3)nc2cc1N] | I-22 |
| 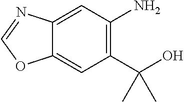 | I-23 |
| 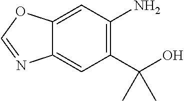 | I-24 |
| 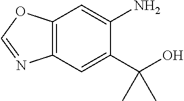 | I-25 |
| 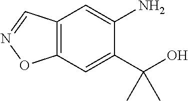 | I-26 |
| 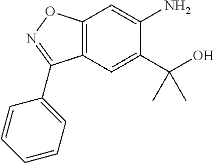 [SMILES: CC(C)(O)c1cc2c(-c3ccccc3)noc2cc1N] | I-27 |
| 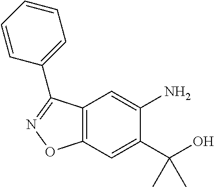 [SMILES: CC(C)(O)c1cc2onc(-c3ccccc3)c2cc1N] | I-28 |
| 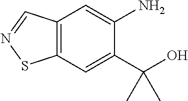 | I-29 |
| 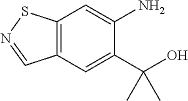 | I-30 |
| 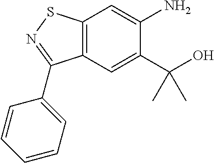 [SMILES: CC(C)(O)c1cc2c(-c3ccccc3)nsc2cc1N] | I-31 |
| 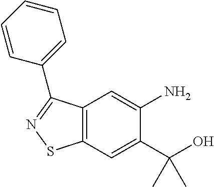 [SMILES: CC(C)(O)c1cc2snc(-c3ccccc3)c2cc1N] | I-32 |
| 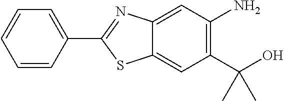 [SMILES: CC(C)(O)c1cc2sc(-c3ccccc3)nc2cc1N] | I-33 |
| 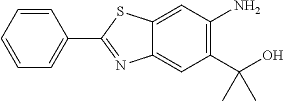 [SMILES: CC(C)(O)c1cc2nc(-c3ccccc3)sc2cc1N] | I-34 |
| 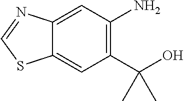 | I-35 |
| 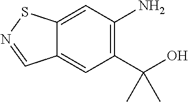 | I-36 |
| 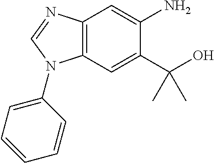 [SMILES: CC(C)(O)c1cc2c(cc1N)ncn2-c1ccccc1] | I-37 |
| 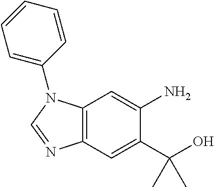 [SMILES: CC(C)(O)c1cc2ncn(-c3ccccc3)c2cc1N] | I-38 |
| 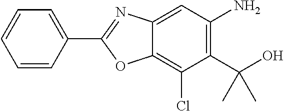 [SMILES: CC(C)(O)c1c(N)cc2nc(-c3ccccc3)oc2c1Cl] | I-39 |
| 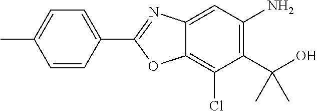 [SMILES: Cc1ccc(-c2nc3cc(N)c(C(C)(C)O)c(Cl)c3o2)cc1] | I-40 |
| 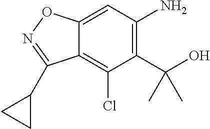 [SMILES: CC(C)(O)c1c(N)cc2onc(C3CC3)c2c1Cl] | I-41 |
| 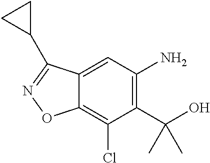 [SMILES: CC(C)(O)c1c(N)cc2c(C3CC3)noc2c1Cl] | I-42 |
| 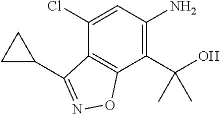 [SMILES: CC(C)(O)c1c(N)cc(Cl)c2c(C3CC3)noc12] | I-43 |
| 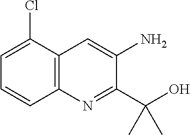 [SMILES: CC(C)(O)c1nc2cccc(Cl)c2cc1N] | I-44 |
| 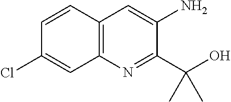 [SMILES: CC(C)(O)c1nc2cc(Cl)ccc2cc1N] | I-45 |
| 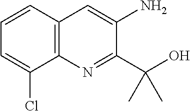 [SMILES: CC(C)(O)c1nc2c(Cl)cccc2cc1N] | I-46 |
| 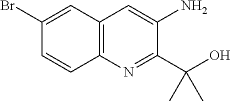 [SMILES: CC(C)(O)c1nc2ccc(Br)cc2cc1N] | I-47 |
| 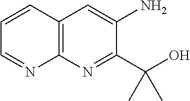 | I-48 |
| 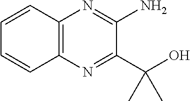 | I-49 |
| 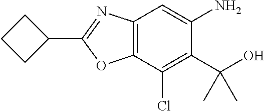 [SMILES: CC(C)(O)c1c(N)cc2nc(C3CCC3)oc2c1Cl] | I-50 |
| 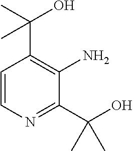 | I-51 |
| 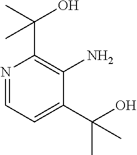 | I-52 |

In some embodiments, the compound is selected from

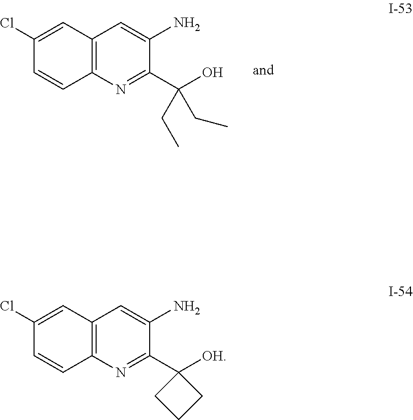 [SMILES: CCC(O)(CC)c1nc2ccc(Cl)cc2cc1N ; Nc1cc2cc(Cl)ccc2nc1C1(O)CCC1]

In some embodiments, compound is a compound of formula VIII:

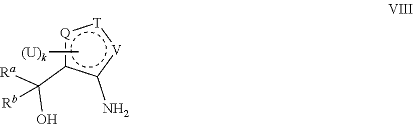

or a pharmaceutically acceptable salt thereof, wherein:
each Q, T, and V is independently selected from N or NH, S, O, CU, and CH;

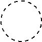

represents two double bonds within the ring, which comply with the valency requirements of the atoms and heteroatoms present in the ring;
k is 0, 1, 2, 3, or 4;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or
R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; and a fused 5- to 6-membered heteroaryl ring containing 1-3 heteroatoms independently selected from nitrogen, oxygen, and sulfur; and
each R is independently selected from hydrogen, deuterium, or an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, or sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; or a 7- to 10-membered bicyclic heteroaryl ring having 1-5 heteroatoms independently selected from nitrogen, oxygen, and sulfur.

Each k, U, and R is as defined and described above.

As defined above and described herein, Q is selected from N or NH, S, O, CU, and CH. In some embodiments, Q is selected from N or NH, S, O, CU, and CH. In some embodiments, Q is N or NH. In some embodiments, Q is S. In some embodiments, Q is O. In some embodiments, Q is CU. In some embodiments, Q is CH.

As defined above and described herein, T is selected from N or NH, S, O, CU, and CH. In some embodiments, T is selected from N or NH, S, O, CU, and CH. In some embodiments, T is N or NH. In some embodiments, T is S. In some embodiments, T is O. In some embodiments, T is CU. In some embodiments, T is CH.

As defined above and described herein, V is selected from N or NH, S, O, CU, and CH. In some embodiments, V is selected from N or NH, S, O, CU, and CH. In some embodiments, V is N or NH. In some embodiments, V is S. In some embodiments, V is O. In some embodiments, V is CU. In some embodiments, V is CH.

As defined above and described herein, k is 0, 1, 2, 3, or 4. In some embodiments k is 0. In some embodiments, k is 1. In some embodiments, k is 2. In some embodiments, k is 3. In some embodiments, k is 4.

As defined above and described herein,

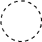

represents two double bonds within the ring, which comply with the valency requirements of the atoms and heteroatoms present in the ring. In some embodiments, the ring formed is thiophene. In some embodiments, the ring formed is oxazole. In some embodiments, the ring formed is isothiazole.

In some embodiments, one or more of Q and V are CH; T is S;

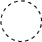

is arranged to form a thiophene; and k is 0. In some embodiments, one or more of Q is CH; T is N or NH; V is O;

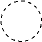

is arranged to form an isoxazole; and k is 0. In some embodiments, one or more of Q is S; T and V are CH;

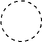

is arranged to form a thiophene; k is 1; and U is —S(O)₂R. In some embodiments, one or more of Q is S; T and V are CH;

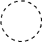

is arranged to form a thiophene; k is 1; and U is —S(O)₂CH₃. In some embodiments, one or more of Q is CH; T is N or NH; V is S;

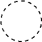

is arranged to form an isothiazole; and k is 0.

In some embodiments, the compound of formula VIII is selected from those depicted in Table 2, below:

TABLE 2
Exemplary Compounds of Formula VIII

| | |
|---|---|
| 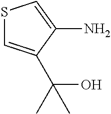 | VIII-1 |
| 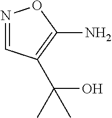 | VIII-2 |
| 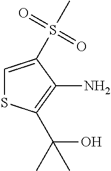 | VIII-3 |
| 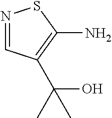 | VIII-4 |

In some embodiments, the compound is a compound of formula IX-A or IX-B:

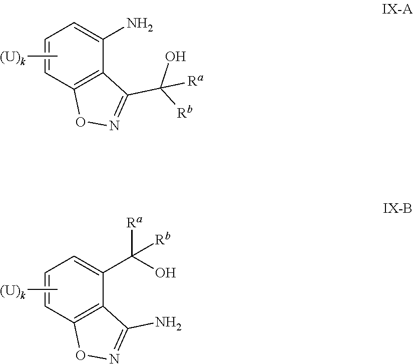

or a pharmaceutically acceptable salt thereof, wherein:
k is 0, 1, 2, 3, or 4;
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms; or
R^{a} and R^{b}, taken together with the carbon atom to which they are attached, form a 3- to 8-membered cycloalkyl or heterocyclyl ring containing 1-2 heteroatoms selected from nitrogen, oxygen, and sulfur;
each U is independently selected from halogen, cyano, —R, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
two occurrences of U on adjacent carbon atoms can form an optionally substituted fused ring, selected from a fused phenyl ring; a fused 5- to 6-membered saturated or partially unsaturated heterocyclic ring containing 1-3 heteroatoms independently selected from nitrogen, oxygen, and sulfur; or a fused 5- to 6-membered heteroaryl ring containing 1-3 heteroatoms independently selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

Each of k, U, and R is as defined and described above.

In some embodiments, the compound is a compound of formulae IX-A or IX-B selected from those depicted in Table 3, below:

TABLE 3
Exemplary Compounds of Formula IX

| | |
|---|---|
| 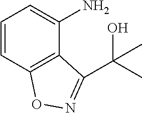 | IX-1 |
| 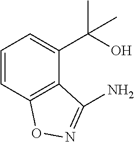 | IX-2 |

Deuterated Compounds

In some embodiments, the compound is a deuterated form of a compound above or a pharmaceutically acceptable salt thereof.

In some embodiments, the compound is a compound of formula X:

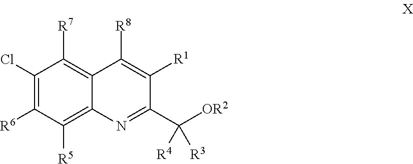

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formula X-A:

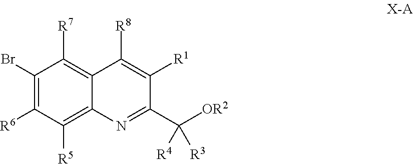

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formulae XI-A or XI-B:

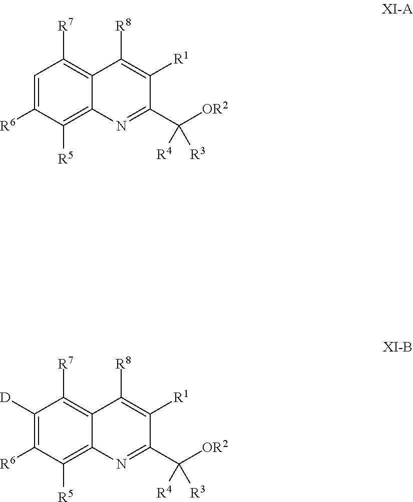

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ in formula XI-A is or contains deuterium.

In some embodiments, the compound is a compound of formulae XII-A, XII-B, or XII-C:

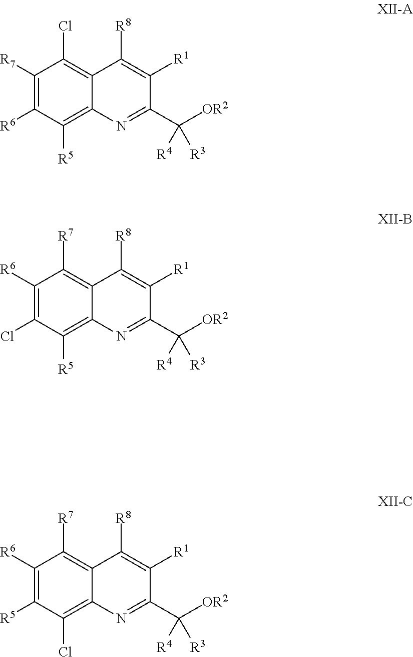

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formula XIII:

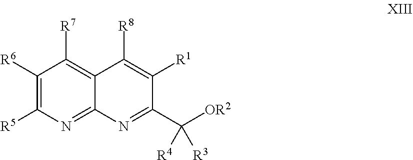

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formula XIV:

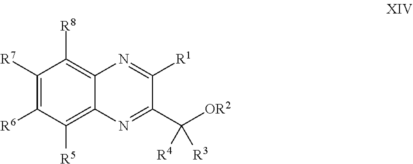

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formulae XV-A or XV-B:

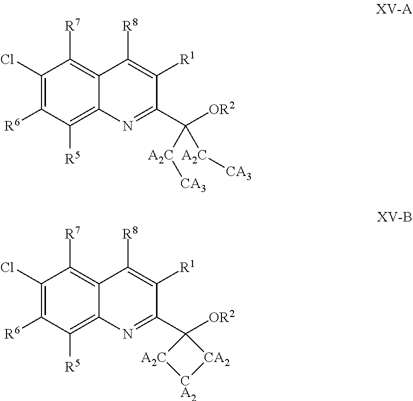

or a pharmaceutically acceptable salt thereof, wherein:
each A is independently hydrogen or deuterium;
R¹ is selected from —NH₂, —NHD, or —ND₂;
R² is selected from hydrogen or deuterium; and
R⁵, R⁶, R⁷, and R⁸ are each independently selected from hydrogen or deuterium; provided that at least one of A, R¹, R², R⁵, R⁶, R⁷, or R⁸ is or contains deuterium.

In some embodiments, the compound is a compound of formulae XVI-A or XVI-B:

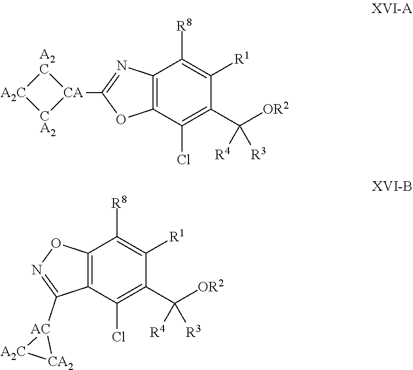

or a pharmaceutically acceptable salt thereof, wherein:
each A is independently hydrogen or deuterium;
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁸ is selected from hydrogen or deuterium;

In some embodiments, the compound is a compound of formulae XVII-A or XVII-B:

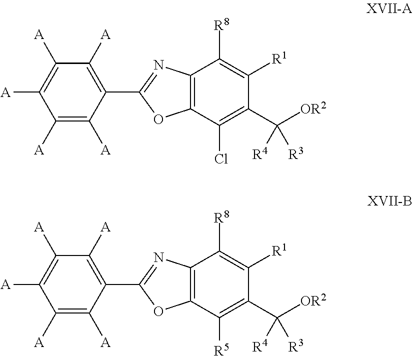

or a pharmaceutically acceptable salt thereof, wherein:
each A is independently hydrogen or deuterium;
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵ and R⁸ are each independently selected from hydrogen or deuterium; provided that at least one of A, R¹, R², R³, R⁴, R⁵, or R⁸ is or contains deuterium.

In some embodiments, the compound is a compound of formula XVIII-A or XVIII-B:

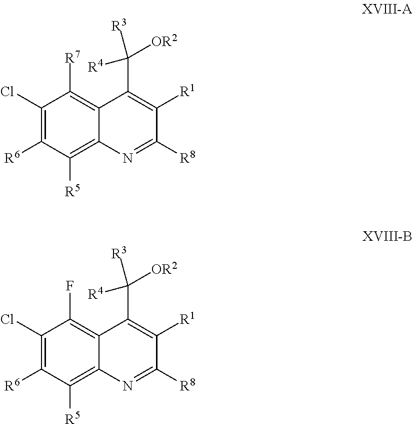

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵, R⁶, R⁷, and R⁸ are each independently selected from hydrogen or deuterium; provided that at least one of R¹, R², R³, R⁴, R⁵, R⁶, R⁷, or R⁸ is or contains deuterium.

In some embodiments, the compound is a compound of formula XIX:

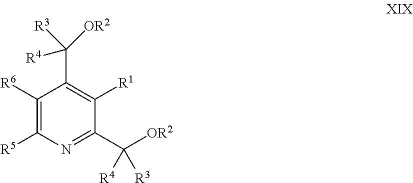

or a pharmaceutically acceptable salt thereof, wherein:
R¹ is selected from —NH₂, —NHD, or —ND₂;
R² is selected from hydrogen or deuterium;
R³ and R⁴ are independently selected from —CH₃, —CH₂D, —CHD₂, or —CD₃; and
R⁵ and R⁶ are each independently selected from hydrogen or deuterium; provided that at least one of R¹, R², R³, R⁴, R⁵, or R⁶ is or contains deuterium.

The following embodiments are applicable to each of the preceding formulae X-XIX.

As defined above and described herein, R¹ is selected from —NH₂, —NHD, or —ND₂.

In some embodiments, R¹ is —NH₂. In some embodiments, R¹ is —NH₂ and at least one of R², R³, R⁴, R⁵, R⁶, R⁷, or R⁸ is or contains deuterium.

In some embodiments, R¹ is —NHD. In some embodiments, R¹ is —NHD and at least one of R², R³, R⁴, R⁵, R⁶, R⁷, or R⁸ is or contains deuterium.

In some embodiments, R¹ is —ND₂. In some embodiments, R¹ is —ND₂ and at least one of R², R³, R⁴, R⁵, R⁶, R⁷, or R⁸ is or contains deuterium.

As defined above and described herein, A is selected from hydrogen or deuterium.

In some embodiments, A is hydrogen. In some embodiments, A is hydrogen and at least one of R¹, R³, R⁴, R⁵, R⁶, R⁷, or R⁸ is or contains deuterium. In some embodiments, A is deuterium. In some embodiments, A is deuterium and at least one of R¹, R³, R⁴, R⁵, R⁶, R⁷, or R⁸ is or contains deuterium.

As defined above and described herein, R² is selected from hydrogen or deuterium.

In some embodiments, R² is hydrogen. In some embodiments, R² is hydrogen and at least one of R¹, R³, R⁴, R⁵, R⁶, R⁷, or R⁸ is or contains deuterium. In some embodiments, R² is deuterium. In some embodiments, R² is deuterium and at least one of R¹, R³, R⁴, R⁵, R⁶, R⁷, or R⁸ is or contains deuterium.

As defined above and described herein, R³ is selected from —CH₃, —CH₂D, —CHD₂, or —CD₃.

In some embodiments, R³ is —CH₃. In some embodiments, R³ is —CH₃ and at least one of R¹, R², R⁴, R⁵, R⁶, R⁷, or R⁸ is or contains deuterium.

In some embodiments, R³ is —CH₂D. In some embodiments, R³ is —CH₂D and at least one of R¹, R², R⁴, R⁵, R⁶, R⁷, or R⁸ is or contains deuterium.

In some embodiments, R³ is —CHD₂. In some embodiments, R³ is —CHD₂ and at least one of R¹, R², R⁴, R⁵, R⁶, R⁷, or R⁸ is or contains deuterium.

In some embodiments, R³ is —CD₃. In some embodiments, R³ is —CD₃ and at least one of R¹, R², R⁴, R⁵, R⁶, R⁷, or R⁸ is or contains deuterium.

As defined above and described herein, R⁴ is selected from —CH₃, —CH₂D, —CHD₂, or —CD₃.

In some embodiments, R⁴ is —CH₃. In some embodiments, R⁴ is —CH₃ and at least one of R¹, R², R³, R⁵, R⁶, R⁷, or R⁸ is or contains deuterium.

In some embodiments, R⁴ is —CH₂D. In some embodiments, R⁴ is —CH₂D and at least one of R¹, R², R³, R⁵, R⁶, R⁷, or R⁸ is or contains deuterium.

In some embodiments, R⁴ is —CHD₂. In some embodiments, R⁴ is —CHD₂ and at least one of R¹, R², R³, R⁵, R⁶, R⁷, or R⁸ is or contains deuterium.

In some embodiments, R⁴ is —CD₃. In some embodiments, R⁴ is —CD₃ and at least one of R¹, R², R³, R⁵, R⁶, R⁷, or R⁸ is or contains deuterium.

As defined above and described herein, R⁵ is selected from hydrogen or deuterium.

In some embodiments, R⁵ is hydrogen. In some embodiments, R⁵ is hydrogen and at least one of R¹, R², R³, R⁴, R⁶, R⁷, or R⁸ is or contains deuterium. In some embodiments, R⁵ is deuterium. In some embodiments, R⁵ is deuterium and at least one of R¹, R², R³, R⁴, R⁶, R⁷, or R⁸ is or contains deuterium.

As defined above and described herein, R⁶ is selected from hydrogen or deuterium.

In some embodiments, R⁶ is hydrogen. In some embodiments, R⁶ is hydrogen and at least one of R¹, R², R³, R⁴, R⁵, R⁷, or R⁸ is or contains deuterium. In some embodiments, R⁶ is deuterium. In some embodiments, R⁶ is deuterium and at least one of R¹, R², R³, R⁴, R⁵, R⁷, or R⁸ is or contains deuterium.

As defined above and described herein, R⁷ is selected from hydrogen or deuterium.

In some embodiments, R⁷ is hydrogen. In some embodiments, R⁷ is hydrogen and at least one of R¹, R², R³, R⁴, R⁵, R⁶, or R⁸ is or contains deuterium. In some embodiments, R⁷ is deuterium. In some embodiments, R⁷ is deuterium and at least one of R¹, R², R³, R⁴, R⁵, R⁶, or R⁸ is or contains deuterium.

As defined above and described herein, R⁸ is selected from hydrogen or deuterium.

In some embodiments, R⁸ is hydrogen. In some embodiments, R⁸ is hydrogen and at least one of R¹, R², R³, R⁴, R⁵, R⁶, or R⁷ is or contains deuterium. In some embodiments, R⁸ is deuterium. In some embodiments, R⁸ is deuterium and at least one of R¹, R², R³, R⁴, R⁵, R⁶, or R⁷ is or contains deuterium.

In some embodiments, the compound is a compound of formulae X, X-A, XI-A, XI-B, XII-A, XII-B, XII-C, XIII, or XIV, or a pharmaceutically acceptable salt thereof, wherein each of R³, R⁴, R⁵, R⁶, R⁷, and R⁸ is as defined above and described herein, and wherein each of R¹ and R² is as defined in an entry set forth in Table 4a, below.

TABLE 4a
Exemplary Compounds of Formulae X, X-A, XI-A, XI-B,
XII-A, XII-B, XII-C, XIII, or XIV

| Entry | R¹ | R² |
|---|---|---|
| i | —NH₂ | H |
| ii | —NH₂ | D |
| iii | —NHD | H |
| iv | —NHD | D |
| v | —ND₂ | H |
| vi | —ND₂ | D |

In some embodiments, the compound is a compound of formulae X, X-A, XI-A, XI-B, XII-A, XII-B, XII-C, XIII, or XIV, or a pharmaceutically acceptable salt thereof, wherein each of R¹, R², R⁵, R⁶, R⁷, and R⁸ is as defined above and described herein, and wherein each of R³ and R⁴ is as defined in an entry set forth in Table 4b, below.

TABLE 4b
Exemplary Compounds of Formulae X, X-A, XI-A, XI-B,
XII-A, XII-B, XII-C, XIII or XIV

| Entry | R³ | R⁴ |
|---|---|---|
| i | —CH₃ | —CH₃ |
| ii | —CH₃ | —CH₂D |
| iii | —CH₃ | —CHD₂ |
| iv | —CH₃ | —CD₃ |
| v | —CH₂D | —CH₃ |
| vi | —CH₂D | —CH₂D |
| vii | —CH₂D | —CHD₂ |
| viii | —CH₂D | —CD₃ |
| ix | —CHD₂ | —CH₃ |
| x | —CHD₂ | —CH₂D |
| xi | —CHD₂ | —CHD₂ |
| xii | —CHD₂ | —CD₃ |
| xiii | —CD₃ | —CH₃ |
| xiv | —CD₃ | —CH₂D |
| xv | —CD₃ | —CHD₂ |
| xvi | —CD₃ | —CD₃ |

In some embodiments, the compound is a compound of formulae X, X-A, XI-A, XI-B, XII-A, XII-B, XII-C, XIII, or XIV, or a pharmaceutically acceptable salt thereof, wherein each of R¹, R², R³, and R⁴ is as defined above and described herein, and wherein each of R⁵, R⁶, R⁷, and R⁸ is as defined in an entry set forth in Table 4c, below.

TABLE 4c
Exemplary Compounds of Formulae X, X-A, XI-A, XI-B,
XII-A, XII-B, XII-C, XIII, or XIV

| Entry | R⁵ | R⁶ | R⁷ | R⁸ |
|---|---|---|---|---|
| i | H | H | H | H |
| ii | H | H | H | D |
| iii | H | H | D | H |
| iv | H | D | H | H |
| v | D | H | H | H |
| vi | H | H | D | D |
| vii | H | D | H | D |
| viii | D | H | H | D |
| ix | H | D | D | H |
| x | D | H | D | H |
| xi | D | D | H | H |
| xii | H | D | D | D |
| xiii | D | H | D | D |
| xiv | D | D | H | D |
| xv | D | D | D | H |
| xvi | D | D | D | D |

In some embodiments, the compound is a compound of formulae X, X-A, XI-A, XI-B, XII-A, XII-B, XII-C, XIII, or XIV, or a pharmaceutically acceptable salt thereof, wherein each of R¹ and R² is as defined in an entry set forth in Table 4a, above, each of R³ and R⁴ is as defined in an entry set forth in Table 4b, above, and each of R⁵, R⁶, R⁷, and R⁸, is as defined in an entry set forth in Table 4c, above.

In some embodiments, the compound is a compound selected from those recited in any of Table 4a, Table 4b, or Table 4c, or a pharmaceutically acceptable salt thereof.

In some embodiments, the compound is a compound of formula X selected from these depicted in Table 5, below.

TABLE 5
Representative Compounds of Formula X

| | |
|---|---|
| 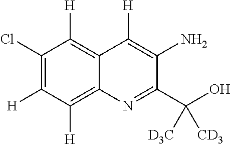 [SMILES: [2H]C([2H])([2H])C(O)(c1nc2ccc(Cl)cc2cc1N)C([2H])([2H])[2H]] | X-1 |
| 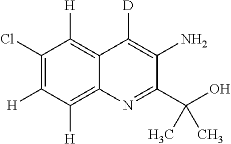 [SMILES: [2H]c1c(N)c(C(C)(C)O)nc2ccc(Cl)cc12] | X-2 |
| 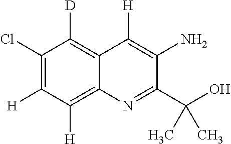 [SMILES: [2H]c1c(Cl)ccc2nc(C(C)(C)O)c(N)cc12] | X-3 |
| 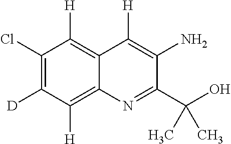 [SMILES: [2H]c1cc2nc(C(C)(C)O)c(N)cc2cc1Cl] | X-4 |
| 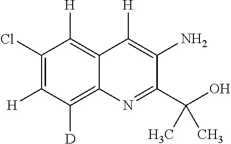 [SMILES: [2H]c1cc(Cl)cc2cc(N)c(C(C)(C)O)nc12] | X-5 |
| 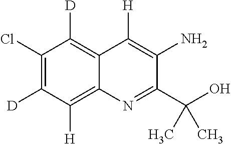 [SMILES: [2H]c1cc2nc(C(C)(C)O)c(N)cc2c([2H])c1Cl] | X-6 |
| 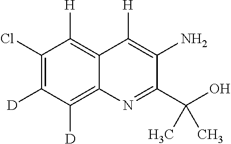 [SMILES: [2H]c1c(Cl)cc2cc(N)c(C(C)(C)O)nc2c1[2H]] | X-7 |
| 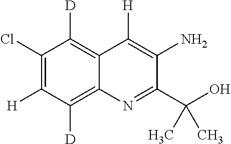 [SMILES: [2H]c1c(Cl)cc([2H])c2nc(C(C)(C)O)c(N)cc12] | X-8 |
| 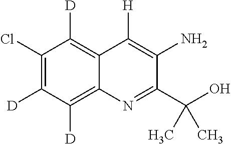 [SMILES: [2H]c1c(Cl)c([2H])c2cc(N)c(C(C)(C)O)nc2c1[2H]] | X-9 |
| 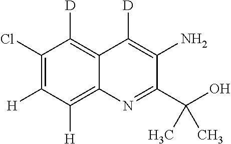 [SMILES: [2H]c1c(Cl)ccc2nc(C(C)(C)O)c(N)c([2H])c12] | X-10 |
| 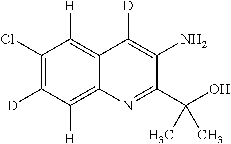 [SMILES: [2H]c1cc2nc(C(C)(C)O)c(N)c([2H])c2cc1Cl] | X-11 |
| 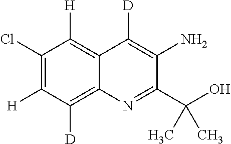 [SMILES: [2H]c1c(N)c(C(C)(C)O)nc2c([2H])cc(Cl)cc12] | X-12 |
| 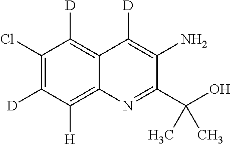 [SMILES: [2H]c1cc2nc(C(C)(C)O)c(N)c([2H])c2c([2H])c1Cl] | X-13 |
| 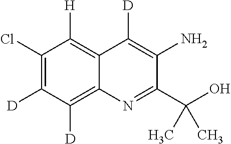 [SMILES: [2H]c1c(Cl)cc2c([2H])c(N)c(C(C)(C)O)nc2c1[2H]] | X-14 |
| 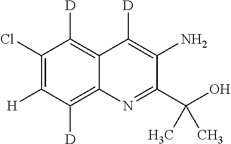 [SMILES: [2H]c1cc(Cl)c([2H])c2c([2H])c(N)c(C(C)(C)O)nc12] | X-15 |
| 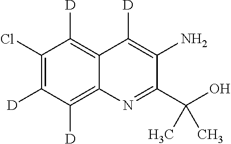 [SMILES: [2H]c1c(Cl)c([2H])c2c([2H])c(N)c(C(C)(C)O)nc2c1[2H]] | X-16 |
| 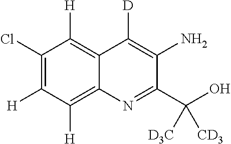 [SMILES: [2H]c1c(N)c(C(O)(C([2H])([2H])[2H])C([2H])([2H])[2H])nc2ccc(Cl)cc12] | X-17 |
| 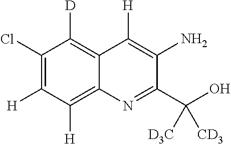 [SMILES: [2H]c1c(Cl)ccc2nc(C(O)(C([2H])([2H])[2H])C([2H])([2H])[2H])c(N)cc12] | X-18 |
| 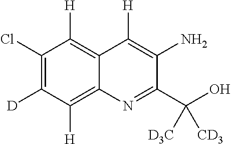 [SMILES: [2H]c1cc2nc(C(O)(C([2H])([2H])[2H])C([2H])([2H])[2H])c(N)cc2cc1Cl] | X-19 |
| 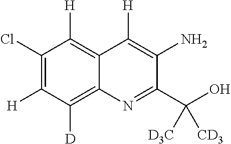 [SMILES: [2H]c1cc(Cl)cc2cc(N)c(C(O)(C([2H])([2H])[2H])C([2H])([2H])[2H])nc12] | X-20 |
| 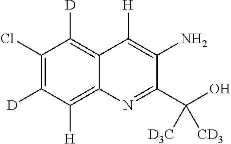 [SMILES: [2H]c1cc2nc(C(O)(C([2H])([2H])[2H])C([2H])([2H])[2H])c(N)cc2c([2H])c1Cl] | X-21 |
| 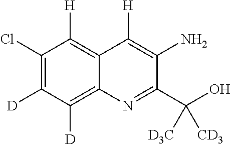 [SMILES: [2H]c1c(Cl)cc2cc(N)c(C(O)(C([2H])([2H])[2H])C([2H])([2H])[2H])nc2c1[2H]] | X-22 |
| 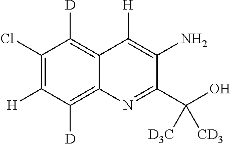 [SMILES: [2H]c1c(Cl)cc([2H])c2nc(C(O)(C([2H])([2H])[2H])C([2H])([2H])[2H])c(N)cc12] | X-23 |
| 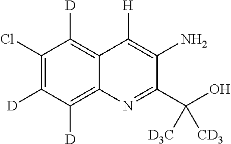 [SMILES: [2H]c1c(Cl)c([2H])c2cc(N)c(C(O)(C([2H])([2H])[2H])C([2H])([2H])[2H])nc2c1[2H]] | X-24 |
| 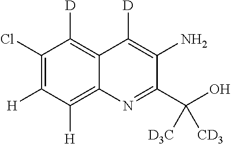 [SMILES: [2H]c1c(Cl)ccc2nc(C(O)(C([2H])([2H])[2H])C([2H])([2H])[2H])c(N)c([2H])c12] | X-25 |
| 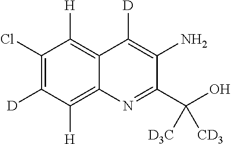 [SMILES: [2H]c1cc2nc(C(O)(C([2H])([2H])[2H])C([2H])([2H])[2H])c(N)c([2H])c2cc1Cl] | X-26 |
| 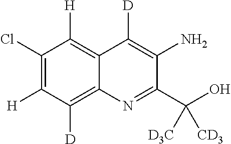 [SMILES: [2H]c1c(N)c(C(O)(C([2H])([2H])[2H])C([2H])([2H])[2H])nc2c([2H])cc(Cl)cc12] | X-27 |
| 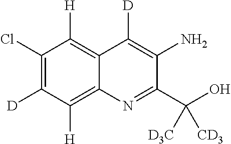 [SMILES: [2H]c1cc2nc(C(O)(C([2H])([2H])[2H])C([2H])([2H])[2H])c(N)c([2H])c2cc1Cl] | X-28 |
| 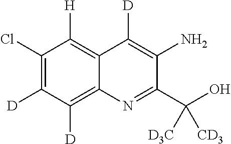 [SMILES: [2H]c1c(Cl)cc2c([2H])c(N)c(C(O)(C([2H])([2H])[2H])C([2H])([2H])[2H])nc2c1[2H]] | X-29 |
| 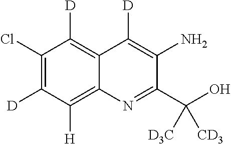 [SMILES: [2H]c1cc2nc(C(O)(C([2H])([2H])[2H])C([2H])([2H])[2H])c(N)c([2H])c2c([2H])c1Cl] | X-30 |
| 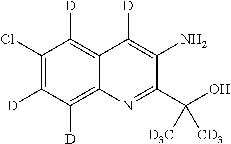 [SMILES: [2H]c1c(Cl)c([2H])c2c([2H])c(N)c(C(O)(C([2H])([2H])[2H])C([2H])([2H])[2H])nc2c1[2H]] | X-31 |

In some embodiments, the compound is a compound depicted in Table 5, above, or a pharmaceutically acceptable salt thereof.

In some embodiments, the present invention provides a deuterium-enriched analogue of a compound depicted in Table 6, below, or a pharmaceutically acceptable salt thereof, in which deuterium is enriched at any available hydrogen.

TABLE 6
Representative Compounds of Formula X

| | |
|---|---|
| 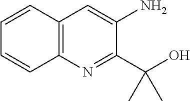 | X-32 |
| 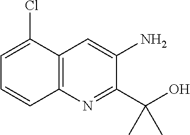 [SMILES: CC(C)(O)c1nc2cccc(Cl)c2cc1N] | X-33 |
| 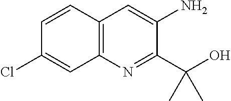 [SMILES: CC(C)(O)c1nc2cc(Cl)ccc2cc1N] | X-34 |
| 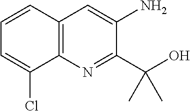 [SMILES: CC(C)(O)c1nc2c(Cl)cccc2cc1N] | X-35 |
| 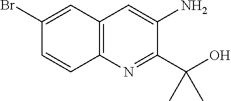 [SMILES: CC(C)(O)c1nc2ccc(Br)cc2cc1N] | X-36 |
| 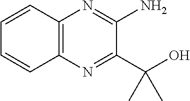 | X-37 |
| 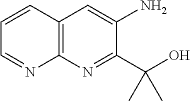 | X-38 |
| 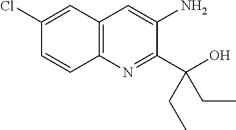 [SMILES: CCC(O)(CC)c1nc2ccc(Cl)cc2cc1N] | X-39 |
| 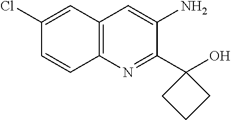 [SMILES: Nc1cc2cc(Cl)ccc2nc1C1(O)CCC1] | X-40 |
| 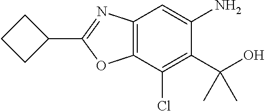 [SMILES: CC(C)(O)c1c(N)cc2nc(C3CCC3)oc2c1Cl] | X-41 |
| 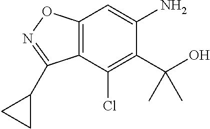 [SMILES: CC(C)(O)c1c(N)cc2onc(C3CC3)c2c1Cl] | X-42 |
| 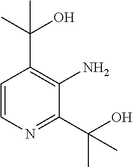 | X-43 |
| 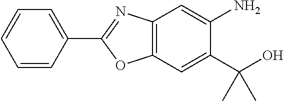 [SMILES: CC(C)(O)c1cc2oc(-c3ccccc3)nc2cc1N] | X-44 |
| 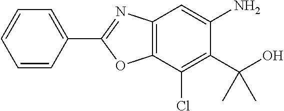 [SMILES: CC(C)(O)c1c(N)cc2nc(-c3ccccc3)oc2c1Cl] | X-45 |
| 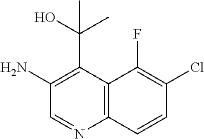 [SMILES: CC(C)(O)c1c(N)cnc2ccc(Cl)c(F)c12] | X-46 |
| 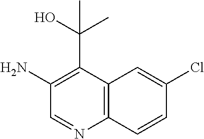 [SMILES: CC(C)(O)c1c(N)cnc2ccc(Cl)cc12] | X-47 |

In some embodiments, the compound is any compound described herein comprising one, two, three, four, five, six, seven, eight, nine, ten, eleven, twelve, or thirteen deuterium atoms.

In some embodiments, provided compounds comprise deuterium in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. As used herein in the context of deuterium enrichment, the term “about” means±2%.

Other Compounds

In some embodiments, the compound is a compound of formula XX:

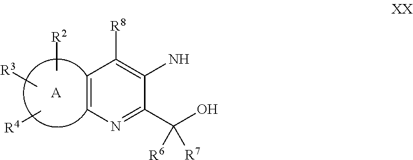

or a pharmaceutically acceptable salt thereof, wherein:
Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, or sulfur; or a 7-membered partially unsaturated heterocyclic or heteroaromatic ring containing 1-3 heteroatoms independently selected from nitrogen, oxygen, or sulfur;
R¹ is H, D, halogen, —CN, —OR, —SR, or optionally substituted C₁₋₆ aliphatic;
each R is independently selected from hydrogen, deuterium, or an optionally substituted group selected from: C₁₋₆ aliphatic, a 3-8 membered saturated or partially unsaturated monocyclic carbocyclic ring, phenyl, an 8-10 membered bicyclic aryl ring, a 3-8 membered saturated or partially unsaturated monocyclic heterocyclic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, a 6-10 membered bicyclic saturated or partially unsaturated heterocyclic ring having 1-5 heteroatoms independently selected from nitrogen, oxygen, or sulfur, or a 7-10 membered bicyclic heteroaryl ring having 1-5 heteroatoms independently selected from nitrogen, oxygen, or sulfur;
R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms; or
   R⁶ and R⁷, taken together with the carbon atom to which they are attached, form a 3-8 membered cycloalkyl or heterocyclyl ring containing 1-2 heteroatoms selected from nitrogen, oxygen, and sulfur.

As defined generally above, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom; or a 6-membered partially unsaturated heterocyclic or heteroaromatic ring containing 1-3 heteroatoms independently selected from nitrogen, oxygen, or sulfur; or a 7-membered partially unsaturated heterocyclic or heteroaromatic ring containing 1-3 heteroatoms independently selected from nitrogen, oxygen, or sulfur.

In some embodiments, Ring A is a 5-membered partially unsaturated heterocyclic or heteroaromatic ring containing 1-3 nitrogen atoms, 1 or 2 oxygen atoms, 1 sulfur atom, or 1 nitrogen and 1 sulfur atom. In some embodiments, Ring A is a 6-membered partially unsaturated heterocyclic or heteroaromatic ring containing 1-3 heteroatoms independently selected from nitrogen, oxygen, or sulfur. In some embodiments, Ring A is a 7-membered partially unsaturated heterocyclic or heteroaromatic ring containing 1-3 heteroatoms independently selected from nitrogen, oxygen, or sulfur.

In some embodiments, Ring A is imidazole or triazole. In some embodiments, Ring A is thiazole. In some embodiments, Ring A is thiophene or furan. In some embodiments, Ring A is pyridine, pyrimidine, pyrazine, pyridazine, or 1,2,4-triazine. In some embodiments, Ring A is pyridine.

As defined generally above, R¹ is H, D, halogen, —CN, —OR, —SR, or optionally substituted C₁₋₆ aliphatic.

In some embodiments, R¹ is H. In some embodiments, R¹ is D. In some embodiments, R¹ is halogen. In some embodiments, R¹ is —CN. In some embodiments, R¹ is —OR. In some embodiments, R¹ is —SR. In some embodiments, R¹ is optionally substituted C₁₋₆ aliphatic.

As described generally above, R² is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R² is absent. In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aryl ring. In some embodiments, R² is an optionally substituted 3-8 membered saturated or partially unsaturated monocyclic heterocyclic ring having 1-4 heteroatoms independently selected from nitrogen, oxygen, or sulfur. In some embodiments, R² is an optionally substituted 5-6 membered monocyclic heteroaryl ring having 1-4 heteroatoms independently selected from nitrogen, oxygen, or sulfur. In some embodiments, R² is an optionally substituted 6-10 membered bicyclic saturated or partially unsaturated heterocyclic ring having 1-5 heteroatoms independently selected from nitrogen, oxygen, or sulfur. In some embodiments, R² is an optionally substituted 7-10 membered bicyclic heteroaryl ring having 1-5 heteroatoms independently selected from nitrogen, oxygen, or sulfur.

In some embodiments, R² is Cl or Br. In some embodiments, R² is Cl.

As defined generally above, R³ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R³ is absent. In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aryl ring. In some embodiments, R³ is an optionally substituted 3-8 membered saturated or partially unsaturated monocyclic heterocyclic ring having 1-4 heteroatoms independently selected from nitrogen, oxygen, or sulfur. In some embodiments, R³ is an optionally substituted 5-6 membered monocyclic heteroaryl ring having 1-4 heteroatoms independently selected from nitrogen, oxygen, or sulfur. In some embodiments, R³ is an optionally substituted 6-10 membered bicyclic saturated or partially unsaturated heterocyclic ring having 1-5 heteroatoms independently selected from nitrogen, oxygen, or sulfur. In some embodiments, R³ is an optionally substituted 7-10 membered bicyclic heteroaryl ring having 1-5 heteroatoms independently selected from nitrogen, oxygen, or sulfur.

In some embodiments, R³ is Cl or Br. In some embodiments, R³ is Cl.

As defined generally above, R⁴ is absent or is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁴ is absent. In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aryl ring. In some embodiments, R⁴ is an optionally substituted 3-8 membered saturated or partially unsaturated monocyclic heterocyclic ring having 1-4 heteroatoms independently selected from nitrogen, oxygen, or sulfur. In some embodiments, R⁴ is an optionally substituted 5-6 membered monocyclic heteroaryl ring having 1-4 heteroatoms independently selected from nitrogen, oxygen, or sulfur. In some embodiments, R⁴ is an optionally substituted 6-10 membered bicyclic saturated or partially unsaturated heterocyclic ring having 1-5 heteroatoms independently selected from nitrogen, oxygen, or sulfur. In some embodiments, R⁴ is an optionally substituted 7-10 membered bicyclic heteroaryl ring having 1-5 heteroatoms independently selected from nitrogen, oxygen, or sulfur.

In some embodiments, R⁴ is Cl or Br. In some embodiments, R⁴ is Cl.

As described generally above, R⁶ is C₁₋₄ aliphatic optionally substituted with 1, 2, or 3 deuterium or halogen atoms.

In some embodiments, R⁶ is C₁₋₄ aliphatic. In some embodiments, R⁶ is C₁₋₄ aliphatic optionally substituted with 1, 2, or 3 deuterium atoms. In some embodiments, R⁶ is C₁₋₄ aliphatic optionally substituted with 1, 2, or 3 halogen atoms.

In some embodiments, R⁶ is C₁₋₄ alkyl. In some embodiments, R⁶ is C₁₋₄ alkyl optionally substituted with 1, 2, or 3 deuterium or halogen atoms. In some embodiments, R⁶ is C₁₋₄ alkyl optionally substituted with 1, 2, or 3 halogen atoms. In some embodiments, R⁶ is methyl or ethyl optionally substituted with 1, 2, or 3 halogen atoms. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic. In some embodiments, R⁷ is C₁₋₄ aliphatic optionally substituted with 1, 2, or 3 deuterium atoms. In some embodiments, R⁷ is C₁₋₄ aliphatic optionally substituted with 1, 2, or 3 halogen atoms.

In some embodiments, R⁷ is C₁₋₄ alkyl. In some embodiments, R⁷ is C₁₋₄ alkyl optionally substituted with 1, 2, or 3 deuterium or halogen atoms. In some embodiments, R⁷ is C₁₋₄ alkyl optionally substituted with 1, 2, or 3 halogen atoms. In some embodiments, R⁷ is methyl or ethyl optionally substituted with 1, 2, or 3 halogen atoms. In some embodiments, R⁷ is methyl.

As defined generally above, in some embodiments, R⁶ and R⁷, taken together with the carbon atom to which they are attached, form a 3-8 membered cycloalkyl or heterocyclyl ring containing 1-2 heteroatoms selected from nitrogen, oxygen, and sulfur.

In some embodiments, R⁶ and R⁷, taken together with the carbon atom to which they are attached, form a 3-8 membered cycloalkyl. In some embodiments, R⁶ and R⁷, taken together with the carbon atom to which they are attached, form a 3-8 membered heterocyclyl ring containing 1-2 heteroatoms selected from nitrogen, oxygen, and sulfur.

In some embodiments, R⁶ and R⁷, taken together with the carbon atom to which they are attached, form a cyclopropyl, cyclobutyl, or cyclopentyl ring. In some embodiments, R⁶ and R⁷, taken together with the carbon atom to which they are attached, form an oxirane, oxetane, tetrahydrofuran, or aziridine.

In some embodiments, R⁶ and R⁷ are methyl.

In some embodiments, the compound is of formula XIX:

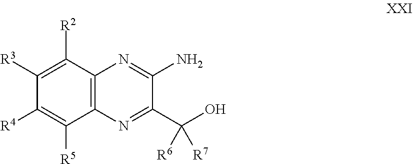

or a pharmaceutically acceptable salt therefor, wherein:
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
each R is independently selected from hydrogen, deuterium, or an optionally substituted group selected from: C₁₋₆ aliphatic, a 3-8 membered saturated or partially unsaturated monocyclic carbocyclic ring, phenyl, an 8-10 membered bicyclic aryl ring, a 3-8 membered saturated or partially unsaturated monocyclic heterocyclic ring having 1-4 heteroatoms independently selected from nitrogen, oxygen, or sulfur, a 5-6 membered monocyclic heteroaryl ring having 1-4 heteroatoms independently selected from nitrogen, oxygen, or sulfur, a 6-10 membered bicyclic saturated or partially unsaturated heterocyclic ring having 1-5 heteroatoms independently selected from nitrogen, oxygen, or sulfur, or a 7-10 membered bicyclic heteroaryl ring having 1-5 heteroatoms independently selected from nitrogen, oxygen, or sulfur;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms; or
   R⁶ and R⁷, taken together with the carbon atom to which they are attached, form a 3-8 membered cycloalkyl or heterocyclyl ring containing 1-2 heteroatoms selected from nitrogen, oxygen, and sulfur.

As described generally above, R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R² is —R. In some embodiments, R² is halogen. In some embodiments, R² is —CN. In some embodiments, R² is —OR. In some embodiments, R² is —SR. In some embodiments, R² is —N(R)₂. In some embodiments, R² is —N(R)C(O)R. In some embodiments, R² is —C(O)N(R)₂. In some embodiments, R² is —N(R)C(O)N(R)₂. In some embodiments, R² is —N(R)C(O)OR. In some embodiments, R² is —OC(O)N(R)₂. In some embodiments, R² is —N(R)S(O)₂R. In some embodiments, R² is —SO₂N(R)₂. In some embodiments, R² is —C(O)R. In some embodiments, R² is —C(O)OR. In some embodiments, R² is —OC(O)R. In some embodiments, R² is —S(O)R. In some embodiments, R² is —S(O)₂R.

In some embodiments, R² is hydrogen. In some embodiments, R² is deuterium. In some embodiments, R² is an optionally substituted C₁₋₆ aliphatic. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aryl ring. In some embodiments, R² is an optionally substituted 3-8 membered saturated or partially unsaturated monocyclic heterocyclic ring having 1-4 heteroatoms independently selected from nitrogen, oxygen, or sulfur. In some embodiments, R² is an optionally substituted 5-6 membered monocyclic heteroaryl ring having 1-4 heteroatoms independently selected from nitrogen, oxygen, or sulfur. In some embodiments, R² is an optionally substituted 6-10 membered bicyclic saturated or partially unsaturated heterocyclic ring having 1-5 heteroatoms independently selected from nitrogen, oxygen, or sulfur. In some embodiments, R² is an optionally substituted 7-10 membered bicyclic heteroaryl ring having 1-5 heteroatoms independently selected from nitrogen, oxygen, or sulfur.

In some embodiments, R² is Cl or Br. In some embodiments, R² is Cl.

As defined generally above, R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R³ is —R. In some embodiments, R³ is halogen. In some embodiments, R³ is —CN. In some embodiments, R³ is —OR. In some embodiments, R³ is —SR. In some embodiments, R³ is —N(R)₂. In some embodiments, R³ is —N(R)C(O)R. In some embodiments, R³ is —C(O)N(R)₂. In some embodiments, R³ is —N(R)C(O)N(R)₂. In some embodiments, R³ is —N(R)C(O)OR. In some embodiments, R³ is —OC(O)N(R)₂. In some embodiments, R³ is —N(R)S(O)₂R. In some embodiments, R³ is —SO₂N(R)₂. In some embodiments, R³ is —C(O)R. In some embodiments, R³ is —C(O)OR. In some embodiments, R³ is —OC(O)R. In some embodiments, R³ is —S(O)R. In some embodiments, R³ is —S(O)₂R.

In some embodiments, R³ is hydrogen. In some embodiments, R³ is deuterium. In some embodiments, R³ is an optionally substituted C₁₋₆ aliphatic. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aryl ring. In some embodiments, R³ is an optionally substituted 3-8 membered saturated or partially unsaturated monocyclic heterocyclic ring having 1-4 heteroatoms independently selected from nitrogen, oxygen, or sulfur. In some embodiments, R³ is an optionally substituted 5-6 membered monocyclic heteroaryl ring having 1-4 heteroatoms independently selected from nitrogen, oxygen, or sulfur. In some embodiments, R³ is an optionally substituted 6-10 membered bicyclic saturated or partially unsaturated heterocyclic ring having 1-5 heteroatoms independently selected from nitrogen, oxygen, or sulfur. In some embodiments, R³ is an optionally substituted 7-10 membered bicyclic heteroaryl ring having 1-5 heteroatoms independently selected from nitrogen, oxygen, or sulfur.

In some embodiments, R³ is Cl or Br. In some embodiments, R³ is Cl.

As defined generally above, R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁴ is —R. In some embodiments, R⁴ is halogen. In some embodiments, R⁴ is —CN. In some embodiments, R⁴ is —OR. In some embodiments, R⁴ is —SR. In some embodiments, R⁴ is —N(R)₂. In some embodiments, R⁴ is —N(R)C(O)R. In some embodiments, R⁴ is —C(O)N(R)₂. In some embodiments, R⁴ is —N(R)C(O)N(R)₂. In some embodiments, R⁴ is —N(R)C(O)OR. In some embodiments, R⁴ is —OC(O)N(R)₂. In some embodiments, R⁴ is —N(R)S(O)₂R. In some embodiments, R⁴ is —SO₂N(R)₂. In some embodiments, R⁴ is —C(O)R. In some embodiments, R⁴ is —C(O)OR. In some embodiments, R⁴ is —OC(O)R. In some embodiments, R⁴ is —S(O)R. In some embodiments, R⁴ is —S(O)₂R.

In some embodiments, R⁴ is hydrogen. In some embodiments, R⁴ is deuterium. In some embodiments, R⁴ is an optionally substituted C₁₋₆ aliphatic. In some embodiments, R⁴ is an optionally substituted 3-8 membered saturated or partially unsaturated monocyclic carbocyclic ring. In some embodiments, R⁴ is an optionally substituted phenyl. In some embodiments, R⁴ is an optionally substituted 8-10 membered bicyclic aryl ring. In some embodiments, R⁴ is an optionally substituted 3-8 membered saturated or partially unsaturated monocyclic heterocyclic ring having 1-4 heteroatoms independently selected from nitrogen, oxygen, or sulfur. In some embodiments, R⁴ is an optionally substituted 5-6 membered monocyclic heteroaryl ring having 1-4 heteroatoms independently selected from nitrogen, oxygen, or sulfur. In some embodiments, R⁴ is an optionally substituted 6-10 membered bicyclic saturated or partially unsaturated heterocyclic ring having 1-5 heteroatoms independently selected from nitrogen, oxygen, or sulfur. In some embodiments, R⁴ is an optionally substituted 7-10 membered bicyclic heteroaryl ring having 1-5 heteroatoms independently selected from nitrogen, oxygen, or sulfur.

In some embodiments, R⁴ is Cl or Br. In some embodiments, R⁴ is Cl.

As defined generally above, R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, or —S(O)₂R.

In some embodiments, R⁵ is —R. In some embodiments, R⁵ is halogen. In some embodiments, R⁵ is —CN. In some embodiments, R⁵ is —OR. In some embodiments, R⁵ is —SR. In some embodiments, R⁵ is —N(R)₂. In some embodiments, R⁵ is —N(R)C(O)R. In some embodiments, R⁵ is —C(O)N(R)₂. In some embodiments, R⁵ is —N(R)C(O)N(R)₂. In some embodiments, R⁵ is —N(R)C(O)OR. In some embodiments, R⁵ is —OC(O)N(R)₂. In some embodiments, R⁵ is —N(R)S(O)₂R. In some embodiments, R⁵ is —SO₂N(R)₂. In some embodiments, R⁵ is —C(O)R. In some embodiments, R⁵ is —C(O)OR. In some embodiments, R⁵ is —OC(O)R. In some embodiments, R⁵ is —S(O)R. In some embodiments, R⁵ is —S(O)₂R.

In some embodiments, R⁵ is hydrogen. In some embodiments, R⁵ is deuterium. In some embodiments, R⁵ is an optionally substituted C₁₋₆ aliphatic. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aryl ring. In some embodiments, R⁵ is an optionally substituted 3-8 membered saturated or partially unsaturated monocyclic heterocyclic ring having 1-4 heteroatoms independently selected from nitrogen, oxygen, or sulfur. In some embodiments, R⁵ is an optionally substituted 5-6 membered monocyclic heteroaryl ring having 1-4 heteroatoms independently selected from nitrogen, oxygen, or sulfur. In some embodiments, R⁵ is an optionally substituted 6-10 membered bicyclic saturated or partially unsaturated heterocyclic ring having 1-5 heteroatoms independently selected from nitrogen, oxygen, or sulfur. In some embodiments, R⁵ is an optionally substituted 7-10 membered bicyclic heteroaryl ring having 1-5 heteroatoms independently selected from nitrogen, oxygen, or sulfur.

In some embodiments, R⁵ is Cl or Br. In some embodiments, R⁵ is Cl.

As described generally above, R⁶ is C₁₋₄ aliphatic optionally substituted with 1, 2, or 3 deuterium or halogen atoms.

In some embodiments, R⁶ is C₁₋₄ aliphatic. In some embodiments, R⁶ is C₁₋₄ aliphatic optionally substituted with 1, 2, or 3 deuterium atoms. In some embodiments, R⁶ is C₁₋₄ aliphatic optionally substituted with 1, 2, or 3 halogen atoms.

In some embodiments, R⁶ is C₁₋₄ alkyl. In some embodiments, R⁶ is C₁₋₄ alkyl optionally substituted with 1, 2, or 3 deuterium or halogen atoms. In some embodiments, R⁶ is C₁-4 alkyl optionally substituted with 1, 2, or 3 halogen atoms. In some embodiments, R⁶ is methyl or ethyl optionally substituted with 1, 2, or 3 halogen atoms. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic. In some embodiments, R⁷ is C₁₋₄ aliphatic optionally substituted with 1, 2, or 3 deuterium atoms. In some embodiments, R⁷ is C₁₋₄ aliphatic optionally substituted with 1, 2, or 3 halogen atoms.

In some embodiments, R⁷ is C₁₋₄ alkyl. In some embodiments, R⁷ is C₁₋₄ alkyl optionally substituted with 1, 2, or 3 deuterium or halogen atoms. In some embodiments, R⁷ is C₁₋₄ alkyl optionally substituted with 1, 2, or 3 halogen atoms. In some embodiments, R⁷ is methyl or ethyl optionally substituted with 1, 2, or 3 halogen atoms. In some embodiments, R⁷ is methyl.

As defined generally above, in some embodiments, R⁶ and R⁷, taken together with the carbon atom to which they are attached, form a 3-8 membered cycloalkyl or heterocyclyl ring containing 1-2 heteroatoms selected from nitrogen, oxygen, and sulfur.

In some embodiments, R⁶ and R⁷, taken together with the carbon atom to which they are attached, form a 3-8 membered cycloalkyl. In some embodiments, R⁶ and R⁷, taken together with the carbon atom to which they are attached, form a 3-8 membered heterocyclyl ring containing 1-2 heteroatoms selected from nitrogen, oxygen, and sulfur.

In some embodiments, R⁶ and R⁷, taken together with the carbon atom to which they are attached, form a cyclopropyl, cyclobutyl, or cyclopentyl ring. In some embodiments, R⁶ and R⁷, taken together with the carbon atom to which they are attached, form an oxirane, oxetane, tetrahydrofuran, or aziridine.

In some embodiments, R⁶ and R⁷ are methyl.

In some embodiments, the compound is of formulae XX-a, XX-b, XX-c, or XX-d:

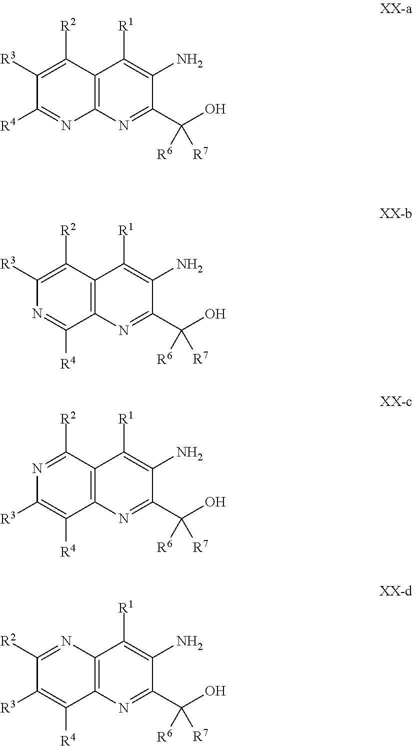

or a pharmaceutically acceptable salt thereof, wherein:
each of R, R¹, R², R³, R⁴, R⁶, and R⁷ is as defined is as defined above and described in embodiments herein, both singly and in combination.

In some embodiments, the compound is of formula XX-a above.

In some embodiments, R¹ and R⁴ are H. In some embodiments, R² is H. In some embodiments, R⁶ and R⁷ are C₁₋₄ alkyl optionally substituted with 1, 2, or 3 deuterium or halogen atoms, or R⁶ and R⁷ are taken together with the carbon to which they are attached to form a 3-8 membered cycloalkyl ring. In some embodiments, R³ is H, C₁₋₄ alkyl, halogen, —NR, —OR, —SR, —CO₂R, or —C(O)R, wherein R is H, optionally substituted C₁₋₄ alkyl, or optionally substituted phenyl.

In some embodiments, the compound is a compound of formulae XX-e, XX-f, XX-g, or XX-h:

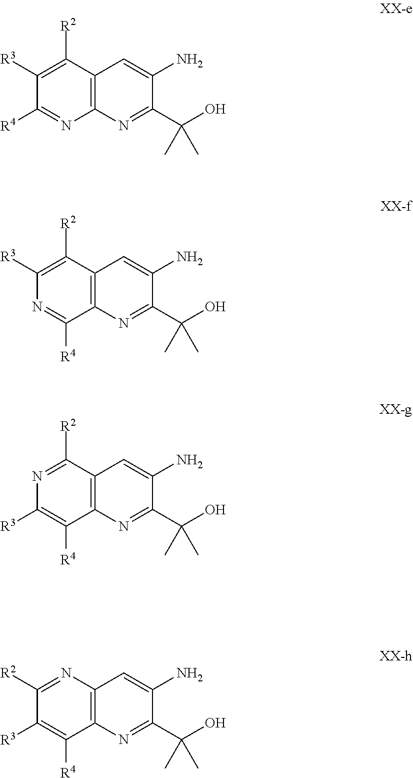

or a pharmaceutically acceptable salt thereof, wherein:
each of R, R¹, R², R³, and R⁴ is as defined is as defined above and described in embodiments herein, both singly and in combination.

In another aspect, the present invention provides a compound of formulae XX-i, XX-j, XX-k, XX-1, XX-m, or XX-n:

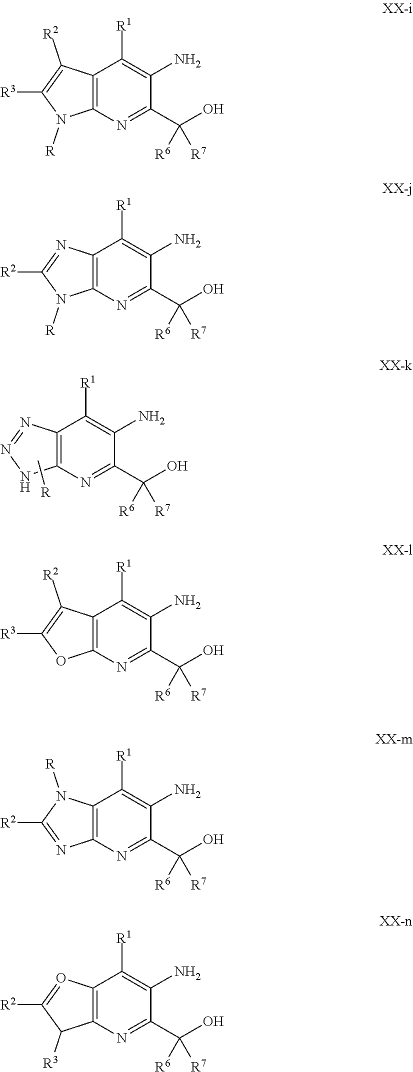

or a pharmaceutically acceptable salt thereof, wherein:
each of R, R¹, R², R³, R⁴, R⁶, and R⁷ is as defined is as defined above and described in embodiments herein, both singly and in combination.

In some embodiments, the compound is a compound of formula XXI-a:

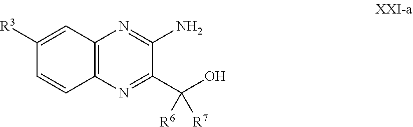

or a pharmaceutically acceptable salt thereof, wherein:
each of R, R³, R⁶, and R⁷ is as defined is as defined above and described in embodiments herein, both singly and in combination.

In another aspect, the present invention provides a compound selected from these depicted in Table 7, below.

TABLE 7
Representative Compounds of Formula XX

| | |
|---|---|
| 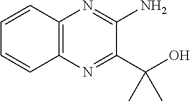 | XX-1 |
| 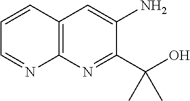 | XX-2 |
| 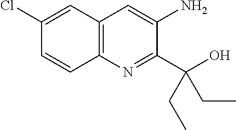 [SMILES: CCC(O)(CC)c1nc2ccc(Cl)cc2cc1N] | XX-3 |
| 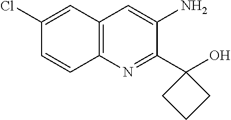 [SMILES: Nc1cc2cc(Cl)ccc2nc1C1(O)CCC1] | XX-4 |
| 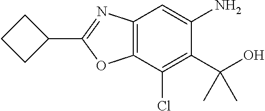 [SMILES: CC(C)(O)c1c(N)cc2nc(C3CCC3)oc2c1Cl] | XX-5 |
| 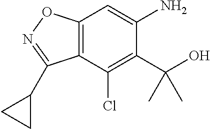 [SMILES: CC(C)(O)c1c(N)cc2onc(C3CC3)c2c1Cl] | XX-6 |
| 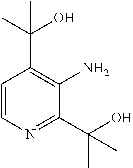 | XX-7 |
| 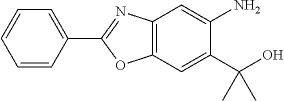 [SMILES: CC(C)(O)c1cc2oc(-c3ccccc3)nc2cc1N] | XX-8 |
| 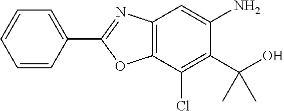 [SMILES: CC(C)(O)c1c(N)cc2nc(-c3ccccc3)oc2c1Cl] | XX-9 |
| 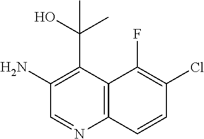 [SMILES: CC(C)(O)c1c(N)cnc2ccc(Cl)c(F)c12] | XX-10 |
| 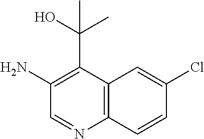 [SMILES: CC(C)(O)c1c(N)cnc2ccc(Cl)cc12] | XX-11 |

In some embodiments, the compound is a compound depicted in Table 7, above, or a pharmaceutically acceptable salt thereof.

In certain embodiments, the compound is any compound described above and herein, or a pharmaceutically acceptable salt thereof.

In some embodiments, the compound is any compound described above and herein in isolated form. As used herein, the term “isolated” means that a compound is provided in a form that is separated from other components that might be present in that compound's usual environment. In certain embodiments, an isolated compound is in solid form. In some embodiments, an isolated compound is at least about 50% pure as determined by a suitable HPLC method. In certain embodiments, an isolated compound is at least about 60%, 70%, 80%, 90%, 95%, 98%, 99%, 99.5%, 99.6%, 99.7%, 99.8%, 99.9%, 99.95%, 99.99%, or 99.999% as determined by a suitable HPLC method. Methods of preparation applicable to certain compounds of the invention are disclosed in US 2013/0190500, published Jul. 25, 2013, which is hereby incorporated by reference.

In some embodiments, the compound is of formula XXII:

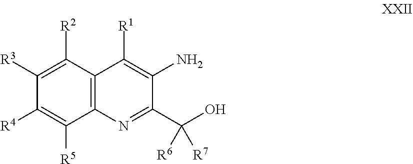

or a pharmaceutically acceptable salt thereof, wherein:
R¹ is H, D, or halogen;
R² is H, D, or halogen;
R³ is H, D, Br, or I;
R⁴ is H, D, or halogen;
R⁵ is H, D, or halogen;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, Br, or I.

In some embodiments, R³ is H. In some embodiments, R³ is D. In some embodiments, R³ is Br. In some embodiments, R³ is I.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R⁶ is C₁₋₄ aliphatic optionally substituted with 1, 2, or 3 deuterium or halogen atoms.

In some embodiments, R⁶ is C₁₋₄ aliphatic substituted with 1, 2, or 3 deuterium or halogen atoms. In some embodiments, R⁶ is C₁₋₄ aliphatic. In some embodiments, R⁶ is C₁₋₄ alkyl. In some embodiments, R⁶ is methyl, ethyl, n-propyl, or isopropyl. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic substituted with 1, 2, or 3 deuterium or halogen atoms. In some embodiments, R⁷ is C₁₋₄ aliphatic. In some embodiments, R⁷ is C₁₋₄ alkyl. In some embodiments, R⁷ is C₁₋₄ alkyl optionally substituted with 1, 2, or 3 fluorine atoms. In some embodiments, R⁷ is methyl, ethyl, n-propyl, or isopropyl. In some embodiments, R⁷ is methyl.

In some embodiments, R⁶ and R⁷ are methyl or ethyl. In some embodiments, R⁶ and R⁷ are methyl.

In some embodiments, the compound is a compound of formula XXII-a:

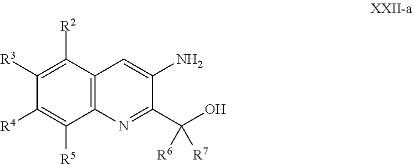

or a pharmaceutically acceptable salt thereof, wherein:
each of R², R³, R⁴, R⁵, R⁶, and R⁷ is as defined is as defined above and described in embodiments herein, both singly and in combination.

In some embodiments, the compound is a compound of formula XXII-b:

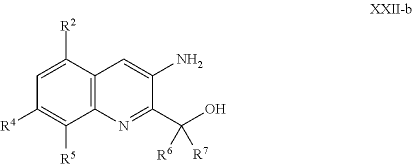

or a pharmaceutically acceptable salt thereof, wherein:
each of R², R⁴, R⁵, R⁶, and R⁷ is as defined is as defined above and described in embodiments herein, both singly and in combination.

In some embodiments, the compound is a compound of formulae XXII-c, XXII-d, XXII-e, or XXII-f:

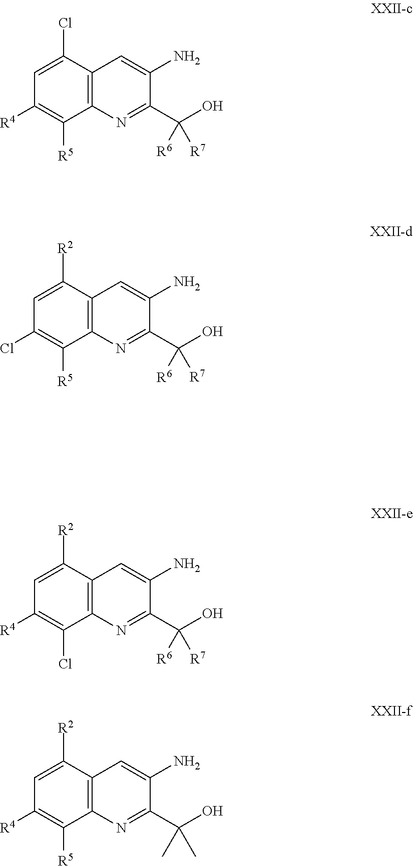

or a pharmaceutically acceptable salt thereof, wherein:
each of R², R⁴, R⁵, R⁶, and R⁷ is as defined is as defined above and described in embodiments herein, both singly and in combination.

In some embodiments, the compound is a compound of formulae XXII-g, XXII-h, XXII-i, or XXII-j:

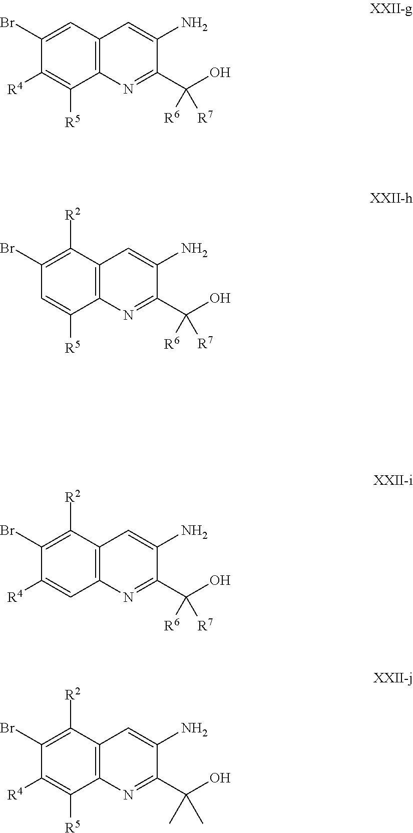

or a pharmaceutically acceptable salt thereof, wherein:
each of R², R⁴, R⁵, R⁶, and R⁷ is as defined is as defined above and described in embodiments herein, both singly and in combination.

In some embodiments, the compound is a compound of formulae XXII-k or XXII-l:

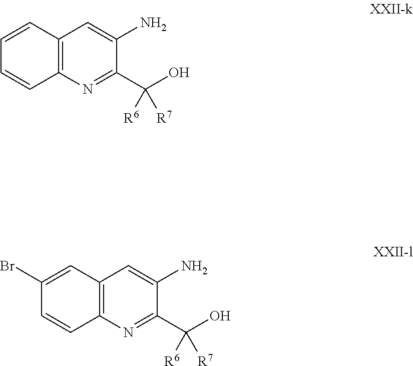

or a pharmaceutically acceptable salt thereof, wherein:
each of R⁶ and R⁷ is as defined is as defined above and described in embodiments herein, both singly and in combination.

In some embodiments, the compound is a compound of formula XXIII:

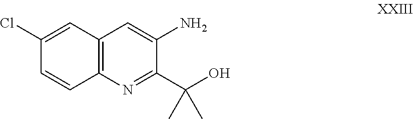 [SMILES: CC(C)(O)c1nc2ccc(Cl)cc2cc1N]

or a pharmaceutically acceptable salt thereof, in combination with at least one compound of formula XXII:

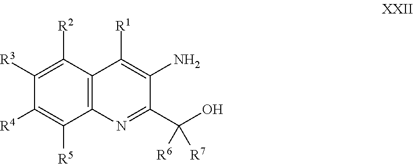

or a pharmaceutically acceptable salt thereof, wherein:
R¹ is H, D, or halogen;
R² is H, D, or halogen;
R³ is H, D, Br, or I;
R⁴ is H, D, or halogen;
R⁵ is H, D, or halogen;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms.

In some embodiments, the composition comprises a compound of formula XXIII, or a pharmaceutically acceptable salt thereof, and at least one compound according to formulae XXII-a, XXII-b, XXII-c, XXII-d, XXII-e, XXII-f, XXII-g, XXII-h, XXII-i, XXII-j, XXII-k, or XXII-l; or a pharmaceutically acceptable salt thereof.

In some embodiments, the composition comprises a compound of formula XXIII:

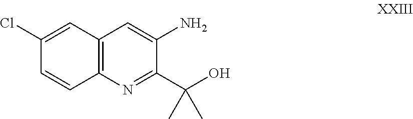 [SMILES: CC(C)(O)c1nc2ccc(Cl)cc2cc1N]

or a pharmaceutically acceptable salt thereof, and a compound selected from the following, or a pharmaceutically acceptable salt thereof:

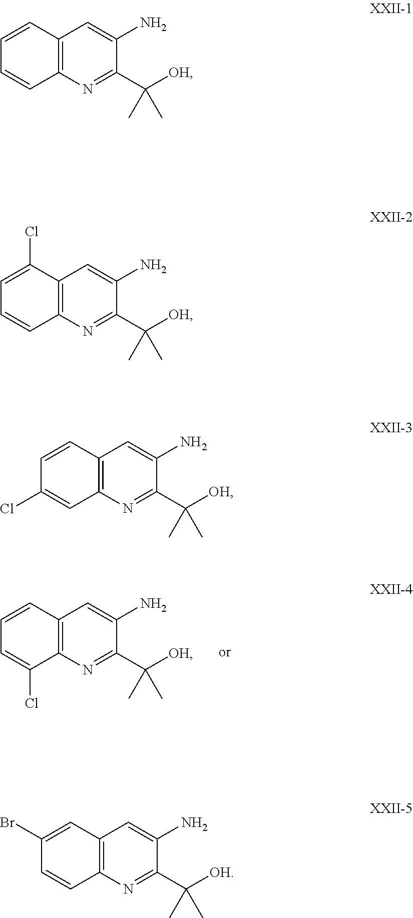 [SMILES: CC(C)(O)c1nc2ccccc2cc1N ; CC(C)(O)c1nc2cccc(Cl)c2cc1N ; CC(C)(O)c1nc2cc(Cl)ccc2cc1N ; CC(C)(O)c1nc2c(Cl)cccc2cc1N ; "or" ; CC(C)(O)c1nc2ccc(Br)cc2cc1N]

In some embodiments, the compound included as an active agent in a disclosed topical formulation is selected from XXII-1, XXII-2, XXII-3, XXII-4, or XXII-5; or a pharmaceutically acceptable salt thereof.

Exemplary Topical, e.g. Dermal, Compositions Containing an Active Agent

In some embodiments, the present invention provides a topical (e.g., dermal) composition comprising any of the above-described active agents in any of the above-described amounts. In some embodiments, the active agent is ADX-102, I-22, I-6, or X-1, or a pharmaceutically acceptable salt thereof. In certain embodiments, the active agent is ADX-102.

In some embodiments, the present invention provides a topical composition comprising or consisting essentially of:

(i) an active agent or pharmaceutically acceptable salt thereof;

(ii) a first solvent;

(iii) optionally a second solvent;

(iv) a penetration enhancer;

(v) optionally one or more of a preservative, solubilizing agent, thickening agent, stabilizing agent, surfactant, skin conditioner, humectant, or second skin penetration enhancer;

(vi) optionally an antioxidant; and

optionally one or more of: an emulsifying agent, a diluent, a pH adjuster, a chelating agent, a coloring agent, or a fragrance.

In some embodiments, the present invention provides a topical composition comprising or consisting essentially of:

(i) an active agent or pharmaceutically acceptable salt thereof;

(ii) a first solvent;

(iii) optionally a second solvent;

(iv) a penetration enhancer;

(v) a preservative;

(vi) a surfactant;

(vii) optionally one or more of a solubilizing agent, thickening agent, stabilizing agent, skin conditioner, or second skin penetration enhancer;

(viii) optionally an antioxidant; and

optionally one or more of: an emulsifying agent, a diluent, a pH adjuster, a chelating agent, a coloring agent, or a fragrance.

In some embodiments, the present invention provides a topical composition comprising or consisting essentially of:

(i) an active agent or pharmaceutically acceptable salt thereof;

(ii) a first solvent;

(iii) optionally a second solvent;

(iv) a penetration enhancer;

(v) a preservative;

(vi) optionally one or more of a solubilizing agent, thickening agent, stabilizing agent, surfactant, skin conditioner, or second skin penetration enhancer;

(vii) an antioxidant; and

optionally one or more of: an emulsifying agent, a diluent, a pH adjuster, a chelating agent, a coloring agent, or a fragrance.

In some embodiments, the present invention provides a topical composition comprising or consisting essentially of:

(i) an active agent or pharmaceutically acceptable salt thereof;

(ii) a first solvent;

(iii) optionally a second solvent;

(iv) a penetration enhancer;

(v) a preservative;

(vi) a surfactant;

(vii) a thickening agent;

(viii) a stabilizing agent;

(ix) optionally one or more of a solubilizing agent, skin conditioner, or second skin penetration enhancer;

(x) optionally an antioxidant; and

optionally one or more of: an emulsifying agent, a diluent, a pH adjuster, a chelating agent, a coloring agent, or a fragrance.

In some embodiments, the present invention provides a topical composition comprising or consisting essentially of:

(i) an active agent or pharmaceutically acceptable salt thereof;

(ii) a first solvent;

(iii) a second solvent;

(iv) a penetration enhancer;

(v) a preservative;

(vi) a surfactant;

(vii) optionally one or more of a solubilizing agent, thickening agent, stabilizing agent, skin conditioner, or second skin penetration enhancer;

(viii) an antioxidant; and

optionally one or more of: an emulsifying agent, a diluent, a pH adjuster, a chelating agent, a coloring agent, or a fragrance.

In some embodiments, the active agent is in the form of its free base, or is substantially in the form of the free base. In some embodiments, the active agent is in the form of a pharmaceutically acceptable salt.

In some embodiments, the present invention provides a dermal composition, wherein the composition does not comprise diisopropyl adipate. In some embodiments, the composition does not comprise benzyl alcohol or is substantially free of benzyl alcohol.

In some embodiments, the composition is substantially free of excipients or solvents that contain aldehydes or present a risk of decomposing to produce aldehyde-containing impurities.

In some embodiments, the solvent is selected from benzyl alcohol, PEG 400, super refined (SR) PEG 400, propylene carbonate, castor oil, glycerol, IPM, IPP, Miglyol 810, Transcutol HP, propylene glycol, ethanol, or a water-alcohol mixture (e.g., 75:25 v/v WFI:ethanol and 75:25 v/v deionised water:ethanol), or a mixture thereof.

In some embodiments, the present invention provides a topical (e.g., dermal) composition, comprising about 0.1 to 7.5% of an active agent; about 12.0 to 48.0% of a first solvent; about 2.0 to 15.0% of a second solvent; about 1.0 to 29.0% of a penetration enhancer; optionally up to about 3.0% of a preservative; optionally up to about 20.0% of a thickening agent; about 1.0 to 15.0% of a surfactant; optionally up to about 10.0% of a stabilizing agent; and optionally up to about 1.0% of an antioxidant.

In some embodiments, the composition comprises up to about 40.0% water. In some embodiments, the composition comprises about 28.0 to 40.0% water.

In some embodiments, the first solvent comprises a polyethylene glycol, polyoxyethylene alkyl ether, polyoxyethylene sorbitan fatty acid ester, or polyoxyethylene stearate.

In some embodiments, the second solvent is present and comprises a glycol solvent (e.g., propylene glycol); and an alkyl alcohol solvent (e.g., ethanol).

In some embodiments, the penetration enhancer is selected from one or more of dimethyl sulfoxide, a glycol, glyceryl monooleate, glycofurol, isopropyl myristate, isopropyl palmitate, lanolin, light mineral oil, linoleic acid, menthol, myristic acid, myristyl alcohol, oleic acid, oleyl alcohol, palmitic acid, a polyoxyethylene alkyl ether, a polyoxylglyceride, a pyrrolidone, lauryl sulfate, thymol, tricaprylin, triolein, acetyltributyl citrate, ethylene vinyl acetate, a polymethacrylate, polyvinyl alcohol, sesame oil, carboxymethyl guar, or stearyl alcohol.

In some embodiments, the penetration enhancer is selected from an alkanol, a glycol, or a polyoxyethylene alkyl ether.

In some embodiments, the penetration enhancer is selected from Transcutol HP, propylene glycol, or a mixture thereof.

In some embodiments, the surfactant is selected from emulsifying wax USP, glyceryl monooleate, a phospholipid, a polyoxyethylene alkyl ether, polyoxyethylene castor oil derivative, a polyoxyethylene sorbitan fatty acid ester, polyoxyethylene stearates, polyoxylglycerides (such as Caprylocaproyl Polyoxylglycerides, Lauroyl Polyoxylglycerides, Linoleoyl Polyoxylglycerides, Oleoyl Polyoxylglycerides, Stearoyl Polyoxylglycerides), polysorbate, a sorbitan ester, triethyl citrate, vitamin E polyethylene glycol succinate, miglyol, a Steareth (Brij) surfactant, a Cetomacrogol, a Myrj, a Span, a Tween, or a Laureth surfactant.

In some embodiments, the surfactant is selected from Steareth 21 (Brij s721), Steareth 2 (Brij S2), Cetomacrogol 1000, Myrj S40, Brij CS25 (Ceteareth 25), Steareth 20 (Brij S20), Span 20, Geleol mono and diglycerides, Span 60, or Tween 60, or a combination thereof.

In some embodiments, the surfactant is a combination of Steareth 2 and Mrij S40; Steareth 2 and Brij CS25; Steareth 2 and Cetomacrogol 1000; Steareth 2 and Steareth 20/Brij S20; Steareth 2 and Steareth 21; Span 20 and Geleol; Span 60 and Tween 60; Cetomacrogol 1000 and Geleol; Brij S20 and Brij S2; or Geleol, Span 20, and Steareth 20/Brij S20.

In some embodiments, the thickening agent is a high molecular weight polyethylene glycol such as PEG 1500 or PEG 4000.

In some embodiments, the preservative is selected from vitamin C, butylated hydroxytoluene (BHT), a sulfite, vitamin E, ascorbyl palmitate, or propyl gallate. In some embodiments, the preservative is benzyl alcohol or phenoxyethanol.

In some embodiments, the antioxidant is a combination of BHT and butylated hydroxyanisole (BHA).

In some embodiments, a preservative and antioxidant is present in the composition. In some embodiments, the preservative and antioxidant comprise phenoxyethanol, BHT, butylated hydroxyanisole (BHA), or a mixture of two or all three thereof.

In some embodiments, the stabilizing agent is selected from white wax, yellow wax (beeswax), ceresin wax, liquid paraffin, and white petrolatum.

In some embodiments, the composition comprises a solvent system described in one of the Tables below, such as Table 28 or Table 29.

In some embodiments, the composition is one of those defined in Table 37, Table 38, or Table 39.

In some embodiments, the composition is one of those defined in Table D, Table E, or Table F below. In some embodiments, the present invention provides a composition defined in Table D, Table E, or Table F for use in treating a disease, disorder, or condition selected from one of those described herein, such as SLS or an ichthyosis. In some embodiments, the present invention provides a composition defined in Table 37, Table 38, or Table 39 for use in treating a disease, disorder, or condition selected from one of those described herein, such as SLS or an ichthyosis.

In some embodiments, the pH of the composition is between about 6.63 and 8.45.

In some embodiments, a provided topical composition has a viscosity or average viscosity of from about 30,000 to about 100,000 Centipoise (“cP”); from about 40,000 to about 90,000 cP; from about 50,000 to about 80,000 cP; from about 55,000 to about 75,000 cP; from about 55,000 to about 70,000 cP; from about 60,000 to about 70,000 cP; or from about 60,000 to about 66,000 cP.

In some embodiments, a provided topical composition has a viscosity or average viscosity of from about 55,000 to about 70,000 centipoise (cP).

In some embodiments, a provided topical composition has a viscosity or average viscosity of from about 30,000 to about 200,000 centipoise (cP).

In some embodiments, the topical, e.g. dermal, pharmaceutical composition comprises one of the combinations of ingredients in each of the weight percentages (% w/w, or wt %), or about the % w/w, provided below in Table D. Where an ingredient is present in a range that includes 0%, this indicates that the ingredient is optionally present. Thus, in some embodiments, such an ingredient is not present. Each ingredient that is optionally present is either present or not present independently of any other optionally present ingredients. In other embodiments, each such ingredient is independently present up to the maximum % indicated for that ingredient.

TABLE D
Composition (% w/w) of exemplary topical pharmaceutical compositions

| | Composition (% w/w) | | | | | | | | | |
|---|---|---|---|---|---|---|---|---|---|---|
| Ingredient/ | Entry | | | | | | | | | |
| Excipient | 1 | 2 | 3 | 4 | 5 | 6 | 7 | 8 | 9 | 10 |
| Active agent | 0.1- | 0.5- | 0.5- | 0.5- | 0.5- | 0.5- | 0.5- | 0.5- | 0.5- | 0.5- |
| such as ADX- | 7.5% | 2.5% | 2.5% | 2.5% | 2.5% | 2.5% | 2.5% | 2.5% | 2.5% | 2.5% |
| 102 or a | | | | | | | | | | |
| pharmaceutically | | | | | | | | | | |
| acceptable | | | | | | | | | | |
| salt thereof | | | | | | | | | | |
| Penetration | 1.0- | 5.0- | 9.0- | 7- | 9.0- | 0.8- | 1.5- | 7.0- | 7.0- | 7.0- |
| enhancer | 29.0% | 26.0% | 23.5% | 23.0% | 21.0% | 4.0% | 20.5% | 18.0% | 16.0% | 12.0% |
| such as | | | | | | | | | | |
| Transcutol | | | | | | | | | | |
| HP, | | | | | | | | | | |
| Propylene | | | | | | | | | | |
| Glycol, or a | | | | | | | | | | |
| mixture | | | | | | | | | | |
| thereof | | | | | | | | | | |
| Purified | 28.0- | 29.0%- | 30.0- | 28.0- | 29.0- | 28.0- | 34.0- | 28.0- | 28.0- | 28.0- |
| Water | 40.0% | 38.0% | 37.0% | 36.0% | 40.0% | 37.0% | 40.0% | 36.0% | 36.0% | 36.0% |
| First solvent | 12.0- | 15.0- | 18.0- | 14.0- | 21.0- | 30.0- | 32.0- | 22.0- | 22.0- | 22.0- |
| such as PEG | 48.0% | 42.0% | 40.0% | 29.0% | 37.0% | 45.0% | 48.0% | 35.0% | 35.0% | 35.0% |
| 400, S.R. | | | | | | | | | | |
| PEG 400, | | | | | | | | | | |
| PEG 200, | | | | | | | | | | |
| PEG 300, | | | | | | | | | | |
| PEG 540, | | | | | | | | | | |
| PEG 600, | | | | | | | | | | |
| PEG 900, | | | | | | | | | | |
| propylene | | | | | | | | | | |
| carbonate, | | | | | | | | | | |
| butyl stearate, | | | | | | | | | | |
| castor oil, | | | | | | | | | | |
| IPM, IPP, | | | | | | | | | | |
| Miglyol 810, | | | | | | | | | | |
| or a mixture | | | | | | | | | | |
| thereof | | | | | | | | | | |
| Second | 0 or | 0 or | 0 or | 0 or | 0 or | 0 or | 0- | 0 or | 0 or | 0 or |
| solvent such | 2.0- | 5.0- | 5.0- | 4.0- | 4.0- | 3.0- | 12% | 4.0- | 4.0- | 4.0- |
| as ethanol, | 15.0% | 13.0% | 13.0% | 14.0% | 15.0% | 15.0% | | 15.0% | 15.0% | 15.0% |
| propanol, | | | | | | | | | | |
| butanol, | | | | | | | | | | |
| s-butanol, or | | | | | | | | | | |
| t-butanol, | | | | | | | | | | |
| propylene | | | | | | | | | | |
| glycol, | | | | | | | | | | |
| ethylene | | | | | | | | | | |
| glycol, or a | | | | | | | | | | |
| mixture | | | | | | | | | | |
| thereof | | | | | | | | | | |
| Preservative | 0- | 0.1- | 0.25- | 0- | 0.25- | 0.25- | 0.25- | 0.25- | 0.25- | 0.25- |
| such as | 3.0% | 3.0% | 2.5% | 3.0% | 2.5% | 2.5% | 2.5% | 2.5% | 2.5% | 2.5% |
| Phenoxyethanol, | | | | | | | | | | |
| benzyl | | | | | | | | | | |
| alcohol, or a | | | | | | | | | | |
| mixture | | | | | | | | | | |
| thereof | | | | | | | | | | |
| Antioxidant | 0- | 0- | 0- | 0- | 0.01- | 0- | 0- | 0- | 0- | 0- |
| such as BHT, | 0.6% | 0.6% | 0.6% | 0.7% | 0.7 | 0.8% | 0.8% | 0.8% | 0.9% | 1.0% |
| BHA, or a | | | | | | | | | | |
| combination | | | | | | | | | | |
| thereof | | | | | | | | | | |
| Solubilizing | 0- | 0- | 0.1- | 0.3- | 0- | 0 | 0- | 0- | 0- | 0- |
| agent such as | 2.0% | 0.75% | 0.7% | 0.7% | 2.0% | | 2.0% | 2.0% | 2.0% | 2.0% |
| Hypromellose | | | | | | | | | | |
| (HPMC) | | | | | | | | | | |
| Surfactant | 1.0- | 2.0- | 2.5- | 4.5- | 4.5- | 5.5- | 0.5- | 1.5- | 0.5- | 0.5- |
| such as | 15.0% | 13.5% | 12.5% | 13.5% | 13.5% | 13.0% | 8.5% | 13.0% | 12.0% | 12.0% |
| Steareth 21 | | | | | | | | | | |
| (Brij s721), | | | | | | | | | | |
| Steareth 2 | | | | | | | | | | |
| (Brij S2), | | | | | | | | | | |
| Cetomacrogol | | | | | | | | | | |
| 1000, | | | | | | | | | | |
| Myrj S40, | | | | | | | | | | |
| Brij CS25, | | | | | | | | | | |
| (Ceteareth | | | | | | | | | | |
| 25), Geleol | | | | | | | | | | |
| mono- and | | | | | | | | | | |
| diglycerides, | | | | | | | | | | |
| Span 20, | | | | | | | | | | |
| Span 60, or | | | | | | | | | | |
| Tween 60 | | | | | | | | | | |
| Thickening | 0.5- | 1.0- | 4.0- | 5.0- | 5.0- | 7.0- | 4.0- | 5.0- | 5.0- | 5.0- |
| agent such as | 18.0% | 15.0% | 15.0% | 11.0% | 11.0% | 11.0% | 9.0% | 11.0% | 11.0% | 11.0% |
| PEG 1500, | | | | | | | | | | |
| PEG 4000, or | | | | | | | | | | |
| HPMC | | | | | | | | | | |
| Stabilizing | 0- | 0- | 2.5- | 0- | 0- | 0- | 2.5- | 2.5- | 2.5- | 2.5- |
| agent such as | 10.00% | 8.50% | 8.50% | 8.50% | 8.50% | 8.50% | 8.50% | 8.50% | 8.50% | 8.50% |
| beeswax, | | | | | | | | | | |
| ceresin wax, | | | | | | | | | | |
| white | | | | | | | | | | |
| petrolatum, or | | | | | | | | | | |
| liquid paraffin | | | | | | | | | | |

In some embodiments, the topical, e.g. dermal, pharmaceutical composition comprises one of the combinations of ingredients in each of the weight percentages (% w/w, or wt %), or about the % w/w, provided below in Table E.

TABLE E
Composition (% w/w) of exemplary topical pharmaceutical compositions

| Ingredient/ | Composition (% w/w) | | | | |
|---|---|---|---|---|---|
| Excipient | Entry 1 | 2 | 3 | 4 | 5 |
| Active agent | 0.1-7.5% | 0.5-2.5% | 0.5-2.5% | 0.5-2.5% | 0.5-2.5% |
| such as | | | | | |
| ADX-102 or a | | | | | |
| pharmaceutically | | | | | |
| acceptable | | | | | |
| salt thereof | | | | | |
| Penetration | 1.0-18.0% | 3.0-17.0% | 5.0-16.0% | 7.0-15.0% | 7.0-12.0% |
| enhancer | | | | | |
| such as | | | | | |
| Transcutol HP | | | | | |
| Solvent/ | 0-11.0% | 2.0-9.0% | 4-7.5% | 0-8.0% | 2.0-9.0% |
| penetration | | | | | |
| enhancer such | | | | | |
| as Propylene | | | | | |
| Glycol | | | | | |
| Purified | 28.0-40.0% | 29.0%-38.0% | 30.0-37.0% | 28.0-36.0% | 29.0-40.0% |
| Water | | | | | |
| Solvent | 12.0-48.0% | 15.0-42.0% | 18.0-40.0% | 14.0-29.0% | 21.0-37.0% |
| such as S.R. | | | | | |
| PEG 400 | | | | | |
| Solvent/ | 2.0-15.0% | 5.0-13.0% | 5.0-13.0% | 4.0-14.0% | 4.0-15.0% |
| penetration | | | | | |
| enhancer/ | | | | | |
| solubilizing | | | | | |
| agent such | | | | | |
| as Ethanol | | | | | |
| Preservative | 0-3.0% | 0.1-3.0% | 0.25-2.5% | 0-3.0% | 0.25-2.5% |
| such as | | | | | |
| Phenoxyethanol | | | | | |
| Antioxidant | 0-0.2% | 0-0.2% | 0-0.2% | 0-0.2% | 0-0.2% |
| such as BHT | | | | | |
| Antioxidant | 0-0.4% | 0-0.4% | 0-0.4% | 0-0.4% | 0.01-0.3 |
| such as BHA | | | | | |
| Thickening | 0-2.0% | 0-0.75% | 0.1-0.7% | 0.3-0.7% | 0-2.0% |
| agent such as | | | | | |
| HPMC | | | | | |
| Thickening | 0.5-18.0% | 1.0-15.0% | 4.0-15.0% | 5.0-11.0% | 5.0-11.0% |
| agent such as | | | | | |
| PEG 1500 | | | | | |
| Surfactant | 0-4.6% | 0-3.0% | 0-1.0% | 0 | 0 |
| such as | | | | | |
| Steareth 21 | | | | | |
| (Brij s721) | | | | | |
| Surfactant | 0-5.6% | 0.5-5.6% | 1.0-4.5% | 0 | 0 |
| such as | | | | | |
| Steareth 2 | | | | | |
| (Brij S2) | | | | | |
| Surfactant | 0-2.6% | 0 | 0 | 0.5-2.0% | 0 |
| such as | | | | | |
| Cetomacrogol | | | | | |
| 1000 | | | | | |
| Surfactant | 0-2.8% | 0 | 0 | 0 | 0 |
| such as | | | | | |
| Myrj S40 | | | | | |
| Surfactant | 0-2.4% | 0.6-2.2% | 0 | 0 | 0 |
| such as | | | | | |
| Brij CS25 | | | | | |
| (Ceteareth 25) | | | | | |
| Surfactant | 0-14.0% | 3.0-10.0% | 4.0-9.0% | 0-8.0% | 3.0-9.0% |
| such as | | | | | |
| Geleol mono- | | | | | |
| and | | | | | |
| diglycerides | | | | | |
| Surfactant | 0-4.0% | 0-4.0% | 1.0-3.0% | 1.0-3.0% | 1.0-3.0% |
| such as | | | | | |
| Span 20 | | | | | |
| Stabilizing | 0-6.5% | 0-6.2% | 1.0-5.0% | 1.0-5.0% | 1.5-4.0% |
| agent such | | | | | |
| as Beeswax | | | | | |
| Stablizing | 0-7.5% | 1.5-7.1% | 1.5-5.5% | 1.5-5.5% | 1.5-5.5% |
| agent such | | | | | |
| as White | | | | | |
| petrolatum | | | | | |
| Stabilizing | 0-10.00% | 0-8.50% | 2.5-8.50% | 0-8.50% | 0-8.50% |
| agent such | | | | | |
| as Liquid | | | | | |
| Paraffin | | | | | |
| Surfactant | 0-3% | 0-3% | 0-3% | 0-3% | 0-3% |
| such as | | | | | |
| Steareth 20/ | | | | | |
| Brij S20 | | | | | |
| Ingredient/ | Composition (% w/w) | | | | |
| Excipient | 6 | 7 | 8 | 9 | 10 |
| Active agent | 0.5-2.5% | 0.5-2.5% | 0.5-2.5% | 0.5-2.5% | 0.5-2.5% |
| such as | | | | | |
| ADX-102 or a | | | | | |
| pharmaceutically | | | | | |
| acceptable | | | | | |
| salt thereof | | | | | |
| Penetration | 0.8-4.0% | 1.5-5.5% | 7.0-12.0% | 7.0-12.0% | 7.0-12.0% |
| enhancer | | | | | |
| such as | | | | | |
| Transcutol HP | | | | | |
| Solvent/ | 0 | 0 | 0 | 0 | |
| penetration | | | | | |
| enhancer such | | | | | |
| as Propylene | | | | | |
| Glycol | | | | | 0 |
| Purified | 28.0-37.0% | 34.0-40.0% | 28.0-36.0% | 28.0-36.0% | 28.0-36.0% |
| Water | | | | | |
| Solvent | 30.0-45.0% | 32.0-48.0% | 22.0-35.0% | 22.0-35.0% | 22.0-35.0% |
| such as S.R. | | | | | |
| PEG 400 | | | | | |
| Solvent/ | 3.0-15.0% | 0-12% | 4.0-15.0% | 4.0-15.0% | 4.0-15.0% |
| penetration | | | | | |
| enhancer/ | | | | | |
| solubilizing | | | | | |
| agent such | | | | | |
| as Ethanol | | | | | |
| Preservative | 0.25-2.5% | 0.25-2.5% | 0.25-2.5% | 0.25-2.5% | 0.25-2.5% |
| such as | | | | | |
| Phenoxyethanol | | | | | |
| Antioxidant | 0.01-0.2% | 0-0.2% | 0-0.2% | 0-0.2% | 0-0.2% |
| such as BHT | | | | | |
| Antioxidant | 0-0.4% | 0-0.4% | 0-0.4% | 0-0.4% | 0-0.4% |
| such as BHA | | | | | |
| Thickening | 0 | 0-2.0% | 0-2.0% | 0-2.0% | 0-2.0% |
| agent such as | | | | | |
| HPMC | | | | | |
| Thickening | 7.0-11.0% | 4.0-9.0% | 5.0-11.0% | 5.0-11.0% | 5.0-11.0% |
| agent such as | | | | | |
| PEG 1500 | | | | | |
| Surfactant | 0 | 0 | 0-3.0% | 0 | 0 |
| such as | | | | | |
| Steareth 21 | | | | | |
| (Brij s721) | | | | | |
| Surfactant | 0-5.6% | 0-5.6% | 0-5.6% | 0-5.6% | 0-5.6% |
| such as | | | | | |
| Steareth 2 | | | | | |
| (Brij S2) | | | | | |
| Surfactant | 0 | 0 | 0 | 0 | 0 |
| such as | | | | | |
| Cetomacrogol | | | | | |
| 1000 | | | | | |
| Surfactant | 0 | 0 | 0.5-2.8% | 0 | 0 |
| such as | | | | | |
| Myrj S40 | | | | | |
| Surfactant | 0.5-2.2% | 0 | 0 | 0.5-2.2% | 0.6-2.0% |
| such as | | | | | |
| Brij CS25 | | | | | |
| (Ceteareth 25) | | | | | |
| Surfactant | 0 | 0 | 0-14.0% | 0-14.0% | 0-14.0% |
| such as | | | | | |
| Geleol mono- | | | | | |
| and | | | | | |
| diglycerides | | | | | |
| Surfactant | 1.0-3.0% | 1.0-3.0% | 1.0-3.0% | 1.0-3.0% | 1.0-3.0% |
| such as | | | | | |
| Span 20 | | | | | |
| Stabilizing | 1.0-5.0% | 1.0-5.0% | 1.0-5.0% | 1.0-5.0% | 0.0-5.0% |
| agent such | | | | | |
| as Beeswax | | | | | |
| Stablizing | 1.5-5.5% | 1.5-5.5% | 1.5-5.5% | 1.5-5.5% | 1.5-5.5% |
| agent such | | | | | |
| as White | | | | | |
| petrolatum | | | | | |
| Stabilizing | 0-8.50% | 2.5-8.50% | 2.5-8.50% | 2.5-8.50% | 2.5-8.50% |
| agent such | | | | | |
| as Liquid | | | | | |
| Paraffin | | | | | |
| Surfactant | 0-3% | 0-3% | 0-3% | 0-3% | 0-3% |
| such as | | | | | |
| Steareth 20/ | | | | | |
| Brij S20 | | | | | |
| | Composition (% w/w) | | | | |
| Excipient | Entry 1 | 2 | 3 | 4 | 5 |
| Active agent | 0.5-2.50% | 0.5-2.50% | 0.5-2.50% | 0.5-9.0% | 0.5-2.50% |
| such as | | | | | |
| ADX-102 | | | | | |
| or a | | | | | |
| pharmaceutically | | | | | |
| acceptable salt | | | | | |
| thereof | | | | | |
| Penetration | 4.0-18.0% | 4.0-20.0% | 0.5-3.5% | 0.25-2.0% | 0.25-2.0% |
| enhancer or | | | | | |
| solvent such as | | | | | |
| Transcutol HP | | | | | |
| Solvent/ | 2.0-11.0% | 0 | 0 | 0 | 0 |
| Penetration | | | | | |
| enhancer | | | | | |
| such as | | | | | |
| Propylene | | | | | |
| Glycol | | | | | |
| Purified Water | 20.0-48.0% | 20.0-48.0% | 20.0-48.0% | 20.0-48.0% | 20.0-39.0% |
| Solvent such as | 13.0-32.0% | 17-38.0% | 24.0-45.0% | 26.0-49.0% | 31.0-58.0% |
| S.R. PEG 400 | | | | | |
| Second solvent | 3.0-15.0% | 4.0-16.0% | 4.5-17.0% | 4.0-16.0% | 4.0-16.0% |
| such as Ethanol | | | | | |
| Preservative | 0.25-2.5% | 0.25-2.5% | 0.25-2.5% | 0.25-2.5% | 0.25-2.5% |
| such as | | | | | |
| Phenoxyethanol | | | | | |
| Antioxidant | 0.01-0.2% | 0.01-0.2% | 0.01-0.2% | 0.01-0.2% | 0.01-0.2% |
| such as BHT | | | | | |
| Antioxidant | 0.01-0.2% | 0.01-0.2% | 0.01-0.2% | 0.01-0.2% | 0.01-0.2% |
| such as BHA | | | | | |
| Thickening | 0-1.1% | 0-1.1% | 0-1.1% | 0 | 0 |
| agent such | | | | | |
| as HPMC | | | | | |
| Thickening | 2.0-9.5% | 2.0-9.5% | 2.0-9.5% | 0.7-4.0% | 0.7-4.0% |
| agent | | | | | |
| such as | | | | | |
| PEG 1500 | | | | | |
| Surfactant | 0 | 0 | 0 | 1.0-6.0% | 1.0-6.0% |
| such as | | | | | |
| Steareth 2 | | | | | |
| (Brij S2) | | | | | |
| Surfactant | 3.0-14.0% | 3.0-14.0% | 3.0-14.0% | 0 | 0 |
| such as | | | | | |
| Geleol mono | | | | | |
| and | | | | | |
| diglycerides | | | | | |
| Surfactant | 1.0-4.0% | 1.0-4.0% | 1.0-4.0% | 0 | 0 |
| such as | | | | | |
| Span 20 | | | | | |
| Stabilizing | 1.2-6.50% | 1.2-6.50% | 1.2-6.50% | 0 | 0 |
| agent such | | | | | |
| as Beeswax | | | | | |
| Stabilizing | 1.2-6.50% | 1.2-6.50% | 1.2-6.50% | 1.2-6.50% | 1.2-6.50% |
| agent such | | | | | |
| as White | | | | | |
| petrolatum | | | | | |
| Stabilizing | 0 | 0 | 0 | 2.0-10.0% | 2.0-10.0% |
| agent such | | | | | |
| as Liquid | | | | | |
| Paraffin | | | | | |
| Surfactant | 0.4-2.0% | 0.4-2.0% | 0.4-2.0% | 0.7-3.5% | 0.7-3.5% |
| such as | | | | | |
| Steareth 20/ | | | | | |
| Brij S20 | | | | | |

In some embodiments, the topical, e.g. dermal, pharmaceutical composition comprises one of the combination of ingredients in each of the weight percentages (% w/w, or wt %), or about the % w/w, provided below in Table F.

TABLE F
Composition (% w/w) of exemplary topical pharmaceutical compositions

| Ingredient/ | Composition (% w/w) | | | | | | | | | |
|---|---|---|---|---|---|---|---|---|---|---|
| Excipient | Entry 1 | 2 | 3 | 4 | 5 | 6 | 7 | 8 | 9 | 10 |
| ADX-102 or a | 1.00 | 1.00 | 1.00 | 1.00 | 1.00 | 1.00 | 1.00 | 1.00 | 1.00 | 1.00 |
| pharmaceutically | | | | | | | | | | |
| acceptable salt | | | | | | | | | | |
| thereof | | | | | | | | | | |
| Transcutol HP | 8.65 | 8.65 | 8.65 | 9.60 | 9.60 | 1.68 | 3.15 | 9.60 | 9.60 | 9.60 |
| Propylene Glycol | 5.50 | 5.50 | 5.50 | — | — | — | — | — | — | — |
| Purified Water | 34.93 | 34.93 | 34.93 | 30.40 | 30.40 | 30.40 | 38.61 | 30.40 | 30.40 | 30.40 |
| S.R. PEG 400 | 19.65 | 19.65 | 19.65 | 28.60 | 28.60 | 36.12 | 36.57 | 28.60 | 28.60 | 28.60 |
| Ethanol | 7.86 | 7.86 | 7.86 | 8.00 | 8.00 | 8.40 | — | 8.00 | 8.00 | 8.00 |
| Phenoxyethanol | 1.00 | 1.00 | 1.00 | 1.00 | 1.00 | 1.00 | 1.00 | 1.00 | 1.00 | 1.00 |
| BHT | 0.05 | 0.05 | 0.05 | 0.10 | 0.10 | 0.10 | — | 0.10 | 0.10 | — |
| BHA | 0.10 | 0.10 | 0.10 | 0.10 | 0.10 | 0.10 | 0.10 | 0.10 | — | 0.10 |
| PEG 1500 | 9.26 | 9.26 | 9.26 | 9.20 | 9.20 | 9.20 | 7.57 | 9.20 | 9.30 | 9.30 |
| Steareth 21 | — | — | — | — | — | — | — | 1.52 | — | — |
| (Brij s721) | | | | | | | | | | |
| Steareth 2 | 2.60 | 2.84 | 2.37 | 2.70 | 2.37 | 2.84 | 2.37 | 2.48 | 2.84 | 2.84 |
| (Brij S2) | | | | | | | | | | |
| Cetomacrogol | — | — | — | 1.30 | — | — | — | — | — | — |
| 1000 | | | | | | | | | | |
| Myrj S40 | 1.40 | — | — | — | — | — | — | — | — | — |
| Brij CS25 | — | 1.16 | — | — | — | 1.16 | — | — | 1.16 | 1.16 |
| (Ceteareth 25) | | | | | | | | | | |
| White | 3.00 | 3.00 | 3.00 | 3.00 | 3.00 | 3.00 | 3.00 | 3.00 | 3.00 | 3.00 |
| petrolatum | | | | | | | | | | |
| Liquid | 5.00 | 5.00 | 5.00 | 5.00 | 5.00 | 5.00 | 5.00 | 5.00 | 5.00 | 5.00 |
| Paraffin | | | | | | | | | | |
| Steareth 20/ | — | — | 1.63 | — | 1.63 | — | 1.63 | — | — | — |
| Brij S20 | | | | | | | | | | |

| | Composition (% w/w) | | | | |
|---|---|---|---|---|---|
| Excipient | Entry 11 | 12 | 13 | 14 | 15 |
| ADX-102 | 1.00 | 1.00 | 1.00 | 5.00 | 1.00 |
| Transcutol HP | 8.65 | 9.60 | 1.68 | 0.80 | 0.80 |
| Propylene | 5.50 | — | — | — | — |
| Glycol | | | | | |
| Purified Water | 34.93 | 30.40 | 30.40 | 30.40 | 30.40 |
| S.R. PEG 400 | 19.65 | 28.60 | 36.12 | 40.90 | 44.90 |
| Ethanol | 7.86 | 8.00 | 8.40 | 8.00 | 8.00 |
| Phenoxyethanol | 1.00 | 1.00 | 1.00 | 1.00 | 1.00 |
| BHT | 0.05 | 0.10 | 0.10 | 0.10 | 0.10 |
| BHA | 0.10 | 0.10 | 0.10 | 0.10 | 0.10 |
| HPMC | 0.50 | 0.50 | 0.50 | — | — |
| PEG 1500 | 4.86 | 4.80 | 4.80 | 1.70 | 1.70 |
| Steareth 2 | — | — | — | 2.37 | 2.37 |
| (Brij S2) | | | | | |
| Geleol mono and | 7.00 | 7.00 | 7.00 | — | — |
| diglycerides | | | | | |
| Span 20 | 2.00 | 2.00 | 2.00 | — | — |
| Beeswax | 3.00 | 3.00 | 3.00 | — | — |
| White | 3.00 | 3.00 | 3.00 | 3.00 | 3.00 |
| petrolatum | | | | | |
| Liquid Paraffin | — | — | — | 5.00 | 5.00 |
| Steareth 20/ | 0.90 | 0.90 | 0.90 | 1.63 | 1.63 |
| Brij S20 | | | | | |

In another aspect, the present invention provides a method of preparing a disclosed topical formulation, comprising the step of: blending the solid and liquid excipients with an active agent or pharmaceutically acceptable salt thereof to produce a homogenous mixture such as a cream, wherein the active agent is dissolved in the mixture.

In some embodiments, the present invention provides a method of preparing a topical formulation, such as an oil-in-water cream formulation, comprising the step of: blending

(i) an antioxidant and/or preservative (where applicable)

(ii) non-volatile and volatile aqueous-phase excipients such as water, a penetration enhancer, or a solvent;

(iii) an active agent or pharmaceutically acceptable salt thereof;

(iv) oil phase excipients such as a surfactant, emulsifier, and stabilizing agent;

(v) optionally, a thickening agent such as PEG 1500;

optionally, warming the mixture to, e.g., 60-70° C.; and

optionally, homogenizing the mixture under appropriate conditions such as with a homogenizer for several minutes at 10,000 RPM.

Methods of Treatment

In one aspect, the present invention provides a method of treating a disease, disorder, or condition, comprising administering topically to a subject in need thereof a disclosed topical (e.g., dermal) pharmaceutical composition.

In some embodiments, the present invention provides a method of treating a skin disease, disorder, or condition, comprising administering topically to a subject in need thereof a disclosed dermal pharmaceutical composition.

In some embodiments, the present invention provides a method of treating a disease, disorder, or condition in which aldehyde toxicity is implicated in the pathogenesis, comprising administering topically to a subject in need thereof a disclosed topical (e.g., dermal) pharmaceutical composition.

In some embodiments, the disease, disorder, or condition is psoriasis.

In some embodiments, the disease, disorder, or condition is psoriatic arthritis.

In some embodiments, the disease, disorder, or condition is atopic dermatitis.

In some embodiments, the disease, disorder, or condition is rosacea.

In some embodiments, the disease, disorder, or condition is gout.

In some embodiments, the disease, disorder, or condition is pseudogout.

In some embodiments, the disease, disorder, or condition is a skin disorder or condition or a cosmetic indication. These include, but are not limited to, psoriasis, topical (discoid) lupus, contact dermatitis, atopic dermatitis, allergic dermatitis, radiation dermatitis, acne vulgaris, Sjögren-Larsson Syndrome (SLS) and/or other ichthyoses, solar elastosis/wrinkles, skin tone firmness, puffiness, eczema, smoke or irritant induced skin changes, dermal incision, and a skin condition associated with a burn or wound. In some embodiments, the disease, disorder, or condition is a condition associated with the toxic effects of blister agents or burns from alkali agents, transepidermal water loss (TEWL), neurogical, and/or motor effects of SLS, SSADHD, and pyridoxine-dependent epilepsy. In some embodiments, the disease, disorder, or condition is succinic semialdehyde dehydrogenase deficiency (SSADHD), also known as 4-hydroxybutyric aciduria or gamma-hydroxybutyric aciduria.

In some embodiments, the disease, disorder, or condition is SLS. In some embodiments, treatment of SLS includes treatment of ichthyoses, neurological effects, motor effects, or TEWL or another symptom or sequela associated with SLS.

In some embodiments, treatment of SLS comprises an improvement in Visual Index for Ichthyosis Severity (VIIS) scaling severity score in the patient, for example as assessed by a medical professional in the target efficacy area. In some embodiments, treatment of SLS comprises a VIIS improvement of at least 0.1 units. In some embodiments, treatment of SLS comprises a VIIS improvement of at least 0.2 units, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, or 3.0 units. In some embodiments, treatment of SLS comprises a VIIS improvement of about 0.1-3 units, 0.2-2.5, 0.3-2.0, 0.4-1.9, 0.5-1.8, 0.6-1.7, 0.7-1.6, 0.8-1.5, 0.5-2.0, 0.5-1.5, or 0.5-1.0 units.

In some embodiments, the patient has a VIIS of Grade 2 or worse.

In some embodiments, treatment of SLS comprises an improvement in at least one of the following:
(i) VIIS scaling erythema score in the target efficacy area
(ii) VIIS scaling and erythema severity score as assessed by a central reader in the target efficacy area
(iii) EQ-5D-5L quality of life assessment
(iv) Clinician global impression of severity
(v) Patient/Observer global impression of severity
(vi) Patient/Observer global impression of bother
(vii) Patient/Observer global impression of treatment
(viii) Patient-reported itching
(ix) Patient/Observer-reported scratching
(x) Patient/Observer Burden of bathing/showering
(xi) Children's Dermatology Life of Quality Index (CDLQI)/Dermatology Life Quality Index (DLQI)
(xii) Lipid biomarkers from skin cells
(xiii) Transepidermal Water Loss (TEWL)

In some embodiments, the disease, disorder, or condition is selected from an age-related disease, disorder, or condition of the skin, as described herein.

Various skin disorders or conditions, such as atopic dermatitis, topical (discoid) lupus, psoriasis and scleroderma, are characterized by high toxic aldehyde levels, such as MDA and HNE levels (Br J Dermatol 149: 248 (2003); JEADV 26: 833 (2012); Clin Rheumatol 25: 320 (2006)). In addition, ichthyosis associated with Sjögren-Larsson Syndrome (SLS) originates from accumulation of fatty aldehydes, which disrupts the normal function and secretion of lamellar bodies (LB) and leads to intercellular lipid deposits in the strateum corneum (SC) and a defective water barrier in the skin (W. B. Rizzo et al. (2010)). In patients with SLS, mutations in the gene encoding fatty aldehyde dehydrogenase, which metabolizes fatty aldehydes, significantly reduce or ablate its activity. Thus, compounds that reduce or eliminate aldehydes, such as the compounds described herein, can be used to treat, prevent, and/or reduction of a risk of skin disorders or conditions in which aldehyde toxicity is implicated in the pathogenesis, such as those described herein. Furthermore, with an improvement to the water barrier and prevention of aldehyde-mediated inflammation (including fibrosis and elastosis (Chairpotto et al. (2005)), many cosmetic indications, such as solar elastosis/wrinkles, skin tone, firmness (puffiness), eczema, smoke or irritant induced skin changes and dermal incision cosmesis, and skin conditions associated with burn and/or wound can be treated using the method of the invention.

In some embodiments, the disease, disorder, or condition is a condition associated with the toxic effects of blister agents or burns from alkali agents. In some embodiments, the blister agent is sulfur mustard, nitrogen mustard, or phosgene oxime. In some embodiments, the alkali agent is lime, lye, ammonia, or a drain cleaner.

In some embodiments, the disease, disorder, or condition is an autoimmune, immune-mediated, inflammatory, cardiovascular, neurological disease, diabetes, metabolic syndrome, or a fibrotic disease.

Administration

The presently described topical compositions can be topically administered in a disclosed formulation, including a dermal cream or paste. A sufficient amount (e.g., an effective amount to treat the disease, disorder, or condition) of the topical preparation can be gently rubbed onto the affected area and surrounding skin, for example, in an amount sufficient to cover an affected area plus a margin of healthy skin or tissue surrounding the affected area, for example, a margin of about 0.5 inches. In some embodiments, such as for treatment of SLS, the treatment area covers significant portions of the skin, or even the entire skin surface, of the subject. In some embodiments, the entire skin surface except for glabrous skin is treated.

A disclosed composition can be applied in a single, one-time application, once a week, biweekly, once a month, or from one to four times daily, for a period of time sufficient to alleviate symptoms or treat the disease, disorder, or condition, for example, for a period of time of one week, from 1 to 54 weeks or more, from 1 to 24 weeks, from 1 to 8 weeks, from 2 to 12 weeks, from 2 to 10 weeks, from 2 to 8 weeks, from 2 to 6 weeks, from 2 to 4 weeks, from 4 to 12 weeks, from 4 to 10 weeks, from 4 to 24 weeks, from 4 to 54 weeks, or chronically or for the lifetime of the subject. The present compositions can be administered, for example, at a frequency of once per day or twice per day. The presently described compositions can be topically administered once per day for a period of time from 1 week to 2 years, from 1 week to 5 years, from 1 month to 6 months, or from 1 month to 12 months, or chronically, i.e., indefinitely. The administration can be for the lifetime of the subject in certain cases.

In some embodiments, a disclosed composition is administered in a pulsed or intermittent dosing schedule. In some embodiments, a disclosed composition is administered (e.g., daily) during a “dosing period,” followed by a “non-dosing period,” followed by a second dosing period. In some embodiments, the dosing period is about 1, 2, 3, 4, 5, 6, 7, 8, 9, or 10 weeks. In some embodiments, the non-dosing period is about 1, 2, 3, 4, 5, 6, 7, 8, 9, or 10 weeks. In some embodiments, a pulsed dosing schedule of about 1:1 dosing:non-dosing periods is followed. In some embodiments, a pulsed dosing of about 2:1, 3:1, 4:1, 5:1, 6:1, 7:1, 8:1, 9:1, or 10:1 dosing:non-dosing periods is followed. In some embodiments, a pulsed dosing of about 1:2, 1:3, 1:4, 1:5, 1:6, 1:7, 1:8, 1:9, or 1:10 dosing:non-dosing periods is followed.

In some embodiments, an effective amount is selected from a dose of about 8 mg/cm² of a dermal cream containing 1% w/w ADX-102 or another active agent disclosed herein, with each application administered approximately 8, 12, 16, or 24 hours apart. In some embodiments, an effective amount is about 0.25-80 mg/cm² of such a dermal cream. In some embodiments, an effective amount is about 1-60, 2-50, 3-40, 4-30, 5-20, 6-10, 0.5-20, 0.75-15, 1-12, 1-11, 1-10, 1-9, 1-8, 1-7, 1-6, 1-5, 1-4, 1.5-20, 2-20, 2.5-20, 3-20, 4.5-20, 5-15, 5-12, 5-10, 5-8, about 1, about 2, about 3, about 4, about 5, about 6, about 7, about 8, about 9, about 10, about 15, about 20, about 25, about 30, about 40, about 50, about 60, about 70, or about 80 mg/cm² of such a dermal cream. Such amounts are scaled appropriately if the dermal cream contains a w/w % of ADX-102 or another active agent in an amount other than 1%, such as 5% or 0.5%.

In some embodiments, the present invention provides a topical, e.g., dermal pharmaceutical composition that provides an improved flux across the skin, e.g. across the stratum corneum. In some embodiments, the composition provides a flux (ng/cm²/√hr) as shown in Table 41 below. In some embodiments, the flux is about 63 to 730 μg/cm²/√h when measured in a Franz chamber with a human skin sample and after application of about 10 mg/cm² of the composition comprising a concentration of about 1% w/w active agent, such as ADX-102. In some embodiments, the flux is about 30-1,200, 50-1,100, 60-1,000, 60-900, 70-800, 100-800, 150-800, 200-800, 300-800, 400-800, 500-800, 600-800, or 700-800 μg/cm²/√h.

In some embodiments, the composition provides improved delivery to the epidermis or dermis or selective partitioning into the epidermis or dermis. In some embodiments, the composition delivers an active agent into the epidermis vs. the dermis in a ratio of about 20:1, 15:1, 12:1, 10:1, 8:1, 6:1, 5:1, 4:1, 3:1, 2:1, 1:1, 1:2, 1:3, 1:4, 1:5, 1:6, 1:8, 1:10, 1:12, 1:15, or 1:20. In some embodiments, the composition delivers less than about 5%, less than about 2.5%, less than about 1.5%, less than about 1%, less than about 0.5%, less than about 0.25%, less than 0.125%, or less than 0.05% of the active agent into the circulation of a subject, i.e. through the epidermis and dermis.

### Examples

The following examples are illustrative of the pharmaceutical compositions and other provided aspects of the present invention and are not intended to be limitations thereon.

### Example 1: Definitions, Abbreviations, and Preliminary and Comparative Studies

The following definitions and abbreviations are used herein.

TABLE 8
List of definitions and abbreviations

| Abbreviation | Definitions |
|---|---|
| μg | Microgram |
| μL | Microliter |
| AO | Antioxidant |
| AOL | Antioxidant Level |
| API | Active Pharmaceutical Ingredient |
| BHA | Butylated hydroxyanisole |
| BHT | Butylated hydroxytoluene |
| ° C. | Degree Celsius |
| ca. | Circa |
| CR | Cream |
| C of A | Certificate of analysis |
| DMI | Dimethyl Isosorbide |
| DMF | Drug Master File |
| DoE | Design of Experiment |
| FDA | Food and Drug Administration (US) |
| g | Gram |
| GLP | Good Laboratory Practice |
| GxP | Good Practice Guidelines |
| GRAS | Generally recognized as safe |
| h | Hour |
| H₂O₂ | Hydrogen peroxide |
| HPLC | High Performance Liquid Chromatography |
| HPMC | Hypromellose |
| IIG | Inactive Ingredients Guide (FDA) |
| IPA | Isopropyl alcohol |
| IPM | Isopropyl myristate |
| IPP | Isopropyl palmitate |
| IVPT | In vitro permeation and penetration testing |
| IVRT | In vitro release testing |
| L | Liter |
| LOD | Limit of Detection |
| LOQ | Limit of Quantification |
| mg | Milligram |
| min | Minute |
| mL | Milliliter |
| mm | Millimeter |
| n | Number of independent replicates |
| N/A | Not applicable |
| nm | Nanometer |
| NMP | N-Methylpyrrolidone |
| OECD | Organization for Economic Co-operation and Development |
| PEG | Polyethylene glycol |
| PLB | Placebo |
| PTFE | Polytetrafluoroethylene |
| QA | Quality Assurance |
| QC | Quality Control |
| R² | Coefficient of determination of a linear regression |
| RPM | Revolutions per minute |
| SLS | Sjogren-Larsson Syndrome |
| SOP | Standard Operating Procedure |
| SR | Super-Refined |
| SS | Solvent System |
| v/v | Volume/volume |
| w/w | Weight/weight |

The purpose of this study was to develop a topical formulation containing active agents such as ADX-102 suitable for the treatment suitable for the treatment of certain diseases and conditions such as those described herein, for example ichthyoses due to Sjögren-Larsson Syndrome. Two general approaches for developing the topical formulation were pursued: first, initially manufacturing a new formulation, using pre-formulation data, and secondly optimizing our comparison formulation to improve the physical and chemical stability.

Solubility experiments were performed using an array of excipients and binary solvent systems, highlighting a range of solvents (phenoxyethanol, ethanol, isopropanol, SR PEG 400, Arlasolve DMI, diisopropyl adipate, IPM, Transcutol HP and benzyl alcohol) and non-solvents (water and glycerol) for ADX-102. ADX-102 was also shown to have solubility of 0.31% in the comparison formulation solvent system, despite the label claim of 1.0% w/w, highlighting a potential reason for the crystallization observed in the comparison formulation. ADX-102 was shown to have good stability in a range of excipients tested (>98.21% peak purity; e.g., benzyl alcohol, phenoxyethanol, SR PEG 400, Transcutol HP, ethanol, water, propylene glycol), with the exception of isopropyl alcohol, oleyl alcohol, SR Arlasolve DMI and diisopropyl adipate (at 50° C.) after t=2 weeks. Antioxidant selection experiments employing a DoE experimental design were performed where BHA and BHT were observed to be the most effective antioxidant/stabilizers for preventing oxidative degradation of ADX-102 and the minimal levels for each antioxidant were determined.

We developed a range of cream formulations at 1% w/w and 5% w/w ADX-102 by use of a DoE and incorporating the appropriate excipients selected following the pre-formulation experiments, where a total of 15 cream formulations were selected for short-term stability testing at 25 and 40° C. Good chemical stability (percentage purity >97%) of ADX-102 was observed in all formulations following storage for 4 weeks at 25 and 40° C., with the exception of CR066 and CR072, which had percentage purity of 93.90 and 94.16% after t=4 weeks at 40° C., due to a large impurity peak at 20 mins. All of the cream formulations had no ADX-102 crystals present, although CR060, CR063 and CR065 were observed to phase separate over the 4-week testing period at 40° C. All remaining formulations had appealing aesthetics (white cream, medium-high viscosity, with a smooth application) at both T=0 and following stability storage over 4 weeks. In addition, a select of formulations (CR062, CR070 and CR079) were also assessed following 9 weeks of storage at 25 and 40° C. where comparable results to T=0 were observed. To summarize, we successfully developed a range of formulations containing ADX-102, which were observed to be chemically and physically stable over 9 weeks of storage at 25 and 40° C. Such formulations will also be suitable for the other aminocarbinol-containing aldehyde trapping compounds disclosed herein.

Introduction

The developed dermal cream can be used to treat skin diseases such as Sjögren-Larsson Syndrome (SLS), which is a rare disease caused by mutations in fatty acid aldehyde dehydrogenase, leading to elevated fatty aldehyde levels and severe ichthyosis (scaly, thickened, dry skin), neurological disorders, and retinal disease.

We first investigated making minimal changes to the first generation formulation, while improving chemical and physical stability. Secondly, we used the pre-formulation data (see below) to develop a completely new, second generation formulation, with a target concentration of 1.00% w/w for the topical treatment of ichthyoses due to Sjögren-Larsson Syndrome and other skin and topical diseases such as those disclosed herein. We also developed a 5% w/w formulation.

Active Agents

ADX-102 is a white to pale yellow powder and has a molecular weight of 236.7 g/mole, with a melting point of 113-115° C. The chemical structure of ADX-102 is shown below.

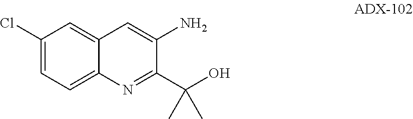 [SMILES: CC(C)(O)c1nc2ccc(Cl)cc2cc1N]

Issues with both chemical and physical stability have been observed in the comparison formulation due to suspected oxidation and degradation of ADX-102 as well as ADX-102 crystals present in the formulation as a suspension.

### Example 2: Summary of Formulation Development and Procedures

The comparison formulation was tested first; its composition is detailed in Table 9 below. The goal was to develop formulations based on the comparison formulation (which is a cream) in an attempt to improve the chemical and physical stability of the formulation. In parallel work described below, we developed a range of novel formulations, using pre-formulation data with target label strength of ADX-102 of 1% w/w. An additional formulation was prepared with drug concentration of 5% w/w, and studies showed that even higher concentrations of drug could be contained in the formulation.

TABLE 9
Composition (% w/w) of the comparison formulation

| Excipient | Composition (% w/w) |
|---|---|
| ADX-102 | 1.00 |
| Transcutol HP | 10.00 |
| Propylene Glycol | 5.00 |
| Span 20 | 2.00 |
| Hypromellose | 0.50 |
| White petrolatum | 3.00 |
| Ceresin Wax | 3.00 |
| Geleol mono and diglycerides | 7.00 |
| PEG 1500 | 5.00 |
| Purified Water | 62.60 |
| Steareth 20/Brij S20 | 0.90 |
| Total | 100.00 |

Characterization of the Developed Active and Placebo Formulations

A selection of the developed active and placebo formulations characterized using the tests summarized below were performed on a selected number of formulations:
Macroscopic appearance.
Absence of crystalline material (applicable to the active)/microscopic appearance.
pH.
Accelerated physical stability.

Short Term Formulation Stability

### Formulation Stability Overview

Following successful formulation development by following the methods described herein, candidate formulations were selected and assessed over the course of a short term stability program at t=0 and following 2, 4 and 9 weeks of storage at 25 and 40° C. The following parameters were evaluated over the stability experiments: macroscopic and microscopic appearance, ADX-102 content and peak purity, and apparent pH.

TABLE 10
Allocation of formulations for short-term stability program

Short term stability testing time points

| Condition | Initial t = 0 | 2 weeks | 4 weeks | 9 weeks |
|---|---|---|---|---|
| 2-8° C. | X | Y | Y | Y |
| 25° C. | | X | X | X |
| 40° C. | | X | X | X |

X = ADX-102 content and related substances, pH, microscopic and microscopic observations.
Y = Back up only, not tested.

### ADX-102 Content and Related Substances

The active and placebo formulations were extracted for ADX-102 (active formulations a total of n=3 for related substances and assay samples and n=1 for placebo formulation) using the following procedure to give a target concentration of ADX-102 of ca. 200 μg/mL.
(i) A known weight (0.20±0.05 g for the 1% w/w ADX-102 formulations or 0.10±0.05 g) for the 5% w/w ADX-102 formulations) of formulation was weighed into a 10 mL or a 25 mL volumetric flask for the 1% w/w ADX-102 and 5% w/w ADX-102 formulations (n=3).
(ii) The volumetric flask was made to volume with sample diluent (90:10 v/v acetonitrile:water).
(iii) The volumetric flask was vortex mixed for 30 seconds to disperse the formulation.
(iv) A PTFE magnetic stirrer was added to the volumetric flask from and the contents of the flask were stirred for a minimum of 2 h.
(v) The extraction solution was transferred to centrifuge tubes and the samples were centrifuged at 25° C. for 10 min at 13,000 RPM (16,060 g-force).
(vi) The remaining supernatant was vialled into amber HPLC vials and was analyzed using the developed assay HPLC method (n=2), and the related substances HPLC method (n=1).

Selection of Candidates for Performance Testing

Following assessment and analysis of both the short-term stability data (chemical and physical) generated as described above, 10 formulation candidates were selected for Performance Testing (In vitro drug Release Studies and In vitro drug permeation and penetration testing).

### Example 3: Pre-Formulation Experiments

Saturated Solubility of ADX-102 in Excipients

The saturated solubility of ADX-102 was investigated in individual excipients (with low aldehyde content/low potential to produce aldehydes upon degradation) suitable for topical application with the results presented in Table 11 and Table 12. In addition, as ADX-102 is an aldehyde trap, excipients with low risk of aldehyde content were assessed, such as super refined PEG 400 and Transcutol HP. ADX-102 was determined to be highly soluble >20.00% w/w in isopropanol, benzyl alcohol, super-refined PEG 400, phenoxyethanol, super-refined DMI, diisopropyl adipate, isopropyl myristate, Transcutol HP and ethanol (determined visually). ADX-102 solubility of between 10-20% w/w was observed in propylene carbonate, castor oil, miglyol 810 and propylene glycol, while ADX-102 solubility in oleyl alcohol, butyl stearate, and isopropyl palmitate was observed to be below 10% w/w. Poor solubility (<1.00% w/w) was apparent in deionised water, irrigation water and glycerol. ADX-102 was found to be soluble in propylene glycol (12.1% w/w (equivalent to 120.09 (111.22-126.29) mg/g)) as determined via the developed HPLC assay method. We had previously determined the solubility of ADX-102 in propylene glycol as 114 mg/g (mg ADX-102 per g mixture), and this generally agreed with the new data in this study. Additionally, the results showed that ADX-102 presented a solubility of >20.00% w/w in ethanol and Transcutol HP, consistent with previous data. The saturated solubility experiments highlighted potential solvents and non-solvents of ADX-102.

TABLE 11
Saturated solubility of ADX-102 (% w/w) in selected
individual excipients

| Excipient | Saturated solubility (% w/w) | |
|---|---|---|
| Isopropanol (Isopropyl alcohol) | ≥20.00 | (visual observation) |
| Benzyl alcohol | ≥20.00 | (visual observation) |
| Super refined PEG 400 | ≥20.00 | (visual observation) |
| Phenoxyethanol | ≥20.00 | (visual observation) |
| Propylene carbonate | 13.14 | (12.57-13.65) |
| Oleyl alcohol | 8.77 | (8.62-8.89) |
| Super refined Dimethyl Isosorbide | ≥20.00 | (visual observation) |
| (Arlasolve DMI) | | |
| Butyl stearate | 8.52 | (8.46-8.57) |
| Castor oil | 10.38 | (9.25-11.14) |
| Diisopropyl adipate | ≥20.00 | (visual observation) |
| Glycerin (Glycerol) | 0.75 | (0.56-1.00) |
| Isopropyl myristate (IPM) | ≥20.00 | (visual observation) |
| Isopropyl palmitate (IPP) | 7.27 | (7.24-7.33) |
| Miglyol 810 (Caprylic/Capric triglyceride | 11.83 | (11.06-12.52) |
| or medium chain triglycerides) | | |
| Water for irrigation | 0.02 | (0.01-0.03) |

Data is presented as the mean with the range in brackets (n = 3).
Data analyzed by the developed HPLC assay method unless otherwise stated.

TABLE 12
Saturated solubility of ADX-102 (% w/w) in individual excipients

| Excipient | Saturated solubility (% w/w) | |
|---|---|---|
| Deionized water | 0.13 | (0.04-0.23) |
| Transcutol HP | ≥20.00 | (visual observation) |
| Propylene glycol | 12.01 | (11.12-12.63) |
| Ethanol | ≥20.00 | (visual observation) |

Data is presented as the mean with the range in brackets (n = 3).
Data analyzed by the developed HPLC assay method unless otherwise stated.

### Saturated Solubility of ADX-102 in the Comparison Formulation

The saturated solubility of ADX-102 was also investigated in the aqueous phase of the comparison formulation and was observed to be 0.31% w/w, as shown in Table 13. It is important to note that the comparison formulation has a nominal ADX-102 concentration of 1.0% w/w, which was higher than that observed in the aqueous phase of the comparison formulation (0.31% w/w). As ADX-102 crystals were observed in the formulation, it appeared that the drug was partly in suspension with only ca. 0.31% w/w in solution. However, it is possible that oil phase excipients may assist in solubilizing ADX-102, allowing it to partition between phases.

TABLE 13
Saturated solubility of ADX-102 (% w/w) in comparison
formulation solvent system

| | Composition | Saturated |
|---|---|---|
| Excipient | (% w/w) | solubility (% w/w) |
| Transcutol HP | 11.90 | 0.31 |
| Propylene Glycol | 5.96 | |
| Purified Water | 74.53 | |
| PEG1500 | 5.95 | |
| Steareth 20/Brij S20 | 1.09 | |

Data analyzed by developed HPLC assay method.
Data is presented as the mean with the range in brackets (n = 3).

### Example 4: Antioxidant and Stabilizer Screening and Selection

ADX-102 Oxidation Investigation

Due to evidence of possible oxidative degradation, a selection of antioxidants and stabilizers were investigated in combination with PEG 400 to determine whether an antioxidant/stabilizer system would improve the stability of ADX-102, in comparison to systems without stabilizers present.

A preliminary study was performed in an attempt to degrade ADX-102 to a suitable level such that oxidation products observed during long-term stability could be detected. To this end, an ADX-102 in PEG 400 stock solution (0.1% w/w) was prepared and degraded with hydrogen peroxide (H₂O₂) and N-methylpyrrolidone (NMP) at 0.1 and 1.0% w/w concentrations, respectively. Additionally, the stocks were stressed at 40 and 70° C. to increase degradation. The degradation of ADX-102 was investigated at t=0, 24 and 48 h and the results are presented in Table 14. The data indicated that both peroxide spiking experiments (H₂O₂ and NMP) resulted in ADX-102 degrading to <70% after t=48 h at 70° C. Sufficient degradation of ADX-102 was observed following spiking with 1.0% w/w H₂O₂ and storage at 70° C. for 48 h condition and was therefore considered suitable for use in antioxidant/stabilizer selection experiments described below.

TABLE 14
Percentage recovery (% w/w) of ADX-102 after stress with peroxide spiking and
high temperature at t = 0, 24 and 48 h

| | Percentage recovery of ADX-102 (%) | | | | |
|---|---|---|---|---|---|
| Peroxide | | T = 24 h | | T = 48 h | |
| Spiking | T = 0 | 40° C. | 70° C. | 40° C. | 70° C. |
| 0.1% H₂O₂ | 100.00 | 90.10 | 86.52 | 73.23 | 70.13 |
| | (96.51-103.12) | (89.37-90.76) | (84.50-88.87) | (73.01-73.42) | (66.86-73.56) |
| 1.0% H₂O₂ | 100.00 | 88.41 | 82.92 | 74.00 | 66.27 |
| | (97.29-104.72) | (88.38-88.44) | (81.73-84.06) | (73.39-75.15) | (63.30-70.22) |
| 0.1% NMP | 100.00 | 89.79 | 86.35 | 74.47 | 70.64 |
| | (96.42-104.49) | (89.01-90.41) | (85.84-87.27) | (73.48-76.45) | (68.97-72.52) |
| 1.0% NMP | 100.00 | 89.43 | 86.02 | 72.13 | 68.52 |
| | (98.96-101.25) | (88.28-91.35) | (85.33-87.28) | (68.87-75.37) | (67.56-69.23) |

Data presented as the mean with the range in brackets (n = 3).
Data analyzed by developed HPLC assay method.

Antioxidant/Stabilizer DoE Mixture Design

Once the ADX-102 had been degraded to a suitable level, a statistical Design of Experiment (DoE) mixture design was performed, with the aim of determining the optimal antioxidant/stabilizer combination to prevent degradation and improve ADX-102 chemical stability under the stress conditions determined (1.0% w/w H₂O₂ spiking at 70° C.). A two-level fractional factorial design was employed that ensured that main factors and two-factor interactions were not confounded. The factors and the solvent system compositions are detailed in Table 15. Antioxidants and stabilizers were assessed in a 0.1% w/w ADX-102 in PEG 400 stock solution at t=0, 24 h, and 2 weeks. The recovery (% w/w) of ADX-102 (with the inclusion antioxidant/stabilizer systems AO1 through A028) was investigated after being stored at the stress conditions (determined during the preliminary study, 1.0% w/w H₂O₂ spiking at 70° C.) and the results are presented in Table 16.

The results show that the highest percentage recovery (from t=0) of ADX-102 was from A04 and A019, with recoveries of 88.00 and 91.49% w/w, respectively, obtained after 24 h (compared with 84.7% from t=0 observed from the peroxide-spiked control). Both of these antioxidant/stabilizer systems contained 0.1% w/w BHA and BHT (levels selected based on the FDA IIG limits at the time of the experiments), where the increased recovery may be due to synergism between these antioxidant/stabilizers. Interestingly, propyl gallate was absent in both AO4 and AO19, which displayed higher percentage recoveries (88.00 and 91.49% from t=0, respectively, after 24 hours) than antioxidant/stabilizer systems containing BHA, BHT and propyl gallate (i.e. AO5 and AO17, 82.86 and 70.27% from t=0, respectively).

From the DoE investigation, it was observed that antioxidant/stabilizer systems containing ascorbic acid displayed the lowest percentage recovery, including the AO3 and AO8 systems (recoveries of 63.00 and 61.94% when compared to t=0, respectively), after 24 h. The results may be due to the ascorbic acid requiring EDTA for stabilization, and hence without EDTA, the ascorbic acid may be reacting with a hydroxyl radical on ADX-102 instead of H₂O₂. The peak purity data (Table 17) was consistent with the ADX-102 recoveries observed (Table 16), with decreases in recovery accompanied by a corresponding decrease in peak purity for the majority of samples across the experimental time frame and conditions. Following 2 weeks of storage at 25° C. in H₂O₂, ADX-102 peak purities of between 5.31-50.99% a/a were observed for all systems investigated.

TABLE 15
Antioxidant systems investigated for ADX-102

| | Theoretical concentration of antioxidant/stabilizers | | | | | |
|---|---|---|---|---|---|---|
| | (% w/w) | | | | | |
| Antioxidant | | | Propyl | Ascorbic | Ascorbyl | α- |
| System | BHA | BHT | gallate | acid | palmitate | Tocopherol |
| AO1 | — | 0.1 | 0.05 | — | — | — |
| AO2 | 0.1 | 0.1 | 0.05 | 0.1 | 0.02 | 0.002 |
| AO3 | 0.1 | — | 0.05 | 0.1 | 0.02 | 0.002 |
| AO4 | 0.1 | 0.1 | — | — | — | 0.002 |
| AO5 | 0.1 | 0.1 | 0.05 | — | 0.02 | — |
| AO6 | 0.1 | — | — | — | — | — |
| AO7 | — | — | 0.05 | 0.1 | 0.02 | — |
| AO8 | — | — | — | 0.1 | 0.02 | 0.002 |
| AO9 | — | — | — | 0.1 | — | — |
| AO10 | 0.1 | — | 0.05 | — | — | 0.002 |
| AO11 | — | — | 0.05 | — | 0.02 | 0.002 |
| AO12 | 0.1 | — | — | 0.1 | 0.02 | — |
| AO13 | — | 0.1 | — | — | 0.02 | — |
| AO14 | — | — | — | — | — | 0.002 |
| AO15 | — | 0.1 | — | 0.1 | 0.02 | 0.002 |
| AO16 | — | 0.1 | 0.05 | — | 0.02 | 0.002 |
| AO17 | 0.1 | 0.1 | 0.05 | 0.1 | — | — |
| AO18 | — | — | — | — | 0.02 | — |
| AO19 | 0.1 | 0.1 | — | 0.1 | 0.02 | — |
| AO20 | — | — | 0.05 | — | — | — |
| AO21 | 0.1 | — | — | — | 0.02 | 0.002 |
| AO22 | — | 0.1 | 0.05 | 0.1 | 0.02 | — |
| AO23 | — | 0.1 | 0.05 | 0.1 | — | 0.002 |
| AO24 | — | 0.1 | — | 0.1 | — | — |
| AO25 | 0.1 | — | 0.05 | — | 0.02 | — |
| AO26 | 0.1 | — | — | 0.1 | — | 0.002 |
| AO27 | — | — | 0.05 | 0.1 | — | 0.002 |
| AO28 | 0.1 | — | 0.05 | 0.1 | — | — |

(—) not included

TABLE 16
Percentage recovery (%) compared to T = 0 of ADX-102 in PEG 400
antioxidant/stabilizer systems following t = 24 h and 2 weeks of
storage at 70° C. with 1.0% w/w H₂O₂ spiking.

| Antioxidant | Percentage (%) recovery of ADX-102 compared to recovery at T = 0. | | |
|---|---|---|---|
| System | T = 0 | T = 24 h | T = 2 weeks |
| AO1 | 100.00 (99.83-100.13) | 85.82 (84.12-87.53) | 35.49 (35.24-35.74) |
| AO2 | 100.00 (99.89-100.07) | 65.79 (65.07-66.51) | 14.09 (14.06-14.11) |
| AO3 | 100.00 (99.86-100.20) | 63.00 (62.40-63.61) | 15.66 (15.47-15.85) |
| AO4 | 100.00 (98.88-100.65) | 88.00 (85.11-90.89) | 41.02 (40.99-41.06) |
| AO5 | 100.00 (99.43-100.77) | 82.86 (82.32-83.40) | 49.74 (49.19-50.29) |
| AO6 | 100.00 (98.59-101.31) | 84.45 (82.63-86.27) | 4.75 (4.67-4.83) |
| AO7 | 100.00 (99.23-100.76) | 63.91 (63.46-64.36) | 7.00 (6.97-7.04) |
| AO8 | 100.00 (99.05-100.52) | 61.94 (61.83-62.05) | 4.44 (4.44-4.44) |
| AO9 | 100.00 (99.87-100.10) | 63.39 (63.23-63.55) | 5.02 (4.84-5.20) |
| AO10 | 100.00 (99.45-100.75) | 84.76 (83.88-85.63) | 30.14 (29.95-30.33) |
| AO11 | 100.00 (99.60-100.47) | 82.99 (82.68-83.30) | 27.95 (27.94-27.97) |
| AO12 | 100.00 (99.57-100.57) | 63.69 (63.42-63.97) | 14.23 (14.13-14.33) |
| AO13 | 100.00 (99.83-100.32) | 82.92 (82.85-82.99) | 33.99 (33.51-34.48) |
| AO14 | 100.00 (99.59-100.29) | 84.89 (84.13-85.66) | 12.49 (0.00-24.97) |
| AO15 | 100.00 (99.16-100.65) | 68.51 (68.07-68.95) | 27.49 (27.08-27.90) |
| AO16 | 100.00 (99.65-100.68) | 84.65 (84.58-84.72) | 45.89 (45.34-46.45) |
| AO17 | 100.00 (99.12-101.52) | 70.27 (69.91-70.63) | 14.77 (14.74-14.80) |
| AO18 | 100.00 (98.89-100.86) | 72.37 (72.08-72.65) | 8.10 (7.43-8.77) |
| AO19 | 100.00 (99.97-100.06) | 91.49 (91.24-91.74) | 14.80 (14.77-14.83) |
| AO20 | 100.00 (99.70-100.50) | 84.21 (83.46-84.97) | 16.01 (15.93-16.08) |
| AO21 | 100.00 (99.63-100.36) | 76.49 (76.16-76.81) | 28.31 (27.79-28.83) |
| AO22 | 100.00 (99.94-100.07) | 65.73 (65.69-65.77) | 10.22 (10.18-10.26) |
| AO23 | 100.00 (99.55-100.61) | 66.93 (65.75-68.10) | 26.40 (26.08-26.71) |
| AO24 | 100.00 (99.75-100.44) | 64.90 (64.65-65.14) | 10.58 (10.54-10.61) |
| AO25 | 100.00 (99.52-100.34) | 83.17 (82.84-83.50) | 39.57 (38.47-40.67) |
| AO26 | 100.00 (99.83-100.24) | 70.48 (69.46-71.51) | 13.56 (13.50-13.63) |
| AO27 | 100.00 (99.72-100.21) | 70.94 (70.47-71.42) | 27.16 (26.56-27.76) |
| AO28 | 100.00 (99.37-100.75) | 64.63 (64.42-64.83) | 23.39 (23.07-23.71) |
| Control | 100.00 (98.96-101.51) | 99.51 (98.54-100.48) | 54.29 (53.36-55.01) |
| (without H₂O₂) | | | |
| Control | 100.00 (98.82-100.88) | 84.70 (84.21-85.20) | 19.81 (19.56-20.07) |
| (with H₂O₂) | | | |

Data presented as the mean with the range in brackets (n = 3).
Data analyzed by developed HPLC assay method.

TABLE 17
Percentage peak purity (% a/a) of ADX-102 in the antioxidant/
stabilizer systems at T = 0, 24 h and 2 weeks following storage
at 70° C. with 1.0% w/w H₂O₂ spiking.

| | Percentage area/area (% a/a) of | | |
|---|---|---|---|
| | the ADX-102 peak in | | |
| Antioxidant | antioxidant/stabilizer systems. | | |
| System | T = 0 | T = 24 h | T = 2 weeks |
| AO1 | 93.70 | 85.71 | 41.38 |
| AO2 | 82.23 | 60.69 | 18.13 |
| AO3 | 81.94 | 58.33 | 16.47 |
| AO4 | 97.73 | 87.57 | 45.59 |
| AO5 | 91.91 | 82.73 | 55.25 |
| AO6 | 98.41 | 88.64 | 5.31 |
| AO7 | 82.64 | 61.72 | 11.98 |
| AO8 | 89.20 | 64.53 | 4.49 |
| AO9 | 88.82 | 62.37 | 5.89 |
| AO10 | 93.73 | 86.23 | 35.20 |
| AO11 | 93.77 | 83.73 | 33.22 |
| AO12 | 84.06 | 14.55 | 20.08 |
| AO13 | 97.98 | 32.63 | 40.02 |
| AO14 | 99.90 | 45.35 | 28.17 |
| AO15 | 88.02 | 29.47 | 32.07 |
| AO16 | 92.96 | 83.86 | 50.99 |
| AO17 | 85.84 | 86.68 | 18.13 |
| AO18 | 97.93 | 63.43 | 8.11 |
| AO19 | 85.97 | 87.04 | 19.76 |
| AO20 | 94.47 | 63.87 | 19.48 |
| AO21 | 96.90 | 86.62 | 32.38 |
| AO22 | 81.38 | 62.24 | 13.97 |
| AO23 | 83.37 | 63.29 | 32.26 |
| AO24 | 87.36 | 65.83 | — |
| AO25 | 90.85 | 82.60 | 45.45 |
| AO26 | 90.10 | 67.67 | 18.40 |
| AO27 | 84.71 | 64.99 | 34.15 |
| AO28 | 81.94 | 61.36 | 29.28 |
| Control | 99.80 | 93.61 | 53.29 |
| (without H₂O₂) | | | |
| Control | 99.84 | 71.13 | 23.52 |
| (with H₂O₂) | | | |

(—) not tested
Data is presented as n = 1.

Chemical Stability of ADX-102 in Excipients

The chemical stability of ADX-102 in an array of individual excipients and binary systems was investigated to determine their potential for use in a topical formulation. Samples were stored at 40 and 50° C. for 2 weeks and the ADX-102 recovery and purity results are presented in Table 18 and Table 19, respectively.

Based on the recovery and purity data generated, the excipients benzyl alcohol, super refined PEG 400, propylene carbonate, castor oil, glycerol, IPM, IPP, Miglyol 810, Transcutol HP, propylene glycol, ethanol and water systems (75:25 v/v WFI:ethanol and 75:25 v/v deionised water:ethanol) presented comparable and consistent values to T=0 following storage at 40 and 50° C. for 2 weeks and were considered suitable for the formulation development experiments described below. It is important to note that the glycerol and castor oil did present variable recovery values, however this is due to the viscosity of the excipients introducing variability into the sample preparation procedure.

A reduction in both purity and recovery from T=0 following 2 weeks of storage at 40 and 50° C. was observed for super refined DMI and oleyl alcohol (which was low at T=0, where it was assumed that the ADX-102 was instantly degraded upon addition to the oleyl alcohol) indicating that these excipients were not suitable for inclusion in large amounts in the formulation development experiments and were thus not investigated further. In addition, the excipients diisopropyl adipate and phenoxyethanol presented purity values below 95% at the t=2 week stability timepoint, therefore diisopropyl adipate was not employed in the formulation development activities. However, as benzyl alcohol presented a risk of degrading and producing aldehydes, and as the maximum level for the phenoxyethanol allowed for a topical formulation is 1% w/w based on the FDA IIG, and the excipient stability experiments are assessing the stability of ADX-102 in 100% phenoxyethanol, the risk of degradation in a representative formulation was low. Furthermore, as benzyl alcohol presented a risk of degrading and producing aldehydes, phenoxyethanol was used for the formulation development experiments as opposed to benzyl alcohol as the preservative. IPA also presented consistent ADX-102 recovery and purity values following 2 weeks of storage at 25 and 40° C. compared to T=0, however as low purity was observed it was uncertain whether this was caused by contamination or interaction of the IPA with the ADX-102, therefore to mitigate an risk of compatibility issues this excipient was not investigated during the formulation development experiments. To summarize, the excipient/ADX-102 stability experiments allowed us to identify the lowest and highest risk excipients for use in the formulation development.

TABLE 18
Percentage recovery (%) of ADX-102 in excipients after storage

| | Percentage (%) recovery of ADX-102 compared to recovery at T = 0 | | | | |
|---|---|---|---|---|---|
| | | T = 1 week | | T = 2 weeks | |
| Excipient | T = 0 | 40° C. | 50° C. | 40° C. | 50° C. |
| Isopropyl alcohol | 95.04 | 99.67 | 98.66 | 99.59 | 98.06 |
| | (87.11-102.96) | (99.14-100.19) | (98.43-98.90) | (98.99-100.19) | (97.97-98.16) |
| Benzyl alcohol | 101.45 | 101.64 | 99.94 | 100.27 | 99.50 |
| | (101.41-101.49) | (101.44-101.84) | (99.16-100.72) | (100.04-100.50) | (98.74-100.26) |
| Super refined PEG | 99.93 | 95.83 | 98.86 | 100.12 | 99.44 |
| 400 | (98.45-101.41) | (94.51-97.15) | (98.25-99.47) | (99.26-100.99) | (99.35-99.52) |
| Phenoxyethanol | 100.37 | 99.41 | 100.85 | 99.47 | 100.46 |
| | (99.15-101.59) | (99.09-99.74) | (100.80-100.89) | (99.29-99.66) | (99.97-100.94) |
| Propylene carbonate | 101.07 | 100.12 | 99.31 | 98.70 | 99.15 |
| | (100.89-101.26) | (99.09-101.15) | (99.07-99.54) | (97.24-100.16) | (98.81-99.49) |
| Oleyl alcohol | 0.65 | 0.08 | 0.89 | 0.88 | 0.89 |
| | (0.65-0.65) | (0.07-0.08) | (0.89-0.89) | (0.86-0.90) | (0.89-0.89) |
| Super refined DMI | 21.03 | 0.39 | 1.07 | 1.03 | 0.95 |
| | (20.81-21.24) | (0.12-0.66) | (0.91-1.23) | (0.88-1.18) | (0.90-1.00) |
| Castor oil | 91.79 | 98.93 | 86.57 | 88.94 | 86.67 |
| | (91.38-92.20) | (87.76-110.10) | (86.16-86.99) | (88.33-89.56) | (86.60-86.75) |
| Diisopropyl adipate | 100.29 | 98.47 | 99.40 | 98.86 | 99.02 |
| | (99.99-100.59) | (98.21-98.72) | (99.36-99.44) | (98.85-98.86) | (98.79-99.25) |
| Glycerol | 97.62 | 109.23 | 107.55 | 105.90 | 109.00 |
| | (97.24-97.99) | (108.99-109.47) | (106.96-108.15) | (105.26-106.53) | (108.23-109.77) |
| Isopropyl myristate | 98.37 | 97.07 | 97.01 | 96.07 | 97.59 |
| (IPM) | (98.37-98.38) | (96.84-97.30) | (96.91-97.10) | (95.85-96.29) | (97.37-97.80) |
| Isopropyl palmitate | 99.29 | 99.56 | 99.51 | 101.37 | 98.87 |
| (IPP) | (98.20-100.39) | (99.27-99.85) | (98.74-100.27) | (100.96-101.78) | (98.66-99.08) |
| Miglyol 810 | 93.13 | 94.19 | 92.11 | 92.97 | 92.32 |
| | (92.92-93.34) | (94.06-94.32) | (92.05-92.17) | (92.85-93.09) | (92.12-92.52) |
| Transcutol HP | 98.60 | 98.45 | 99.25 | 99.65 | 97.96 |
| | (98.49-98.70) | (98.38-98.52) | (99.10-99.41) | (99.46-99.83) | (97.90-98.02) |
| Propylene glycol | 95.71 | 96.48 | 96.46 | 96.61 | 96.26 |
| | (95.41-96.02) | (96.23-96.73) | (96.24-96.68) | (96.59-96.63) | (95.38-97.13) |
| Ethanol | 98.87 | 98.99 | 99.37 | 99.58 | 98.88 |
| | (98.11-99.62) | (98.34-99.64) | (99.25-99.49) | (99.37-99.80) | (98.46-99.30) |
| 75:25 v/v Water | 93.15 | 93.22 | 103.04 | 94.23 | 91.49 |
| for irrigation:Ethanol | (92.72-93.57) | (92.91-93.54) | (93.65-112.43) | (94.20-94.26) | (91.35-91.64) |
| 75:25 v/v | 99.29 | 94.90 | 99.33 | 94.64 | 93.65 |
| Deionized | (97.95-100.63) | (94.79-95.01) | (91.01-107.65) | (94.16-95.12) | (93.49-93.80) |
| water:Ethanol | | | | | |

Data presented as the mean with the range in brackets (n = 3).
Data analyzed by developed HPLC assay method.

TABLE 19
Percentage peak purity (% a/a) of ADX-102 in excipients

| | Percentage purity (% a/a) of ADX-102 | | | | |
|---|---|---|---|---|---|
| | | T = 1 week | | T = 2 weeks | |
| Excipient | T = 0 | 40° C. | 50° C. | 40° C. | 50° C. |
| Isopropyl alcohol | 7.12 | 7.20 | 8.29 | 7.41 | 6.79 |
| Benzyl alcohol | 98.51 | 97.88 | 97.58 | 98.22 | 98.21 |
| Super refined PEG 400 | 99.93 | 99.33 | 98.98 | 98.32 | 97.59 |
| Phenoxyethanol | 100.00 | 99.94 | 99.94 | 93.15 | 92.21 |
| Propylene carbonate | 99.95 | 99.94 | 99.93 | 99.95 | 99.95 |
| Oleyl alcohol | 100.00 | 20.85 | 25.99 | 32.77 | 52.30 |
| Super refined DMI | 21.51 | 0.10 | 0.10 | 0.08 | 0.10 |
| Castor oil | 100.00 | 99.83 | 99.80 | 98.87 | 99.69 |
| Diisopropyl adipate | 99.63 | 99.78 | 99.95 | 99.51 | 93.07 |
| Glycerol | 99.64 | 99.49 | 99.41 | 98.54 | 99.04 |
| Isopropyl myristate | 99.94 | 99.77 | 99.72 | 99.67 | 99.25 |
| (IPM) | | | | | |
| Isopropyl palmitate (IPP) | 99.79 | 99.96 | 99.95 | 99.96 | 99.74 |
| Miglyol 810 | 99.81 | 99.90 | 99.90 | 99.92 | 99.87 |
| Transcutol HP | 99.90 | 99.93 | 99.93 | 99.90 | 99.58 |
| Propylene glycol | 99.58 | 99.41 | 99.56 | 99.36 | 99.34 |
| Ethanol | 99.88 | 99.35 | 99.34 | 99.29 | 99.02 |
| 75:25 v/v WFI:Ethanol | 99.72 | 99.73 | 99.91 | 99.56 | 99.60 |
| 75:25 v/v Deionized | 97.82 | 99.91 | 99.91 | 99.76 | 99.57 |
| water:Ethanol | | | | | |

Data is presented as n = 1.

### Example 5: Formulation Development

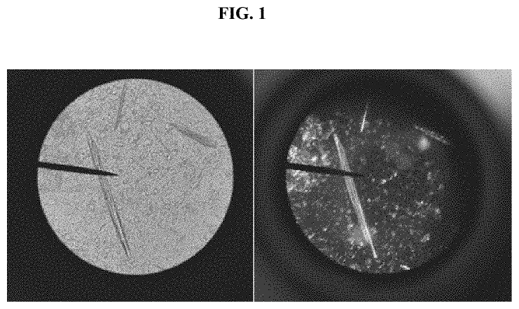

The comparison formulation was also characterized and the results are shown in Table 20. The formulation was a pink cream with a high visual viscosity. The formulation showed ADX-102 crystals under polarized and non-polarized light, as depicted in FIG. 1. The apparent pH of the formulation was measured as 6.31. The comparison formulation was assayed for ADX-102 and the percentage recovery of ADX-102 was 99.21% w/w. The peak purity data was consistent with the recovery observed (99.56% a/a). The ADX-102 content in the formulation was found to be 1.05% w/w, which following the saturated solubility of ADX-102 in the comparison solvent system of 0.31% w/w, highlighting the fact that ˜0.7% of ADX-102 is in the oil phase/suspended in the formulation rather than in solution based on the ADX-102 crystals present.

TABLE 20
Characterization of the comparison formulation

| | | | | | Percentage | Percentage |
|---|---|---|---|---|---|---|
| | | | Microscopy | | (%) | purity |
| | | Apparent | | Non- | recovery of | (% a/a) of |
| | Macroscopy | pH | Polarized | polarized | ADX-102 | ADX-102 |
| Comparison | Pink cream, | 6.31 | ADX-102 | ADX-102 | 99.21 | 99.56 |
| formulation | with high | | crystals | crystals | (97.57-101.94) | |
| | visual | | observed | observed | | |
| | viscosity | | | | | |

Data analyzed by developed HPLC assay method. Percentage purity (% a/a) of ADX-102 is presented as n = 1. Data presented as the mean with the range in brackets (n = 3).

### Placebo Formulation Development

Based on the results from the pre-formulation experiments, a selection of placebo formulations containing variations in water content, surfactant system and thickeners were prepared as displayed in Table 21.

Initially, in an effort to design a cream to maximize drug loading, CR001-003 were prepared to assess the impact of water content on formulation characteristics as it was identified as a non-solvent during the solubility experiments. CR001-CR003 were white in color and of a medium to high viscosity. Following the characterization, the assessment of water concentration was continued with the preparation of CR004-CR007, each with varying concentration of Transcutol® HP and PEG 1500. While compositional differences were evident, visual appearance did not appear to change (i.e., placebo formulations were white in color, medium-high viscosity and with homogenous distribution of oil droplets). Penetration enhancers were further evaluated in placebo formulations CR008 (low water) and CR009 (high water), substituting propylene glycol (present in the comparison formulation) for DMI. However, despite the differences the formulations did not change in visual appearance from the previously developed creams.

The surfactant system and oil phase used in the comparison formulation were investigated and modified (in parallel with water content) during the development of the placebo formulations CR011-14 and CR017-18. The Span 20 and Geleol™ system employed in the comparison formulation was altered using an HLB approach to modify the concentration of the surfactants to accommodate the oil phase (CR010 and CR011) however, the resulting formulations were low viscosity solutions with either a foam-like layer (CR010, presumed to be flocullants of HPMC) or large white particulates suspended in the solution (CR011, thought to be HPMC). Alternative systems, employing cetomacrogol 1000 in place of Span 20 (CR012 and CR013) and a Span 60/Tween 60 system (CR014) was investigated to assess the compatibility of the surfactants with each other and the solvent system. As observed in CR011, formulations appeared to be turbid/white solutions with white particulates in suspension, indicating that HPMC was not compatible.

A de novo approach was also assessed. CR017 and CR018, employing a different oil phase (liquid paraffin and white petrolatum), thickener (PEG 1500 only) and surfactant system (Brij S2 and Brij S721) to the comparison formulation, were prepared. The resulting formulation was white, had a homogenous distribution of emulsion droplets, and had a medium-high viscosity.

To investigate an alternative thickener to PEG 1500, a higher molecular weight PEG (PEG 4000) was used in place of PEG 1500 in CR015 and CR016 (based on the CR02 and CR010 formulations, respectively). CR015 appeared to be similar in appearance to CR002 (off-white, medium-high viscosity), while CR016 was notably different to CR010 which had been observed to be a low viscosity solution (as opposed to an off-white, medium-high viscosity cream).

The formulations which demonstrated suitable aesthetic appearance upon preparation were stored at 40° C. and visually assessed after 1 week, where no change in appearance and no immediate evidence of phase separation was observed.

TABLE 21
Prototype placebo formulations investigated

| | Composition of formulation (% w/w) | | | | | | | | |
|---|---|---|---|---|---|---|---|---|---|
| | PLB | PLB | PLB | PLB | PLB | PLB | PLB | PLB | PLB |
| Excipient | CR001 | CR002 | CR003 | CR004 | CR005 | CR006 | CR007 | CR008 | CR009 |
| ADX-102 | — | — | — | — | — | — | — | — | — |
| Transcutol ® | 10.06 | 10.06 | 10.06 | 20.06 | 30.06 | 10.05 | 10.05 | 10.06 | 10.06 |
| HP | | | | | | | | | |
| Arlasolve ™ | — | — | — | — | — | — | — | 5.06 | 5.06 |
| DMI | | | | | | | | | |
| Propylene | 5.06 | 5.06 | 5.06 | 5.06 | 5.06 | 5.05 | 5.05 | — | — |
| Glycol | | | | | | | | | |
| Purified | 50.06 | 40.06 | 30.06 | 40.06 | 30.06 | 40.05 | 30.05 | 40.06 | 30.06 |
| Water | | | | | | | | | |
| SR PEG 400 | — | — | — | — | — | 10.05 | 20.05 | — | — |
| PEG 1500 | 18.66 | 28.66 | 38.66 | 18.66 | 18.66 | 18.65 | 18.65 | 28.66 | 38.66 |
| Steareth 20/ | 0.96 | 0.96 | 0.96 | 0.96 | 0.96 | 0.95 | 0.95 | 0.96 | 0.96 |
| Brij S20 | | | | | | | | | |
| Cetomacrogol | 3.06 | 3.06 | 3.06 | 3.06 | 3.06 | 3.05 | 3.05 | 3.06 | 3.06 |
| 1000 | | | | | | | | | |
| Span 20 | 2.06 | 2.06 | 2.06 | 2.06 | 2.06 | 2.05 | 2.05 | 2.06 | 2.06 |
| White | 3.06 | 3.06 | 3.06 | 3.06 | 3.06 | 3.05 | 3.05 | 3.06 | 3.06 |
| petrolatum | | | | | | | | | |
| Geleol ® | 7.06 | 7.06 | 7.06 | 7.06 | 7.06 | 7.05 | 7.05 | 7.06 | 7.06 |
| mono and | | | | | | | | | |
| diglycerides | | | | | | | | | |
| Total | 100.00 | 100.00 | 100.00 | 100.00 | 100.00 | 100.00 | 100.00 | 100.00 | 100.00 |
| Visual | White, | White, | White, | White, | White, | White, | White, | White, | White, |
| appearance | medium | medium | medium | medium | medium | medium | medium | medium | medium |
| upon | high | high | high | high | high | high | high | high | high |
| preparation | viscosity | viscosity | viscosity | viscosity | viscosity | viscosity | viscosity | viscosity | viscosity |
| Visual | No change from preparation | | | | | | | | |
| appearance | | | | | | | | | |
| after | | | | | | | | | |
| T = 1 week | | | | | | | | | |
| at 40° C. | | | | | | | | | |
| | Composition of formulation (% w/w) | | | | | | | | |
| | PLB | PLB | PLB | PLB | PLB | PLB | PLB | PLB | PLB |
| Excipient | CR010 | CR011 | CR012 | CR013 | CR014 | CR015 | CR016 | CR017 | CR018 |
| ADX-102 | — | — | — | — | — | — | — | — | — |
| Transcutol | 10.00 | 10.00 | 10.00 | 10.00 | 16.00 | 10.00 | 10.00 | 10.00 | 10.00 |
| HP | | | | | | | | | |
| Propylene | 5.00 | 5.00 | 5.00 | 5.00 | 5.00 | 5.00 | 5.00 | 5.00 | 5.00 |
| Glycol | | | | | | | | | |
| Purified | 40.00 | 30.00 | 40.00 | 30.00 | 30.00 | 30.00 | 40.00 | 30.00 | 30.00 |
| Water | | | | | | | | | |
| S.R. PEG 400 | — | 10.00 | — | 10.00 | — | — | — | 6.00 | 5.00 |
| PEG 1500 | 37.00 | 37.00 | 37.00 | 37.00 | 40.00 | — | — | 37.00 | 37.00 |
| Steareth 20 | — | — | — | — | — | 0.90 | — | — | — |
| Cetomacrogol | — | — | 1.26 | 1.26 | — | 3.00 | — | — | — |
| 1000 | | | | | | | | | |
| Span 20 | 2.86 | 2.86 | — | — | — | 2.00 | 2.86 | — | — |
| Hypromellose | 1.00 | 1.00 | 1.00 | 1.00 | 1.00 | — | — | — | — |
| White | 3.00 | 3.00 | 3.00 | 3.00 | 4.00 | 3.00 | 3.00 | 3.00 | 3.00 |
| petrolatum | | | | | | | | | |
| Liquid | — | — | — | — | — | — | — | 5.00 | 5.00 |
| Paraffin | | | | | | | | | |
| Geleol | 1.14 | 1.14 | 2.74 | 2.74 | — | 7.00 | 1.14 | — | — |
| mono and | | | | | | | | | |
| diglycerides | | | | | | | | | |
| Span 60 | — | — | — | — | 3.10 | — | — | — | — |
| Tween 60 | — | — | — | — | 0.90 | — | — | — | — |
| PEG 4000 | — | — | — | — | — | 39.10 | 38.00 | — | — |
| Steareth 21 | — | — | — | — | — | — | — | 1.52 | 1.90 |
| Steareth 2 | — | — | — | — | — | — | — | 2.48 | 3.10 |
| Total | 100.00 | 100.00 | 100.00 | 100.00 | 100.00 | 100.00 | 100.00 | 100.00 | 100.00 |
| Visual | Off white, | Turbid | Turbid | Turbid | White | Off white, | Off white, | White, | White, |
| appearance | low | solution | solution | solution | solution | medium- | low | medium | medium |
| upon | viscosity | with large | with large | with large | with small | low | viscosity | high | high |
| preparation | solution | solid white | white solid | clear solid | solid | viscosity | | viscosity | viscosity |
| | with | particulates | particulates | particulates | particulates | | | | cream |
| | foam layer | | | | | | | | |

| | | |
|---|---|---|
| Visual | Not assessed due to appearance upon preparation | No change from preparation |
| appearance | | |
| after | | |
| T = 1 week | | |
| at 40° C. | | |

(—) not included.

Following the visual assessment of placebo formulations, a DoE mixture design for ADX-102 aqueous phase solvent systems was prepared, as shown in Table 22. This was performed in order to assess the solubility of ADX-102 in solvent systems and to confirm that at least a 1.0% w/w and a 5% w/w concentration of ADX-102 in the aqueous phase of the formulation was obtainable. The following excipient levels were assessed during the solvent system DoE experiments:
Water: 0-50%
Transcutol® HP: 0-20%
SR PEG 400: 0-60%
Ethanol: 0-20%
Propylene glycol: 0-15%

The results of the saturated solubility in the DoE solvent systems are presented in Table 23. Solvent systems with 50:50 v/v SR-PEG 400: water content (SS03, SS08 and SS13) presented ADX-102 solubility of ca. <1.43% w/w, confirming that ADX-102 presented poor water solubility. Furthermore, solvents systems which had a water composition of 45% w/w (SS12, SS14 and SS15), produced solubility results of ca. 4% w/w ADX-102. Therefore, the results indicate that to achieve a target concentration of higher than 1% w/w ADX-102 in a formulation, a water content of less than 50% w/w using the aqueous phase excipients outlined above would be generally desirable.

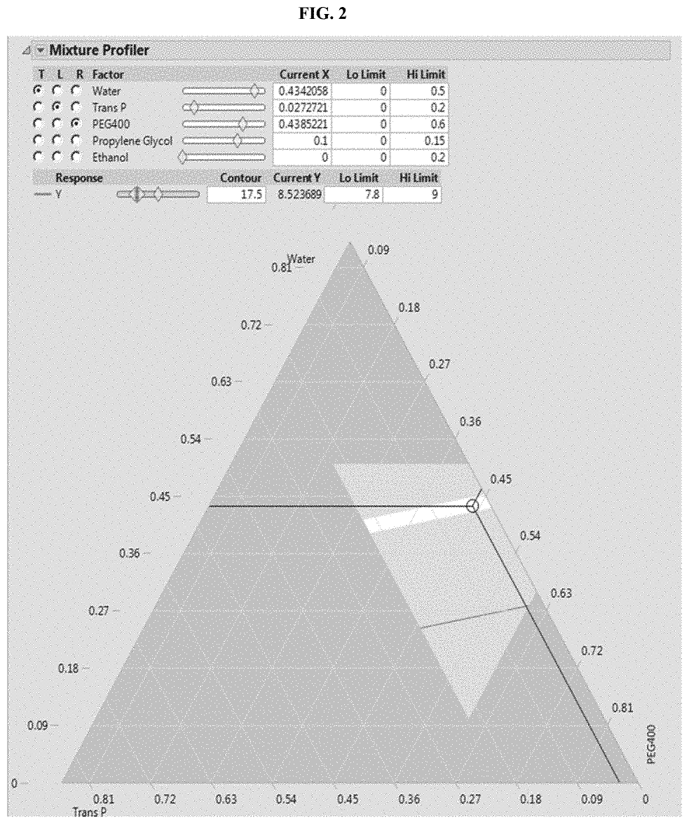

In addition, the data generated during the initial DoE solvent mixture design (example of the output is presented in FIG. 2) was used to develop solvent systems (SS16-SS22, Table 24) with a theoretical saturated solubility of ADX-102 of >7.8% w/w which were suitable for a 5% w/w ADX-102 formulation. The solubility of ADX-102 was then assessed in the aforementioned solvent systems and it was found that two systems, SS19 and SS21 (ADX-102 saturated solubility of 8.19 and 7.80% w/w, respectively) achieved the required solubility of ADX-102 to prepare a cream formulation of approx. 5% w/w ADX-102.

SS19, along with SS20 (SS21 was not used due to the high levels of Transcutol® HP), were used as a base for further solvent system development. SS23-SS26 were prepared, broadly based on these systems, with varying levels of excipient and added antioxidant/stabilizer, and preservative. The compositions of these solvent systems are detailed in Table 25. Additionally, penetration enhancers were varied in the developed solvent system, with SS23 (based on SS19) including no Transcutol® and SS25 including diisopropyl adipate, and in the case of SS24, a humectant (glycerol). It should be noted that SS25 became turbid following the addition of diisopropyl adipate. The saturated solubility of ADX-102 was therefore assessed in only SS23, SS24 and SS26, where the solubility of the drug was observed to be between 6.67-7.94% w/w in the systems, suggesting that these solvent systems could also be employed to prepare a cream formulation of approx. 5% w/w ADX-102.

Additional solvent systems (SS33-SS36, Table 26), for use in a 1% w/w formulation, were prepared based on the solubility data generated during the pre-formulation experiments and the DoE data gathered during solvent system development and were employed in the formulation development.

TABLE 22
Composition (% w/w) of solvent systems based on the DoE investigated

| | Composition of solvent system (% w/w) | | | | | | | |
|---|---|---|---|---|---|---|---|---|
| Excipient | SS01 | SS02 | SS03 | SS04 | SS05 | SS06 | SS07 | SS08 |
| Water | 5.00 | 5.00 | 50.00 | 21.46 | 21.46 | — | 5.00 | 50.00 |
| Transcutol ® | 20.00 | — | — | 18.65 | 18.65 | 20.00 | 20.00 | — |
| HP | | | | | | | | |
| SR PEG 400 | 60.00 | 60.00 | 50.00 | 26.28 | 26.28 | 60.00 | 60.00 | 50.00 |
| Propylene | 15.00 | 15.00 | — | 14.95 | 14.95 | — | 15.00 | — |
| glycol | | | | | | | | |
| Ethanol | — | 20.00 | — | 18.65 | 18.65 | 20.00 | — | — |
| Total | 100.00 | 100.00 | 100.00 | 100.00 | 100.00 | 100.00 | 100.00 | 100.00 |

Composition (% w/w) of solvent systems based on the DoE investigated

| | Composition of solvent system (% w/w) | | | | | | |
|---|---|---|---|---|---|---|---|
| Excipient | SS09 | SS10 | SS11 | SS12 | SS13 | SS14 | SS15 |
| Water | — | 21.46 | 5.00 | 45.00 | 50.00 | 45.00 | 45.00 |
| Transcutol ® | 20.00 | 18.65 | — | 20.00 | — | 20.00 | 20.00 |
| HP | | | | | | | |
| SR PEG 400 | 60.00 | 26.28 | 60.00 | — | 50.00 | — | — |
| Propylene | — | 14.95 | 15.00 | 15.00 | — | 15.00 | 15.00 |
| glycol | | | | | | | |
| Ethanol | 20.00 | 18.65 | 20.00 | 20.00 | — | 20.00 | 20.00 |
| Total | 100.00 | 100.00 | 100.00 | 100.00 | 100.00 | 100.00 | 100.00 |

Saturated solubility of ADX-102 (% w/w) in solvent systems based on
the DoE tested

| Solvent System | ADX-102 saturated solubility (% w/w) | |
|---|---|---|
| SS01 | ≥30.00 | (visual observation) |
| SS02 | ≥30.00 | (visual observation) |
| SS03 | 1.43 | (1.42-1.44) |
| SS04 | 19.65 | (19.33-19.91) |
| SS05 | 21.53 | (21.36-21.72) |
| SS06 | ≥30.00 | (visual observation) |
| SS07 | ≥30.00 | (visual observation) |
| SS08 | 1.47 | (1.45-1.49) |
| SS09 | ≥30.00 | (visual observation) |
| SS10 | 18.17 | (17.84-18.50) |
| SS11 | ≥30.00 | (visual observation) |
| SS12 | 4.75 | (4.66-4.88) |
| SS13 | 1.43 | (1.42-1.44) |
| SS14 | 4.84 | (4.74-4.93) |
| SS15 | 4.55 | (4.46-4.60) |

(—) not included.
Data is presented as the mean with the range in brackets (n = 3). Data analyzed by the developed HPLC assay method unless otherwise stated.

TABLE 23
Composition of solvent systems for a 5% w/w ADX-102 formulation

| | Composition of final solvent system (% w/w) | | | | | | |
|---|---|---|---|---|---|---|---|
| Excipient | SS16 | SS17 | SS18 | SS19 | SS20 | SS21 | SS22 |
| Water | 39 | 41 | 44 | 38 | 38 | 38 | 42 |
| Transcutol ® | 2 | 18 | 2 | 12 | 1 | 18 | 1 |
| HP | | | | | | | |
| SR PEG 400 | 59 | 31 | 44 | 40 | 51 | 24 | 37 |
| Ethanol | 0 | 0 | 0 | 10 | 10 | 10 | 10 |
| Propylene glycol | 0 | 10 | 10 | 0 | 0 | 10 | 10 |
| Total | 100 | 100 | 100 | 100 | 100 | 100 | 100 |
| Saturated | 4.42 | 4.52 | 2.54 | 8.19 | 6.82 | 7.80 | 3.80 |
| solubility of | | | | | | | |
| ADX-102 | | | | | | | |
| (% w/w) | | | | | | | |

TABLE 24
Composition of solvent systems for a 5% w/w ADX-102 formulation, with
preservative, antioxidant and humectants

| | Composition of final solvent systems (% w/w) | | | |
|---|---|---|---|---|
| Excipient | SS23 | SS24 | SS25 | SS26 |
| H₂O | 38 | 38 | 38 | 38 |
| Transcutol ® HP | — | 1 | 1 | 1 |
| SR PEG 400 | 50.8 | 39.8 | 39.8 | 49.8 |
| Ethanol | 10 | 10 | 10 | 10 |
| Propylene glycol | — | — | — | — |
| Glycerol | — | 10 | — | — |
| DIPA | — | — | 10 | — |
| BHT | 0.1 | 0.1 | 0.1 | 0.1 |
| BHA | 0.1 | 0.1 | 0.1 | 0.1 |
| Phenoxyethanol | 1 | 1 | 1 | 1 |
| Total | 100 | 100 | 100 | 100 |
| Rationale | Based on | Based on | Based on | Based on |
| | SS19, | SS19, with | SS19, with | SS20, with |
| | without | humectant | diisopropyl | antioxidant |
| | Transcutol ® | (glycerol) | adipate in | and |
| | HP and | | place of | preservative |
| | increased | | Transcutol ® | |
| | SR PEG | | | |
| | 400 | | | |
| Saturated solubility of | 6.67 | 7.84 | N/A - | 6.84 |
| ADX-102 (% w/w) | | | solution | |
| | | | turbid upon | |
| | | | addition of | |
| | | | diisopropyl | |
| | | | adipate | |

TABLE 25
Composition of solvent systems for use in a 1% w/w
ADX-102 formulation

Composition of solvent system
% w/w

| Excipient | SS33 | SS34 | SS35 | SS36 |
|---|---|---|---|---|
| ADX-102 | 1.00 | 1.00 | 1.00 | 1.00 |
| Transcutol ® HP | 8.65 | 9.60 | 8.65 | 1.68 |
| Propylene Glycol | 5.50 | 0.00 | 5.50 | 0.00 |
| Purified Water | 34.93 | 30.40 | 30.40 | 30.40 |
| SR. PEG 400 | 19.65 | 28.60 | 24.18 | 36.12 |
| Ethanol | 7.86 | 8.00 | 7.86 | 8.40 |
| Phenoxyethanol | 1.00 | 1.00 | 1.00 | 1.00 |
| BHT | 0.05 | 0.10 | 0.10 | 0.10 |
| BHA | 0.10 | 0.10 | 0.10 | 0.10 |
| Total | 78.74* | 78.80* | 78.79* | 78.80* |

*note compositions do not add up to 100% as these account for the oil phase in a final cream formulation.
Note:
inclusion of ADX-102 at 1.28% w/w.

### Antioxidant Level Exploration

A statistical Design of Experiment (DoE) was performed, as detailed in Table 26, with the aim of determining the optimal antioxidant/stabilizer level of BHA and BHT to prevent degradation and improve ADX-102 chemical stability under lower stress conditions determined (1.0% w/w H₂O₂ spiking at 40° C.). Antioxidants listed in Table 26 were assessed in SS34 with 0.1% w/w ADX-102, which was selected as SS34 presented the greatest risk of ADX-102 oxidation.

The antioxidant/stabilizer level justification experiment was performed at t=0, 24 h, and 48 h. The recovery (% w/w) and peak purity (% a/a) of ADX-102 (with the inclusion of BHA/BHT antioxidant/stabilizer systems) was investigated after being stored at the stress conditions (1.0% w/w H₂O₂ spiking at 40° C.) and the results are presented in Table 27. The stress conditions from the original antioxidant/stabilizer DoE investigation were altered by reducing the temperature from 70° C. to 40° C. to limit the levels of degradation in an attempt to better reproduce the levels of oxidation that would be typical during development work.

The results show that the highest percentage recovery (% w/w) of ADX-102 was observed from SS34 AOL2 with 94.05% w/w ADX-102 recovered after t=48 h, where this system contained 0.08 and 0.02% w/w BHT and BHA respectively, when compared to the other systems tested.

TABLE 26
Antioxidant level systems with BHA/BHT investigated for ADX-102

Theoretical Concentration (% w/w)

| Antioxidant | Solvent | | | |
|---|---|---|---|---|
| System | System (SS34) | BHT | BHA | Total |
| SS34 AOL1 | 99.93 | — | 0.07 | 100.00 |
| SS34 AOL2 | 99.90 | 0.08 | 0.02 | 100.00 |
| SS34 AOL3 | 99.86 | 0.04 | 0.10 | 100.00 |
| SS34 AOL4 | 99.82 | 0.10 | 0.08 | 100.00 |
| SS34 AOL5 | 99.98 | 0.02 | — | 100.00 |
| SS34 0.1% | 100.00 | — | — | 100.00 |
| (Control) | | | | |
| SS34 PLB | Placebo 99.80 | 0.10 | 0.10 | 100.00 |
| (Control) | | | | |

TABLE 27
Percentage recovery (%) of ADX-102 in PEG 400 antioxidant/
stabilizer systems following 24 h and 48 h storage at 40° C.
with 1.0% w/w H₂O₂ spiking

| | Percentage (%) recovery of ADX-102 compared | | |
|---|---|---|---|
| Antioxidant | to recovery at T = 0. | | |
| System | T = 0 | T = 24 h | T = 48 h |
| SS34 AOL1 | 100.00 | 93.12 | 93.78 |
| | (97.65-102.59) | (91.24-94.29) | (91.30-95.95) |
| SS34 AOL2 | 100.00 | 94.49 | 94.05 |
| | (98.67-100.79) | (93.35-95.21) | (91.61-95.36) |
| SS34 AOL3 | 100.00 | 92.52 | 91.57 |
| | (99.52-100.96) | (90.62-94.97) | (90.35-92.96) |
| SS34 AOL4 | 100.00 | 92.95 | 91.96 |
| | (99.87-100.12) | (91.58-95.39) | (88.71-93.76) |
| SS34 AOL5 | 100.00 | 94.60 | 88.95 |
| | (99.08-100.60) | (93.76-95.12) | (88.36-89.61) |
| SS34 0.1% | 100.00 | 90.78 | 88.85 |
| (Control) | (99.27-100.62) | (88.35-93.35) | (88.54-89.40) |

Data presented as the mean with the range in brackets (n = 3).
Data analyzed by HPLC.

### Development of Active Cream Formulations

Development of a 1.00% w/w ADX-102 Formulation

The solvent systems developed for a 1% w/w ADX-102 are displayed in Table 28 and were investigated as final formulations. These solvent systems varied in the levels of Transcutol HP, propylene glycol (both below 10% w/w) and SR PEG 400 present. The most notable difference between the developed solvent systems and the comparison formulation solvent system was the level of water present, in which the comparison formulation solvent system contains 62.6% w/w compared to the 30-40% w/w levels under investigation. The water content in the comparison formulation was too high to achieve a 1% w/w formulation (as shown above, with a measured saturated solubility of only 0.31% w/w), therefore the water content was reduced to 30-40% w/w. This was to mitigate the risk of ADX-102 precipitation during stability (something which was observed with the comparison formulation) whilst ensuring that the drug was close to the saturated solubility value to allow for sufficient release of the ADX-102 from the formulation.

In all the solvent systems, phenoxyethanol was included at 1.00% w/w (1.05% limit on the FDA IIG) as a preservative. Antioxidants were also included. Based on the antioxidant selection experiments, the experiments demonstrated that BHT and BHA were the optimal antioxidants selected and that the levels of 0.08 and 0.02% w/w, respectively were sufficient to minimize oxidation of ADX-102. However, where feasible (depending on the excipients present in the solvent system and solubility of BHT/BHA in the system) both BHA and BHT at 0.1% w/w content were selected to further negate the possibility of ADX-102 oxidation should any of the BHT/BHA be sacrificial during shelf life storage. An example of which was solvent system SS37, in which ethanol is omitted from the aqueous phase resulting in an increased water content, compared to the other solvent systems (38.61% w/w). However, due to the absence of ethanol in SS37, BHT was observed to precipitate out of solution, consequently BHT has not been included in the composition for SS37; however BHA remained soluble at 0.10% w/w and was therefore included.

Using the solvent systems SS33, SS34, SS36 and SS37, active formulations CR060-CR069 (Table 29) were developed with the aim of producing an optimized 1% w/w cream formulation which was both chemically and physically stable, as well as aesthetically pleasing. The formulations contain various surfactants in combination such as Brij S2, Myrj S40 and Cetomacrogol 1000 where the levels were selected based on the HLB values of oil phase components, to mitigate the risk of phase separation in the formulations.

Formulations CR070-CR072 were developed based on the comparison formulation with some changes to both the aqueous phase (inclusion of ethanol and SR PEG 400 to increase drug solubility) and oil phase (replacement of ceresin wax with beeswax as this is present on the FDA IIG). CR070, CR071 and CR072 were developed with SS33, SS34 and SS36 respectively, to observe the differences in formulation characteristics.

TABLE 28
Composition (% w/w) of solvent systems for 1% w/w
ADX-102 formulations

Composition of solvent
systems (% w/w)

| Excipient | SS33 | SS34 | SS36 | SS37 |
|---|---|---|---|---|
| ADX-102 | 1.00 | 1.00 | 1.00 | 1.00 |
| Transcutol HP | 8.65 | 9.60 | 1.68 | 3.15 |
| Propylene Glycol | 5.50 | — | — | — |
| Purified Water | 34.93 | 30.40 | 30.40 | 38.61 |
| S.R. PEG 400 | 19.65 | 28.60 | 36.12 | 36.57 |
| Ethanol | 7.86 | 8.00 | 8.40 | — |
| Phenoxyethanol | 1.00 | 1.00 | 1.00 | 1.00 |
| BHT | 0.05 | 0.10 | 0.10 | — |
| BHA | 0.10 | 0.10 | 0.10 | 0.10 |
| Total | 78.74 | 78.80 | 78.80 | 80.43 |

TABLE 29
Composition (% w/w) of active formulations containing 1% w/w ADX-102

| | Composition (% w/w) | | | | | | | | | |
|---|---|---|---|---|---|---|---|---|---|---|
| Excipient | CR060 | CR061 | CR062 | CR063 | CR064 | CR065 | CR066 | CR067 | CR068 | CR069 |
| ADX-102 | 1.00 | 1.00 | 1.00 | 1.00 | 1.00 | 1.00 | 1.00 | 1.00 | 1.00 | 1.00 |
| Transcutol HP | 8.65 | 8.65 | 8.65 | 9.60 | 9.60 | 1.68 | 3.15 | 9.60 | 9.60 | 9.60 |
| Propylene | 5.50 | 5.50 | 5.50 | — | — | — | — | — | — | — |
| Glycol | | | | | | | | | | |
| Purified Water | 34.93 | 34.93 | 34.93 | 30.40 | 30.40 | 30.40 | 38.61 | 30.40 | 30.40 | 30.40 |
| S.R. PEG 400 | 19.65 | 19.65 | 19.65 | 28.60 | 28.60 | 36.12 | 36.57 | 28.60 | 28.60 | 28.60 |
| Ethanol | 7.86 | 7.86 | 7.86 | 8.00 | 8.00 | 8.40 | — | 8.00 | 8.00 | 8.00 |
| Phenoxyethanol | 1.00 | 1.00 | 1.00 | 1.00 | 1.00 | 1.00 | 1.00 | 1.00 | 1.00 | 1.00 |
| BHT | 0.05 | 0.05 | 0.05 | 0.10 | 0.10 | 0.10 | — | 0.10 | 0.10 | — |
| BHA | 0.10 | 0.10 | 0.10 | 0.10 | 0.10 | 0.10 | 0.10 | 0.10 | — | 0.10 |
| PEG 1500 | 9.26 | 9.26 | 9.26 | 9.20 | 9.20 | 9.20 | 7.57 | 9.20 | 9.30 | 9.30 |
| Steareth 21 | — | — | — | — | — | — | — | 1.52 | — | — |
| (Brij s721) | | | | | | | | | | |
| Steareth 2 | 2.60 | 2.84 | 2.37 | 2.70 | 2.37 | 2.84 | 2.37 | 2.48 | 2.84 | 2.84 |
| (Brij S2) | | | | | | | | | | |
| Cetomacrogol | 1000 | — | — | — | 1.30 | — | — | — | — | — |
| Myrj S40 | 1.40 | — | — | — | — | — | — | — | — | — |
| Brij CS25 | — | 1.16 | — | — | — | 1.16 | — | — | 1.16 | 1.16 |
| (Ceteareth 25) | | | | | | | | | | |
| White | 3.00 | 3.00 | 3.00 | 3.00 | 3.00 | 3.00 | 3.00 | 3.00 | 3.00 | 3.00 |
| petrolatum | | | | | | | | | | |
| Liquid | 5.00 | 5.00 | 5.00 | 5.00 | 5.00 | 5.00 | 5.00 | 5.00 | 5.00 | 5.00 |
| Paraffin | | | | | | | | | | |
| Steareth 20/ | — | — | 1.63 | — | 1.63 | — | 1.63 | — | — | — |
| Brij S20 | | | | | | | | | | |
| Total | 100.00 | 100.00 | 100.00 | 100.00 | 100.00 | 100.00 | 100.00 | 100.00 | 100.00 | 100.00 |
| Solvent system | SS33 | SS33 | SS33 | SS34 | SS34 | SS36 | SS37 | SS34 | SS34 | SS34 |
| Notable | Myrj | Brij | Brij | C | Brij | Brij | Brij | Brij | Brij | Brij |
| excipient | S40/ | CS25/ | S20/ | 1000/ | S20/ | CS25/ | S20/S2. | 721/ | CS25/ | CS25/ |
| changes | Brij. | S2 | S2 | Brij | S2 | S2 | No | S2 | S2, | S2, |
| | S2 | | | S2 | | | BHT | | BHT | BHA |
| | | | | | | | | | only | only |

| | Theoretical Composition (% w/w) | | |
|---|---|---|---|
| Excipient | CR070 | CR071 | CR072 |
| ADX-102 | 1.00 | 1.00 | 1.00 |
| Transcutol HP | 8.65 | 9.60 | 1.68 |
| Propylene Glycol | 5.50 | — | — |
| Purified Water | 34.93 | 30.40 | 30.40 |
| S.R. PEG 400 | 19.65 | 28.60 | 36.12 |
| Ethanol | 7.86 | 8.00 | 8.40 |
| Phenoxyethanol | 1.00 | 1.00 | 1.00 |
| BHT | 0.05 | 0.10 | 0.10 |
| BHA | 0.10 | 0.10 | 0.10 |
| HPMC | 0.50 | 0.50 | 0.50 |
| PEG 1500 | 4.86 | 4.80 | 4.80 |
| Steareth 2 | — | — | — |
| (Brij S2) | | | |
| Geleol mono | 7.00 | 7.00 | 7.00 |
| and diglycerides | | | |
| Span 20 | 2.00 | 2.00 | 2.00 |
| Beeswax | 3.00 | 3.00 | 3.00 |
| White petrolatum | 3.00 | 3.00 | 3.00 |
| Liquid Paraffin | — | — | — |
| Steareth 20/ | 0.90 | 0.90 | 0.90 |
| Brij S20 | | | |
| Total | 100.00 | 100.00 | 100.00 |
| Solvent system | SS33 | SS34 | SS36 |
| Notable excipient | Beeswax, | Beeswax, | Beeswax, |
| changes | HPMC | HPMC | HPMC |

### Development of a 5.00% w/w ADX-102 Formulation

The solvent system SS26 was developed in order to manufacture a 5% w/w formulation. The solvent system was developed using the DoE matrix to obtain an optimized formulation with 5% w/w ADX-102 content. A saturated solubility experiment was performed using the solvent system SS26, and ADX-102 was found to have a saturated solubility of 6.85% w/w (6.84-6.86% w/w) as shown in Table 30. The cream formulation CR079 (Table 31) was developed using the SS26 solvent system, incorporating 5% w/w ADX-102 into the formulation and employing the Brij S2/Brij S20 oil phase. Furthermore, for comparative purposes the concentration of ADX-102 in this formulation was reduced to 1% w/w while maintaining the same formulation composition (formulation CR080).

Formulations were characterized by apparent pH, visual appearance, accelerated physical stability (determined via centrifugation) and microscopic characteristics were recorded.

All the developed active creams had an apparent pH of between 6.63 and 8.45 and were white in color, had medium/high visual viscosity, were easily applied, and had a smooth texture. ADX-102 crystals were absent from all the developed formulations under both polarized and non-polarized light. Additionally, all the formulations showed no sign of phase separation after 16 mins following centrifugation, with the exception of CR068, CR071 and CR072, which appeared to phase separate after 10, 8, and 6 mins, respectively.

TABLE 30
Composition and solubility of ADX-102 (% w/w) in
the solvent system SS26

| | Composition of solvent systems (% w/w) |
|---|---|
| Excipient | SS26 |
| ADX-102 | 5.00 |
| Transcutol HP | 0.80 |
| Propylene glycol | — |
| Purified Water | 30.40 |
| S.R. PEG 400 | 40.80 |
| Ethanol | 8.00 |
| Phenoxyethanol | 1.00 |
| BHT | 0.10 |
| BHA | 0.10 |
| Total | 86.20 |
| ADX-102 saturated | 6.85 (6.84-6.86) |
| solubility (% w/w) | |

TABLE 31
Composition (% w/w) of active formulations with 5% w/w ADX-102

| | Theoretical Composition (% w/w) | |
|---|---|---|
| Excipient | CR079 | CR080 |
| ADX-102 | 5.00 | 1.00 |
| Transcutol HP | 0.80 | 0.80 |
| Purified Water | 30.40 | 30.40 |
| S.R. PEG 400 | 40.90 | 44.90 |
| Ethanol | 8.00 | 8.00 |
| Phenoxyethanol | 1.00 | 1.00 |
| BHT | 0.10 | 0.10 |
| BHA | 0.10 | 0.10 |
| PEG 1500 | 1.70 | 1.70 |
| Steareth 2 (Brij S2) | 2.37 | 2.37 |
| White petrolatum | 3.00 | 3.00 |
| Liquid Paraffin | 5.00 | 5.00 |
| Steareth 20/Brij S20 | 1.63 | 1.63 |
| Total | 100.00 | 100.00 |
| Solvent system | SS26 | SS26 |
| Notable excipient changes | 5% formulation | 1% formulation (not |
| | | optimized) |

### Short Term Stability

The 15 formulations CR060-CR080 described above were assessed in short term stability testing experiments, where macroscopic and microscopic appearance, physical and chemical stability were assessed at t=0, 2, 4, and 9 weeks (CR062, CR070 and CR079 only) at 25 and 40° C. At each timepoint, the following parameters were assessed:
ADX-102 content and related substances
Apparent pH
Microscopic appearance
Macroscopic appearance

The percentage recovery and purity of ADX-102 in certain formulations is presented below following 4 weeks of storage at 25 and 40° C. for all formulations tested and at 9 weeks of storage for CR062, CR070 and CR079.

Following 4 weeks and 9 weeks of storage (for CR062, CR070 and CR079 only) at 25 and 40° C., all formulations were observed to present consistent ADX-102 recovery values to T=0, ranging between 96.20-106.01%, with the exception of CR066 where the ADX-102 recovery value was observed of 87.09% following 4 weeks of storage at 40° C.

The recovery data is also reflected in the ADX-102 purity data below, where again consistent purity were observed for all formulations assessed to T=0 (<1.2% a/a reduction from T=0 in ADX-102 purity) following 4/9 weeks of storage at 25 and 40° C., with the exception of CR066, where an ADX-102 purity of 93.90% a/a was observed following storage at 40° C. for 4 weeks compared to 95.37% a/a at T=0 (total reduction of 1.47% a/a ADX-102). With regard to CR066, this formulation did not contain BHT while also containing the highest level of water of all of the formulations tested.

TABLE 32
Percentage recovery of ADX-102 in developed active formulations

| | Percentage (%) recovery of ADX-102 compared to recovery | | | | | | |
|---|---|---|---|---|---|---|---|
| | at T = 0 | | | | | | |
| | | T = 2 weeks | | T = 4 weeks | | T = 9 weeks | |
| Formulation | T = 0 | 25° C. | 40° C. | 25° C. | 40° C. | 25° C. | 40° C. |
| CR060 | 100.00 | 100.87 | 97.35 | 98.75 | 97.11 | N/A | |
| | (99.44- | (99.78- | (95.73- | (97.77- | (91.86- | | |
| | 100.53) | 102.34) | 99.72) | 99.35) | 106.27) | | |
| CR061 | 100.00 | 100.15 | 97.05 | 99.32 | 96.44 | N/A | |
| | (99.81- | (99.59- | (95.47- | (98.87- | (95.64- | | |
| | 100.36) | 100.64) | 98.92) | 99.85) | 97.86) | | |
| CR062 | 100.00 | 98.42 | 102.20 | 97.29 | 100.49 | 100.06 | 105.17 |
| | (99.56- | (97.66- | (101.41- | (96.67- | (98.84- | (99.76 | (104.67 |
| | 100.31) | 99.17) | 102.66) | 97.79) | 102.37) | 100.38) | 105.56) |
| CR063 | 100.00 | 98.91 | 106.03 | 99.56 | 98.08 | N/A | |
| | (99.07- | (97.48- | (105.98- | (99.20- | (97.97- | | |
| | 101.04) | 100.52) | 106.07) | 99.90) | 98.15) | | |
| CR064 | 100.00 | 98.34 | 103.65 | 98.86 | 103.62 | N/A | |
| | (99.89- | (97.88- | (103.57- | (98.75- | (102.49- | | |
| | 100.15) | 99.05) | 103.72) | 98.97) | 104.84) | | |
| CR065 | 100.00 | 99.15 | 100.26 | 98.57 | 96.20 | N/A | |
| | (98.99- | (98.38- | (99.80- | (98.17- | (95.05- | | |
| | 100.74) | 99.63) | 100.51) | 98.97) | 97.73) | | |
| CR066 | 100.00 | 99.79 | 96.90 | 98.92 | 87.09 | N/A | |
| | (99.76- | (98.69- | (95.58- | (98.31- | (84.64- | | |
| | 100.15) | 100. 90) | 98.22) | 99.33) | 90.47) | | |
| CR067 | 100.00 | 98.95 | 102.09 | 99.20 | 102.07 | N/A | |
| | (99.72- | (97.97- | (100.81- | (97.84- | (101.23- | | |
| | 100.26) | 100.53) | 103.43) | 100.05) | 103.11) | | |
| CR068 | 100.00 | 99.46 | 99.47 | 98.72 | 99.78 | N/A | |
| | (98.89- | (99.22- | (97.31- | (97.17- | (97.45- | | |
| | 101.27) | 99.82) | 101.84) | 100.10) | 101.43) | | |
| CR069 | 100.00 | 100.04 | 99.72 | 99.97 | 98.85 | N/A | |
| | (99.64- | (99.58- | (98.60- | (99.56- | (97.65- | | |
| | 100.63) | 100.35) | 101.08) | 100.66) | 100.45) | | |
| | Percentage (%) recovery of ADX-102 compared to recovery | | | | | | |
| | at T = 0 | | | | | | |
| | | T = 2 weeks | | T = 4 weeks | | T = 9 weeks | |
| Formulation | T = 0 | 25° C. | 40° C. | 25° C. | 40° C. | 25° C. | 40° C. |
| CR070 | 100.00 | 98.87 | 97.57 | 100.21 | 97.62 | 101.52 | 99.02 |
| | (99.66- | (98.63- | (95.90- | (98.25- | (97.34- | (100.63- | (98.07- |
| | 100.58) | 99.06) | 100.00) | 103.32) | 98.01) | 102.47) | 99.81) |
| CR071 | 100.00 | 100.87 | 99.60 | 101.43 | 98.06 | N/A | |
| | (99.35- | (99.89- | (98.57- | (99.95- | (96.10- | | |
| | 100.44) | 101.74) | 100.28) | 102.74) | 99.92) | | |
| CR072 | 100.00 | 99.88 | 99.25 | 100.92 | 99.10 | N/A | |
| | (97.61- | (99.30- | (98.27- | (100.80- | (98.08- | | |
| | 101.54) | 100.32) | 99.81) | 101.15) | 99.82) | | |
| CR079 | 100.00 | 97.02 | 102.98 | 98.09 | 102.48 | 100.85 | 106.01 |
| | (99.35- | (95.71- | (101.98- | (97.98- | (101.90- | (99.70- | (105.35- |
| | 100.93) | 98.07) | 104.67) | 98.20) | 102.85) | 102.89) | 106.34) |

| | | | | | | |
|---|---|---|---|---|---|---|
| CR080 | 100.00 | 99.93 | 99.98 | 98.70 | 101.41 | N/A |
| | (99.17- | (99.78- | (98.62- | (98.41- | (101.07- | |
| | 100.61) | 100.08) | 101.16) | 98.85) | 101.79) | |

Data presented as the mean with the range in brackets (n = 3).

TABLE 33
Percentage peak purity (% a/a) of ADX-102 in developed
active formulations

| | Percentage area/area (% a/a) of the ADX-102 drug peak | | | | | | |
|---|---|---|---|---|---|---|---|
| Formu- | | T = 2 weeks | | T = 4 weeks | | T = 9 weeks | |
| lation | T = 0 | 25° C. | 40° C. | 25° C. | 40° C. | 25° C. | 40° C. |
| CR060 | 99.21 | 99.09 | 99.02 | 99.14 | 98.85 | N/A | |
| CR061 | 99.19 | 99.25 | 99.33 | 99.31 | 99.11 | N/A | |
| CR062 | 99.92 | 99.26 | 99.24 | 99.24 | 99.07 | 99.18 | 98.86 |
| CR063 | 98.92 | 99.08 | 99.01 | 98.99 | 98.91 | N/A | |
| CR064 | 98.96 | 99.06 | 99.03 | 99.04 | 98.97 | N/A | |
| CR065 | 98.83 | 98.86 | 98.90 | 98.86 | 98.69 | N/A | |
| CR066 | 95.37 | 95.23 | 94.91 | 95.01 | 93.90 | N/A | |
| CR067 | 98.99 | 99.03 | 99.06 | 99.03 | 98.92 | N/A | |
| CR068 | 98.93 | 99.01 | 98.92 | 99.02 | 98.77 | N/A | |
| CR069 | 99.04 | 99.01 | 99.01 | 98.98 | 98.94 | N/A | |
| CR070 | 99.29 | 99.20 | 99.11 | 99.16 | 98.78 | 99.07 | 98.31 |
| CR071 | 99.01 | 98.98 | 98.94 | 98.97 | 98.76 | N/A | |
| CR072 | 95.34 | 95.17 | 94.51 | 94.43 | 94.16 | N/A | |
| CR079 | 99.60 | 99.63 | 99.62 | 99.57 | 99.49 | 99.47 | 99.39 |

| | | | | | | |
|---|---|---|---|---|---|---|
| CR080 | 98.13 | 98.36 | 98.35 | 98.26 | 97.84 | N/A |

Data presented as n = 1.

### Apparent pH

The apparent pH for the prototype formulations is provided below following 4 weeks of storage at 25 and 40 OC for all formulations tested and 9 weeks of storage for CR062, CR070 and CR079. The initial apparent pH for all active formulations was between 6.63-8.45. The apparent pH values for most formulations remained consistent over the 4/9 weeks of stability testing, with only slight variability observed, attributed to the sensitivity of the pH probe used to take the measurements and the low aqueous content of the formulations.

TABLE 34
Apparent pH values from placebo and active formulations

| | Apparent pH of developed formulations | | | | | | | | | | | | | |
|---|---|---|---|---|---|---|---|---|---|---|---|---|---|---|
| | | | T = 2 weeks | | | | T = 4 weeks | | | | T = 9 weeks | | | |
| | T = 0 | | 25° C. | | 40° C. | | 25° C. | | 40° C. | | 25° C. | | 40° C. | |
| Formulation | Placebo | Active | Placebo | Active | Placebo | Active | Placebo | Active | Placebo | Active | Placebo | Active | Placebo | Active |
| CR060 | 6.60 | 7.12 | 6.55 | 7.23 | 6.55 | 7.06 | 6.58 | 6.99 | 6.59 | 6.90 | N/A | | | |
| CR061 | 6.86 | 7.25 | 6.77 | 7.16 | 6.39 | 7.96 | 6.84 | 7.66 | 6.73 | 7.56 | N/A | | | |
| CR062 | 6.92 | 7.47 | 6.96 | 7.24 | 6.18 | 7.75 | 6.95 | 7.22 | 5.91 | 7.81 | 7.59 | 7.73 | 7.08 | 7.24 |
| CR063 | 7.02 | 7.26 | 6.88 | 7.12 | 6.62 | 7.31 | 6.75 | 7.56 | 7.12 | 6.64 | N/A | | | |
| CR064 | 6.52 | 8.45 | 6.76 | 7.30 | 6.81 | 8.06 | 6.75 | 7.58 | 7.16 | 8.03 | N/A | | | |
| CR065 | 6.99 | 7.97 | 6.85 | 7.66 | 6.52 | 8.04 | 6.85 | 7.43 | 6.63 | 7.50 | N/A | | | |
| CR066 | 6.24 | 6.63 | 6.16 | 6.38 | 5.88 | 6.14 | 6.22 | 6.29 | 6.14 | 5.45 | N/A | | | |
| CR067 | 6.68 | 7.53 | 6.74 | 7.44 | 6.45 | 7.52 | 6.09 | 7.48 | 6.42 | 7.69 | N/A | | | |
| CR068 | 6.84 | 7.25 | 6.80 | 7.31 | 6.83 | 7.68 | 6.60 | 7.54 | 6.49 | 7.51 | N/A | | | |
| CR069 | 6.96 | 7.12 | 6.71 | 7.53 | 6.72 | 8.10 | 6.50 | 7.64 | 6.45 | 7.18 | N/A | | | |
| CR070 | 6.54 | 6.69 | 6.60 | 6.83 | 6.57 | 7.00 | 6.57 | 6.84 | 6.21 | 6.53 | 6.38 | 6.77 | 5.90 | 6.60 |
| CR071 | 7.20 | 7.25 | 6.76 | 7.02 | 6.76 | 7.29 | 6.55 | 7.02 | 5.97 | 6.66 | N/A | | | |
| CR072 | 6.66 | 6.87 | 6.76 | 6.71 | 6.32 | 6.53 | 6.57 | 6.69 | 5.87 | 6.07 | N/A | | | |
| CR079 | 7.80 | 7.69 | 7.93 | 7.40 | 7.25 | 7.32 | 6.89 | 6.78 | 6.48 | 6.80 | 7.17 | 7.56 | 7.58 | 6.58 |

| | | | | | | |
|---|---|---|---|---|---|---|
| CR080 | 8.38 | 7.71 | 7.44 | 7.05 | 7.31 | N/A |

### Microscopic Appearance

Microscopic observations of the formulations were made using light microscopy with a magnification of 400× (polarized and non-polarized light) following 4 weeks of storage at 25 and 40° C. for all formulations tested and 9 weeks of storage for CR062, CR070 and CR079.

At t=0, all formulations under non-polarized light presented medium-sized, evenly distributed emulsion droplets, however following storage at t=2 weeks at 40° C., CR060 was observed to have small oil droplets which were very close together. Following t=4 weeks at 40° C., CR065 and CR066 also had small oil droplets which were tightly packed under non-polarized light, suggesting that the oil droplets were beginning to coalesce. During the short-term stability testing over 4/9 weeks, no ADX-102 crystals were observed in any of the active or placebo formulations under polarized light.

### Macroscopic Appearance

The prototype formulations were assessed for macroscopic appearance following 4 weeks of storage at 25 and 40° C. for all formulations tested and 9 weeks of storage for CR062, CR070 and CR079.

At t=0, all of the formulations had a smooth application, C060-C078 were of medium visual viscosity and C079 and C080 were of high visual viscosity, and all formulations were white in color. Following storage at 25° C. for 2 weeks, all of the active and vehicle formulations were visually similar to t=0. However at 40° C. the formulations CR060 and CR063 appeared to have phase separated. After t=4 weeks of storage, CR060 and CR063 remained phase separated, however the presence of an oil layer indicating phase separation was also observed in CR065 when stored at 40° C. While there were some formulations which had phase separated at 40° C., it should be noted that 40° C. is an accelerated condition, and no phase separation occurred at 25° C. Additionally, after t=4 weeks (25 and 40° C.), it should be noted that CR071 exhibited a slightly gritty texture. All other formulations stored at 4/9 weeks at 25 and 40° C. were visually observed to be physically stable, white emulsions with a medium to high viscosity.

### Super-Refined PEG 400 Versus Non-Refined PEG 400

The use and requirement of non-super refined PEG 400 as an excipient was investigated in view of to the higher cost of SR PEG 400. Saturated solubility and chemical stability of ADX-102 in PEG 400 (non-super refined) was performed as described above.

The results of the saturated solubility showed that ADX-102 has >20.00% w/w saturated solubility in both grades of PEG 400, suggesting that use of either grade should not affect the concentration of ADX-102 in the formulation.

The chemical stability of ADX-102 in both grades of PEG 400 was performed and the results are presented in Table 35 in comparison with the percentage recovery of ADX-102 from super-refined PEG 400. Following 2 weeks of storage at 50° C., the recovery of non-super refined PEG 400 decreased to 92.31% (from 100%); however, the recovery of SR PEG 400 remained consistent following 2 weeks of storage at 40 and 50° C. (100.12 and 99.44%, respectively) when compared to T=0 (99.93%). The peak purity data (Table 36) was consistent with the recoveries observed, where low peak purity of non-super refined PEG 400 following t=2 weeks at 50° C. (92.31% a/a) was observed, compared to the super-refined grade of PEG 400 under the same condition (97.59% a/a). The difference in percentage recovery between the 2 grades of PEG 400 was most likely due to the aldehyde content in SR PEG 400 (Supplier: Croda) being 0 ppm, and in non-super refined PEG 400 (Supplier: Merck) the aldehyde content was <30 ppm. Accordingly, the decision was made that SR-PEG 400 would be used during further studies.

TABLE 35
Percentage recovery of ADX-102 in SR or non-SR PEG 400

| | Percentage (%) recovery of ADX-102 compared | | | | |
|---|---|---|---|---|---|
| | to theoretical concentration | | | | |
| | | T = 1 week | | T = 2 weeks | |
| Excipient | T = 0 | 40° C. | 50° C. | 40° C. | 50° C. |
| SR PEG 400 | 99.93 | 95.83 | 98.86 | 100.12 | 99.44 |
| | (98.45- | (94.51- | (98.25- | (99.26- | (99.35- |
| | 101.41) | 97.15) | 99.47) | 100.99) | 99.52) |
| PEG 400 | 99.50 | 97.51 | 95.98 | 94.38 | 92.31 |
| | (99.29- | (97.11- | (95.56- | (93.94- | (91.64- |
| | 99.89) | 98.01) | 96.25) | 94.63) | 92.92) |

Data presented as the mean with the range in brackets (n = 3).
Data analyzed by developed HPLC assay method.

TABLE 36
Percentage peak purity (% a/a) of ADX-102 in SR or non-SR PEG 400

| | Percentage peak purity (% a/a) of ADX-102 | | | | |
|---|---|---|---|---|---|
| | compared to theoretical concentration | | | | |
| | | T = 1 week | | T = 2 weeks | |
| Excipient | T = 0 | 40° C. | 50° C. | 40° C. | 50° C. |
| SR PEG 400 | 99.93 | 99.33 | 98.98 | 98.32 | 97.59 |
| PEG 400 | 99.91 | 97.99 | 94.98 | 96.85 | 89.91 |

Data is presented as n = 1.

### Formulation Selection for Performance Testing

Certain formulation candidates developed for formulation stability were selected for performance testing (In vitro drug release studies and In vitro drug permeation and penetration testing).

Table 37 highlights the formulations selected for performance testing experiments. The ten formulation candidates were CR061, CR062, CR064, CR065, CR069, CR070, CR071, CR079, CR080 and the comparison formulation.

Table 38 highlights additional formulations that were prepared and tested.

TABLE 37
Composition (% w/w) of active formulations selected for performance testing

| | Composition (% w/w) | | | | | | | | | |
|---|---|---|---|---|---|---|---|---|---|---|
| Excipient | CR060 | CR061 | CR062 | CR063 | CR064 | CR065 | CR066 | CR067 | CR068 | CR069 |
| ADX-102 | 1.00 | 1.00 | 1.00 | 1.00 | 1.00 | 1.00 | 1.00 | 1.00 | 1.00 | 1.00 |
| Transcutol HP | 8.65 | 8.65 | 8.65 | 9.60 | 9.60 | 1.68 | 3.15 | 9.60 | 9.60 | 9.60 |
| Propylene Glycol | 5.50 | 5.50 | 5.50 | — | — | — | — | — | — | — |
| Purified Water | 34.93 | 34.93 | 34.93 | 30.40 | 30.40 | 30.40 | 38.61 | 30.40 | 30.40 | 30.40 |
| S.R. PEG 400 | 19.65 | 19.65 | 19.65 | 28.60 | 28.60 | 36.12 | 36.57 | 28.60 | 28.60 | 28.60 |
| Ethanol | 7.86 | 7.86 | 7.86 | 8.00 | 8.00 | 8.40 | — | 8.00 | 8.00 | 8.00 |
| Phenoxyethanol | 1.00 | 1.00 | 1.00 | 1.00 | 1.00 | 1.00 | 1.00 | 1.00 | 1.00 | 1.00 |
| BHT | 0.05 | 0.05 | 0.05 | 0.10 | 0.10 | 0.10 | — | 0.10 | 0.10 | — |
| BHA | 0.10 | 0.10 | 0.10 | 0.10 | 0.10 | 0.10 | 0.10 | 0.10 | — | 0.10 |
| PEG 1500 | 9.26 | 9.26 | 9.26 | 9.20 | 9.20 | 9.20 | 7.57 | 9.20 | 9.30 | 9.30 |
| Steareth 21 (Brij | — | — | — | — | — | — | — | 1.52 | — | — |
| s721) | | | | | | | | | | |
| Steareth 2 (Brij S2) | 2.60 | 2.84 | 2.37 | 2.70 | 2.37 | 2.84 | 2.37 | 2.48 | 2.84 | 2.84 |
| Cetomacrogol 1000 | — | — | — | 1.30 | — | — | — | — | — | — |
| Myrj S40 | 1.40 | — | — | — | — | — | — | — | — | — |
| Brij CS25 (Ceteareth | — | 1.16 | — | — | — | 1.16 | — | — | 1.16 | 1.16 |
| 25) | | | | | | | | | | |
| White petrolatum | 3.00 | 3.00 | 3.00 | 3.00 | 3.00 | 3.00 | 3.00 | 3.00 | 3.00 | 3.00 |
| Liquid Paraffin | 5.00 | 5.00 | 5.00 | 5.00 | 5.00 | 5.00 | 5.00 | 5.00 | 5.00 | 5.00 |
| Steareth 20/Brij S20 | — | — | 1.63 | — | 1.63 | — | 1.63 | — | — | — |
| Total | 100.00 | 100.00 | 100.00 | 100.00 | 100.00 | 100.00 | 100.00 | 100.00 | 100.00 | 100.00 |
| Solvent system | SS33 | SS33 | SS33 | SS34 | SS34 | SS36 | SS37 | SS34 | SS34 | SS34 |
| Notable excipient | Myrj | Brij | Brij S20/ | C | Brij S20/ | Brij | Brij S20/ | Brij | Brij | Brij |
| changes | S40/Brij. | CS25/S2 | S2 | 1000/Brij | S2 | CS25/S2 | S2. No | 721/S2 | CS25/S2, | CS25/S2, |
| | S2 | | | S2 | | | BHT | | BHT only | BHA only |
| Selected? | Not | Selected | Selected | Not | Selected | Selected | Not | Not | Not | Selected |
| | selected | | | selected | | | selected | selected | selected | |

| | Composition (% w/w) | | | | |
|---|---|---|---|---|---|
| Excipient | CR070 | CR071 | CR072 | CR079 | CR080 |
| ADX-102 | 1.00 | 1.00 | 1.00 | 5.00 | 1.00 |
| Transcutol HP | 8.65 | 9.60 | 1.68 | 0.80 | 0.80 |
| Propylene Glycol | 5.50 | — | — | — | — |
| Purified Water | 34.93 | 30.40 | 30.40 | 30.40 | 30.40 |
| S.R. PEG 400 | 19.65 | 28.60 | 36.12 | 40.90 | 44.90 |
| Ethanol | 7.86 | 8.00 | 8.40 | 8.00 | 8.00 |
| Phenoxyethanol | 1.00 | 1.00 | 1.00 | 1.00 | 1.00 |
| BHT | 0.05 | 0.10 | 0.10 | 0.10 | 0.10 |
| BHA | 0.10 | 0.10 | 0.10 | 0.10 | 0.10 |
| HPMC | 0.50 | 0.50 | 0.50 | — | |
| PEG 1500 | 4.86 | 4.80 | 4.80 | 1.70 | 1.70 |
| Steareth 2 (Brij S2) | — | — | — | 2.37 | 2.37 |
| Geleol mono and | 7.00 | 7.00 | 7.00 | — | — |
| diglycerides | | | | | |
| Span 20 | 2.00 | 2.00 | 2.00 | — | — |
| Beeswax | 3.00 | 3.00 | 3.00 | — | — |
| White petrolatum | 3.00 | 3.00 | 3.00 | 3.00 | 3.00 |
| Liquid Paraffin | — | — | — | 5.00 | 5.00 |
| Steareth 20/Brij S20 | 0.90 | 0.90 | 0.90 | 1.63 | 1.63 |
| Total | 100.00 | 100.00 | 100.00 | 100.00 | 100.00 |
| Solvent system | SS33 | SS34 | SS36 | SS26 | SS26 |
| Notable excipient | Beeswax, HPMC | Beeswax, HPMC | Beeswax, HPMC | 5% formulation | Corresponding 1% |
| changes | | | | | formulation (not |
| | | | | | optimized) |
| Selected? | Selected | Selected | Not selected | Selected | Selected |

TABLE 38
Composition of Additional Formulations Tested

| | Theoretical composition (% w/w) of ADX-102 active formulations | | | | | | | | |
|---|---|---|---|---|---|---|---|---|---|
| Excipient | CR90 | CR94 | CR95 | CR98 | CR103 | CR104 | CR109 | CR110 | CR111 |
| ADX-102 | 1 | 1 | 1 | 1 | 1 | 1 | 1 | 1 | 1 |
| Transcutol HP | 8.65 | 8.65 | 8.65 | 9.95 | 8.65 | 8.65 | 8.65 | 8.65 | 9.65 |
| Propylene Glycol | 5.5 | 5.5 | 5.5 | 7.36 | 5.5 | 5.5 | 5.5 | 5.5 | — |
| Purified Water | 34.93 | 34.93 | 34.93 | 35.46 | 34.93 | 34.93 | 34.85 | 34.93 | 35.46 |
| Super Refined PEG 400 | 19.748 | 19.728 | 19.778 | 20.9 | 19.65 | 19.65 | 19.778 | 19.778 | 20.748 |
| Ethanol | 7.86 | 7.86 | 7.86 | — | 7.86 | 7.86 | 7.86 | 7.86 | 8.86 |
| Phenoxyethanol | 1 | 1 | 1 | 1 | 1 | 1 | 1 | 1 | 1 |
| BHT | 0.05 | 0.05 | — | — | — | — | 0.05 | — | — |
| Alpha tocopherol | 0.002 | 0.002 | 0.002 | — | 0.002 | 0.002 | — | 0.002 | 0.002 |
| Propyl gallate | — | — | — | 0.05 | — | — | 0.05 | — | — |
| Ascorbyl palmitate | — | 0.02 | 0.02 | 0.02 | 0.02 | 0.02 | — | 0.02 | 0.02 |
| HPMC | 0.5 | 0.5 | 0.5 | 0.5 | 0.5 | 0.75 | 0.5 | 0.5 | 0.5 |
| PEG 1500 | 4.86 | 4.86 | 4.86 | 4.86 | 4.86 | — | 4.86 | 4.86 | 4.86 |
| Geleol mono and diglycerides | 7 | 7 | 7 | 10 | 7 | 7 | 7 | 8 | 9 |
| Span 20 (Sorbitan | 2 | 2 | 2 | 2 | 2 | 2 | 2 | — | 2 |
| monolaurate) | | | | | | | | | |
| Beeswax | 3 | 3 | 3 | 3 | 6.128 | 3 | 3 | 3 | 3 |
| White petrolatum | 3 | 3 | 3 | 3 | — | 7.738 | 3 | 3 | 3 |
| Steareth 20/Brij S20 | 0.9 | 0.9 | 0.9 | 0.9 | 0.9 | 0.9 | 0.9 | 1.9 | 0.9 |
| Total | 100 | 100 | 100 | 100 | 100 | 100 | 100 | 100 | 100 |

### CONCLUSION

We developed analytical methods for the pre-formulation and formulation development experiments performed in this study. As described above, we completed solubility experiments using an array of excipients and binary solvent systems, highlighting a range of solvents (phenoxyethanol, ethanol, isopropanol, SR PEG 400, Arlasolve DMI, diisopropyl adipate, IPM, Transcutol HP and benzyl alcohol) and non-solvents (water and glycerol) for ADX-102. ADX-102 was also shown to have solubility of 0.31% in the comparison solvent system, despite its nominal 1.0% w/w amount, highlighting a potential reason for the crystallization observed in the comparison formulation. ADX-102 was shown to have good stability in a range of excipients tested (>98.21% peak purity; e.g., benzyl alcohol, phenoxyethanol, SR PEG 400, Transcutol HP, ethanol, water, propylene glycol) with the exception of isopropyl alcohol, oleyl alcohol, SR Arlasolve DMI and diisopropyl adipate (at 50° C.) after t=2 weeks. Antioxidant selection experiments were performed where BHA and BHT were observed to be the most effective antioxidant/stabilizers for preventing oxidative degradation of ADX-102 and the minimal levels for each antioxidant were determined.

We developed a range of cream formulations at 1% w/w and 5% w/w ADX-102 incorporating the appropriate excipients selected following the pre-formulation experiments, where a total of 15 cream formulations were selected for short-term stability testing at 25 and 40° C. Good chemical stability (percentage purity >97%) of ADX-102 was observed in all formulations following storage for 4 weeks at 25 and 40° C., with the exception of CR066 and CR072, which had percentage purity of 93.90 and 94.16% after t=4 weeks at 40° C., due to a large impurity peak eluting at 20 min. All of the cream formulations had no ADX-102 crystals present, although CR060, CR063 and CR065 were observed to phase separate over the 4-week testing period at 40° C. All remaining formulations had appealing aesthetics (white cream, medium-high viscosity, with a smooth application) at both T=0 and following stability storage over 4 weeks. In addition, a select of formulations (CR062, CR070 and CR079) were also assessed following 9 weeks of storage at 25 and 40° C. where comparable results to T=0 were observed. Thus, we successfully developed a range of formulations containing ADX-102 which were observed to be chemically and physically stable over 9 weeks of storage at 25 and 40° C.

### Example 6: In Vitro Drug Release Testing (IVRT)

### Introduction

The aim of this study was to perform in vitro drug release testing on the selected formulations.

The previously developed HPLC method for the assay and quantification of ADX-102 was used. A suitable receiver fluid (20% PEG 400 in 50:50 v/v ethanol:water) and candidate membranes (cellulose acetate and PVDF) were selected following method development experiments.

A small scale IVRT experiment was performed with two membranes to establish the experimental parameters to be employed in the full scale experiment, in which steady-state release of the drug was achieved between the 1- and 6-h time points. Following the small scale investigation, we employed sampling time points of t=0, 1, 2, 4, 5, and 6 h and finalized cellulose acetate as the membrane for the full scale experiments.

Steady-state release rates ranging from 63.14 μg/cm²/√h to 449.82 μg/cm²/√h following the applications of CR064 and the comparison formulation, respectively, to the membrane. A full scale IVRT experiment was performed and, consistent with the findings of the small scale experiment, drug was detected in the receiver fluid from t=1 h following the application of all formulations. Steady-state release rates ranging from 63.14 μg/cm²/√h to 449.82 μg/cm²/√h were observed following the application of test formulations. The release rate of ADX-102 from the comparison formulation was significantly higher than the newly-developed formulations (p<0.05); however, this may be due to the drug being at maximum thermodynamic activity, as evidenced by the presence of drug crystals in the formulation. When the release rates of the newly-developed formulations were considered, CR070, CR071, and CR079 were found to release up to 7 fold, 6.5 fold, and 6 fold more ADX-102, respectively, into the receiver fluid than other newly-developed formulations. Furthermore, statistically higher (p<0.05) release rates of the drug were observed in CR070 and CR071, when compared to all other newly-developed formulations (with the exception of the 5% w/w ADX-102 formulation CR079), which was attributed both to the similarity of the formulations to the comparator and the absence/lower levels of excipient (e.g., PEG 1500) which may present a more complex matrix from which the drug must release. Following the full scale IVRT investigation, the in vitro drug release testing model and HPLC analytical method for the assay of ADX-102 was validated in accordance with the FDA's SUPAC—SS guidelines.

We assessed the in vitro drug (ADX-102) release of 9 topical formulations developed as described above and the comparison formulation, which are suitable for the treatment of ichthyosis due to Sjögren-Larsson Syndrome (SLS), among other conditions. The formulations investigated were: CR061, CR062, CR064, CR065, CR069, CR070, CR071, CR079, CR080, and the comparison formulation, with concentrations of drug ranging from 1% w/w to 5% w/w.

In summary, we successfully performed IVRT experiments to assess the release of ADX-102 from the developed formulations and have demonstrated release of the compound from the vehicle. It was also found that CR070 and CR071 demonstrated the highest steady state release of all the formulations developed during this study. Additionally, the IVRT model employed, including the HPLC analytical method for the assay of ADX-102, was validated in accordance with the FDA's SUPAC—SS guidelines.

### Small Scale In Vitro Drug Release Testing

For the preliminary small scale in vitro drug release experiments, a single representative test formulation [the comparison formulation (1% w/w ADX-102)], was employed to establish the sampling protocol to adequately profile the release of the drug across the synthetic membrane in the full scale investigations.

The small scale in vitro drug release experiment was performed as follows:
(i) Individually calibrated Franz cells were employed with an average surface area and volume of 2 cm² and 10 mL, respectively.
(ii) Cellulose acetate membrane was mounted between the donor and receiver chamber of Franz cells; selected based on the findings of the drug membrane binding and back diffusion experiments.
(iii) The receiver chambers of assembled Franz cells were filled with either ethanol and water (50:50 v/v) or 20% PEG 400 in ethanol and water (50:50 v/v).
(iv) The Franz cells were equilibrated in a water bath pre-calibrated to 32° C., for 30 min prior to dosing.
(v) An infinite dose (300 mg) of the selected formulation was applied to the surface the membrane of each Franz cells (2 replicate cells for the active formulation in each receiver fluid). An additional Franz cell (n=1) was mounted with cellulose acetate membrane but was not dosed with the formulation to serve as a blank.
(vi) The Franz cells were occluded using Parafilm® and protected from light for the duration of experimental period.
(vii) Aliquots of the receiver fluid (1 mL) were removed at the following time points t=0, 1, 2, 4, 6, 24, 30, and 48 h and transferred to deep well 96-well plates.
(viii) At each sampling time point, the receiver fluid removed was replaced with fresh pre-warmed receiver fluid.
(ix) Samples were analyzed using the HPLC UV method.

### Full Scale In Vitro Drug Release Testing

The developed procedures were employed for the full scale in vitro drug release experiment. The full-scale investigation assessed drug release of ADX-102 from ten formulations across a cellulose acetate membrane. The formulations were employed with six replicate cells per formulation, a single replicate of the placebo formulation and a single control blank Franz cell were employed.

As such, the parameters employed for the full-scale skin drug release experiments, informed by the findings of the small scale investigations, were as follows:
1. Synthetic membrane: Cellulose acetate
2. Receiver fluid system: 20% PEG 400 in 50:50 v/v ethanol:water.
3. Sampling time points: t=0, 1, 2, 4, 5, and 6 h.

Validation of the In Vitro Drug Release Model

A representative formulation was employed to validate the in vitro release experimental methods in accordance with the FDA's SUPAC-SS guidelines. The formulation selected was CR070 1.00% w/w ADX-102 cream. Sensitivity of the in vitro model to different levels of ADX-102 was evaluated as follows. CR070 1.00% ADX-102 cream formulation was manufactured to 80% and 120% of the label strength (0.8% and 1.2% w/w, respectively). Each formulation was tested (six replicate cells per formulation). A single operator was tasked with conducting the entire parameter test.

The CR070 formulations (10 g batches) at different strengths were prepared as follows:
(i) Antioxidants (BHT and BHA) were added to a suitably sized glass vial.
(ii) The relevant non-volatile and volatile aqueous-phase excipients were sequentially added to the vial and the contents stirred until the contents were dissolved/homogenous.
(iii) ADX-102 was added to the vial and the contents stirred until dissolved.
(iv) HPMC was slowly added (where applicable) to the vial while the aqueous phase was constantly stirred.
(v) To a separate suitably sized glass vial, the relevant oil phase excipients (and PEG 1500) were added, the contents of which were heated in a pre-calibrated water bath at 70° C. until melted.
(vi) Once the oil phase excipients from Step (v) had melted, the aqueous phase from Step
(iv) was added to the pre-calibrated water bath at 70° C. until a target temperature of ca. 60° C. was achieved.
(vii) The oil phase was added directly to the vial containing the aqueous phase and the sample was homogenized for 2 mins at 10,000 RPM.
(viii) Following homogenization, the samples were stirred until cooled to room temperature.

TABLE 39
Composition of CR070 formulations with different
concentrations of ADX-102

| | Composition (% w/w) | | |
|---|---|---|---|
| | CR070 | CR070 | CR070 |
| Excipient | (1%) | (1.2%) | (0.8%) |
| ADX-102 | 1.00 | 1.20 | 0.80 |
| Transcutol HP | 8.65 | 8.65 | 8.65 |
| Propylene Glycol | 5.50 | 5.50 | 5.50 |
| Purified Water | 34.93 | 34.93 | 34.93 |
| S.R. PEG 400 | 19.65 | 19.45 | 19.85 |
| Ethanol | 7.86 | 7.86 | 7.86 |
| Phenoxyethanol | 1.00 | 1.00 | 1.00 |
| BHT | 0.05 | 0.05 | 0.05 |
| BHA | 0.10 | 0.10 | 0.10 |
| HPMC | 0.50 | 0.50 | 0.50 |
| PEG 1500 | 4.86 | 4.86 | 4.86 |
| Geleol mono and diglycerides | 7.00 | 7.00 | 7.00 |
| Span 20 | 2.00 | 2.00 | 2.00 |
| Beeswax | 3.00 | 3.00 | 3.00 |
| White petrolatum | 3.00 | 3.00 | 3.00 |
| Steareth 20/Brij S20 | 0.90 | 0.90 | 0.90 |
| Total | 100.00 | 100.00 | 100.00 |

HPLC Method Validation

In order to validate the IVRT model, the HPLC method developed for the assay and detection of ADX-102 was successfully validated in the receiver fluid 20% PEG 400 in 50:50 v/v ethanol:water according to standard procedures recommended by the FDA.

### Results and Discussion

HPLC UV Method Implementation for the Assay and Quantification of ADX-102

The analytical methods employed for the quantification of ADX-102 and ADX-102 related substances in samples following the in vitro drug release experiments was developed during an earlier study described above. The analytical method was deemed to pass all specification criteria.

In Vitro Drug Release Testing Method Development

The solubility of the drug in a range of receiver fluid systems was determined to ensure that drug release was not limited by the solubility of ADX-102 in the receptor compartment of Franz cells. The receiver fluids selected for investigation were: ethanol and water (50:50 v/v), 2% Brij 98 in ethanol and water (50:50 v/v), 20% PEG in water, 20% PEG 400 in ethanol and water (50:50 v/v) and 2% Tween 80 in water. The findings are summarized below.

TABLE 40
The solubility of ADX-102 in a range of potential receiver fluid systems

| Receiver | | Solubility |
|---|---|---|
| Fluid | Composition | (mg/mL) |
| 1 | Ethanol and water (50:50 v/v) | 23.548 |
| 2 | 2% Brij 98 in ethanol and water (50:50 v/v) | 28.31 |
| 3 | 20% PEG 400 in water | 1.31 |
| 4 | 20% PEG 400 in ethanol and water (50:50 v/v) | 20.46 |
| 5 | 2% Tween 80 in water | 26.32 |

The solubility of ADX-102 in the investigated receiver fluid systems ranged from 1.31 mg/mL to 28.35 mg/mL. The test formulations contain 1-5% w/w ADX-102, and thus if it was assumed that 10% of the applied drug released from the formulation based on an application of 1 g (infinite dose), the maximum concentration that could be achieved in the receiver fluid was 1-5 mg/mL. As such, the solubility of ADX-102 in receiver fluid 1 (23.55 mg/mL), receiver fluid 2 (28.31 mg/mL) and receiver fluid 4 (20.46 mg/mL) were thought to be suitable as sink conditions would be maintained for the duration of an IVRT experiment. The aforementioned receiver fluids were therefore employed for the analysis of the chemical stability of ADX-102.

### Chemical Stability of ADX-102 in the Potential Receiver Fluid Systems

The chemical stability of ADX-102 over 6 days in a range of receiver fluid systems was determined to ascertain the potential for the compound to degrade in the receiver fluid over the experimental period or during storage. The selected receiver fluid systems for investigation were: ethanol and water (50:50 v/v), 2% Brij 98 in ethanol and water (50:50 v/v) and 20% PEG 400 in ethanol and water (50:50 v/v). The chemical stability at 24 h, 48 h, 5 days and 6 days across three storage conditions (2-8° C. and 32° C.) was calculated as the percent of ADX-102 recovered when compared to drug quantification at t=0.

ADX-102 was sufficiently stable (range of 92-106% recovery of the drug from t=0) in the ethanol and water (50:50 v/v) and 20% PEG 400 in ethanol and water (50:50 v/v) when stored at 2-8° C. and 32° C. for up to 6 days (receiver fluid systems 1 and 4, respectively). The drug, ADX-102, demonstrated inferior chemical stability in 2% Brij 98 in ethanol and water (50:50 v/v) when stored at 2-8° C. and 32° C. for up to 6 days, with drug recoveries ranging from 76-92%. In an attempt to ensure maximum stability of the compound in the receiver fluid, it was deemed appropriate to select ethanol and water (50:50 v/v) and 20% PEG 400 in ethanol and water (50:50 v/v) as potential receiver fluid systems for the in vitro drug release testing, subject to the outcome of the drug-membrane binding investigations.

Binding of ADX-102 to Synthetic Membranes

Prior to commencing the small scale in vitro drug release testing, the potential for the drug, when present in a range of receiver fluid systems, to bind to a range of synthetic membranes was investigated. The systems selected were receiver fluid 1, 2 and 4 based on the findings of the drug solubility and chemical stability investigations. The synthetic membranes selected for investigation were: PTFE membrane (0.18 μm pore size), PVDF (0.45 μm pore size), cellulose membrane and nylon membrane. A receiver fluid comprising ethanol and water (50:50 v/v) or 20% PEG 400 in ethanol and water (50:50 v/v) was deemed appropriate to employ for the small scale in vitro drug release testing.

Assessment of Back Diffusion of the Investigated Receiver into the Donor Chamber of Franz Cells

Following the membrane binding experiments, back diffusion of ethanol and water (50:50 v/v) or 20% PEG 400 in ethanol and water (50:50 v/v) across cellulose acetate, nylon, PVDF membranes were investigated.

Back diffusion of both receiver fluids into the donor chamber of Franz cells was observed with nylon membrane and PVDF membrane. As such, it was deemed appropriate to employ cellulose acetate as the synthetic membrane for the small scale in vitro drug release testing given the low drug binding and prevention of back diffusion of the receiver fluids across this membrane.

### Small Scale In Vitro Drug Release Testing

A small scale in vitro drug release investigation was conducted to establish the sampling protocol to adequately profile ADX-102 release across a synthetic membrane and select the most suitable receiver fluid to be employed during the full scale in vitro drug release testing experiments. For the preliminary small scale in vitro drug release experiments, a single representative test formulation, the comparison formulation (1% w/w ADX-102), was employed.

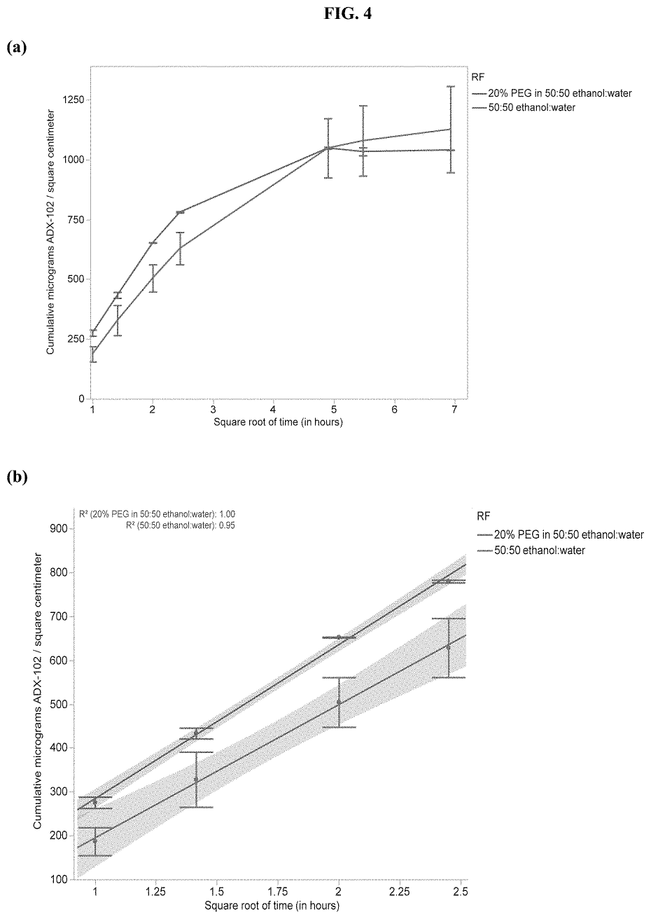

Following the application of the comparison formulation to the surface of the membrane (cellulose acetate), ADX-102 was detected in both receiver fluid systems [ethanol and water (50:50 v/v) and 20% PEG 400 in ethanol and water (50:50 v/v)] at the earliest time point (from 1 h onwards). The results of the small scale drug release experiment indicate that drug release was linear with respect to the square root of time over the first six hours of the experimental period as shown in FIG. 4A. Accordingly, we planned sampling of the receiver fluid during the full scale in vitro drug release experiments at 0, 1, 2, 4, 5, and 6 h to adequately profile the release of ADX-102 from the investigated formulations as a plateau was observed at the later time points (i.e., between 24 and 48 h.) Furthermore, it was deemed suitable to employ 20% PEG 400 in ethanol and water (50:50 v/v) as the receiver fluid system, due to the lower variability exhibited by the replicates using this receiver fluid, along with a cellulose acetate membrane to conduct the full scale in vitro drug release experiments.

### Full Scale In Vitro Drug Release Testing

A full scale in vitro drug release experiment was next conducted. The full scale investigation aimed to profile the release of ADX-102 from 10 formulations across cellulose acetate.

The cumulative amount of ADX-102 released across cellulose acetate over the square root of time was measured. Consistent with the findings of the small scale experiment, drug was detected in the receiver fluid from the earliest time point (t=1 h). From the release profiles constructed, the steady-state drug release across cellulose acetate over 6 h (μg/cm²/√h) was calculated and the findings presented below. The rank order, from highest to lowest steady-state release rate of ADX-102, is as follows:
Comparison Formulation >CR070>CR071>CR079>CR080>CR062>CR061>CR069>CR065>CR064

The drug steady-state release rate ranged from 63.14 μg/cm²/h to 727.25 μg/cm²/√h following the testing of the nine newly developed formulations selected from further screening. Following the application of the comparison formulation, steady-state drug release was observed to be up to 11 fold higher than all other investigated formulations (p<0.05). Of the newly-developed formulations, the greatest steady-state drug release was observed from CR070 (449.81 μg/cm²/√h), which was significantly higher (p<0.05) than all other newly-developed formulations, with the exception of CR071, for which there was no statistical difference (p >0.5). Following the application of CR071 and CR070 to the membrane, up to 6.5-fold greater release rates of were observed when compared to all the other ADX-102 newly-developed formulations ranked 4 and lower (p<0.05) was observed. CR080, CR062, CR061, CR069, CR065 and CR064 performed similarly (p >0.05) when the release rate of ADX-102 cellulose acetate was compared (63.14-91.73 μg/cm²/√h; p >0.05).

The release data generated for the comparison formulation may be attributable to the fact that drug in the formulation was present at levels in excess of the saturated solubility of the drug in the formulation (i.e., the thermodynamic activity of the drug was maximal), as evidenced by the ADX-102 crystals observed in the formulation. Conversely, drug in the newly-developed formulations was present below levels of saturation and were at approximately 80% of the saturated solubility of the drug in formulation (with the exception of CR080, which was a non-thermodynamically optimized version of the 5% w/w ADX-102 formulation, CR079).

The performance of the CR070 and CR071 formulations may be attributed to the absence of Brij S2 and/or CS25, liquid paraffin and high levels of PEG 1500. These excipients were present in the formulations from which lower release rates were found (i.e., CR061-CR069) and it is thought that these excipients presented a more complex matrix from which the drug must release, when compared to the surfactants and oils used in CR070, CR071, and the comparison formulation. Additionally, in terms of formulation composition, CR070 and CR071 were similar to the comparison formulation; however, stability of the drug in these formulations compared to the comparison formulation has been demonstrated (see Examples above). Furthermore, the higher release rate of drug from CR079 is likely the result of a higher concentration of ADX-102 (5% w/w) present in the formulation, when taking into account that there were not any other significant compositional changes compared to CR080 (1% formulation) indicating that the drug in this formulation had a higher thermodynamic activity. The findings of the full scale in vitro drug release experiment therefore indicate that all formulations successfully release the drug from the formulation. Of the newly-developed formulations, of particular interest are 3 formulations (CR070, CR071, and CR079) which, on application to cellulose acetate, released up to 7-fold, 6.5-fold and 6-fold higher amounts of ADX-102 into the receiver fluid than the other tested newly-developed formulations.

TABLE 41
Calculated release (μg/cm²/√h) of ADX-102 across
cellulose acetate membrane

| | | Steady-state | |
|---|---|---|---|
| | % API | release rate | |
| Formulation | (w/w) | (μg/cm²/√h) | Rank |
| CR061 Active | 1 | 82.92833 | 7 |
| CR062 Active | 1 | 86.39000 | 6 |
| CR064 Active | 1 | 63.14167 | 10 |
| CR065 Active | 1 | 75.89000 | 9 |
| CR069 Active | 1 | 80.66333 | 8 |
| CR070 Active | 1 | 449.81667 | 2 |
| CR071 Active | 1 | 410.73333 | 3 |
| CR079 Active | 5 | 378.55000 | 4 |
| CR080 Active | 1 | 91.73333 | 5 |
| Comparator | 1 | 727.25000 | 1 |

### In Vitro Release Testing Model Validation

Following the full scale experiment, we performed a validation of the IVRT model used in the study. As such, a full in vitro drug release testing model validation was conducted in accordance with the FDA's SUPAC-SS guidelines. The CR070 formulation was selected for the purpose of this investigation. We also assessed the sensitivity of the IVRT model to changes in the concentration of ADX-102 by employing CR070 with 1.2%, 1% and 0.8% w/w ADX-102 (n=6 per formulation).

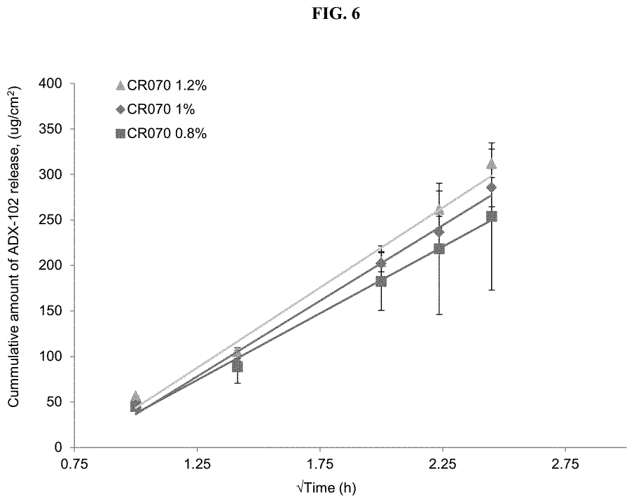

FIG. 6 demonstrates that differences were observed in the ADX-102 release rates between the test formulations. Therefore it can be assumed that the in vitro drug release model was sensitive to the concentration of ADX-102 in the formulations (CR070 1% of 113.89 g/cm²/√h, CR070 0.8% 170.72 μg/cm²/√h and CR070 1.2% 210.54 μg/cm²/√h).

### Example 7: In Vitro Permeation and Penetration Study of ADX-102 Across Healthy Human Skin Using MedFlux-HT™ System

### Study Aim

The aim of this study was to assess the in vitro skin permeation and penetration of ADX-102. This was achieved as follows:
(i) Freshly excised human skin (dermatomed to 500±100 m thickness) from one skin donor was mounted between the donor and receptor compartment of the MedFlux-HT™ diffusion cell (with an exposed dosing surface area of ˜1 cm² for each replicate).
(ii) The skin was dosed with ca. 10 mg with each formulation to achieve a dose of ˜10 mg/cm².
(iii) Receiver fluid was automatically collected into a 96-well plate at 2 hour intervals over the course of 24 h and analyzed using a single LC-MS/MS analytical method.
(iv) Following the 24 h in vitro drug permeation experiment, the residual formulation was removed from the surface of the skin and then the skin surface was stripped using tape up to 5 times to remove the stratum corneum. The epidermis was then heat-separated from the dermis by placing the skin into an incubator at 60° C. for 2 min, followed by manual separation using gloved hands.
(v) For each active formulation, n=6 repetitions per formulation were performed, however only a single repetition was performed for the respective placebo formulations and the blank.

### Example 8: In Vitro Permeation and Penetration Study of ADX-102 Across Healthy Human Skin Using MedFlux-HT™ System—Full Scale and Variable Dosing

### Study Aim

The aim of this study is to assess the in vitro skin permeation and penetration of:
(i) CR070 1.00% w/w ADX-102 dermal cream at 10 mg/cm², 20 mg/cm² and 40 mg/cm²
(ii) The comparison formulation at 10 mg/cm².

### Full-Scale Experimental Design

Freshly excised human skin (dermatomed to 500±100 m thickness) from one skin donor was mounted between the donor and receptor compartment of the MedFlux-HT™ diffusion cell (with an exposed dosing surface area of ˜1 cm² for each replicate).

The skin was dosed with ca. 10/20/40 mg with each formulation to achieve a dose of ˜10, 20 or 40 mg/cm².

Receiver fluid was automatically collected into a 96-well plate at 2 hour intervals over the course of 24 h and analyzed using a single LC-MS/MS analytical method.

Following the 24 h in vitro drug permeation experiment, the residual formulation was removed from the surface of the skin and then the skin surface was stripped with tape up to 5 times to remove the stratum corneum. The epidermis was then heat-separated from the dermis by placing the skin into an incubator at 60° C. for 2 min, followed by manual separation using gloved hands.

For each active formulation, n=6 repetitions per formulation were performed, however only a single repetition was performed for the respective placebo formulations and the blank.

### Example 9: In Vitro Permeation and Penetration Study ADX-102 Across Healthy Human Skin Using MedFlux-HT™ System—Variable Dosing

### Study Aim

The aim of this study is to assess the in vitro skin permeation and penetration of:
(i) CR070 1.00% w/w ADX-102 dermal cream at 10 mg/cm², 20 mg/cm² and 40 mg/cm²
(ii) The comparison formulation.

### Full-Scale Experimental Design

Freshly excised human skin (dermatomed to 500±100 m thickness) from one skin donor was mounted between the donor and receptor compartment of the MedFlux-HT™ diffusion cell (with an exposed dosing surface area of ˜1 cm² for each replicate).

The skin was dosed with ca. 10/20/40 mg with each formulation to achieve a dose of ˜10, 20 or 40 mg/cm².

Receiver fluid was automatically collected into a 96-well plate at 2 hour intervals over the course of 24 h and analyzed using a single LC-MS/MS analytical method.

Following the 24 h in vitro drug permeation experiment, the residual formulation was removed from the surface of the skin and then the skin surface was stripped with tape up to 5 times to remove the stratum corneum. The epidermis was then heat-separated from the dermis by placing the skin into an incubator at 60° C. for 2 min, followed by manually separation using gloved hands.

For each active formulation, n=6 repetitions per formulation was performed, however only a single repetition was performed for the respective placebo formulations and the blank.

### Example 10: A Phase 3 Randomized, Double-Blind, Vehicle-Controlled, Parallel Group Trial to Evaluate the Safety and Efficacy of ADX-102 1% Topical Dermal Cream in Subjects with Sjögren-Larsson Syndrome (SLS)

### Introduction

Sjögren-Larsson Syndrome

ADX-102 1% is being developed for the treatment of ichthyosis associated with Sjögren-Larsson Syndrome (SLS). SLS (ICD Q87.1) is a rare, chronically-debilitating, autosomal-recessive disorder characterized by generalized ichthyosis, cognitive deficit in a majority of patients, and spastic diplegia or tetraplegia. SLS is caused by mutations in the ALDH3A2 gene that encodes fatty aldehyde dehydrogenase (FALDH), an enzyme that catalyzes the oxidation of aliphatic aldehydes to fatty acids.

In most patients, ichthyosis (dry, thickened, scaly, erythematous skin that is pruritic and, due to frequent excoriation, friable) is moderate or severe, particularly prominent in flexure areas, and involves much of the body surface except the face and glabrous skin. The dermal symptoms are generally recalcitrant to therapy, and are associated with significant daily physical and emotional burden and social stigma for patients and caregivers. The cutaneous symptoms are apparent at birth in almost all patients; they become more established by several months of age and remain throughout life. Developmental delay, spasticity, and other neurological symptoms become apparent over the first 2 years of life. The onset of neurologic symptoms usually prompts recognition of a diagnosis of SLS rather than another type of ichthyotic disorder.

SLS ichthyosis pathophysiology is well understood: fatty aldehydes disrupt dermal function, particularly the epidermal fatty moisture barrier, resulting in pathologic water loss, cutaneous desiccation, and compensatory keratinocytic hypertrophy. In epidermal cells, FALDH deficiency results in impaired oxidation of long-chain fatty aldehydes to fatty acids. The consequent accumulation of aldehydes disrupts the normal function and secretion of lamellar bodies and leads to intercellular lipid deposits in the stratum corneum and a defective water barrier in the skin layer, resulting in the cutaneous symptoms of SLS. Ichthyosis related to SLS is a serious disease and there is no currently approved treatment that targets the underlying disease. Bathing several hours per day and over-the-counter moisturizing creams are the main interventions used to attempt to treat the symptoms, but have only limited and variable results, lasting for no more than a few hours. Keratolytic agents, salicylic acid, and urea are also considered for treatment. Patients with severe ichthyosis who are unresponsive to topical agents typically receive treatment with systemic retinoids, which are associated with a variety of toxicities. Patients are not usually maintained on retinoids due to concerns about toxicity, particularly in pediatric patients. No clinical intervention has been well studied in SLS patients, and therapeutic efficacy is generally variable. There is considerable social stigma in SLS since the skin appearance resembles disseminated cutaneous infectious disease and scales often emit a foul odor due to bacterial overgrowth and putrefaction.

The severity of ichthyosis may be measured using the Ichthyosis Severity Score (ISS). The ISS consists of five sub-scores, each graded on a five-point scale (0=clear, 1=almost clear, 2=mild, 3=moderate, 4=severe): global, scaling, erythema, lichenification, and excoriation.

### Potential Benefit

As described above, SLS ichthyosis is generally recalcitrant to therapy. Phase 2 clinical trial results in SLS patients treated over ˜3% BSA with ADX-102 1% for 8 weeks demonstrated that ADX-102 was effective in treating the ichthyosis associated with SLS.

The Phase 3 study will assess the activity of ADX-102 1% after 24-weeks of dosing over all skin area affected by ichthyosis (approximately 80-90% BSA in a typical SLS patient).

### Study Objectives and Endpoints

Primary Objective

To evaluate the efficacy of ADX-102 1% topical dermal cream on ichthyosis associated with SLS.

Secondary Objective

To evaluate the safety and secondary efficacy endpoints of ADX-102 1% topical dermal cream in subjects with SLS.

Primary Endpoints

The primary efficacy endpoint is the Visual Index for Ichthyosis Severity (VIIS) scaling severity score as assessed by the Investigator in the target efficacy area.

Secondary Endpoints

Secondary endpoints include:
(i) VIIS scaling erythema score in the target efficacy area
(ii) VIIS scaling and erythema severity score as assessed by a central reader in the target efficacy area
(iii) EQ-5D-5L quality of life assessment
(iv) Clinician global impression of severity
(v) Patient/Observer global impression of severity
(vi) Patient/Observer global impression of bother
(vii) Patient/Observer global impression of treatment
(viii) Patient-reported itching
(ix) Patient/Observer-reported scratching
(x) Patient/Observer Burden of bathing/showering
(xi) Children's Dermatology Life of Quality Index (CDLQI)/Dermatology Life Quality Index (DLQI)
(xii) Lipid biomarkers from skin cells
(xiii) Transepidermal Water Loss (TEWL)

### Investigational Plan

Overall Study Design and Plan

This is a two-part, randomized, double-blind, vehicle-controlled, parallel-group Phase 3 clinical trial with an initial assessment of increasing BSA treatment (Part 1). After the completion of Part 1, a separate set of eligible subjects will be enrolled into Part 2.

### Part 1 Design:

Part 1 will evaluate the safety, tolerability, and efficacy of treatment to the affected area of ichthyosis initially on approximately 20% BSA and then increasing up to 90% BSA over 24 weeks of exposure and will confirm an appropriate sample size for Part 2.

Subjects will be consented and screened for eligibility. Subjects will be randomized in a 2:1 ratio to receive ADX-102 1% or vehicle.

Subjects or caregivers will apply study drug (i.e., ADX-102 1% or vehicle) to a treatment area identified by the Investigator.

The initial treatment area will be defined as approximately 20% BSA with Grade 2 or higher on the VIIS scaling severity score, as assessed by the Investigator, and will include one of the body sites as defined in the VIIS tool (target efficacy area; dorsal foot cannot be selected as the target efficacy area). From Week 1 through Week 12, subjects will receive treatment once daily in the initial treatment area only.

If the Investigator determines that there are no safety or tolerability issues at the end of Week 12, the treatment area will increase to all of the affected areas of ichthyosis on one-half of the body (approximately 40-45% BSA in a typical SLS patient). From Week 13 through Week 20, subjects will receive treatment once daily on the expanded treatment area.

If there are still no safety or tolerability issues at the end of Week 20, the treatment area will further increase to all affected areas of ichthyosis on the entire body (approximately, 80-90% BSA in a typical SLS patient). Subjects will receive treatment once daily treatment on all affected areas from Week 21 through Week 24.

Subjects completing the 24 weeks of study drug treatment will then be monitored for an additional 4 weeks for safety.

Subjects who are randomized in Part 1 will not be eligible to participate in Part 2.

### Part 2 Design:

Part 2 will evaluate the safety, tolerability, and efficacy of treatment to the affected area of ichthyosis on the body (approximately 80-90% BSA in a typical SLS patient) over 24 weeks of exposure.

Subjects will be consented and screened for eligibility. Qualified subjects will be randomized in a 2:1 ratio to either ADX-102 1% or vehicle. The appropriate sample size for Part 2 will be confirmed in Part 1. Subjects or caregivers will apply study drug (i.e., ADX-102 1% or vehicle) once daily to all affected areas of ichthyosis on the body (approximately 80-90% of BSA in a typical SLS patient).

The target efficacy area for all efficacy analyses at Week 24 will be one of the body sites as defined in the VIIS tool with Grade 2 or higher on the VIIS scaling severity as assessed by the Investigator. Dorsal foot cannot be selected as the target efficacy area.

### Study Treatment

Description of Study Treatment

ADX-102 (2-[3-amino-6-chloro-quinolin-2-yl]-propan-2-ol) 1% w/w drug product (active) is supplied by the Sponsor as a white-to-off-white topical dermal cream. Vehicle drug product is formulated identically to ADX-102 active drug product without the active ingredient.

Treatment Administration

A standard amount of study drug for the percent BSA under treatment will be measured and applied to the treatment area once daily to achieve a dose of approximately 8 mg/cm², with each application administered approximately 24 hours apart. Study drug should only be administered to the treatment area, as directed by the Investigator, during the study.

At scheduled study visits at the site, subjects may not bathe or apply study drug or as-needed topical symptom control therapy (emollients) to the treatment area within 12 hours of examination or photographic assessment. Subjects should refrain from applying as-needed topical symptom control therapy and bathing within 4 hours of applying study drug to the treatment area. The treated area should be covered by loose clothing to minimize light exposure.

### Efficacy Analyses

A conclusion concerning efficacy will be based on evaluation of data in Part 2 of the study. A primary efficacy analysis will be conducted as well as a number of secondary efficacy analyses.

The primary efficacy endpoint (VIIS scaling severity as assessed by the Investigator in the target efficacy area) as well as all secondary efficacy variables that are captured on a continuous or semi-continuous scale (e.g., VIIS erythema score, central reader assessed VIIS scales, EQ-5D-5L, clinical global impression of severity, Patient/Observer impression scales, Patient-Reported Itching, CDLQI/DLQI, lipid biomarkers and TEWL) will be expressed as baseline to ending change scores. Two-sided 95% confidence intervals (CIs) will be computed and used descriptively for all variables but also to evaluate change relative to a standard for some variables (i.e., by determining whether the CI includes or excludes the standard).

The two-sided 95% CI for VIIS scaling severity will serve as the primary analysis and will be used to evaluate this primary endpoint as follows. An efficacy claim will be made if: 1) the lower confidence interval is above zero, 2) the mid-point of the confidence interval is at least 0.5 units is size, and 3) at least 50% of subjects have favorable change scores of one or greater.

The primary and secondary scores measured on a continuous or semi-continuous scale also will be evaluated using Mixed Model Repeated Measures to assess the effects of treatment (active vs. vehicle), time (per collection schedule), and treatment×time. In addition to descriptive statistics for the raw data used in each model, the LS means and standard errors will be reported for each main effect and interaction, as well as for treatment contrasts (i.e., active LS mean minus vehicle LS mean) at each data collection time point. If informative, covariates may be added to one or more models. Statistical significance unadjusted for multiplicity for main effects, interactions and contrasts will be reported. However, neither the main effect of treatment nor the treatment contrasts at time points are expected to be statistically significant (p<0.05) due to power restrictions at the between group level imposed by recruiting realities.

Response over time will be converted to one or more Area Under the Curve parameters and evaluated using a generalized linear model. VIIS outcome scores and other continuous or semi-continuous outcome variables will be evaluated using a Mixed Model Repeated Measures (MMRM) analysis. Conversion of VIIS or other scales to indicate response (yes/no) will occur (e.g., 0.5 VIIS improvement, 1 unit VIIS improvement) and will be evaluated as secondary endpoints. Logistic or Generalized Estimating Equation (GEE) modeling will be used to evaluate incidence of response overall and/or by time. Time to Event analysis (e.g., Kaplan-Meier with log rank test, proportional hazards [PH] modeling) will be used to evaluate time to response.

To increase statistical power, covariates may be incorporated into the computation of 95% confidence intervals (e.g., through the use of a mean centered covariate in a regression model to predict the mean pre-treatment to post-treatment difference) and/or incorporated into logistic, GEE, PH, MMRM and generalized linear models.

Categorical variables other than responder status will be descriptively summarized in contingency tables. Selective inferential analysis at each time point or across time points will be conducted using non-parametric or exact statistics with or without further stratification as described in the SAP.

### REFERENCES

1. Rizzo W B, S'Aulis D, Jennings M A, Crumrine D A, Williams M L, Elias P M. Ichthyosis in Sjögren-Larsson syndrome reflects defective barrier function due to abnormal lamellar body structure and secretion. Arch Dermatol Res. 2010 August; 302(6):443-51.
2. Rizzo W B. The role of fatty aldehyde dehydrogenase in epidermal structure and function. Dermatoendocrinol. 2011 April-June; 3(2): 91-99.
3. Rizzo W B. Fatty aldehyde and fatty alcohol metabolism: review and importance for epidermal structure and function. Biochim. Biophys. Acta., 2014. 1841(3): 377-89.
4. Rizzo W B, Bailey Z, S'Aulis D., Ergasheva N. Aldehyde Trapping Agent NS2 Blocks Formation of Fatty Aldehyde Adducts with Phosphatidylethanolamine and Suggests Potential Therapeutic Approach for Sjögren-Larsson Syndrome. [Abstract] Mol Genet and Metab, 2015 March; 114 (3): Page 362A.
5. Maeda A, Maeda T, Golczak M, Chou S, Desai, A, Hoppel C L, Matsuyama S, Palczewski K. Involvement of all-trans-retinal in acute light-induced retinopathy of mice. J Biol. Chem. 2009 May; 284 (22): 15173-15183.
6. Levey A S, Stevens L A, Schmid C H, Zhang Y L, Castro A F, 3rd, Feldman H I, et al. A new equation to estimate glomerular filtration rate. Ann Intern Med. 2009; 150(9):604-12.
7. Schwartz G J, Work, D F. Measurement and Estimation of GFR in Children and Adolescents. Clin J Am Soc Nephrol. 2009, November; 4 (11): 1832-1843.
8. Division of AIDS, National Institute of Allergy and Infectious Diseases, National Institutes of Health, US Department of Health and Human Services. Division of AIDS (DAIDS) Table for Grading the Severity of Adult and Pediatric Adverse Events V2.0, 2014 November.
9. US Department of Health and Human Services. Common Terminology Criteria for Adverse Events (CTCAE) V5.0. 2017 November.
10. Jellinger P S, Handelsman Y, et al. American Association of Clinical Endocrinologists and American College of Endocrinology Guidelines for Management of Dyslipemia and Prevention of Cardiovascular Disease. Endocrine Practice. 2017 April; (23) suppl 2.

We claim:

1. A topical composition comprising:
(i) an active agent selected from 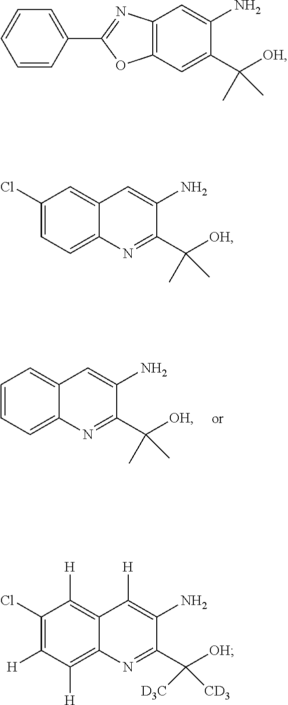 [SMILES: CC(C)(O)c1cc2oc(-c3ccccc3)nc2cc1N ; CC(C)(O)c1nc2ccc(Cl)cc2cc1N ; CC(C)(O)c1nc2ccccc2cc1N ; [2H]C([2H])([2H])C(O)(c1nc2ccc(Cl)cc2cc1N)C([2H])([2H])[2H]]
or a pharmaceutically acceptable salt thereof;
(ii) a first solvent;
(iii) optionally a second solvent;
(iv) a penetration enhancer;
(v) a preservative;
(vi) optionally one or more of a solubilizing agent, thickening agent, stabilizing agent, surfactant, skin conditioner, or second skin penetration enhancer;
(vii) an antioxidant; and
optionally one or more of: an emulsifying agent, a diluent, a pH adjuster, a chelating agent, a coloring agent, or a fragrance.

2. A topical composition comprising:
(i) an active agent selected from 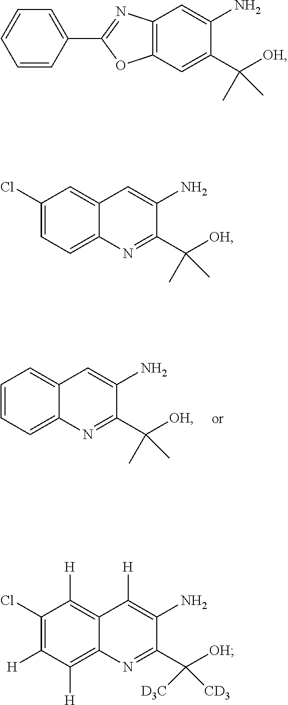 [SMILES: CC(C)(O)c1cc2oc(-c3ccccc3)nc2cc1N ; CC(C)(O)c1nc2ccc(Cl)cc2cc1N ; CC(C)(O)c1nc2ccccc2cc1N ; [2H]C([2H])([2H])C(O)(c1nc2ccc(Cl)cc2cc1N)C([2H])([2H])[2H]]
or a pharmaceutically acceptable salt thereof;
(ii) a first solvent;
(iii) optionally a second solvent;
(iv) a penetration enhancer;
(v) a preservative;
(vi) a surfactant;
(vii) a thickening agent;
(viii) a stabilizing agent;
(ix) optionally one or more of a solubilizing agent, skin conditioner, or second skin penetration enhancer;
(x) optionally an antioxidant; and
optionally one or more of: an emulsifying agent, a diluent, a pH adjuster, a chelating agent, a coloring agent, or a fragrance.

3. The topical composition of claim 2, comprising about 0.1 to 7.5% of the active agent; about 12.0 to 48.0% of a first solvent; about 2.0 to 15.0% of a second solvent; about 1.0 to 29.0% of a penetration enhancer; optionally up to about 3.0% of a preservative; optionally up to about 20.0% of a thickening agent; about 1.0 to 15.0% of a surfactant; optionally up to about 10.0% of a stabilizing agent; and optionally up to about 1.0% of an antioxidant.

4. (canceled)

5. The composition of claim 3, wherein the composition is substantially free of excipients or solvents that contain aldehydes or present a risk of decomposing, or are known to decompose, to produce aldehyde-containing impurities.

6. The composition of claim 3, wherein the first solvent comprises a polyethylene glycol, polyoxyethylene alkyl ether, polyoxyethylene sorbitan fatty acid ester, or polyoxyethylene stearate.

7. The composition of claim 3, wherein the first solvent is selected from Transcutol, PEG 400, S.R. PEG 400, PEG 200, PEG 300, PEG 540, PEG 600, PEG 900, propylene carbonate, butyl stearate, castor oil, IPM, IPP, Miglyol 810, or a mixture thereof.

8. The composition of claim 7, wherein the second solvent is present and comprises a glycol solvent and an alkyl alcohol solvent.

9. The composition of claim 8, wherein the first and second solvent are a mixture of SR PEG 400, propylene glycol, and ethanol.

10. (canceled)

11. The composition of claim 9, wherein the composition comprises about 28.0 to 40.0% water.

12. The composition of claim 6, wherein the penetration enhancer is selected from one or more of dimethyl sulfoxide, a glycol, glyceryl monooleate, glycofurol, isopropyl myristate, isopropyl palmitate, lanolin, light mineral oil, linoleic acid, menthol, myristic acid, myristyl alcohol, oleic acid, oleyl alcohol, palmitic acid, a polyoxyethylene alkyl ether, a polyoxylglyceride, a pyrrolidone, lauryl sulfate, thymol, tricaprylin, triolein, acetyltributyl citrate, ethylene vinyl acetate, a polymethacrylate, polyvinyl alcohol, sesame oil, carboxymethyl guar, or stearyl alcohol.

13. (canceled)

14. The composition of claim 6, wherein the penetration enhancer is selected from Transcutol HP, propylene glycol, or a mixture thereof.

15. The composition of claim 6, wherein the surfactant is selected from emulsifying wax USP, glyceryl monooleate, a phospholipid, a polyoxyethylene alkyl ether, polyoxyethylene castor oil derivative, a polyoxyethylene sorbitan fatty acid ester, polyoxyethylene stearates, polyoxylglycerides, polysorbate, a sorbitan ester, triethyl citrate, vitamin E polyethylene glycol succinate, miglyol, a Steareth (Brij) surfactant, a Cetomacrogol, a Myrj, a Span, a Tween, or a Laureth surfactant.

16. The composition of claim 6, wherein the surfactant is selected from Steareth 21 (Brij s721), Steareth 2 (Brij S2), Cetomacrogol 1000, Myrj S40, Brij CS25 (Ceteareth 25), Steareth 20 (Brij S20), Span 20, Geleol mono and diglycerides, Span 60, or Tween 60, or a combination thereof.

17. The composition of claim 6, wherein the surfactant is a combination of Steareth 2 and Mrij S40; Steareth 2 and Brij CS25; Steareth 2 and Cetomacrogol 1000; Steareth 2 and Steareth 20/Brij S20; Steareth 2 and Steareth 21; Span 20 and Geleol; Span 60 and Tween 60; Cetomacrogol 1000 and Geleol; Brij S20 and Brij S2; or Geleol, Span 20, and Steareth 20/Brij S20.

18. The composition of claim 6, wherein the thickening agent is a high molecular weight polyethylene glycol selected from PEG 1500 or PEG 4000.

19. The composition of claim 6, wherein the preservative is selected from vitamin C, butylated hydroxytoluene (BHT), butylated hydroxyanisole (BHA), a sulfite, vitamin E, ascorbyl palmitate, or propyl gallate.

20. (canceled)

21. The composition of claim 6, wherein the preservative and antioxidant comprise phenoxyethanol, BHT, BHA, or a mixture of two or all three thereof.

22-23. (canceled)

24. A method of treating a skin disease, disorder, or condition selected from psoriasis, topical (discoid) lupus, contact dermatitis, atopic dermatitis, allergic dermatitis, radiation dermatitis, acne vulgaris, Sjögren-Larsson Syndrome (SLS) and/or other ichthyoses, neurogical and/or motor effects of SLS, SSADHD, pyridoxine-dependent epilepsy, or a cosmetic indication selected from solar elastosis/wrinkles, skin tone firmness, puffiness, eczema, smoke or irritant induced skin changes, dermal incision, or a skin condition associated with a burn or wound, comprising administering to a subject in need thereof a composition of claim 3.

25. The method of claim 24, wherein the method treats a skin disease, disorder, or condition selected from psoriasis, scleroderma, topical (discoid) lupus, contact dermatitis, atopic dermatitis, allergic dermatitis, radiation dermatitis, acne vulgaris, or Sjögren-Larsson Syndrome and/or other ichthyoses.

26. (canceled)

27. The method of claim 24, wherein the method treats Sjögren-Larsson Syndrome (SLS) and/or other ichthyoses.

28-32. (canceled)

33. A method of treating an autoimmune, immune-mediated, inflammatory, cardiovascular, neurological, metabolic, or fibrotic disease, disorder, or condition, comprising administering to a subject in need thereof a composition of claim 3.

34. A method of treating a skin disease, disorder, or condition, comprising administering to a subject in need thereof a composition of claim 3.

35-39. (canceled)

40. A topical formulation selected from one of those described in Table 37, Table 38, or Table 39.
